(19) United States
(12) Patent Application Publication
(10) Pub. No.: US 2017/0104163 A1
(43) Pub. Date: Apr. 13, 2017

(54) COMPOSITION FOR ORGANIC OPTOELECTRIC DIODE, ORGANIC OPTOELECTRIC DIODE, AND DISPLAY DEVICE

(71) Applicant: SAMSUNG SDI CO., LTD., Yongin-si, Gyeonggi-do (KR)

(72) Inventors: Han-ILL LEE, Suwon-si, Gyeonggi-do (KR); Dong-Wan RYU, Suwon-si, Gyeonggi-do (KR); Jin-Hyun LUI, Suwon-si, Gyeonggi-do (KR); Chang-Ju SHIN, Suwon-si, Gyeonggi-do (KR); Eun-Sun YU, Suwon-si, Gyeonggi-do (KR); Sung-Hyun JUNG, Suwon-si, Gyeonggi-do (KR)

(73) Assignee: SAMSUNG SDI CO., LTD., Yongin-si, Gyeonggi-do (KR)

(21) Appl. No.: 15/317,468

(22) PCT Filed: Dec. 11, 2014
(86) PCT No.: PCT/KR2014/012216

§ 371 Date: Dec. 9, 2016

(30) Foreign Application Priority Data
Jul. 21, 2014 (KR) 10-2014-0091994

(51) Int. Cl. H01L 51/00; C09K 11/06; C07D 209/86; C07D 403/10; C07D 239/74; C07D 239/70; C09K 11/02; C07D 251/24

(52) U.S. Cl. CPC H01L 51/0067; C09K 11/025; C09K 11/06; H01L 51/0085; C07D 251/24; H01L 51/0054; C07D 403/10; H01L 51/0072; C07D 239/74; C07D 239/70; C07D 209/86; H01L 51/5016

(57) ABSTRACT

The present invention relates to: a composition for an organic optoelectric diode, containing a first host compound represented by Chemical Formula I and a second host compound represented by Chemical Formula II; an organic optoelectric diode comprising the composition for an organic optoelectric diode; and a display device.

### TECHNICAL FIELD

A composition for an organic optoelectric diode, an organic optoelectric diode, and a display device are disclosed.

### BACKGROUND ART

An organic optoelectric diode is a device that converts electrical energy into photoenergy, and vice versa.

An organic optoelectric diode may be classified as follows in accordance with its driving principles. One is an optoelectric diode where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectric diode may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode converts electrical energy into light by applying current to an organic light emitting material and has a structure in which an organic layer is interposed between an anode and a cathode. the organic layer may include a light-emitting layer and optionally an auxiliary layer, and the auxiliary layer may be, for example at least one selected from a hole injection layer, a hole transport layer, an electron blocking layer, an electron transport layer, an electron injection layer, and a hole blocking layer for improving efficiency and stability of an organic light emitting diode.

Performance of an organic light emitting diode may be affected by characteristics of the organic layer, and among them, may be mainly affected by characteristics of an organic material of the organic layer.

Particularly, development for an organic material being capable of increasing hole and electron mobility and simultaneously increasing electrochemical stability is needed so that the organic light emitting diode may be applied to a large-size flat panel display.

### DISCLOSURE

### Technical Problem

An embodiment provides a composition for an organic optoelectric diode capable of realizing an organic optoelectric diode having high efficiency and a long life-span.

Another embodiment provides a composition for an organic optoelectric diode, which includes the composition.

Yet another embodiment provides a display device including the organic optoelectric diode.

### Technical Solution

According to an embodiment, a composition for an organic optoelectric diode includes a first host compound represented by Chemical Formula I and a second host compound represented by Chemical Formula II.

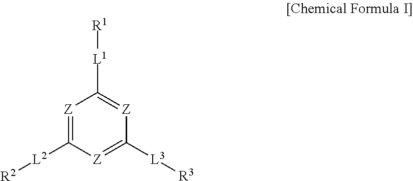

In Chemical Formula I,

Z's are independently N or CR^{a},

at least two of three Z's are N,

R¹ to R³ and R^{a} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C2 to C30 alkoxycarbonyl group, a substituted or unsubstituted C2 to C30 alkoxycarbonylamino group, a substituted or unsubstituted C7 to C30 aryloxycarbonylamino group, a substituted or unsubstituted C1 to C30 sulfamoylamino group, a substituted or unsubstituted C2 to C30 alkenyl group, a substituted or unsubstituted C2 to C30 alkynyl group, a substituted or unsubstituted C3 to C40 silyl group, a substituted or unsubstituted C3 to C40 silyloxy group, a substituted or unsubstituted C1 to C30 acyl group, a substituted or unsubstituted C1 to C20 acyloxy group, a substituted or unsubstituted C1 to C20 acylamino group, a substituted or unsubstituted C1 to C30 sulfonyl group, a substituted or unsubstituted C1 to C30 alkylthiol group, a substituted or unsubstituted C6 to C30 arylthiol group, a substituted or unsubstituted C1 to C30 ureide group, a halogen, a halogen-containing group, a cyano group, a hydroxyl group, an amino group, a nitro group, a carboxyl group, a ferrocenyl group, or a combination thereof,

adjacent two selected from R¹ to R³ and R^{a} are linked to each other to provide a ring,

L¹ to L³ are independently a single bond, a substituted or unsubstituted C1 to C30 alkylene group, a substituted or unsubstituted C3 to C30 cycloalkylene group, a substituted or unsubstituted C6 to C30 arylene group, a substituted or unsubstituted C2 to C30 heteroarylene group, a substituted or unsubstituted C6 to C30 aryleneamine group, a substituted or unsubstituted C1 to C30 alkoxylene group, a substituted or unsubstituted C1 to C30 aryloxylene group, a substituted or unsubstituted C2 to C30 alkenylene group, a substituted or unsubstituted C2 to C30 alkynylene group, or a combination thereof, and

when the L¹ to L³ are all single bonds, all the R¹ to R³ are not hydrogen,

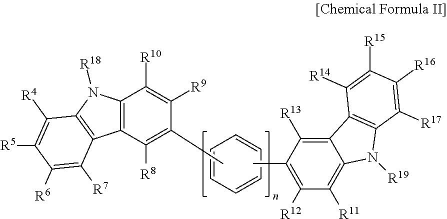

wherein, in Chemical Formula II,

R⁴ to R¹⁷ are independently hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,

adjacent two of R⁴ to R¹⁰ and R¹¹ to R¹⁷ are linked to each other to provide a ring,

R¹⁸ and R¹⁹ are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heteroaryl group, a substituted or unsubstituted C6 to C30 arylamine group, a substituted or unsubstituted C1 to C30 alkoxy group, a substituted or unsubstituted C2 to C30 alkoxycarbonyl group, a substituted or unsubstituted C2 to C30 alkoxycarbonylamino group, a substituted or unsubstituted C7 to C30 aryloxycarbonylamino group, a substituted or unsubstituted C1 to C30 sulfamoylamino group, a substituted or unsubstituted C2 to C30 alkenyl group, a substituted or unsubstituted C2 to C30 alkynyl group, a substituted or unsubstituted C3 to C40 silyl group, a substituted or unsubstituted C3 to C40 silyloxy group, a substituted or unsubstituted C1 to C30 acyl group, a substituted or unsubstituted C1 to C20 acyloxy group, a substituted or unsubstituted C1 to C20 acylamino group, a substituted or unsubstituted C1 to C30 sulfonyl group, a substituted or unsubstituted C1 to C30 alkylthiol group, a substituted or unsubstituted C6 to C30 arylthiol group, a substituted or unsubstituted C1 to C30 ureide group, a halogen, a halogen-containing group, a cyano group, a hydroxyl group, an amino group, a nitro group, a carboxyl group, a ferrocenyl group, or a combination thereof, and

n is an integer ranging from 1 to 4.

According to another embodiment, provided is an organic optoelectric diode including an anode and a cathode facing each other, and at least one organic layer between the anode and the cathode, wherein the organic layer includes the composition.

Another embodiment provides a display device including the organic optoelectric diode.

### Advantageous Effects

An organic optoelectric diode having high efficiency long life-span may be realized.

### DESCRIPTION OF THE DRAWINGS

FIGS. 1 and 2 are cross-sectional views showing organic light emitting diodes according to embodiments.

### DESCRIPTION OF SYMBOLS

100, 200: organic light emitting diode
105: organic layer
110: cathode
120: anode
130: light-emitting layer
140: hole auxiliary layer

### BEST MODE

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to one substituted with deuterium, a halogen, a hydroxy group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C3 to C30 cycloalkyl group, a C3 to C30 heterocycloalkyl group, a C6 to C30 aryl group, a C6 to C30 heteroaryl group, a C1 to C20 alkoxy group, a fluoro group, a C1 to C10 trifluoroalkyl group such as a trifluoromethyl group, or a cyano group, instead of at least one hydrogen of a substituent or a compound.

In addition, two adjacent substituents of the substituted halogen, hydroxy group, amino group, substituted or unsubstituted C1 to C20 amine group, nitro group, substituted or unsubstituted C3 to C40 silyl group, C1 to C30 alkyl group, C1 to C10 alkylsilyl group, C3 to C30 cycloalkyl group, C3 to C30 heterocycloalkyl group, C6 to C30 aryl group, C6 to C30 heteroaryl group, C1 to C20 alkoxy group, fluoro group, C1 to C10 trifluoroalkyl group such as trifluoromethyl group and the like, or cyano group may be fused with each other to form a ring. For example, the substituted C6 to C30 aryl group may be fused with another adjacent substituted C6 to C30 aryl group to form a substituted or unsubstituted fluorene ring.

In the present specification, when specific definition is not otherwise provided, “hetero” refers to one including at least one hetero atom selected from the group consisting of N, O, S, P, and Si, and remaining carbons in one functional group.

In the present specification, when a definition is not otherwise provided, “alkyl group” refers to an aliphatic hydrocarbon group. The alkyl group may be “a saturated alkyl group” without any double bond or triple bond.

The alkyl group may be a C1 to C30 alkyl group. More specifically, the alkyl group may be a C1 to C20 alkyl group or a C1 to C10 alkyl group. For example, a C1 to C4 alkyl group may have 1 to 4 carbon atoms in an alkyl chain which may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, “aryl group” refers to a substituent including all element of the cycle having p-orbitals which form conjugation, and may be monocyclic, polycyclic or fused ring polycyclic (i.e., rings sharing adjacent pairs of carbon atoms) functional group.

In the present specification, “heterocyclic group” may include at least one hetero atom selected from N, O, S, P, and Si in a cyclic compound such as an aryl group, a cycloalkyl group, a fused ring thereof, or a combination thereof, and remaining carbons. When the heterocyclic group is a fused ring, the entire ring or each ring of the heterocyclic group may include one or more heteroatoms. Accordingly, the heterocyclic group is a general concept of a heteroaryl group.

More specifically, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthrylene group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted fluorenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a combination thereof, or a fused form of combinations thereof, but are not limited thereto.

In the present specification, a single bond refers to a direct bond not by carbon or a hetero atom except carbon, and specifically the meaning that L is a single bond means that a substituent linked to L directly bonds with a central core. That is, in the present specification, the single bond does not refer to methylene that is bonded via carbon.

In the specification, hole characteristics refer to an ability to donate an electron to form a hole when an electric field is applied and that a hole formed in the anode may be easily injected into the light-emitting layer, and a hole formed in a light-emitting layer may be easily transported into an anode and transported in the light-emitting layer due to conductive characteristics according to a highest occupied molecular orbital (HOMO) level.

In addition, electron characteristics refer to an ability to accept an electron when an electric field is applied and that an electron formed in a cathode may be easily injected into the light-emitting layer, and an electron formed in a light-emitting layer may be easily transported into a cathode and transported in the light-emitting layer due to conductive characteristics according to a lowest unoccupied molecular orbital (LUMO) level.

Hereinafter, a composition according to an embodiment is described.

A composition according to an embodiment may include a first host, a second host, and a dopant.

The second host includes a linking group connected with one to four phenylenes and thus may have a more flexible molecular structure than bicarbazole directly connected with no linking group, and this flexible molecular structure effectively may prevent compounds from being stacked and thus improves film characteristics and resultantly, may increase process stability and simultaneously decrease a deposition temperature.

However, the second host has a LUMO energy level of greater than or equal to about −1.3 eV with a reference to a calculation value according to a B3LYP/6-31G method by using a program Gaussian 09 with a super computer GAIA (IBM power 6), and accordingly, when applied alone, electron injection may be difficult.

In order to easily inject electrons, a compound should have a LUMO energy level of less than or equal to about 1.5 eV when calculated according to a B3LYP/6-31G method by using a program Gaussian 09 with a super computer GAIA (IBM power 6), but the first host compound includes at least two N's in the central core and has a LUMO energy level of less than or equal to about −1.5 eV, and accordingly, the second host compound is used with the first host compound and thus may compensate electron characteristics of a device and resultantly, realize an organic optoelectric diode having high efficiency•long life-span.

The first host compound may be represented by Chemical Formula I.

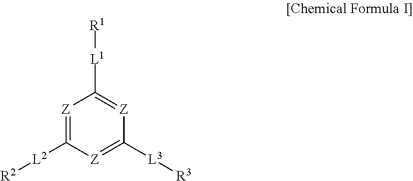

In Chemical Formula I,

Z's are independently N or CR^{a},

at least two of three Z's are N,

R¹ to R³ and R^{a} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C2 to C30 alkoxycarbonyl group, a substituted or unsubstituted C2 to C30 alkoxycarbonylamino group, a substituted or unsubstituted C7 to C30 aryloxycarbonylamino group, a substituted or unsubstituted C1 to C30 sulfamoylamino group, a substituted or unsubstituted C2 to C30 alkenyl group, a substituted or unsubstituted C2 to C30 alkynyl group, a substituted or unsubstituted C3 to C40 silyl group, a substituted or unsubstituted C3 to C40 silyloxy group, a substituted or unsubstituted C1 to C30 acyl group, a substituted or unsubstituted C1 to C20 acyloxy group, a substituted or unsubstituted C1 to C20 acylamino group, a substituted or unsubstituted C1 to C30 sulfonyl group, a substituted or unsubstituted C1 to C30 alkylthiol group, a substituted or unsubstituted C6 to C30 arylthiol group, a substituted or unsubstituted C1 to C30 ureide group, a halogen, a halogen-containing group, a cyano group, a hydroxyl group, an amino group, a nitro group, a carboxyl group, a ferrocenyl group, or a combination thereof,

adjacent two selected from R¹ to R³ and R^{a} are linked to each other to provide a ring,

L¹ to L³ are independently a single bond, a substituted or unsubstituted C1 to C30 alkylene group, a substituted or unsubstituted C3 to C30 cycloalkylene group, a substituted or unsubstituted C6 to C30 arylene group, a substituted or unsubstituted C2 to C30 heteroarylene group, a substituted or unsubstituted C6 to C30 aryleneamine group, a substituted or unsubstituted C1 to C30 alkoxylene group, a substituted or unsubstituted C1 to C30 aryloxylene group, a substituted or unsubstituted C2 to C30 alkenylene group, a substituted or unsubstituted C2 to C30 alkynylene group, or a combination thereof, and

when the L¹ to L³ are all single bonds, all the R¹ to R³ are not hydrogen.

The first host compound may be, for example represented by one of Chemical Formulae I-1 to I-5 according to a position of N.

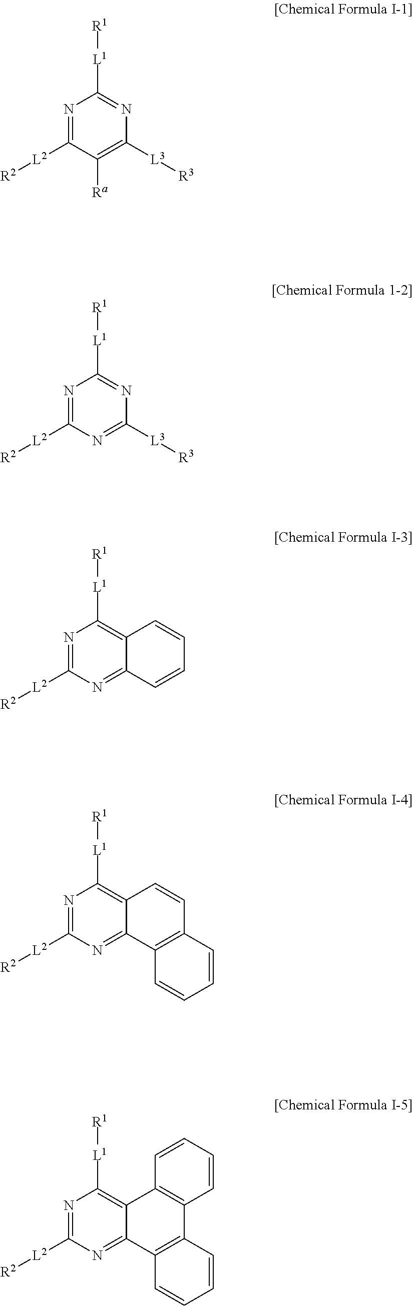

In Chemical Formulae I-1 to I-5, R¹ to R³, R^{a} and L¹ to L³ are the same as described above.

For example, in Chemical Formulae I-1 to I-5, R¹ to R³ and R^{a} may independently be hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C3 to C40 silyl group, a substituted or unsubstituted C1 to C30 alkylthiol group, a substituted or unsubstituted C6 to C30 arylthiol group, a substituted or unsubstituted C1 to C30 ureide group, a halogen, a cyano group, a hydroxyl group, an amino group, a nitro group, a carboxyl group, a ferrocenyl group, or a combination thereof, and

L¹ to L³ are independently a single bond, a substituted or unsubstituted C1 to C30 alkylene group, a substituted or unsubstituted C3 to C30 cycloalkylene group, a substituted or unsubstituted C6 to C30 arylene group, a substituted or unsubstituted C2 to C30 heteroarylene group, or a combination thereof.

When the L¹ to L³ are all single bonds, all the R¹ to R³ are not hydrogen.

The first host compound includes a ring containing at least two nitrogens and thus may have a structure easily accepting electrons when an electric field is applied thereto and accordingly, lower a driving voltage of an organic optoelectric diode manufactured by applying the first host compound.

For example, L¹ to L³ of the first host compound represented by Chemical Formula I may independently be a single bond, a substituted or unsubstituted C6 to C30 arylene group, a substituted or unsubstituted C2 to C30 heteroarylene group, or a combination thereof.

For example, the substituted or unsubstituted C6 to C30 arylene group may be a substituted or unsubstituted phenylene group, a substituted or unsubstituted biphenyl group, a substituted or unsubstituted terphenyl group, a substituted or unsubstituted quaterphenyl group. Specifically, the terphenyl group may be an o-terphenyl group, a m-terphenyl group, or a p-terphenyl group, the quaterphenyl group may be a linear quaterphenyl group or a branched iso-quaterphenyl group, tert-quaterphenyl group, 2-quaterphenyl group, and the like.

The L¹ to L³ of the first host compound represented by Chemical Formula I may independently be a single bond or selected from substituted or unsubstituted groups of Group I.

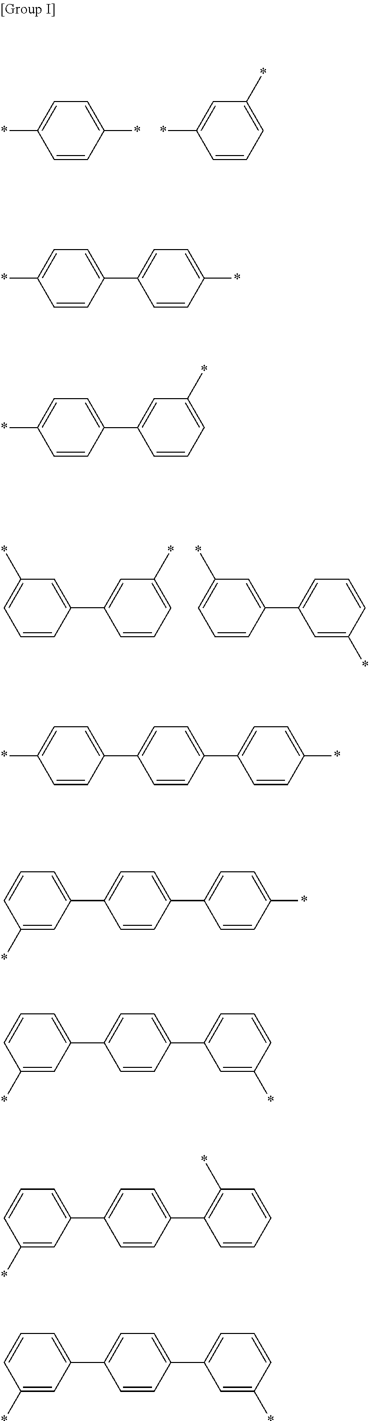

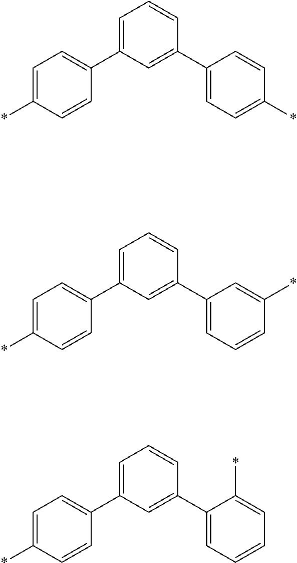 [SMILES: *c1ccc(-c2cccc(-c3ccc(*)cc3)c2)cc1 ; *c1ccc(-c2cccc(-c3cccc(*)c3)c2)cc1 ; *c1ccc(-c2cccc(-c3ccccc3*)c2)cc1]

In Group I, * is a linking point.

In addition, the R¹ to R³ and R^{a} of the first host compound represented by Chemical Formula I may independently be hydrogen, a substituted or unsubstituted C6 to C30 aryl group, a substituted or unsubstituted C2 to C30 heterocyclic group, or a combination thereof.

Specifically, the substituted or unsubstituted C6 to C30 aryl group may be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted 1H-phenalenyl group, a substituted or unsubstituted pyrenyl group, a substituted or unsubstituted fluorenyl group, a substituted or unsubstituted triphenylene group, or a combination thereof, and

the substituted or unsubstituted C2 to C30 heterocyclic group may be a substituted or unsubstituted carbazolyl group, a substituted or unsubstituted benzofuranyl group, a substituted or unsubstituted benzothiophenyl group, a substituted or unsubstituted dibenzofuranyl group, a substituted or unsubstituted dibenzothiophenyl group, or a combination thereof, and more specifically, the substituted or unsubstituted C6 to C30 aryl group and the substituted or unsubstituted C2 to C30 heterocyclic group may be selected from substituted or unsubstituted groups of Group II.

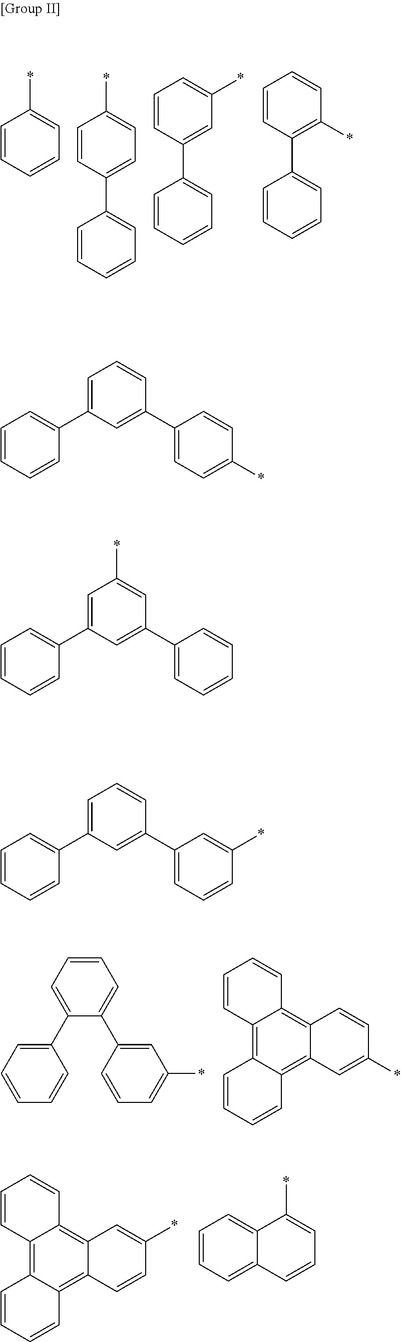

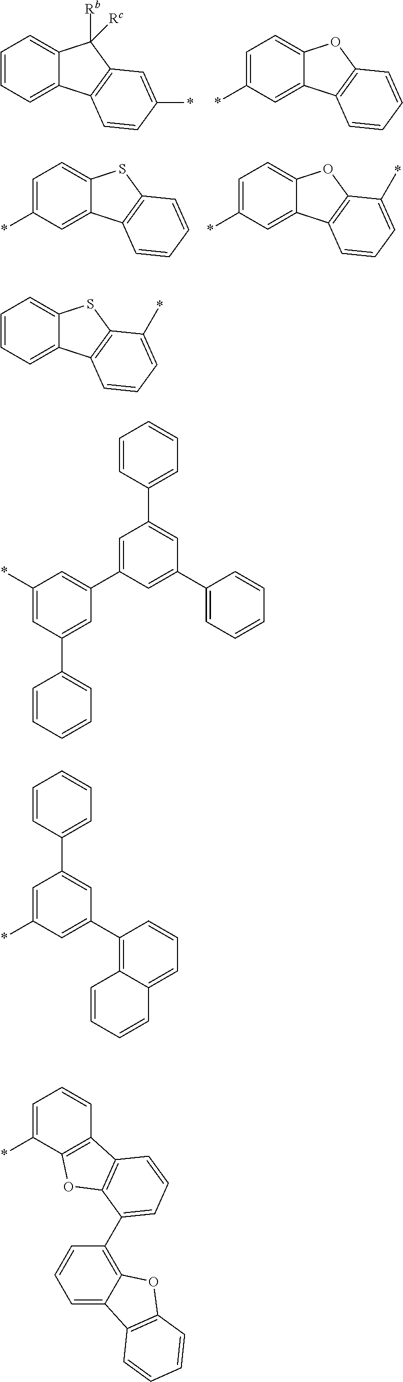

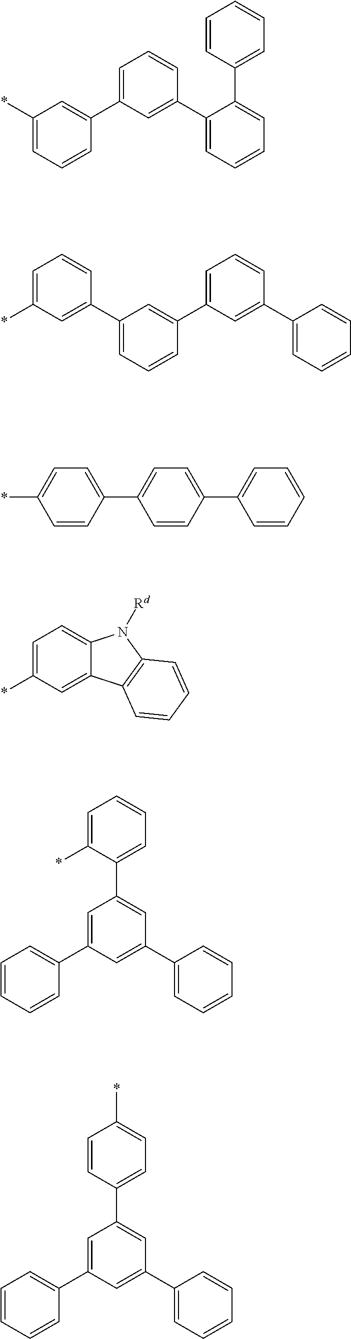

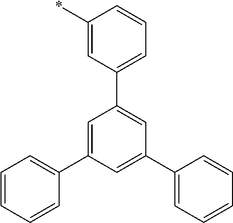 [SMILES: *c1cccc(-c2cc(-c3ccccc3)cc(-c3ccccc3)c2)c1]

In Group II,

R^{b} to R^{d} are independently hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof, and * is a linking point.

A LUMO energy level of the first host compound may be less than or equal to −1.5 eV.

The first host compound having the LUMO energy level within the ranges is a compound having strong electron characteristics, and may realize bipolar characteristics with the second host compound having strong hole characteristics.

The first host compound may be, for example selected from compounds of Group III, but is not limited thereto.

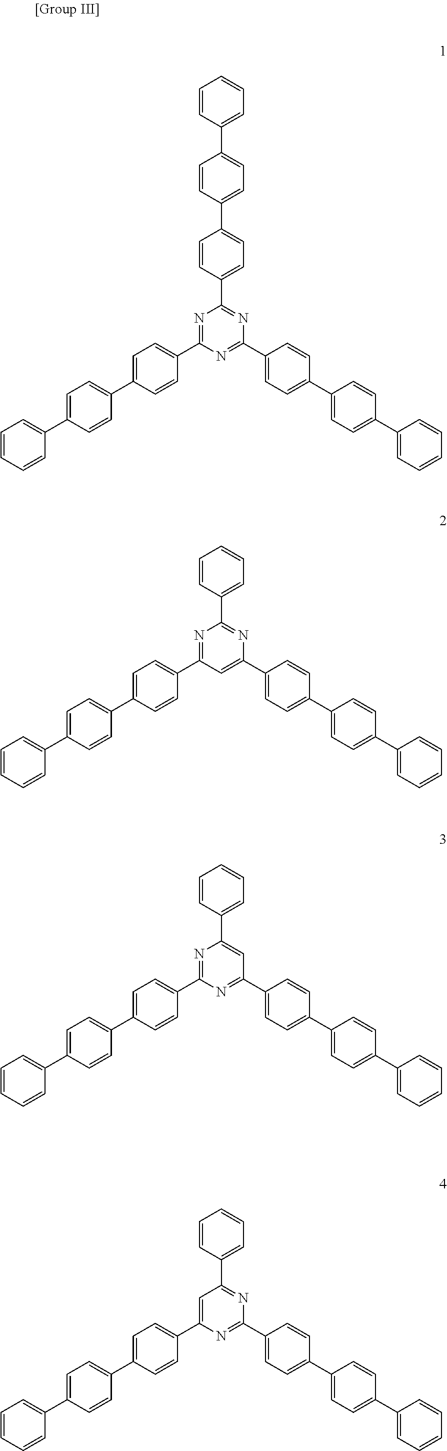 [SMILES: c1ccc(-c2ccc(-c3ccc(-c4nc(-c5ccc(-c6ccc(-c7ccccc7)cc6)cc5)nc(-c5ccc(-c6ccc(-c7ccccc7)cc6)cc5)n4)cc3)cc2)cc1 ; c1ccc(-c2ccc(-c3ccc(-c4cc(-c5ccc(-c6ccc(-c7ccccc7)cc6)cc5)nc(-c5ccccc5)n4)cc3)cc2)cc1 ; c1ccc(-c2ccc(-c3ccc(-c4cc(-c5ccccc5)nc(-c5ccc(-c6ccc(-c7ccccc7)cc6)cc5)n4)cc3)cc2)cc1 ; c1ccc(-c2ccc(-c3ccc(-c4cc(-c5ccccc5)nc(-c5ccc(-c6ccc(-c7ccccc7)cc6)cc5)n4)cc3)cc2)cc1]

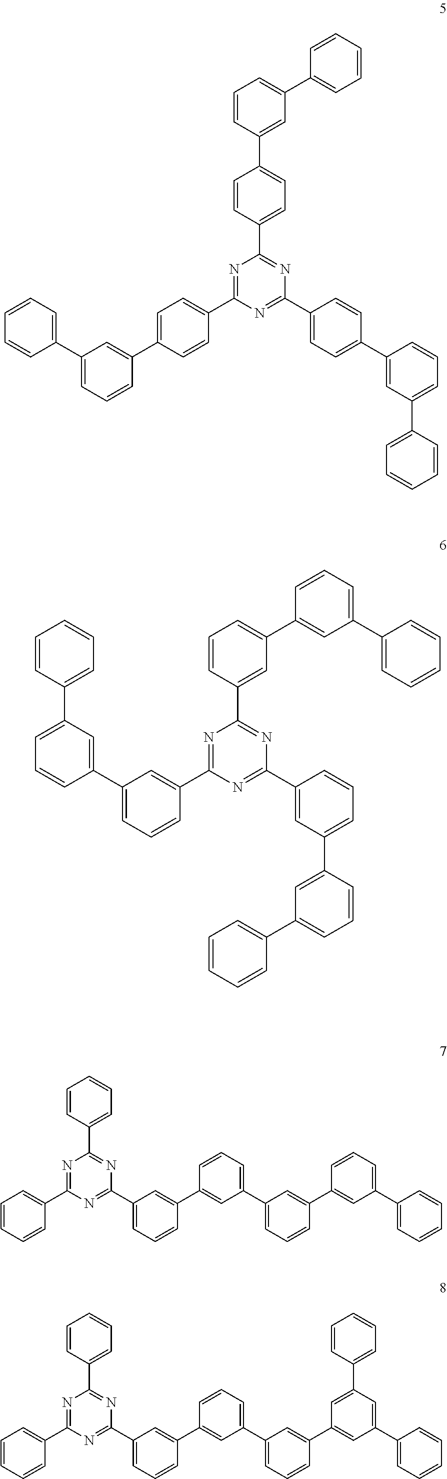 [SMILES: c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1 ; c1ccc(-c2cccc(-c3cccc(-c4nc(-c5cccc(-c6cccc(-c7ccccc7)c6)c5)nc(-c5cccc(-c6cccc(-c7ccccc7)c6)c5)n4)c3)c2)cc1 ; c1ccc(-c2cccc(-c3cccc(-c4cccc(-c5cccc(-c6nc(-c7ccccc7)nc(-c7ccccc7)n6)c5)c4)c3)c2)cc1 ; c1ccc(-c2cc(-c3ccccc3)cc(-c3cccc(-c4cccc(-c5cccc(-c6nc(-c7ccccc7)nc(-c7ccccc7)n6)c5)c4)c3)c2)cc1]

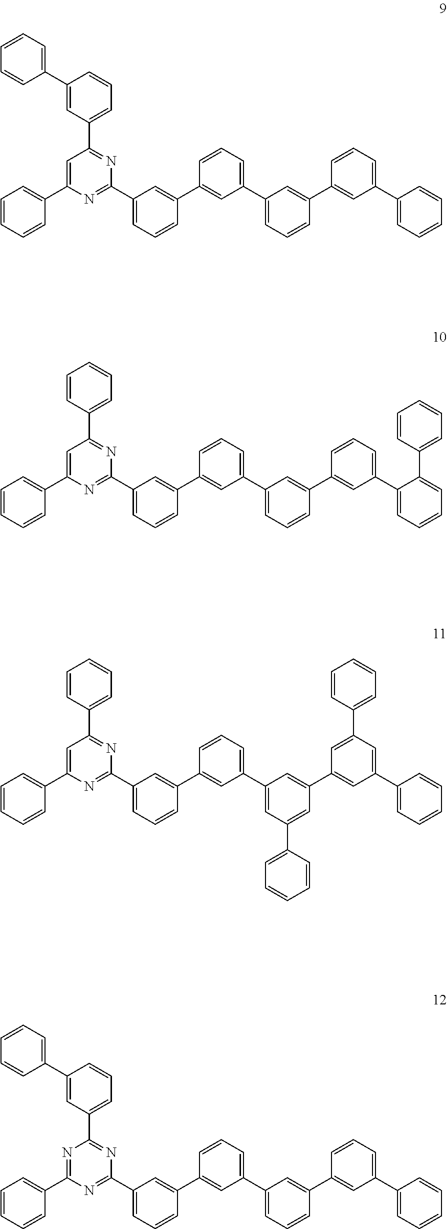 [SMILES: c1ccc(-c2cccc(-c3cccc(-c4cccc(-c5cccc(-c6nc(-c7ccccc7)cc(-c7cccc(-c8ccccc8)c7)n6)c5)c4)c3)c2)cc1 ; c1ccc(-c2cc(-c3ccccc3)nc(-c3cccc(-c4cccc(-c5cccc(-c6cccc(-c7ccccc7-c7ccccc7)c6)c5)c4)c3)n2)cc1 ; c1ccc(-c2cc(-c3ccccc3)cc(-c3cc(-c4ccccc4)cc(-c4cccc(-c5cccc(-c6nc(-c7ccccc7)cc(-c7ccccc7)n6)c5)c4)c3)c2)cc1 ; c1ccc(-c2cccc(-c3cccc(-c4cccc(-c5cccc(-c6nc(-c7ccccc7)nc(-c7cccc(-c8ccccc8)c7)n6)c5)c4)c3)c2)cc1]

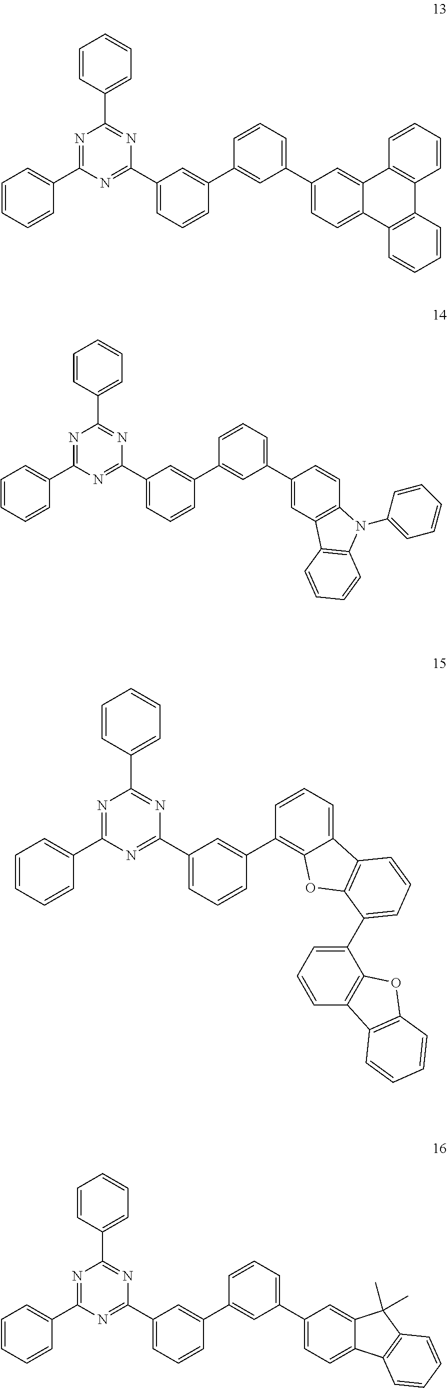 [SMILES: c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c(c5)c5ccccc5n6-c5ccccc5)c4)c3)n2)cc1 ; c1ccc(-c2nc(-c3ccccc3)nc(-c3cccc(-c4cccc5c4oc4c(-c6cccc7c6oc6ccccc67)cccc45)c3)n2)cc1 ; CC1(C)c2ccccc2-c2ccc(-c3cccc(-c4cccc(-c5nc(-c6ccccc6)nc(-c6ccccc6)n5)c4)c3)cc21]

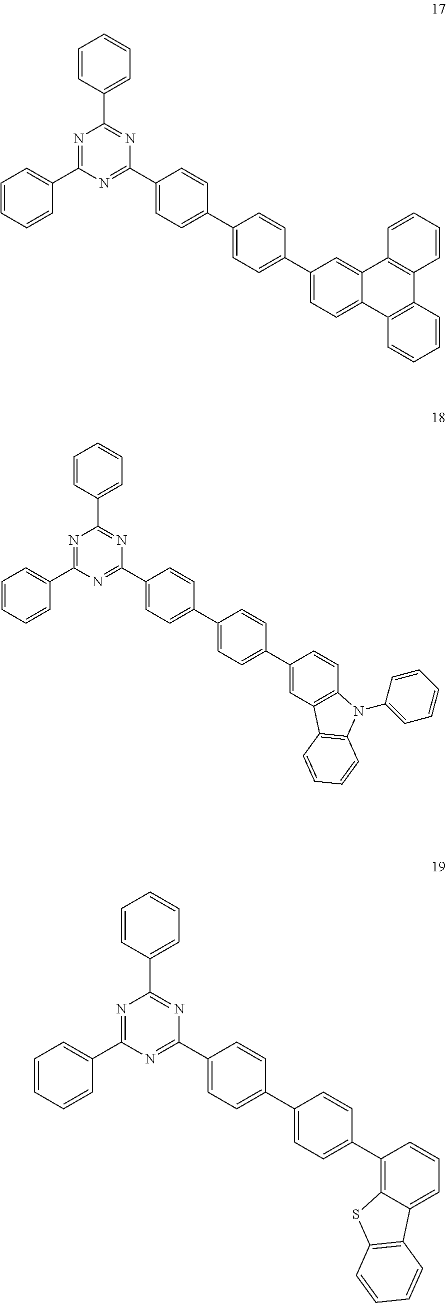 [SMILES: c1ccc(-c2nc(-c3ccccc3)nc(-c3ccc(-c4ccc(-c5ccc6c7ccccc7c7ccccc7c6c5)cc4)cc3)n2)cc1 ; c1ccc(-c2nc(-c3ccccc3)nc(-c3ccc(-c4ccc(-c5ccc6c(c5)c5ccccc5n6-c5ccccc5)cc4)cc3)n2)cc1 ; c1ccc(-c2nc(-c3ccccc3)nc(-c3ccc(-c4ccc(-c5cccc6c5sc5ccccc56)cc4)cc3)n2)cc1]

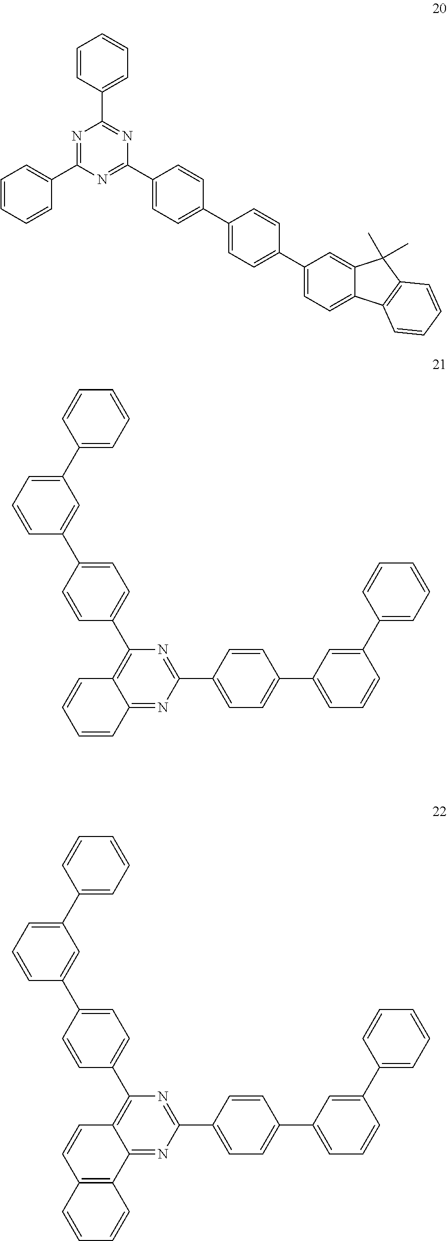 [SMILES: CC1(C)c2ccccc2-c2ccc(-c3ccc(-c4ccc(-c5nc(-c6ccccc6)nc(-c6ccccc6)n5)cc4)cc3)cc21 ; c1ccc(-c2cccc(-c3ccc(-c4nc(-c5ccc(-c6cccc(-c7ccccc7)c6)cc5)c5ccccc5n4)cc3)c2)cc1 ; c1ccc(-c2cccc(-c3ccc(-c4nc(-c5ccc(-c6cccc(-c7ccccc7)c6)cc5)c5ccc6ccccc6c5n4)cc3)c2)cc1]

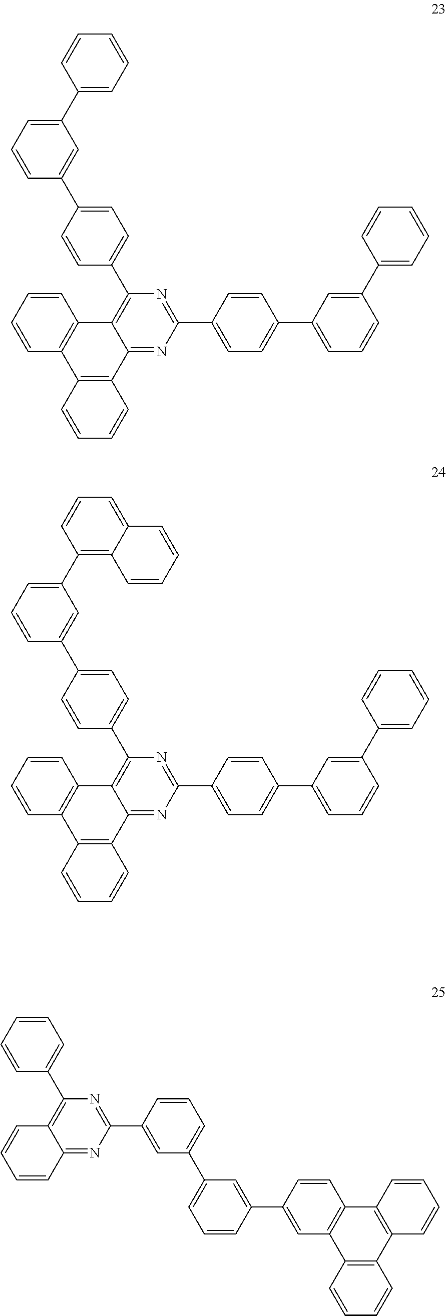 [SMILES: c1ccc(-c2cccc(-c3ccc(-c4nc(-c5ccc(-c6cccc(-c7ccccc7)c6)cc5)c5c6ccccc6c6ccccc6c5n4)cc3)c2)cc1 ; c1ccc(-c2cccc(-c3ccc(-c4nc(-c5ccc(-c6cccc(-c7cccc8ccccc78)c6)cc5)c5c6ccccc6c6ccccc6c5n4)cc3)c2)cc1 ; c1ccc(-c2nc(-c3cccc(-c4cccc(-c5ccc6c7ccccc7c7ccccc7c6c5)c4)c3)nc3ccccc23)cc1]

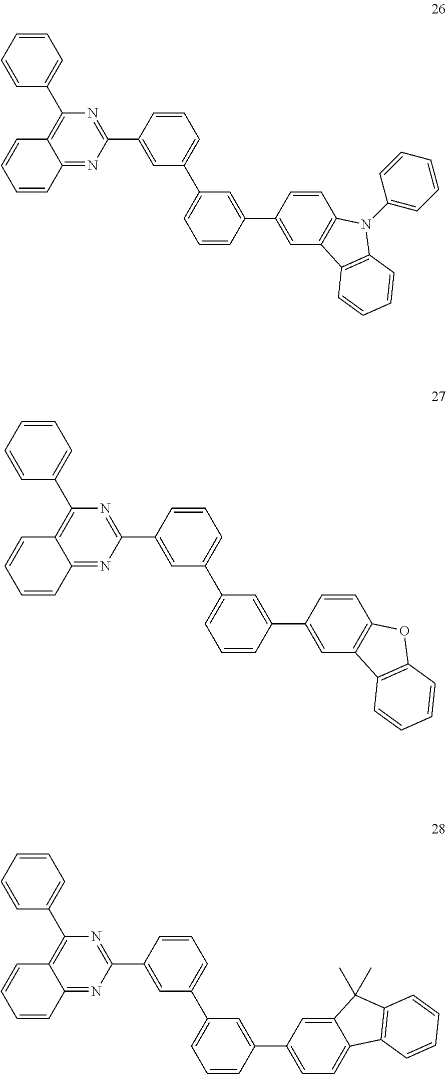 [SMILES: c1ccc(-c2nc(-c3cccc(-c4cccc(-c5ccc6c(c5)c5ccccc5n6-c5ccccc5)c4)c3)nc3ccccc23)cc1 ; c1ccc(-c2nc(-c3cccc(-c4cccc(-c5ccc6oc7ccccc7c6c5)c4)c3)nc3ccccc23)cc1 ; CC1(C)c2ccccc2-c2ccc(-c3cccc(-c4cccc(-c5nc(-c6ccccc6)c6ccccc6n5)c4)c3)cc21]

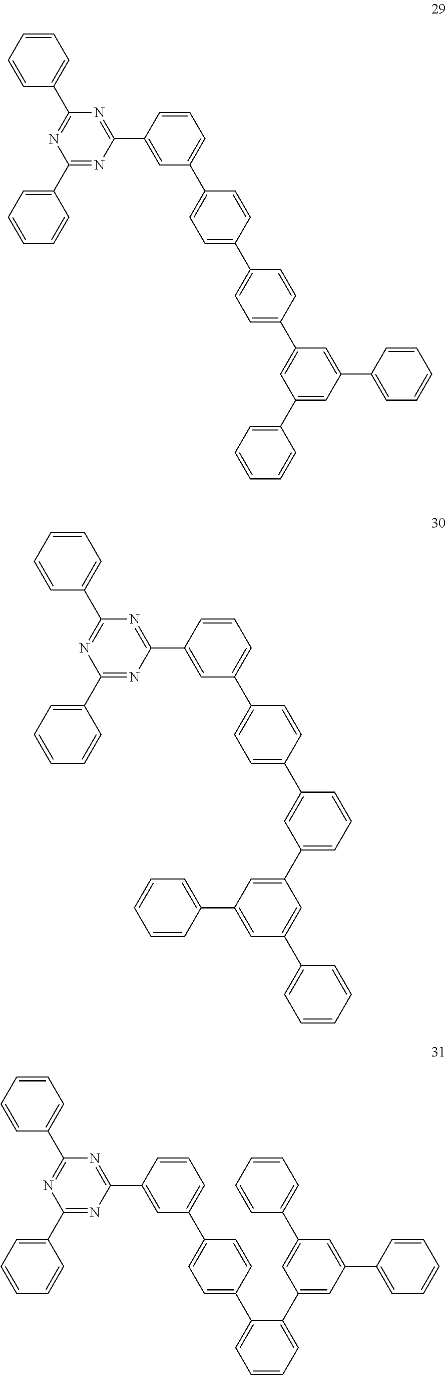 [SMILES: c1ccc(-c2cc(-c3ccccc3)cc(-c3ccc(-c4ccc(-c5cccc(-c6nc(-c7ccccc7)nc(-c7ccccc7)n6)c5)cc4)cc3)c2)cc1 ; c1ccc(-c2cc(-c3ccccc3)cc(-c3cccc(-c4ccc(-c5cccc(-c6nc(-c7ccccc7)nc(-c7ccccc7)n6)c5)cc4)c3)c2)cc1 ; c1ccc(-c2cc(-c3ccccc3)cc(-c3ccccc3-c3ccc(-c4cccc(-c5nc(-c6ccccc6)nc(-c6ccccc6)n5)c4)cc3)c2)cc1]

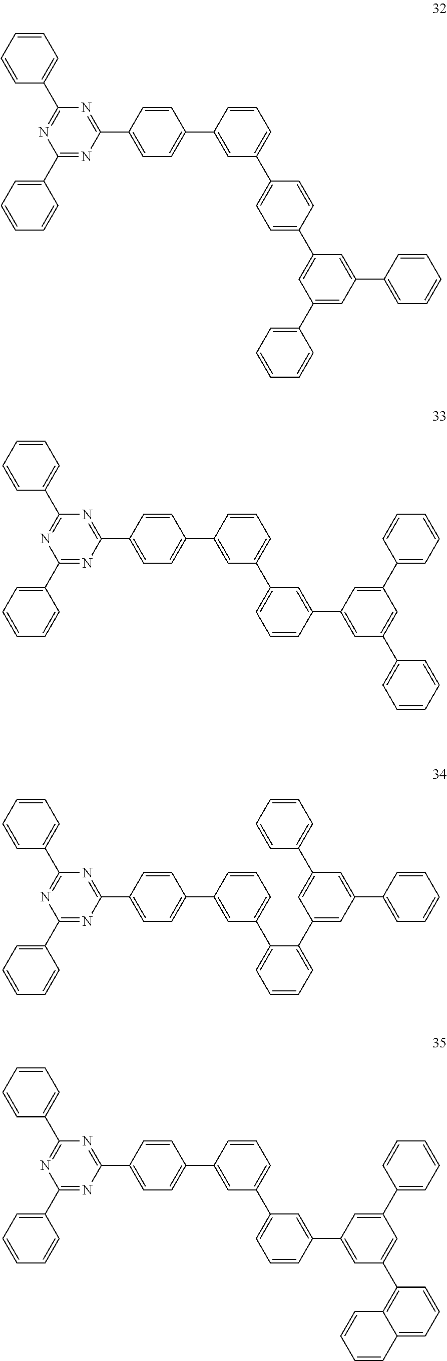 [SMILES: c1ccc(-c2cc(-c3ccccc3)cc(-c3ccc(-c4cccc(-c5ccc(-c6nc(-c7ccccc7)nc(-c7ccccc7)n6)cc5)c4)cc3)c2)cc1 ; c1ccc(-c2cc(-c3ccccc3)cc(-c3cccc(-c4cccc(-c5ccc(-c6nc(-c7ccccc7)nc(-c7ccccc7)n6)cc5)c4)c3)c2)cc1 ; c1ccc(-c2cc(-c3ccccc3)cc(-c3ccccc3-c3cccc(-c4ccc(-c5nc(-c6ccccc6)nc(-c6ccccc6)n5)cc4)c3)c2)cc1 ; c1ccc(-c2cc(-c3cccc(-c4cccc(-c5ccc(-c6nc(-c7ccccc7)nc(-c7ccccc7)n6)cc5)c4)c3)cc(-c3cccc4ccccc34)c2)cc1]

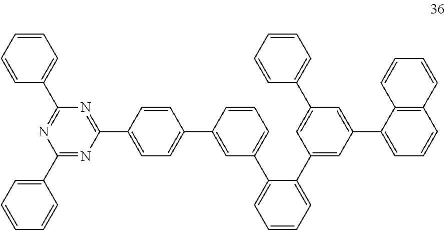 [SMILES: c1ccc(-c2cc(-c3ccccc3-c3cccc(-c4ccc(-c5nc(-c6ccccc6)nc(-c6ccccc6)n5)cc4)c3)cc(-c3cccc4ccccc34)c2)cc1]

The second host compound is represented by Chemical Formula II.

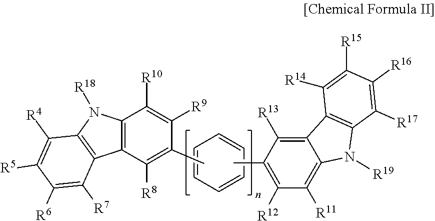

In Chemical Formula II,

R⁴ to R¹⁷ are independently hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,

adjacent two of R⁴ to R¹⁰ and R¹¹ to R¹⁷ are linked to each other to provide a ring,

R¹⁸ and R¹⁹ are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heteroaryl group, a substituted or unsubstituted C6 to C30 arylamine group, a substituted or unsubstituted C1 to C30 alkoxy group, a substituted or unsubstituted C2 to C30 alkoxycarbonyl group, a substituted or unsubstituted C2 to C30 alkoxycarbonylamino group, a substituted or unsubstituted C7 to C30 aryloxycarbonylamino group, a substituted or unsubstituted C1 to C30 sulfamoylamino group, a substituted or unsubstituted C2 to C30 alkenyl group, a substituted or unsubstituted C2 to C30 alkynyl group, a substituted or unsubstituted C3 to C40 silyl group, a substituted or unsubstituted C3 to C40 silyloxy group, a substituted or unsubstituted C1 to C30 acyl group, a substituted or unsubstituted C1 to C20 acyloxy group, a substituted or unsubstituted C1 to C20 acylamino group, a substituted or unsubstituted C1 to C30 sulfonyl group, a substituted or unsubstituted C1 to C30 alkylthiol group, a substituted or unsubstituted C6 to C30 arylthiol group, a substituted or unsubstituted C1 to C30 ureide group, a halogen, a halogen-containing group, a cyano group, a hydroxyl group, an amino group, a nitro group, a carboxyl group, a ferrocenyl group, or a combination thereof, and

n is an integer ranging from 1 to 4.

The second host compound includes a linking group connected with one to four phenylenes and has a flexible molecular structure and thus may be effectively prevented from stacking and advantageous during a deposition process.

In addition, the second host compound is applied with the first host compound and thus may appropriately balance hole and electron flows and improve efficiency of an organic optoelectric diode manufactured by applying a composition including the first and second host compounds.

The second host compound may be represented by one of Chemical Formulae II-1 to II-16 according to kinds of intermediate linking groups.

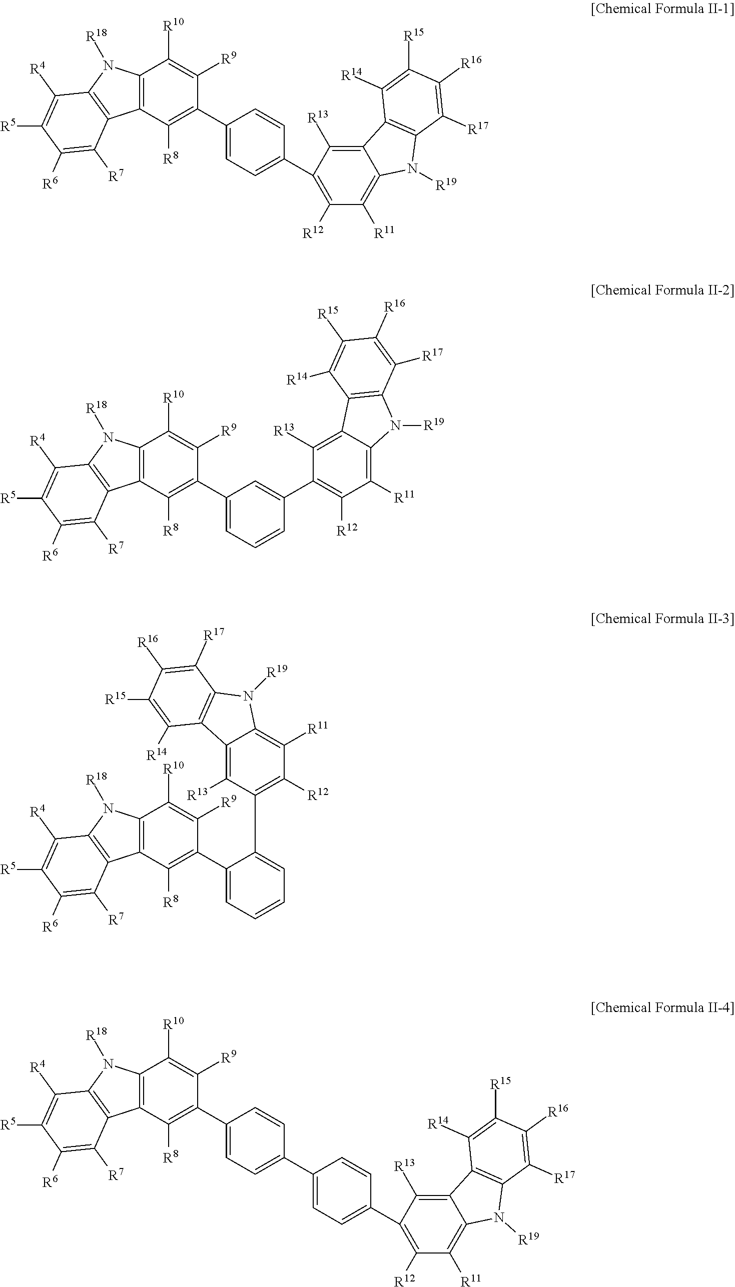

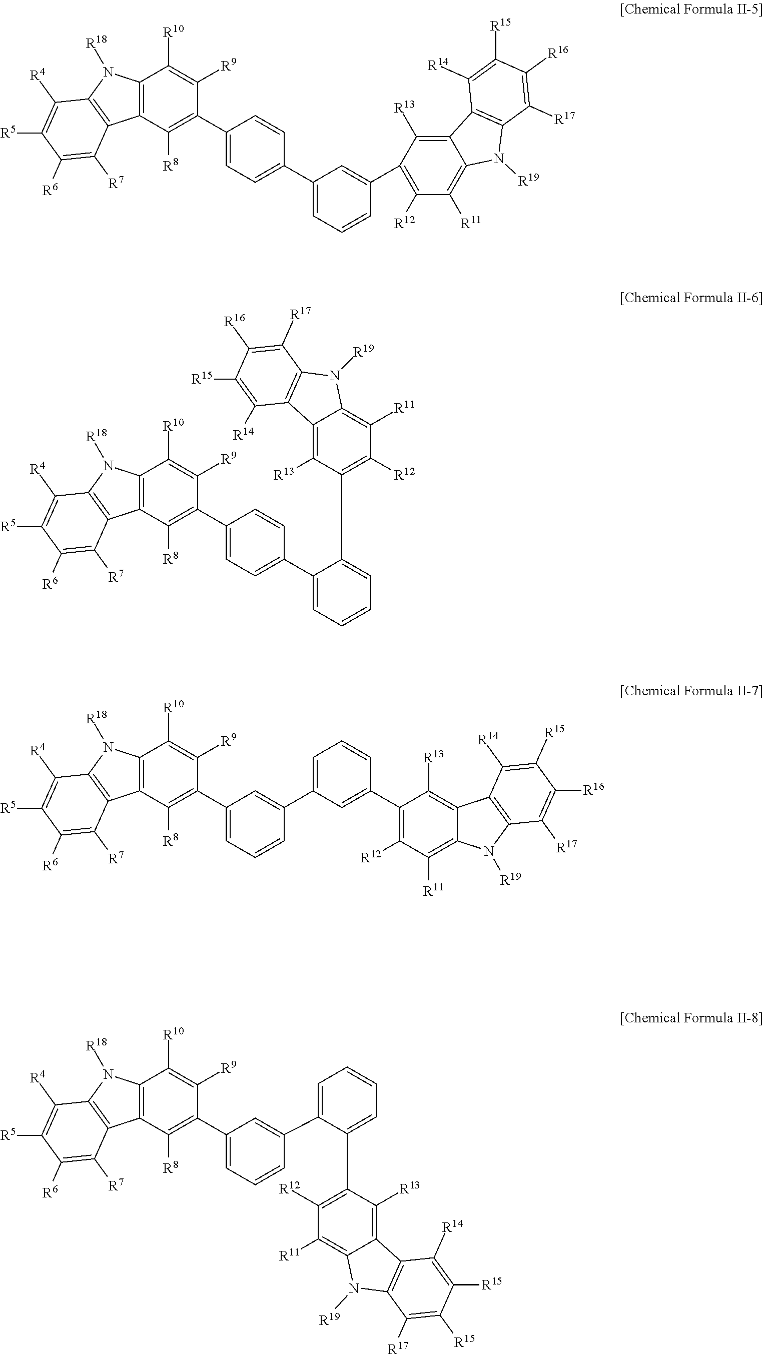

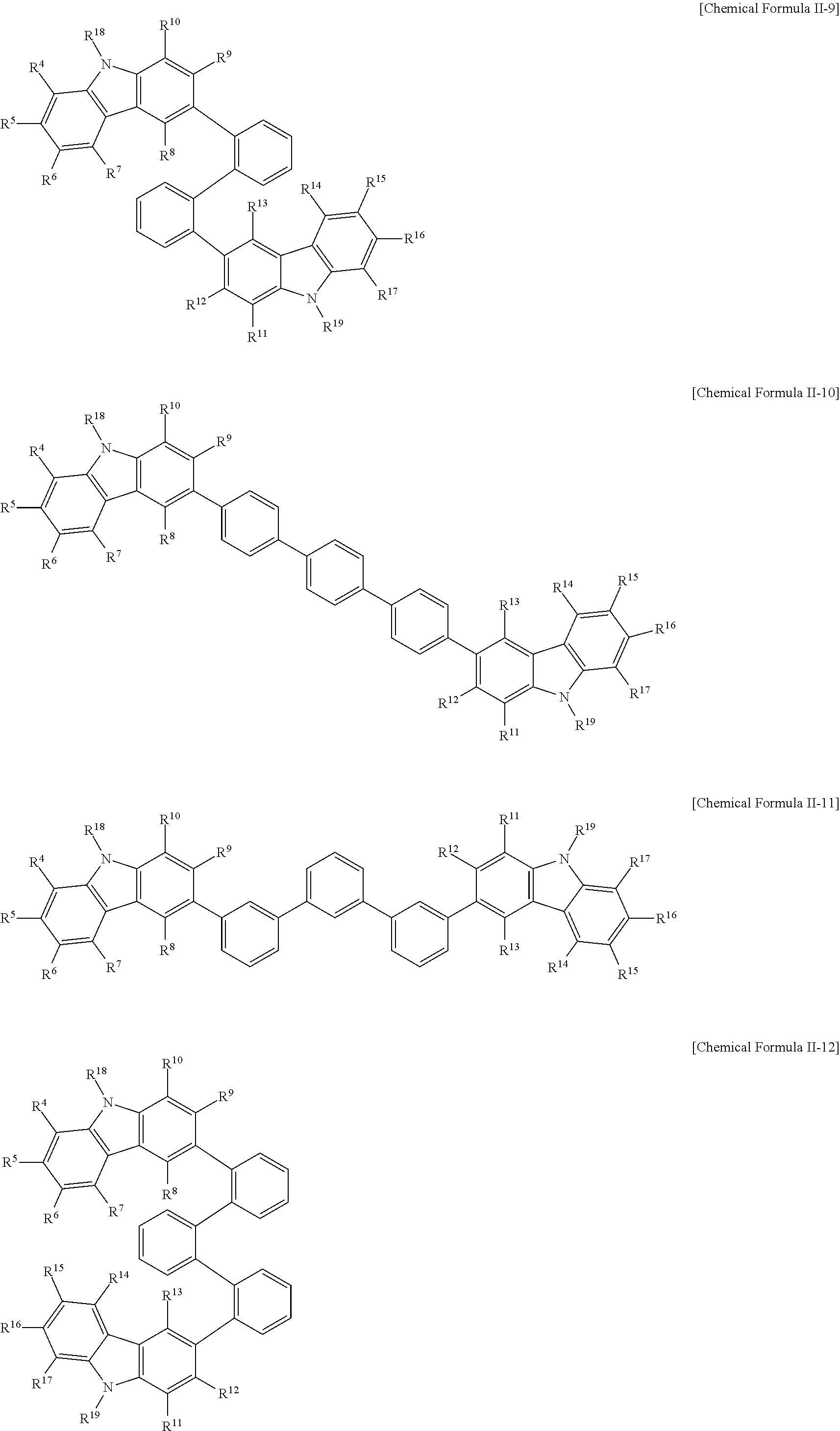

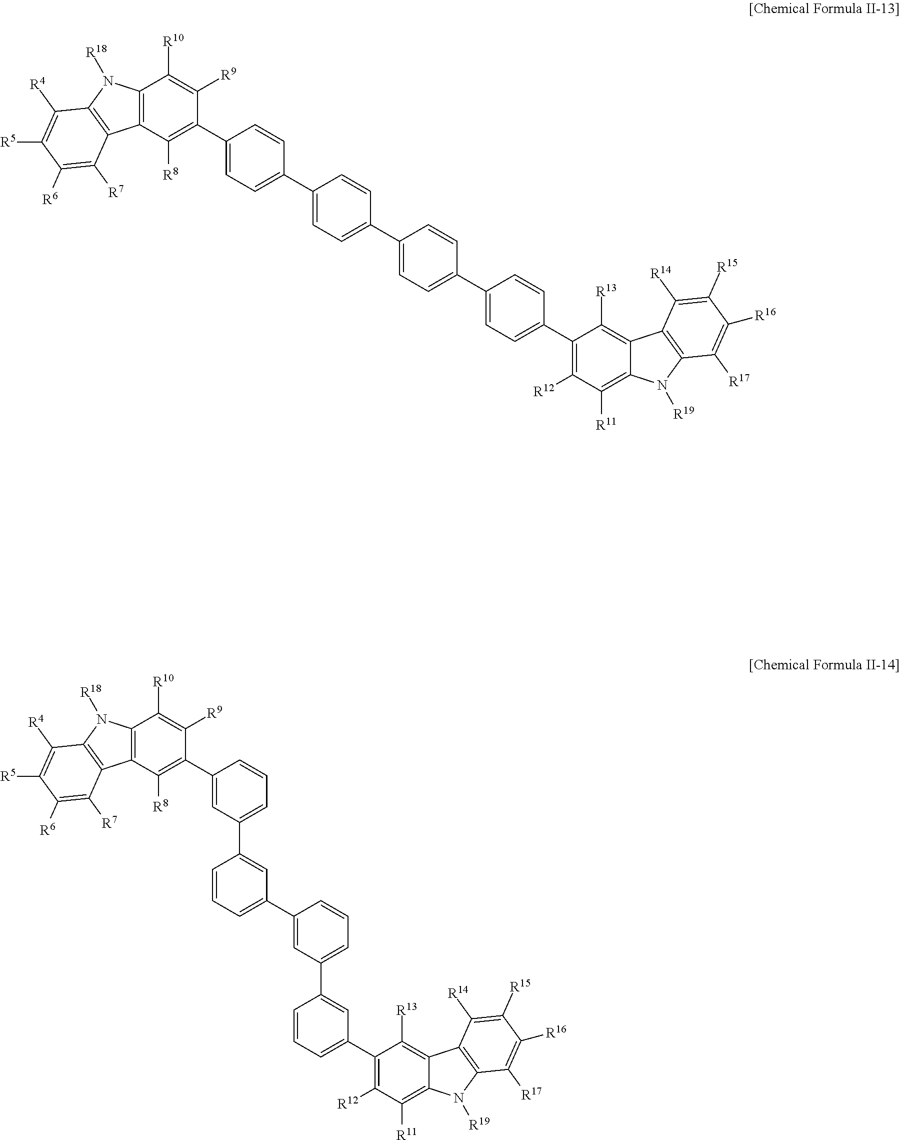

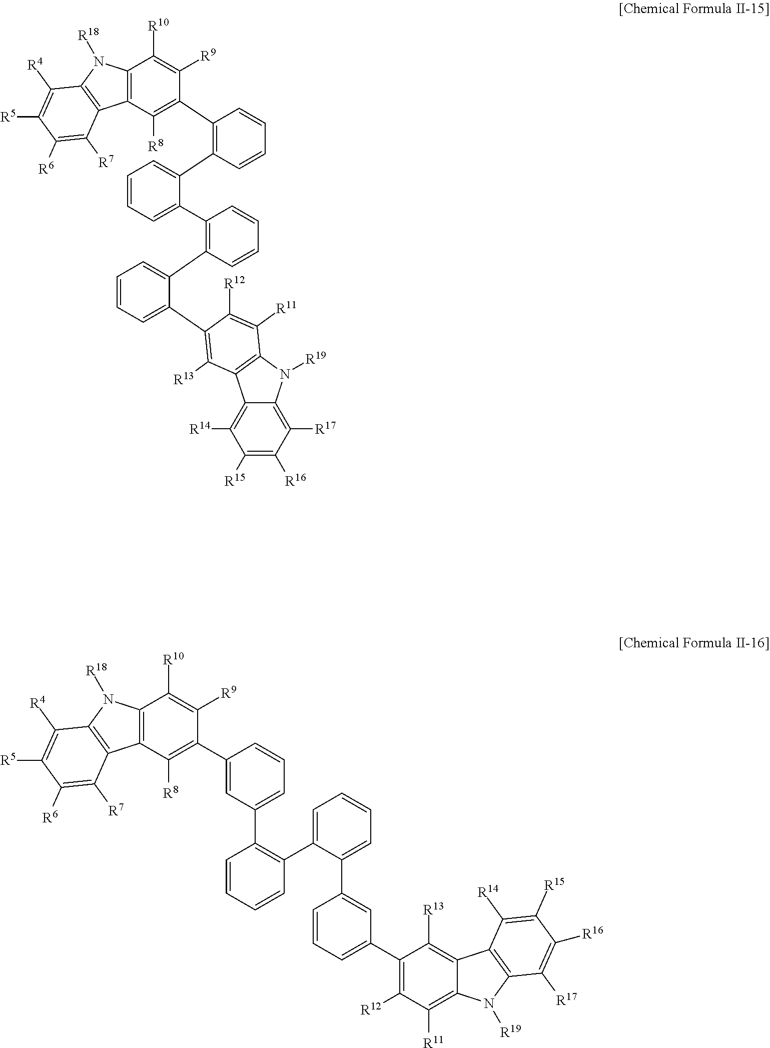

In Chemical Formulae II-1 to II-16, R⁴ to R¹⁹ are the same as described above.

In addition, for example, in Chemical Formulae II-1 to II-16, the R⁴ to R¹⁷ may independently be hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof, adjacent two of R⁴ to R¹⁰ and R¹¹ to R¹⁷ are linked to each other to provide a ring, and R¹⁸ and R¹⁹ are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heteroaryl group, a substituted or unsubstituted C6 to C30 arylamine group, a substituted or unsubstituted C1 to C30 alkylthiol group, a substituted or unsubstituted C6 to C30 arylthiol group, or a combination thereof.

Specifically, the R¹⁸ and R¹⁹ may independently be hydrogen, deuterium, a substituted or unsubstituted C6 to C30 aryl group, or a substituted or unsubstituted C2 to C30 heteroaryl group, and more specifically, the substituted or unsubstituted C6 to C30 aryl group may be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted 1H-phenalenyl group, a substituted or unsubstituted pyrenyl group, a substituted or unsubstituted fluorenyl group, a substituted or unsubstituted triphenylene group, or a combination thereof, and the substituted or unsubstituted C2 to C30 heteroaryl group may be a substituted or unsubstituted pyridyl group, a substituted or unsubstituted pyrimidinyl group, a substituted or unsubstituted triazinyl group, or a combination thereof.

In addition, the second host compound may be represented by one of Chemical Formulae II-17 to II-39 according to substituents of R¹⁸ and R¹⁹.

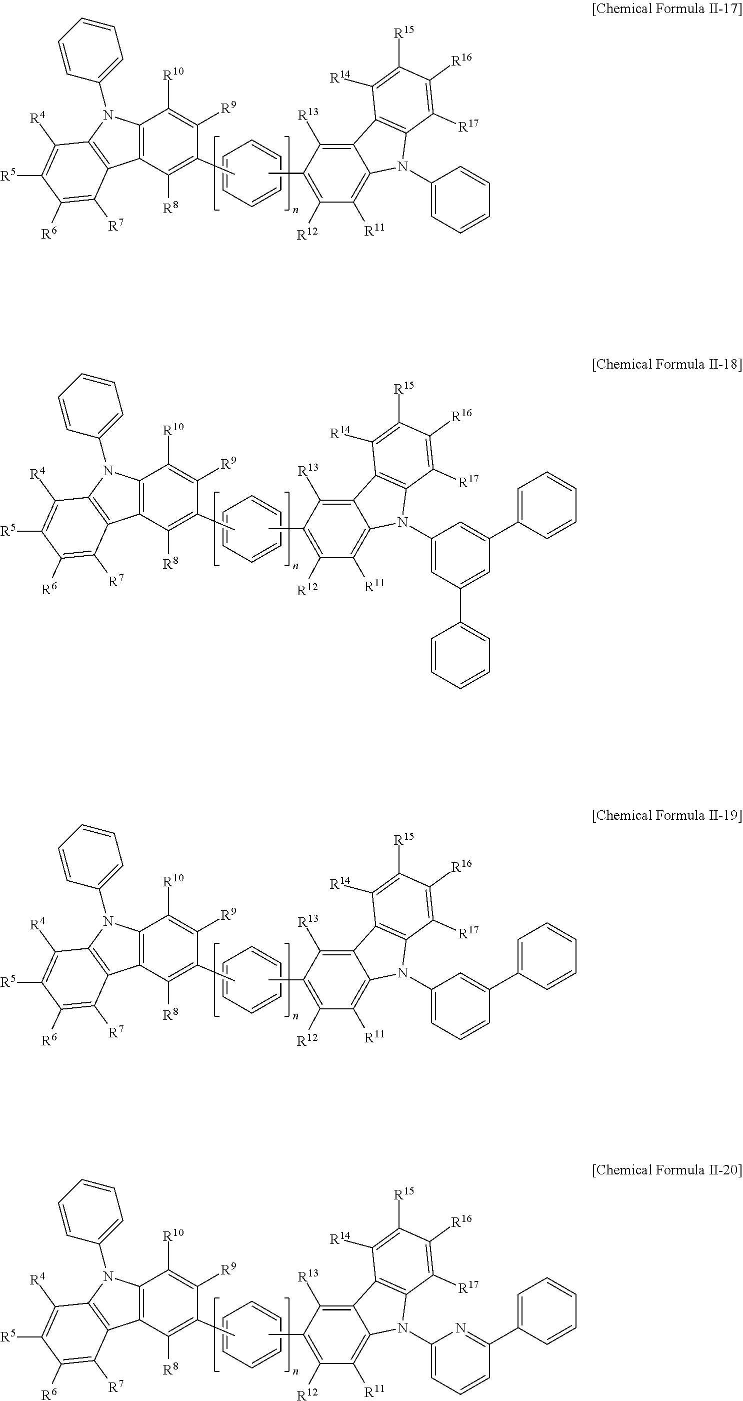

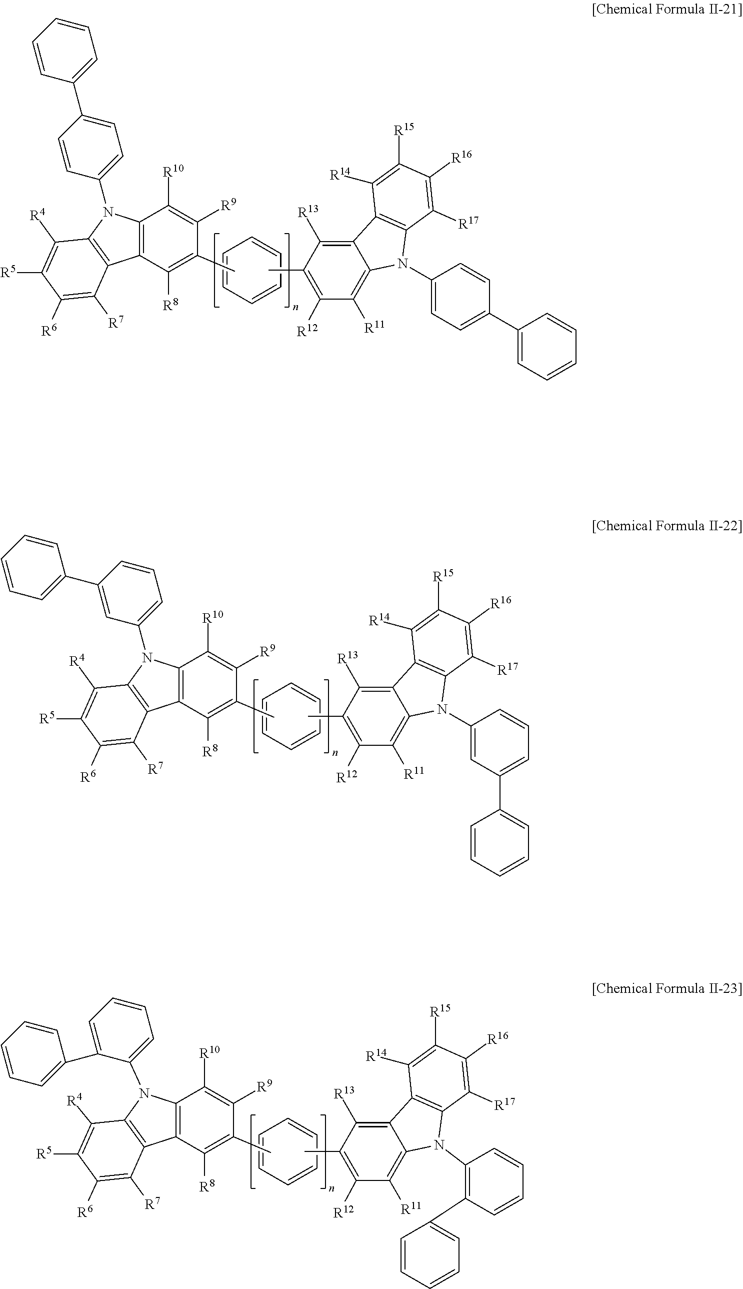

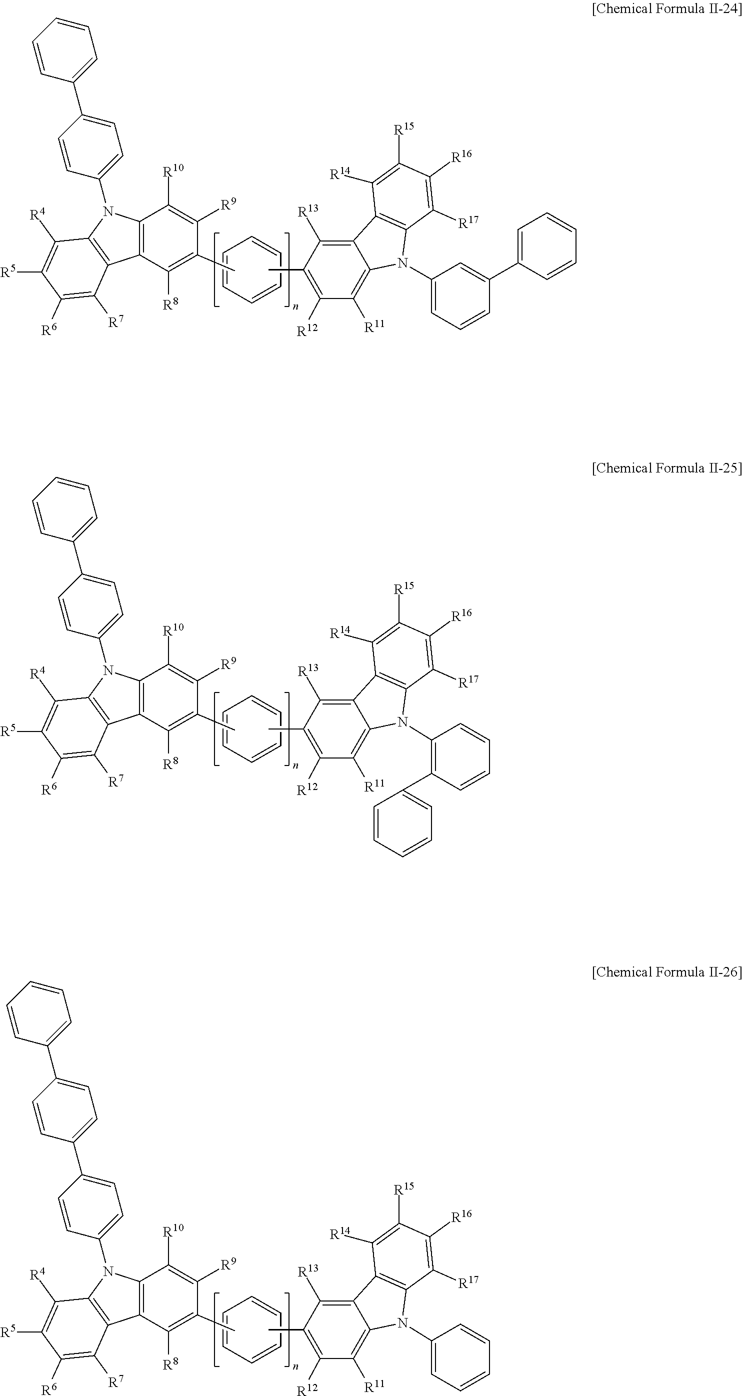

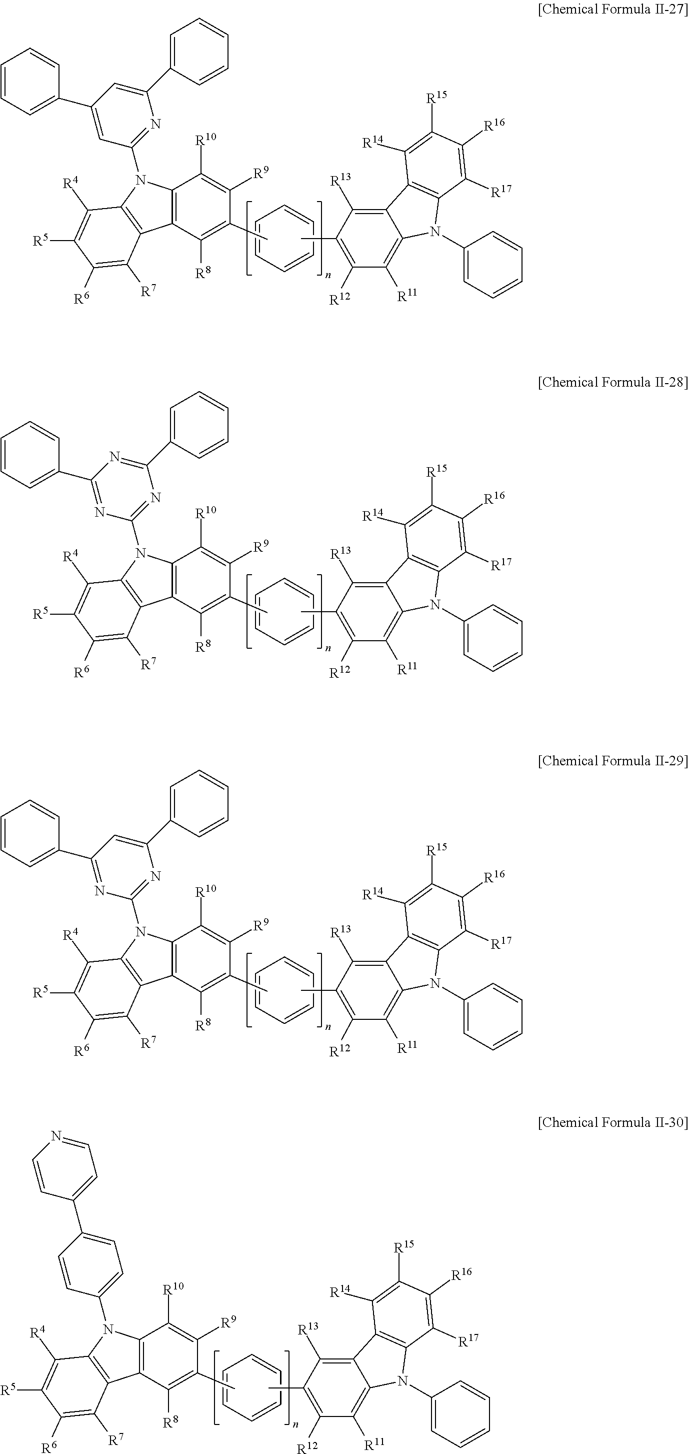

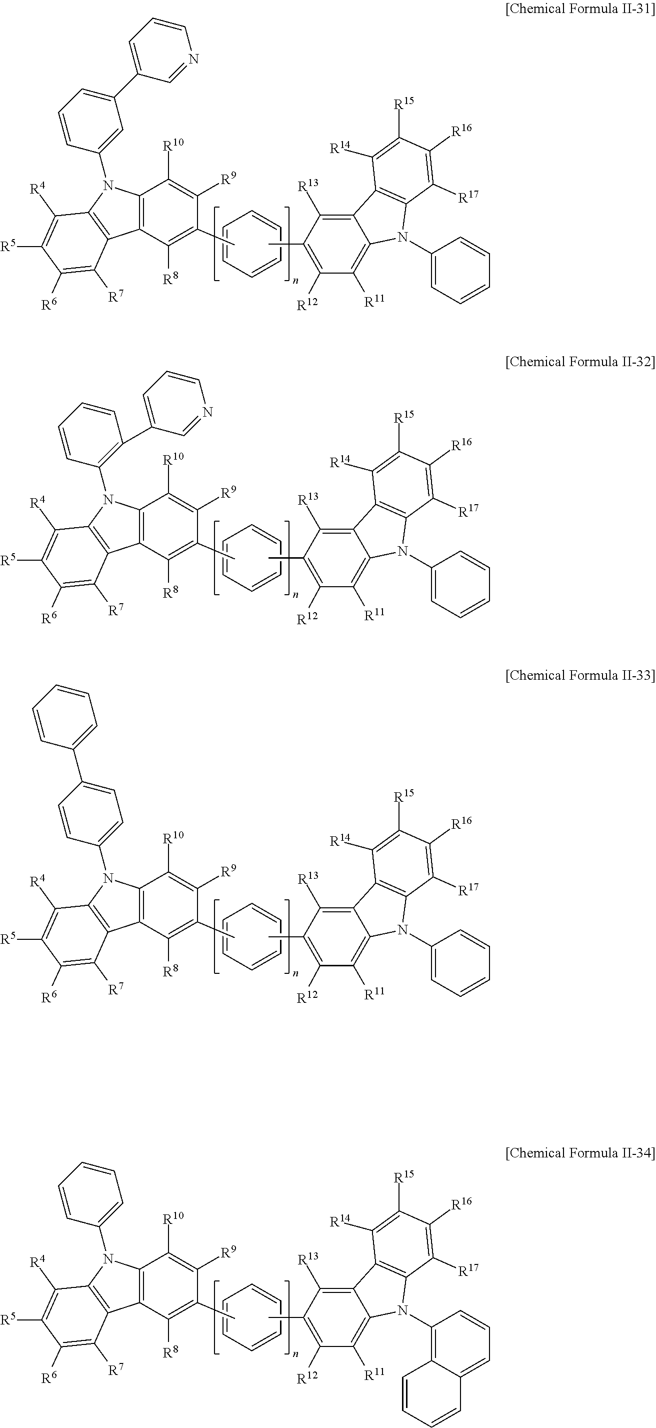

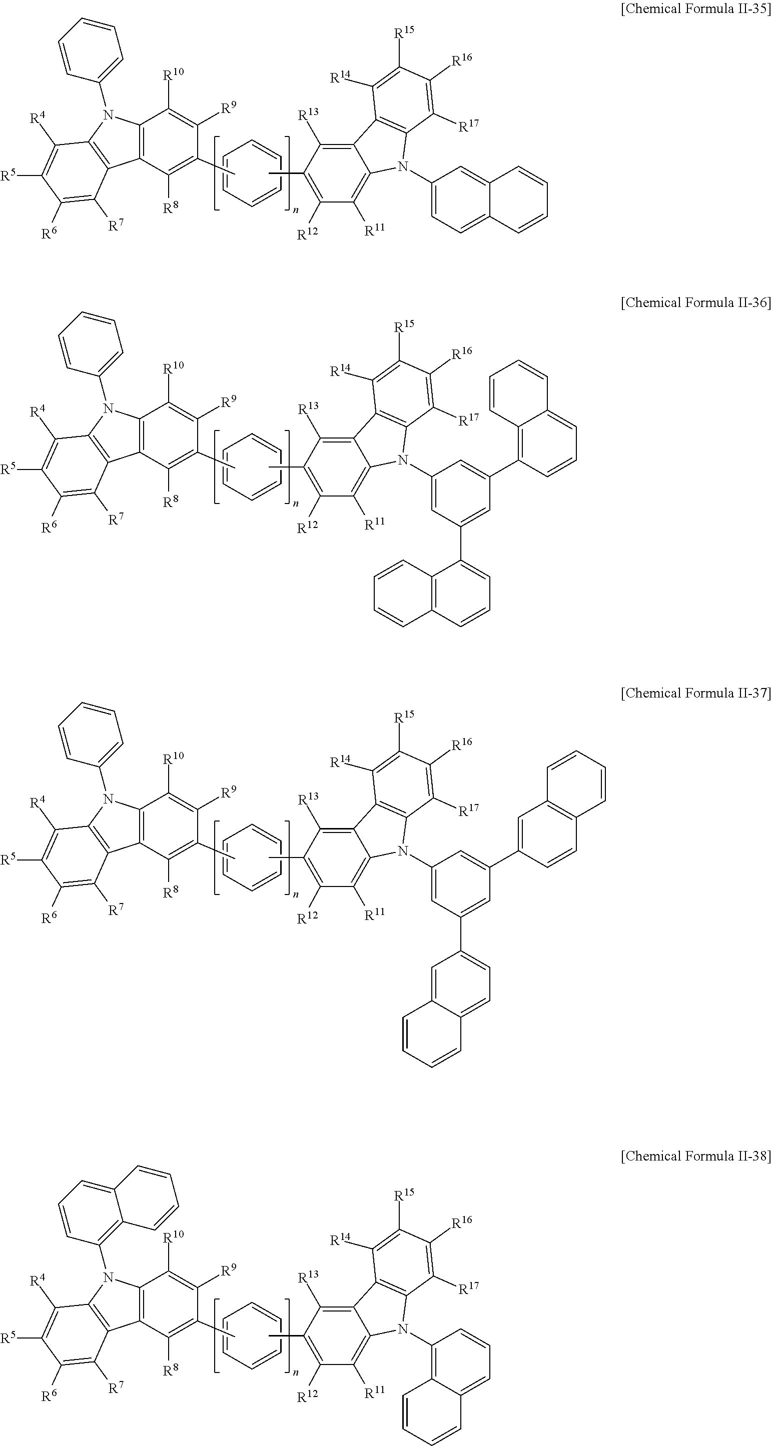

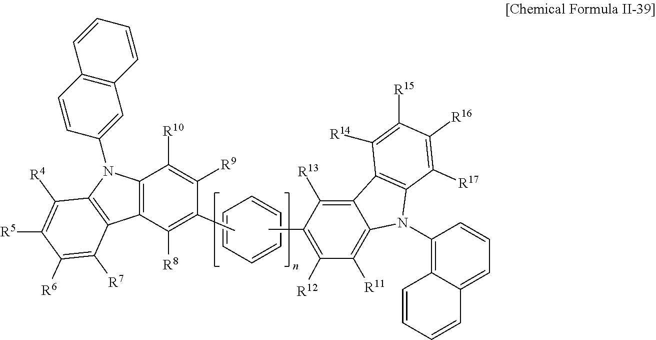

In Chemical Formulae II-17 to II-39, R⁴ to R¹⁷ and n are the same as described above.

For example, in Chemical Formulae II-17 to II-39, R⁴ to R¹⁷ may independently be hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,

adjacent two of R⁴ to R¹⁰ and R¹¹ to R¹⁷ are linked to each other to provide a ring, and

n is an integer of 1 to 4.

The R⁴ to R¹⁷ of Chemical Formula II may independently be hydrogen, deuterium, or a substituted or unsubstituted C6 to C30 aryl group.

Specifically, the substituted or unsubstituted C6 to C30 aryl group may be a substituted or unsubstituted phenyl group, a substituted or unsubstituted biphenyl group, a substituted or unsubstituted o-terphenyl group, a substituted or unsubstituted p-terphenyl group, a substituted or unsubstituted m-terphenyl group, a substituted or unsubstituted iso-quaterphenyl group, a substituted or unsubstituted tert-quaterphenyl group, 2-quaterphenyl group, a substituted or unsubstituted naphthyl group, or a combination thereof, but is not limited thereto.

The second host compound may be, for example selected from compounds of Group IV, but is not limited thereto.

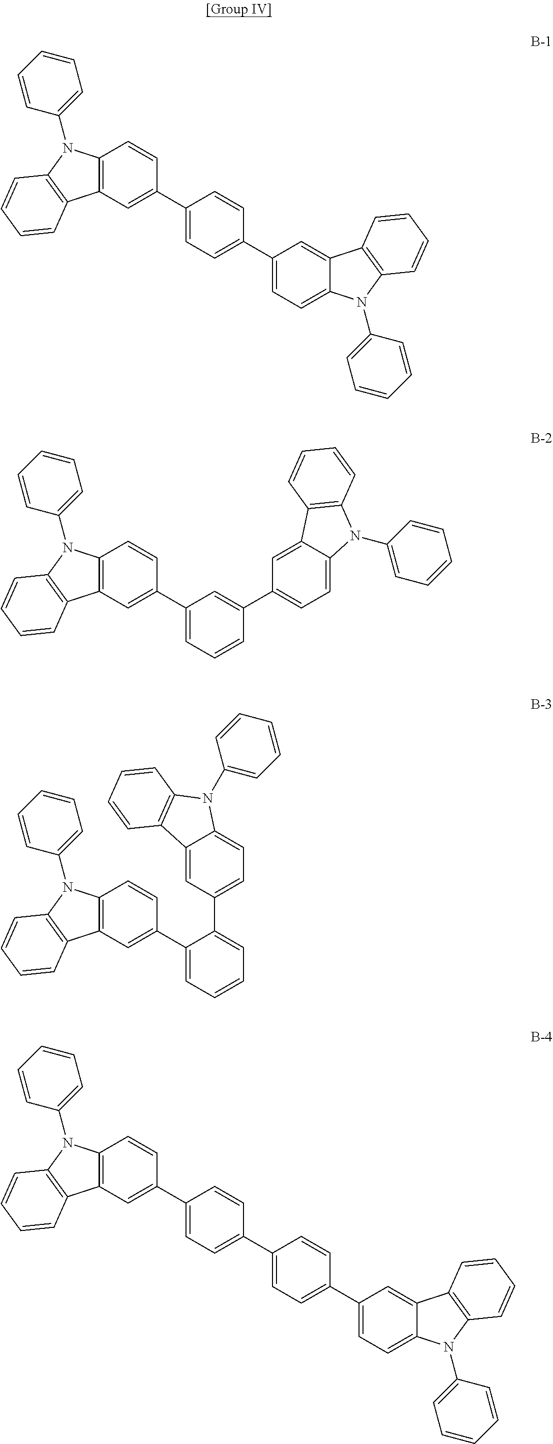 [SMILES: c1ccc(-n2c3ccccc3c3cc(-c4ccc(-c5ccc6c(c5)c5ccccc5n6-c5ccccc5)cc4)ccc32)cc1 ; c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccccc4-c4ccc5c(c4)c4ccccc4n5-c4ccccc4)ccc32)cc1 ; c1ccc(-n2c3ccccc3c3cc(-c4ccc(-c5ccc(-c6ccc7c(c6)c6ccccc6n7-c6ccccc6)cc5)cc4)ccc32)cc1]

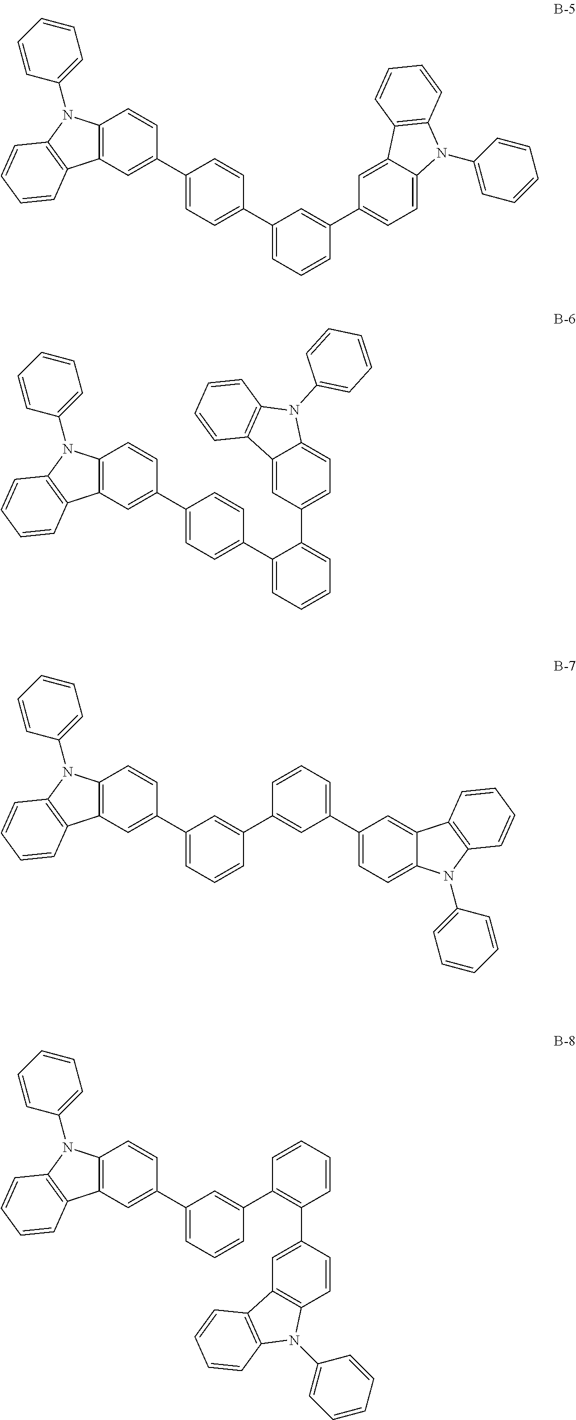 [SMILES: c1ccc(-n2c3ccccc3c3cc(-c4ccc(-c5cccc(-c6ccc7c(c6)c6ccccc6n7-c6ccccc6)c5)cc4)ccc32)cc1 ; c1ccc(-n2c3ccccc3c3cc(-c4ccc(-c5ccccc5-c5ccc6c(c5)c5ccccc5n6-c5ccccc5)cc4)ccc32)cc1 ; c1ccc(-n2c3ccccc3c3cc(-c4cccc(-c5cccc(-c6ccc7c(c6)c6ccccc6n7-c6ccccc6)c5)c4)ccc32)cc1 ; c1ccc(-n2c3ccccc3c3cc(-c4cccc(-c5ccccc5-c5ccc6c(c5)c5ccccc5n6-c5ccccc5)c4)ccc32)cc1]

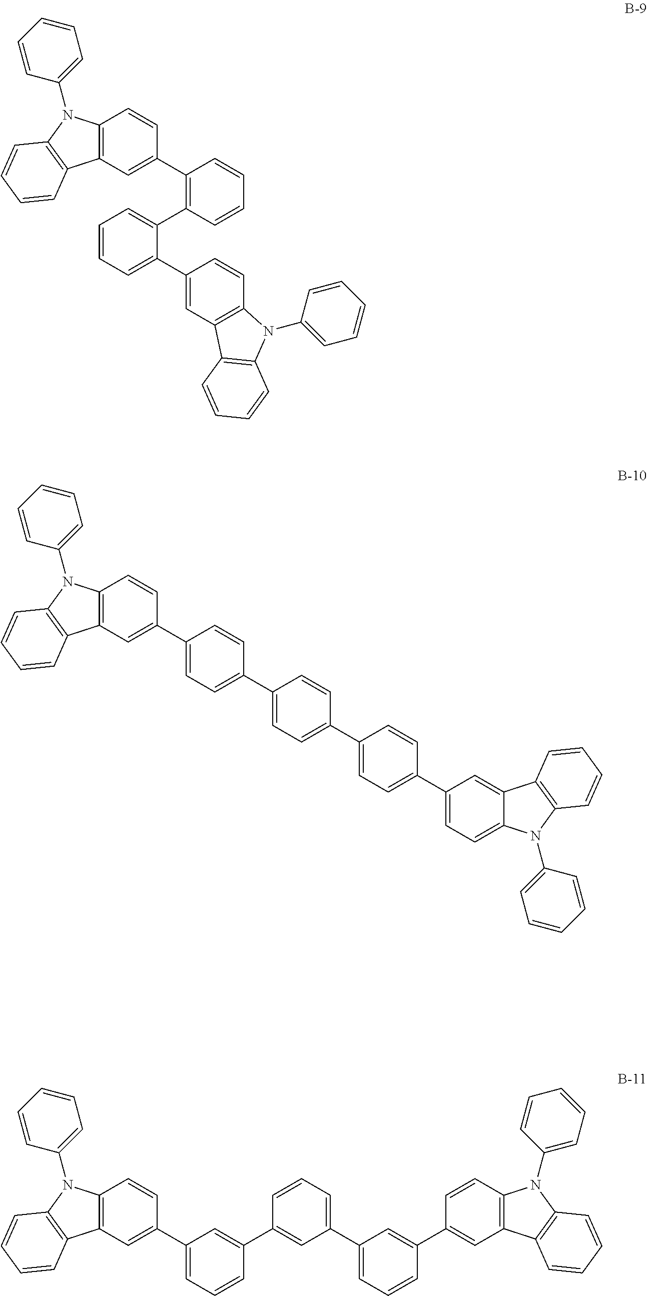 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-n2c3ccccc3c3cc(-c4ccc(-c5ccc(-c6ccc(-c7ccc8c(c7)c7ccccc7n8-c7ccccc7)cc6)cc5)cc4)ccc32)cc1 ; c1ccc(-n2c3ccccc3c3cc(-c4cccc(-c5cccc(-c6cccc(-c7ccc8c(c7)c7ccccc7n8-c7ccccc7)c6)c5)c4)ccc32)cc1]

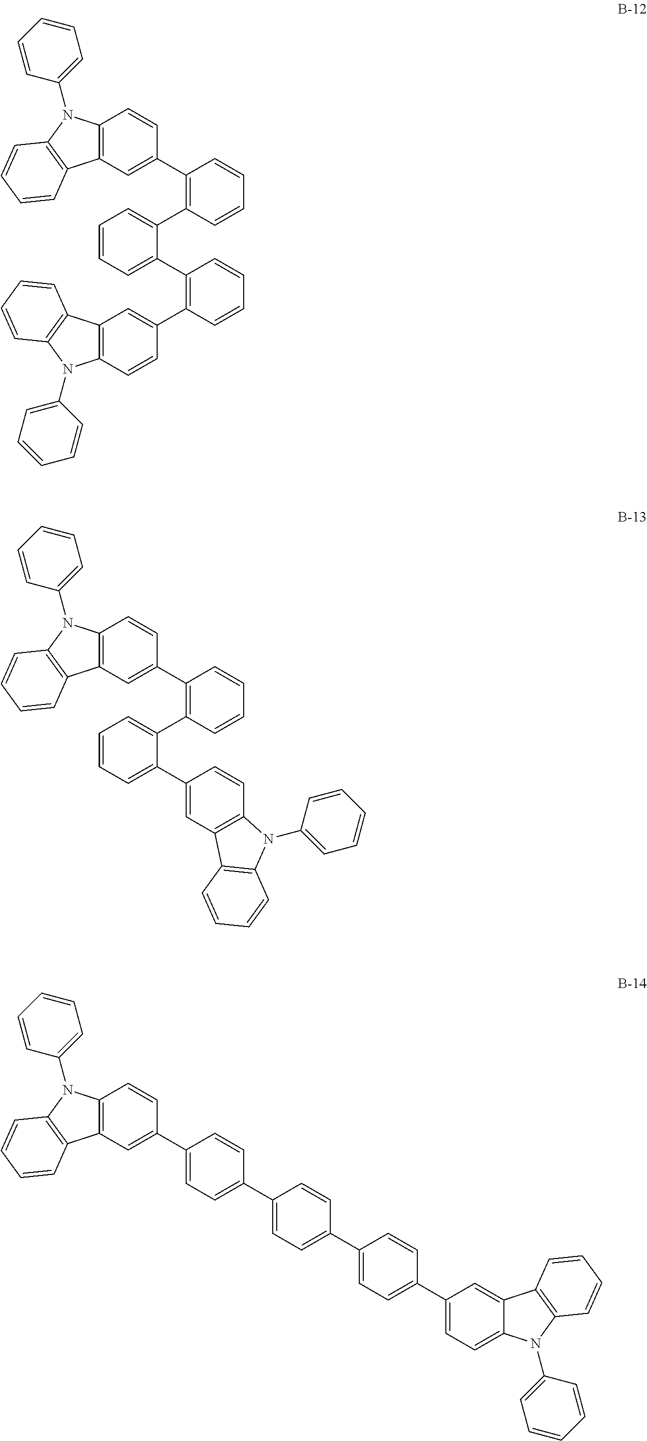 [SMILES: c1ccc(-n2c3ccccc3c3cc(-c4ccccc4-c4ccccc4-c4ccccc4-c4ccc5c(c4)c4ccccc4n5-c4ccccc4)ccc32)cc1 ; c1ccc(-n2c3ccccc3c3cc(-c4ccccc4-c4ccccc4-c4ccc5c(c4)c4ccccc4n5-c4ccccc4)ccc32)cc1 ; c1ccc(-n2c3ccccc3c3cc(-c4ccc(-c5ccc(-c6ccc(-c7ccc8c(c7)c7ccccc7n8-c7ccccc7)cc6)cc5)cc4)ccc32)cc1]

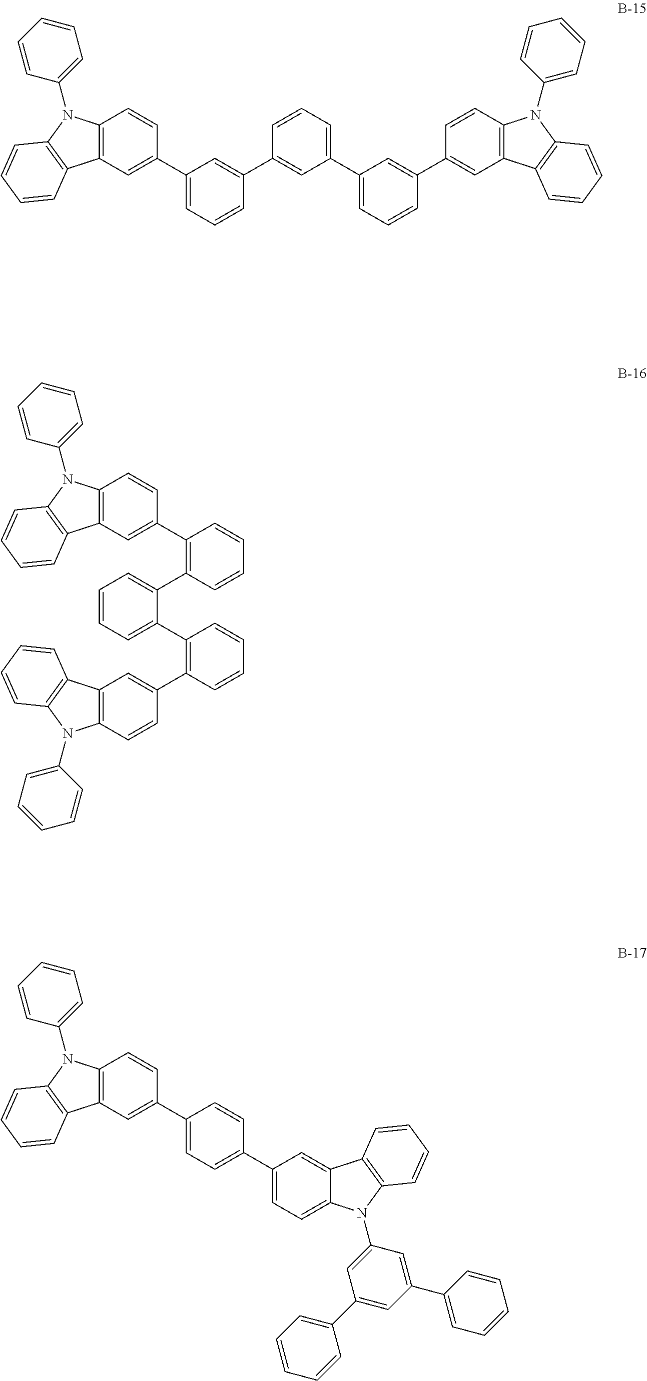 [SMILES: c1ccc(-n2c3ccccc3c3cc(-c4cccc(-c5cccc(-c6cccc(-c7ccc8c(c7)c7ccccc7n8-c7ccccc7)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4ccccc4)ccc32)cc1 ; c1ccc(-c2cc(-c3ccccc3)cc(-n3c4ccccc4c4cc(-c5ccc(-c6ccc7c(c6)c6ccccc6n7-c6ccccc6)cc5)ccc43)c2)cc1]

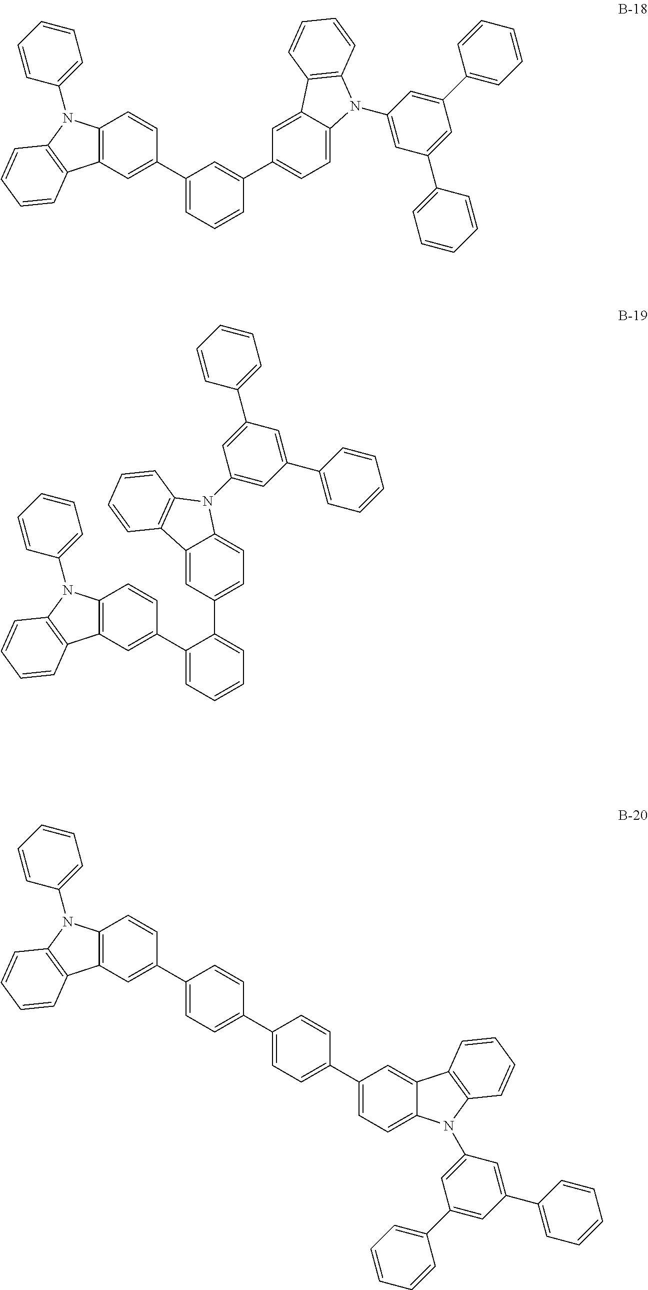 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2cc(-c3ccccc3)cc(-n3c4ccccc4c4cc(-c5ccccc5-c5ccc6c(c5)c5ccccc5n6-c5ccccc5)ccc43)c2)cc1 ; c1ccc(-c2cc(-c3ccccc3)cc(-n3c4ccccc4c4cc(-c5ccc(-c6ccc(-c7ccc8c(c7)c7ccccc7n8-c7ccccc7)cc6)cc5)ccc43)c2)cc1]

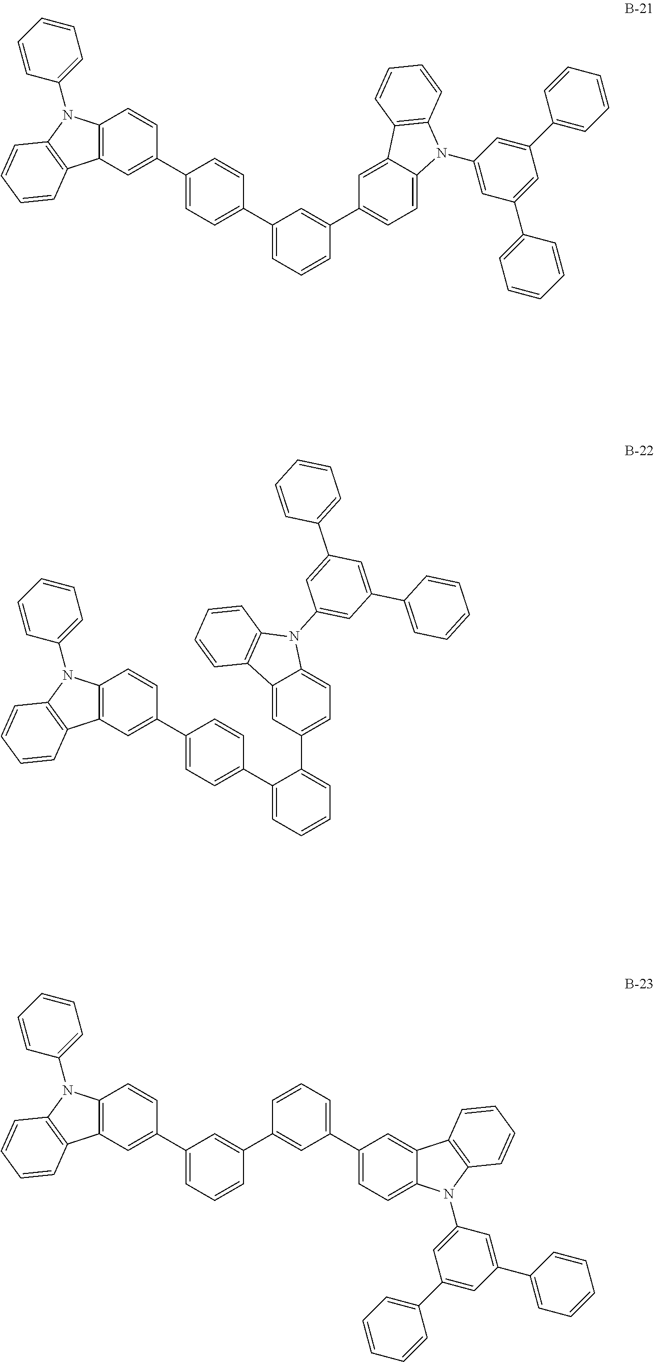 [SMILES: c1ccc(-c2cc(-c3ccccc3)cc(-n3c4ccccc4c4cc(-c5cccc(-c6ccc(-c7ccc8c(c7)c7ccccc7n8-c7ccccc7)cc6)c5)ccc43)c2)cc1 ; c1ccc(-c2cc(-c3ccccc3)cc(-n3c4ccccc4c4cc(-c5ccccc5-c5ccc(-c6ccc7c(c6)c6ccccc6n7-c6ccccc6)cc5)ccc43)c2)cc1 ; c1ccc(-c2cc(-c3ccccc3)cc(-n3c4ccccc4c4cc(-c5cccc(-c6cccc(-c7ccc8c(c7)c7ccccc7n8-c7ccccc7)c6)c5)ccc43)c2)cc1]

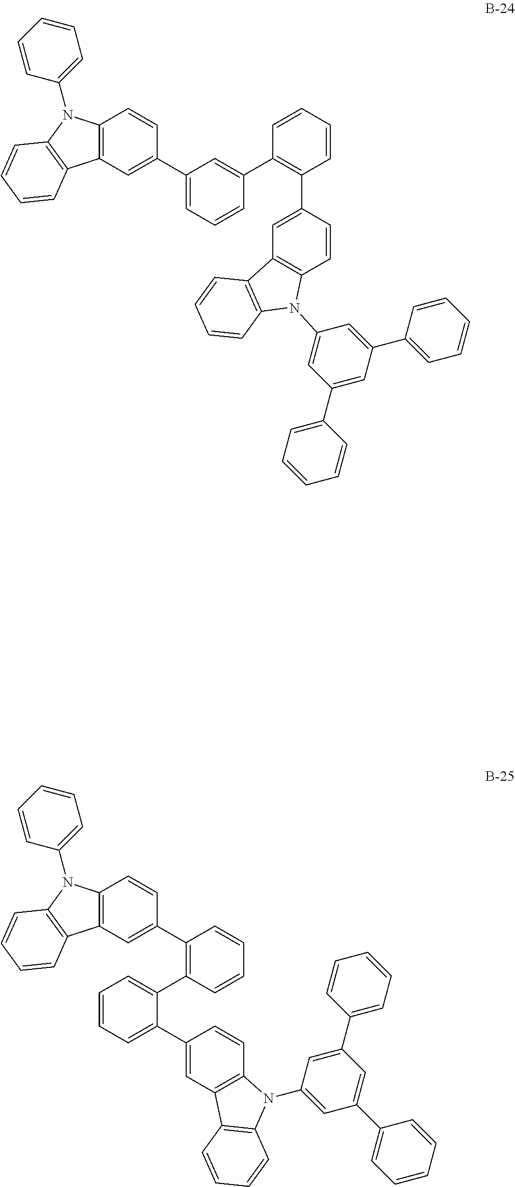 [SMILES: c1ccc(-c2cc(-c3ccccc3)cc(-n3c4ccccc4c4cc(-c5ccccc5-c5cccc(-c6ccc7c(c6)c6ccccc6n7-c6ccccc6)c5)ccc43)c2)cc1 ; c1ccc(-c2cc(-c3ccccc3)cc(-n3c4ccccc4c4cc(-c5ccccc5-c5ccccc5-c5ccc6c(c5)c5ccccc5n6-c5ccccc5)ccc43)c2)cc1]

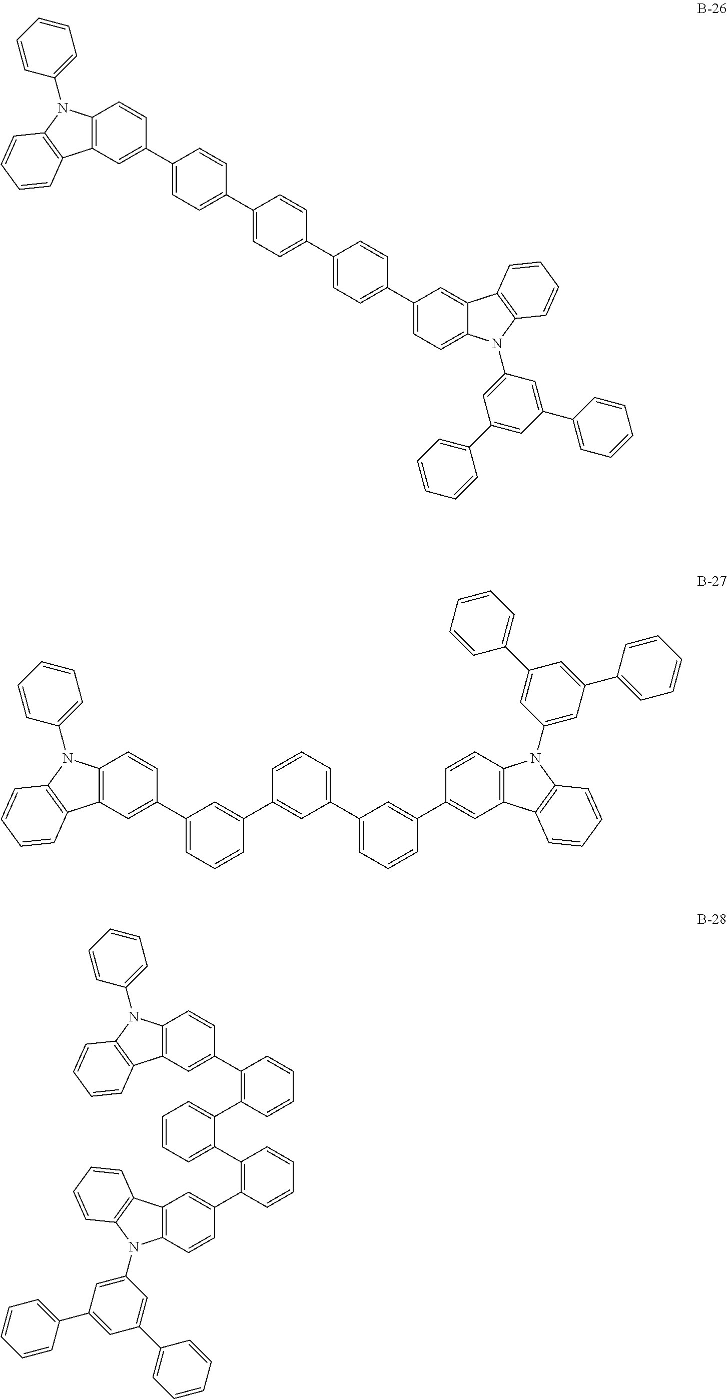 [SMILES: c1ccc(-c2cc(-c3ccccc3)cc(-n3c4ccccc4c4cc(-c5ccc(-c6ccc(-c7ccc(-c8ccc9c(c8)c8ccccc8n9-c8ccccc8)cc7)cc6)cc5)ccc43)c2)cc1 ; c1ccc(-c2cc(-c3ccccc3)cc(-n3c4ccccc4c4cc(-c5cccc(-c6cccc(-c7cccc(-c8ccc9c(c8)c8ccccc8n9-c8ccccc8)c7)c6)c5)ccc43)c2)cc1 ; c1ccc(-c2cc(-c3ccccc3)cc(-n3c4ccccc4c4cc(-c5ccccc5-c5ccccc5-c5ccccc5-c5ccc6c(c5)c5ccccc5n6-c5ccccc5)ccc43)c2)cc1]

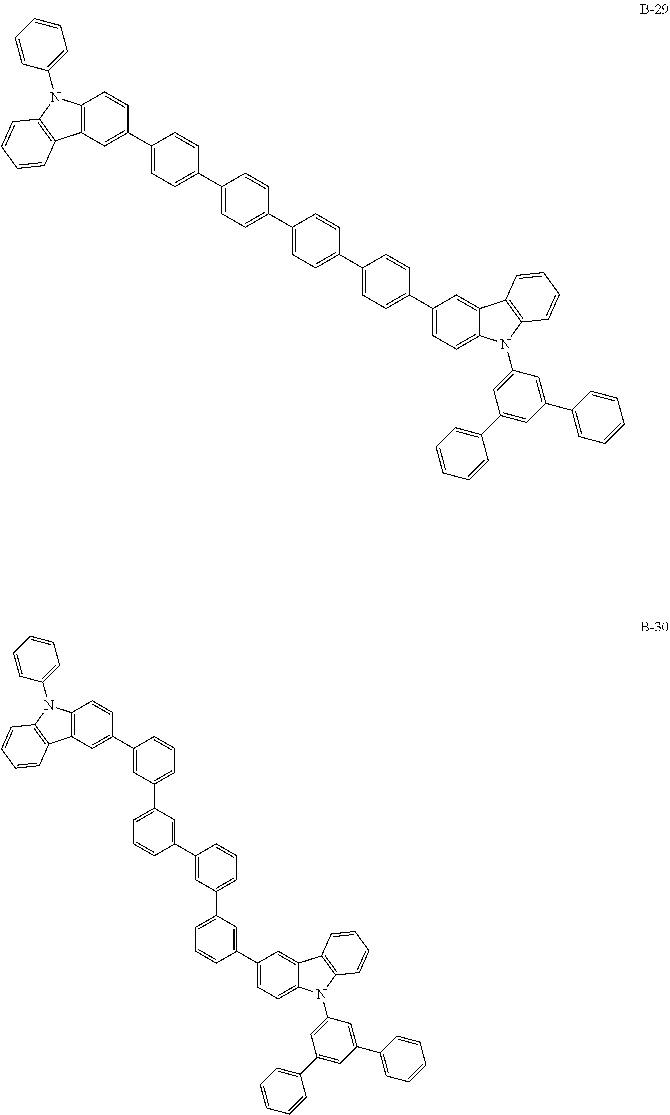 [SMILES: c1ccc(-c2cc(-c3ccccc3)cc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)c2)cc1 ; c1ccc(-c2cc(-c3ccccc3)cc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)c2)cc1]

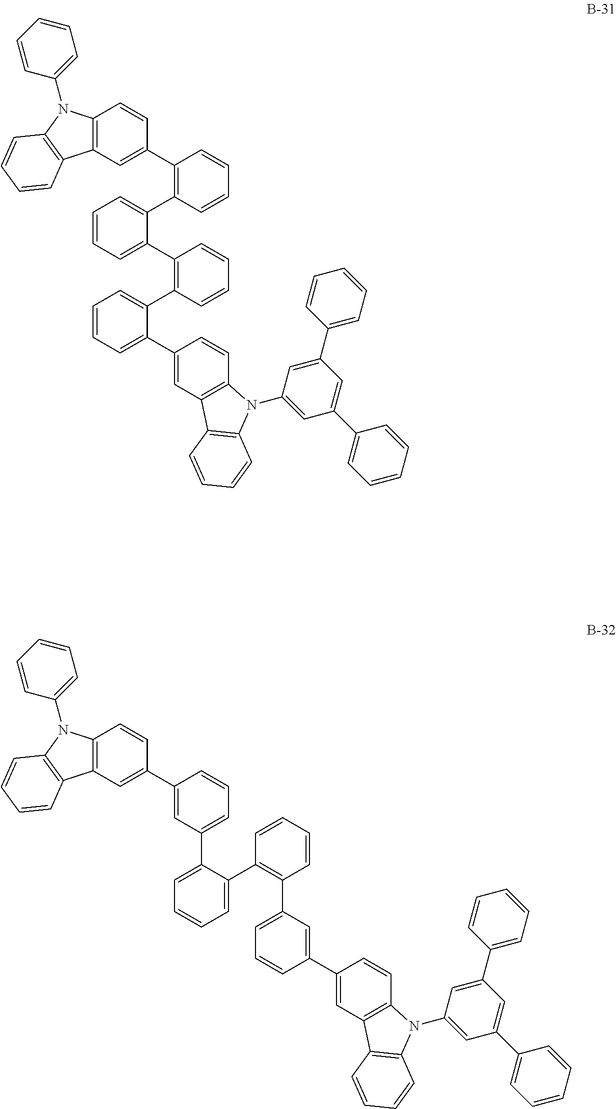 [SMILES: c1ccc(-c2cc(-c3ccccc3)cc(-n3c4ccccc4c4cc(-c5ccccc5-c5ccccc5-c5ccccc5-c5ccccc5-c5ccc6c(c5)c5ccccc5n6-c5ccccc5)ccc43)c2)cc1 ; c1ccc(-c2cc(-c3ccccc3)cc(-n3c4ccccc4c4cc(-c5cccc(-c6ccccc6-c6ccccc6-c6cccc(-c7ccc8c(c7)c7ccccc7n8-c7ccccc7)c6)c5)ccc43)c2)cc1]

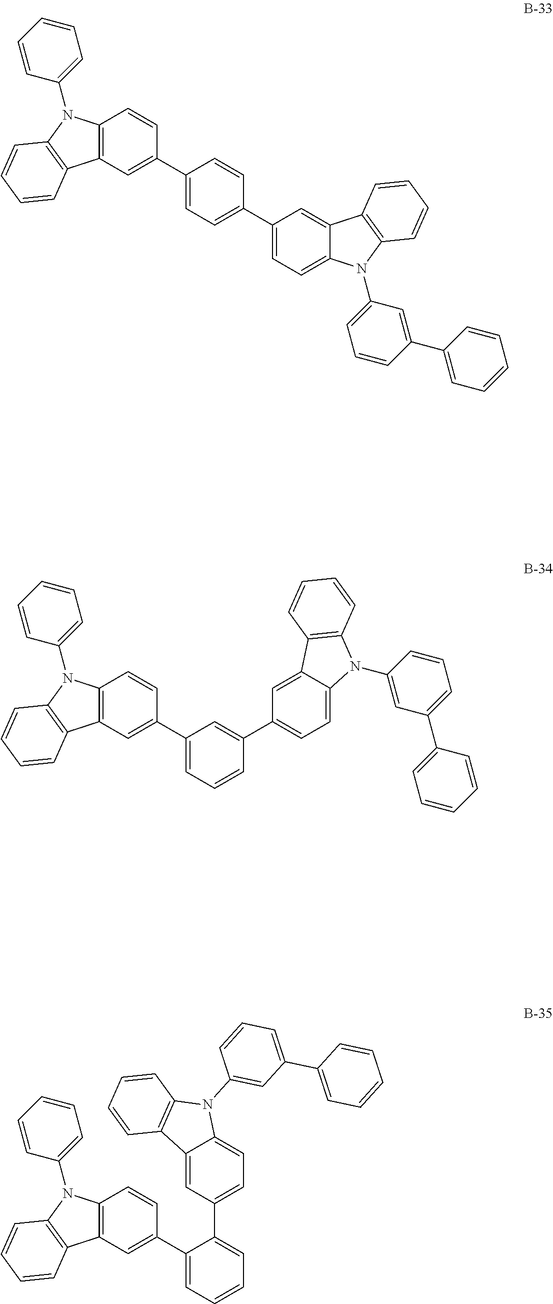 [SMILES: c1ccc(-c2cccc(-n3c4ccccc4c4cc(-c5ccc(-c6ccc7c(c6)c6ccccc6n7-c6ccccc6)cc5)ccc43)c2)cc1 ; c1ccc(-c2cccc(-n3c4ccccc4c4cc(-c5cccc(-c6ccc7c(c6)c6ccccc6n7-c6ccccc6)c5)ccc43)c2)cc1 ; c1ccc(-c2cccc(-n3c4ccccc4c4cc(-c5ccccc5-c5ccc6c(c5)c5ccccc5n6-c5ccccc5)ccc43)c2)cc1]

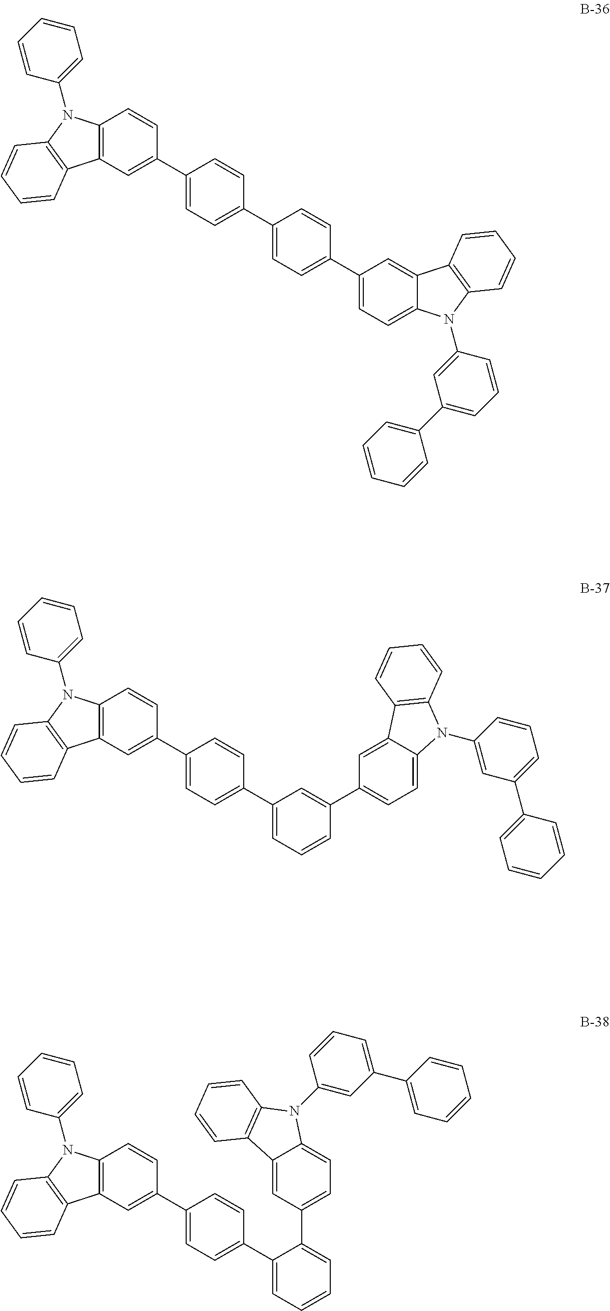 [SMILES: c1ccc(-c2cccc(-n3c4ccccc4c4cc(-c5ccc(-c6ccc(-c7ccc8c(c7)c7ccccc7n8-c7ccccc7)cc6)cc5)ccc43)c2)cc1 ; c1ccc(-c2cccc(-n3c4ccccc4c4cc(-c5cccc(-c6ccc(-c7ccc8c(c7)c7ccccc7n8-c7ccccc7)cc6)c5)ccc43)c2)cc1 ; c1ccc(-c2cccc(-n3c4ccccc4c4cc(-c5ccccc5-c5ccc(-c6ccc7c(c6)c6ccccc6n7-c6ccccc6)cc5)ccc43)c2)cc1]

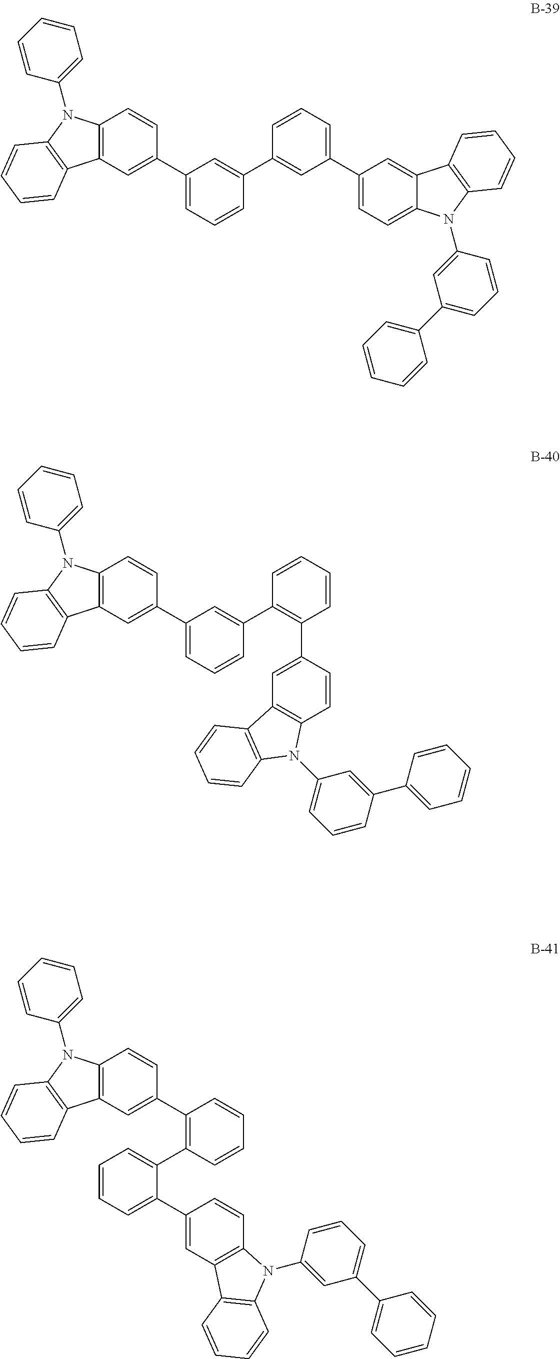 [SMILES: c1ccc(-c2cccc(-n3c4ccccc4c4cc(-c5cccc(-c6cccc(-c7ccc8c(c7)c7ccccc7n8-c7ccccc7)c6)c5)ccc43)c2)cc1 ; c1ccc(-c2cccc(-n3c4ccccc4c4cc(-c5ccccc5-c5cccc(-c6ccc7c(c6)c6ccccc6n7-c6ccccc6)c5)ccc43)c2)cc1 ; c1ccc(-c2cccc(-n3c4ccccc4c4cc(-c5ccccc5-c5ccccc5-c5ccc6c(c5)c5ccccc5n6-c5ccccc5)ccc43)c2)cc1]

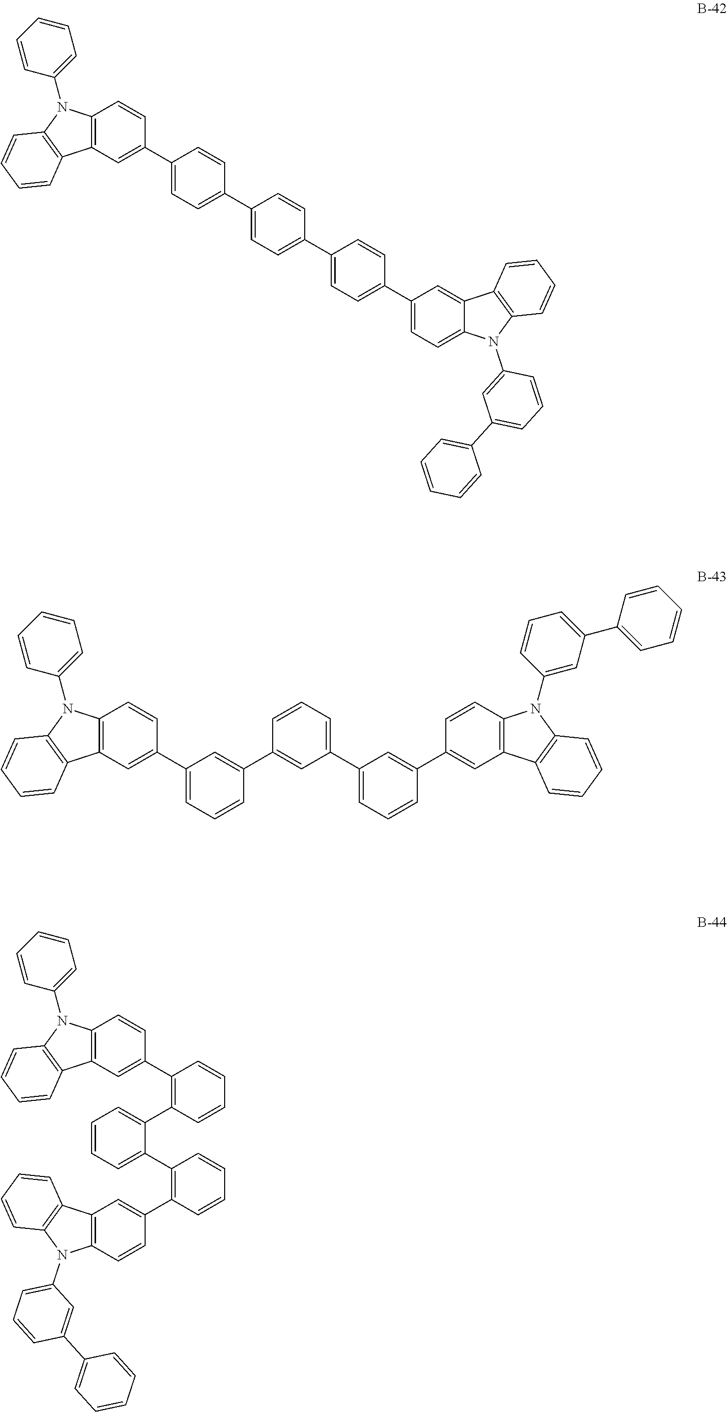 [SMILES: c1ccc(-c2cccc(-n3c4ccccc4c4cc(-c5ccc(-c6ccc(-c7ccc(-c8ccc9c(c8)c8ccccc8n9-c8ccccc8)cc7)cc6)cc5)ccc43)c2)cc1 ; c1ccc(-c2cccc(-n3c4ccccc4c4cc(-c5cccc(-c6cccc(-c7cccc(-c8ccc9c(c8)c8ccccc8n9-c8ccccc8)c7)c6)c5)ccc43)c2)cc1 ; c1ccc(-c2cccc(-n3c4ccccc4c4cc(-c5ccccc5-c5ccccc5-c5ccccc5-c5ccc6c(c5)c5ccccc5n6-c5ccccc5)ccc43)c2)cc1]

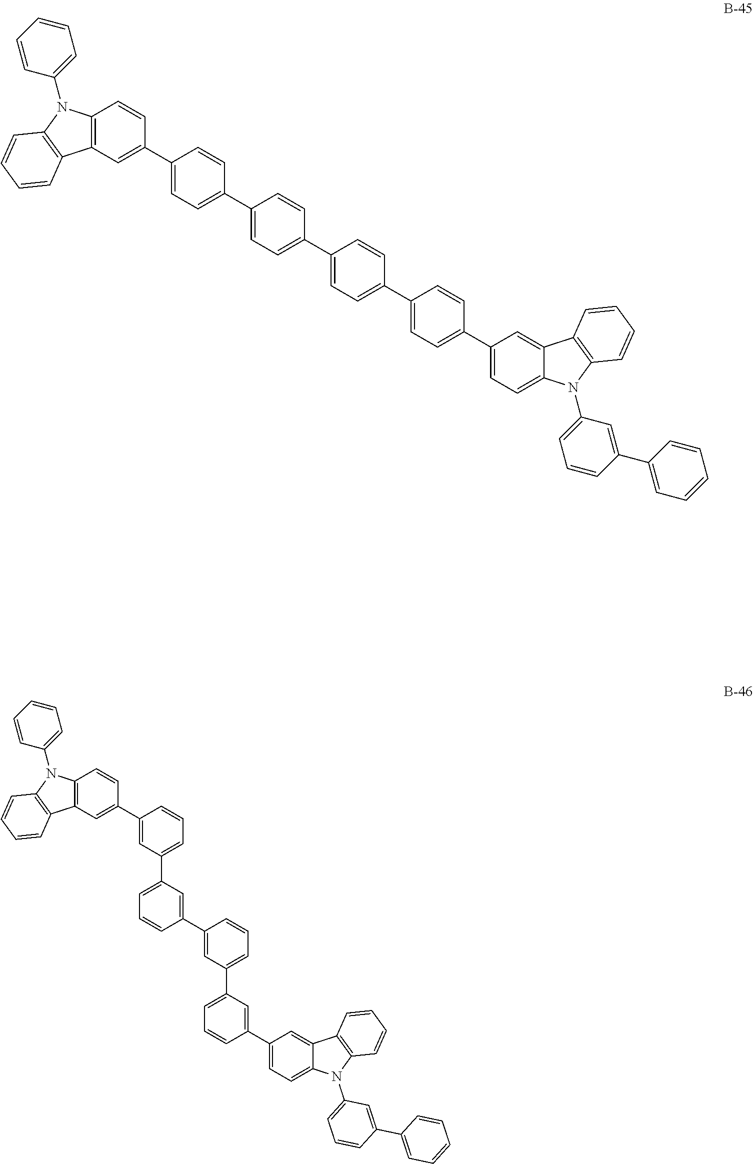 [SMILES: c1ccc(-c2cccc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)c2)cc1 ; c1ccc(-c2cccc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)c2)cc1]

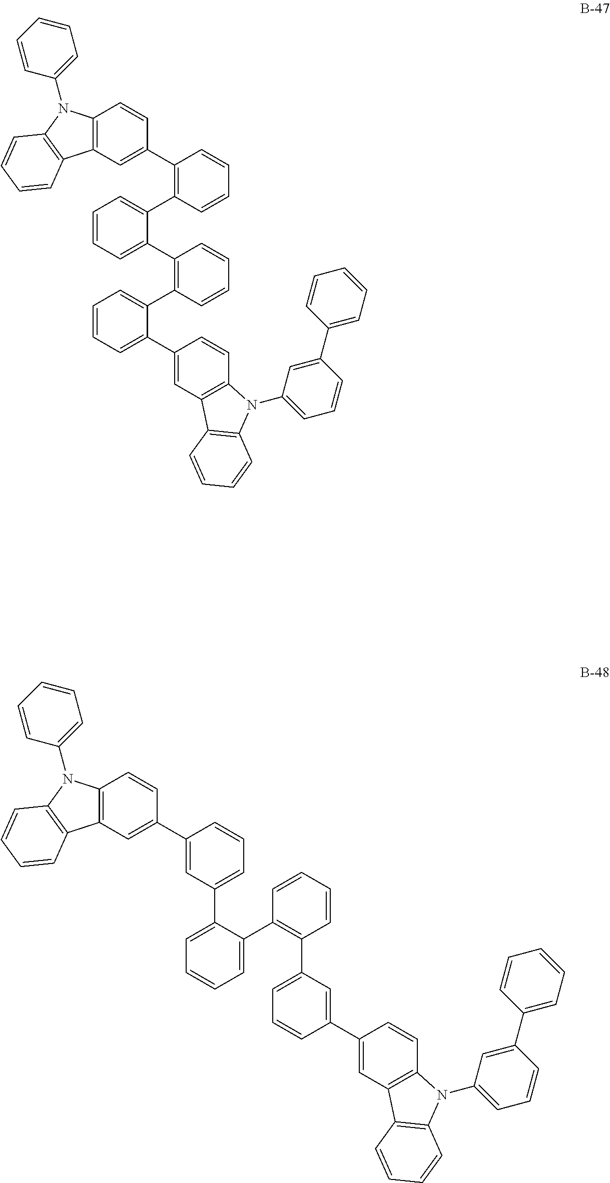 [SMILES: c1ccc(-c2cccc(-n3c4ccccc4c4cc(-c5ccccc5-c5ccccc5-c5ccccc5-c5ccccc5-c5ccc6c(c5)c5ccccc5n6-c5ccccc5)ccc43)c2)cc1 ; c1ccc(-c2cccc(-n3c4ccccc4c4cc(-c5cccc(-c6ccccc6-c6ccccc6-c6cccc(-c7ccc8c(c7)c7ccccc7n8-c7ccccc7)c6)c5)ccc43)c2)cc1]

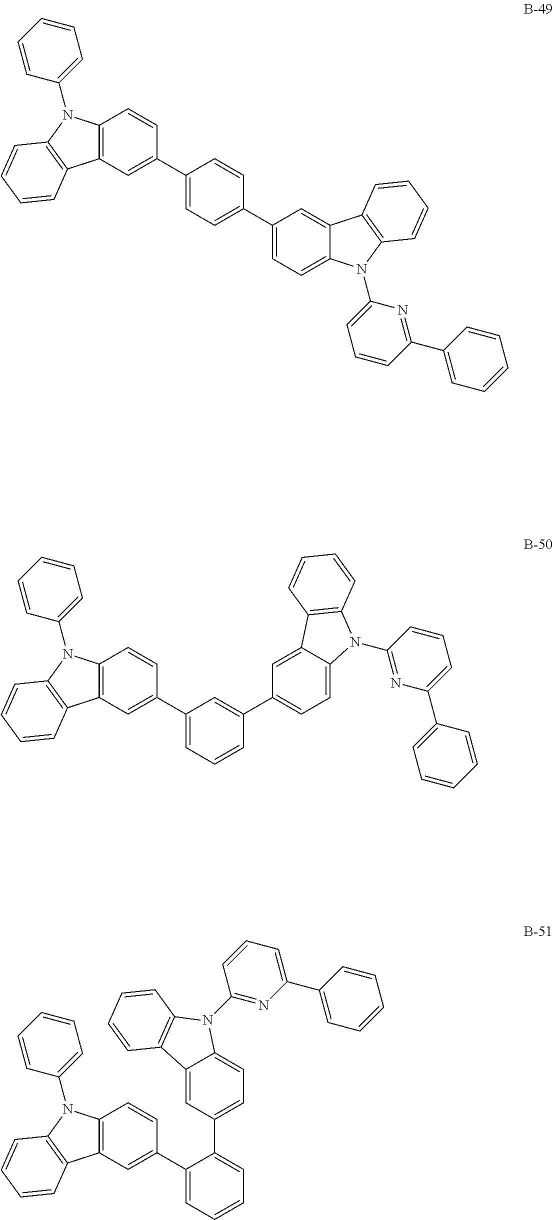 [SMILES: c1ccc(-c2cccc(-n3c4ccccc4c4cc(-c5ccc(-c6ccc7c(c6)c6ccccc6n7-c6ccccc6)cc5)ccc43)n2)cc1 ; c1ccc(-c2cccc(-n3c4ccccc4c4cc(-c5cccc(-c6ccc7c(c6)c6ccccc6n7-c6ccccc6)c5)ccc43)n2)cc1 ; c1ccc(-c2cccc(-n3c4ccccc4c4cc(-c5ccccc5-c5ccc6c(c5)c5ccccc5n6-c5ccccc5)ccc43)n2)cc1]

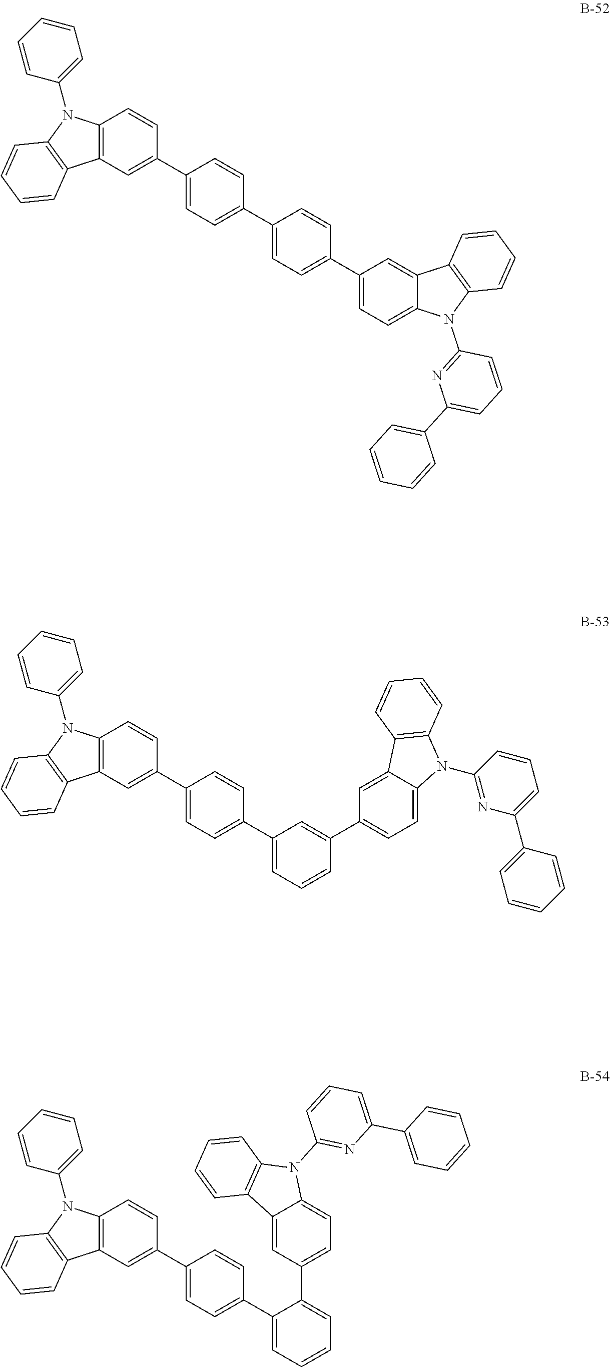 [SMILES: c1ccc(-c2cccc(-n3c4ccccc4c4cc(-c5ccc(-c6ccc(-c7ccc8c(c7)c7ccccc7n8-c7ccccc7)cc6)cc5)ccc43)n2)cc1 ; c1ccc(-c2cccc(-n3c4ccccc4c4cc(-c5cccc(-c6ccc(-c7ccc8c(c7)c7ccccc7n8-c7ccccc7)cc6)c5)ccc43)n2)cc1 ; c1ccc(-c2cccc(-n3c4ccccc4c4cc(-c5ccccc5-c5ccc(-c6ccc7c(c6)c6ccccc6n7-c6ccccc6)cc5)ccc43)n2)cc1]

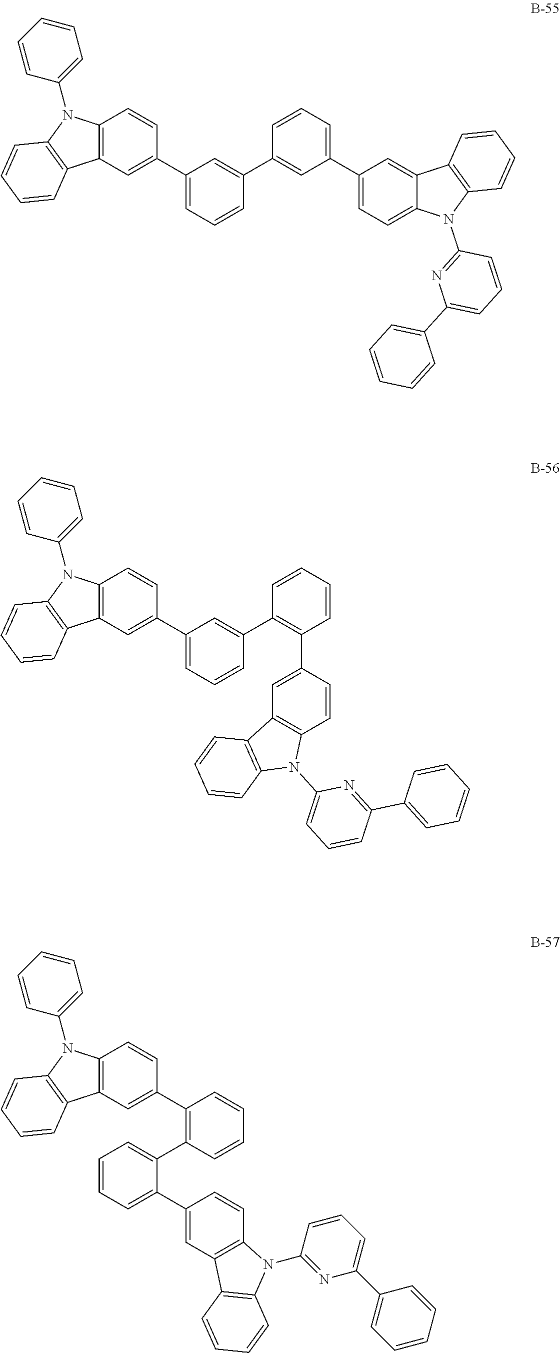 [SMILES: c1ccc(-c2cccc(-n3c4ccccc4c4cc(-c5cccc(-c6cccc(-c7ccc8c(c7)c7ccccc7n8-c7ccccc7)c6)c5)ccc43)n2)cc1 ; c1ccc(-c2cccc(-n3c4ccccc4c4cc(-c5ccccc5-c5cccc(-c6ccc7c(c6)c6ccccc6n7-c6ccccc6)c5)ccc43)n2)cc1 ; c1ccc(-c2cccc(-n3c4ccccc4c4cc(-c5ccccc5-c5ccccc5-c5ccc6c(c5)c5ccccc5n6-c5ccccc5)ccc43)n2)cc1]

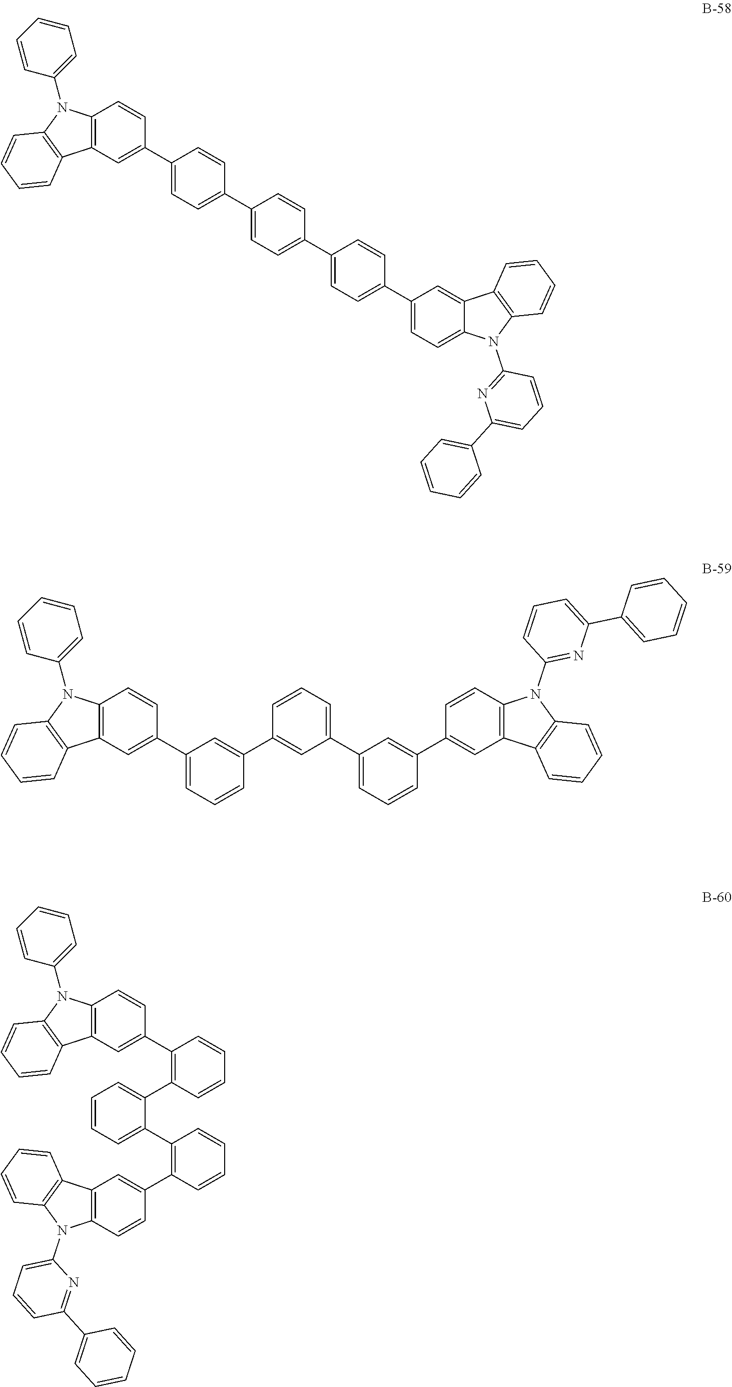 [SMILES: c1ccc(-c2cccc(-n3c4ccccc4c4cc(-c5ccc(-c6ccc(-c7ccc(-c8ccc9c(c8)c8ccccc8n9-c8ccccc8)cc7)cc6)cc5)ccc43)n2)cc1 ; c1ccc(-c2cccc(-n3c4ccccc4c4cc(-c5cccc(-c6cccc(-c7cccc(-c8ccc9c(c8)c8ccccc8n9-c8ccccc8)c7)c6)c5)ccc43)n2)cc1 ; c1ccc(-c2cccc(-n3c4ccccc4c4cc(-c5ccccc5-c5ccccc5-c5ccccc5-c5ccc6c(c5)c5ccccc5n6-c5ccccc5)ccc43)n2)cc1]

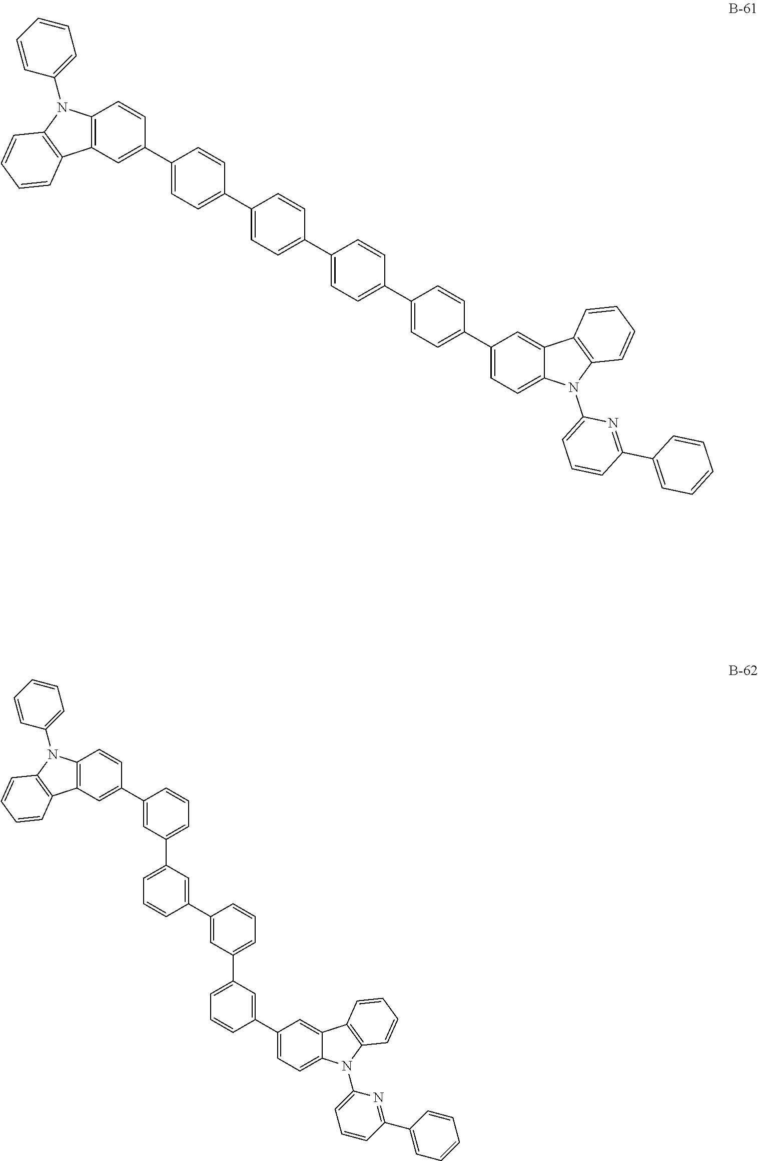 [SMILES: c1ccc(-c2cccc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)n2)cc1 ; c1ccc(-c2cccc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)n2)cc1]

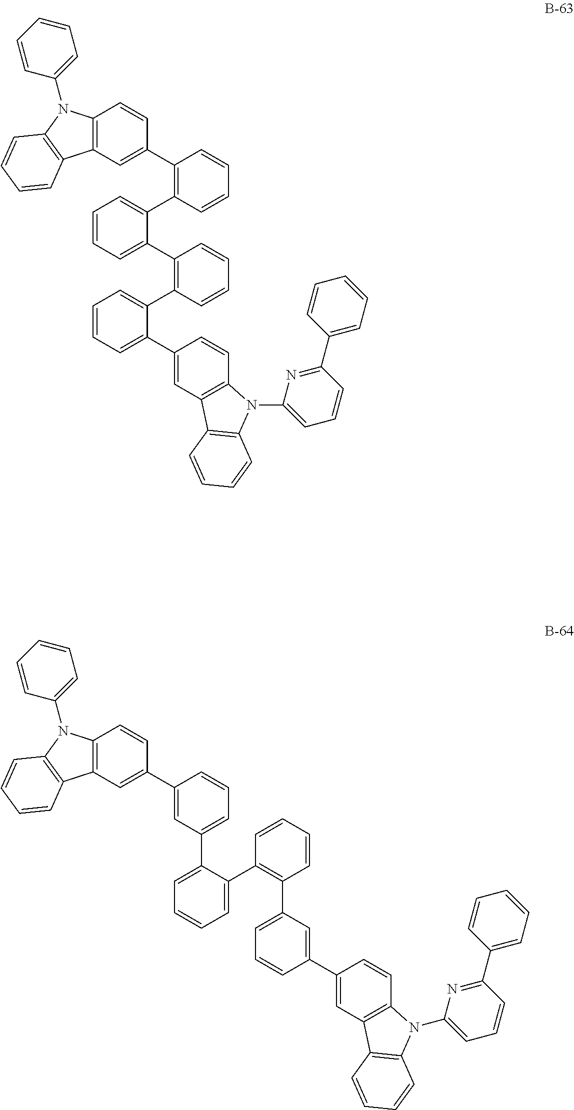 [SMILES: c1ccc(-c2cccc(-n3c4ccccc4c4cc(-c5ccccc5-c5ccccc5-c5ccccc5-c5ccccc5-c5ccc6c(c5)c5ccccc5n6-c5ccccc5)ccc43)n2)cc1 ; c1ccc(-c2cccc(-n3c4ccccc4c4cc(-c5cccc(-c6ccccc6-c6ccccc6-c6cccc(-c7ccc8c(c7)c7ccccc7n8-c7ccccc7)c6)c5)ccc43)n2)cc1]

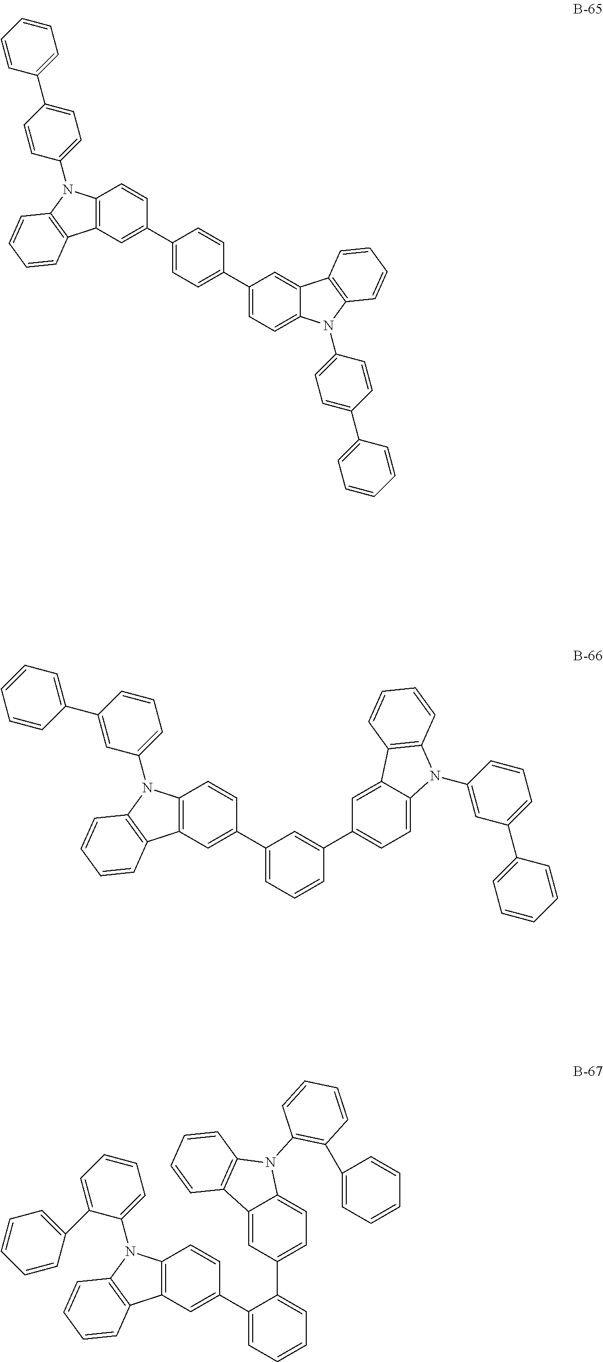 [SMILES: c1ccc(-c2ccc(-n3c4ccccc4c4cc(-c5ccc(-c6ccc7c(c6)c6ccccc6n7-c6ccc(-c7ccccc7)cc6)cc5)ccc43)cc2)cc1 ; c1ccc(-c2cccc(-n3c4ccccc4c4cc(-c5cccc(-c6ccc7c(c6)c6ccccc6n7-c6cccc(-c7ccccc7)c6)c5)ccc43)c2)cc1 ; c1ccc(-c2ccccc2-n2c3ccccc3c3cc(-c4ccccc4-c4ccc5c(c4)c4ccccc4n5-c4ccccc4-c4ccccc4)ccc32)cc1]

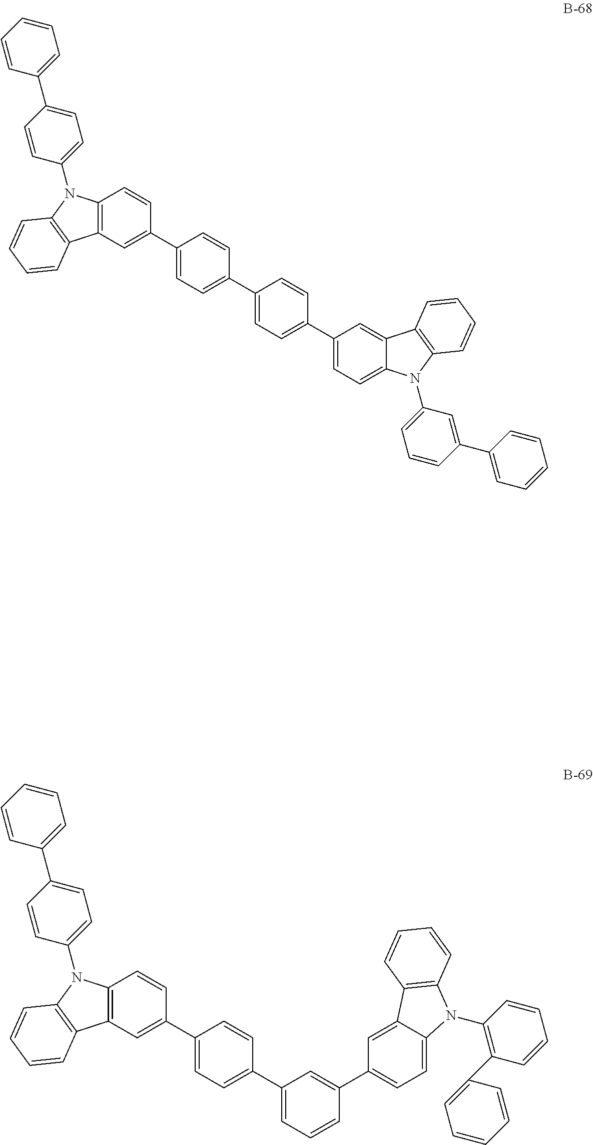 [SMILES: c1ccc(-c2ccc(-n3c4ccccc4c4cc(-c5ccc(-c6ccc(-c7ccc8c(c7)c7ccccc7n8-c7cccc(-c8ccccc8)c7)cc6)cc5)ccc43)cc2)cc1 ; c1ccc(-c2ccc(-n3c4ccccc4c4cc(-c5ccc(-c6cccc(-c7ccc8c(c7)c7ccccc7n8-c7ccccc7-c7ccccc7)c6)cc5)ccc43)cc2)cc1]

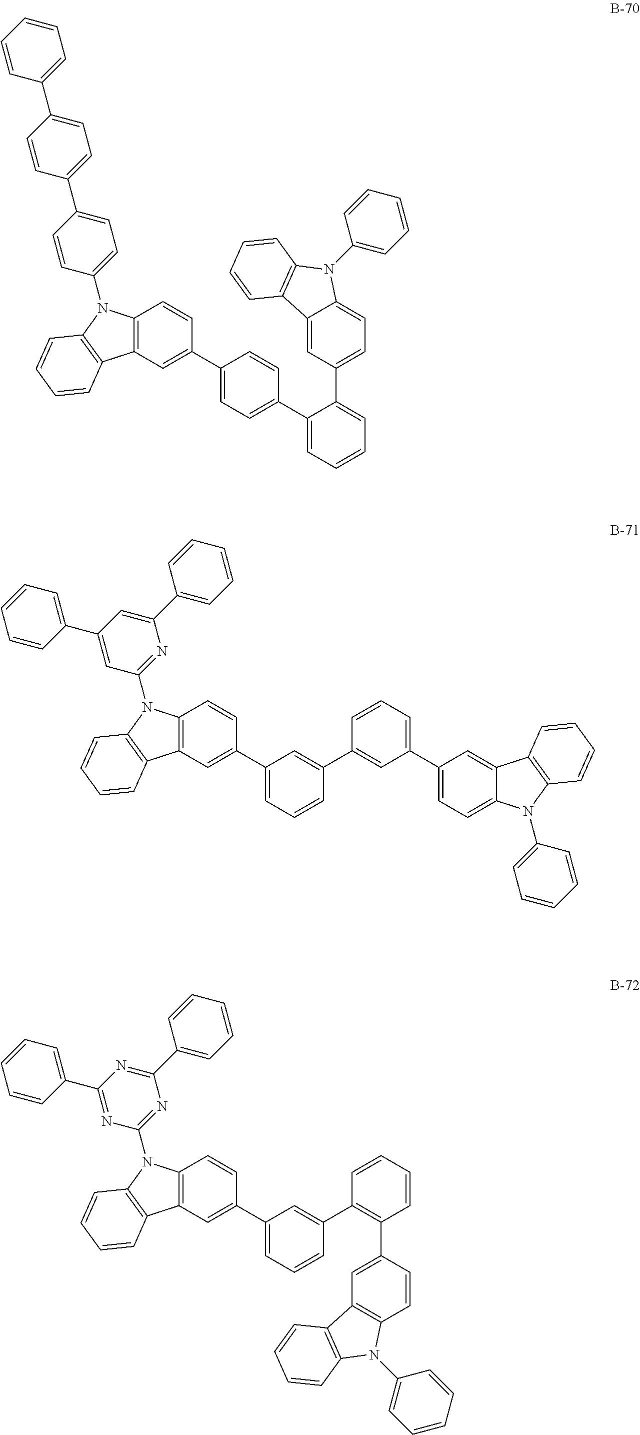 [SMILES: c1ccc(-c2ccc(-c3ccc(-n4c5ccccc5c5cc(-c6ccc(-c7ccccc7-c7ccc8c(c7)c7ccccc7n8-c7ccccc7)cc6)ccc54)cc3)cc2)cc1 ; c1ccc(-c2cc(-c3ccccc3)nc(-n3c4ccccc4c4cc(-c5cccc(-c6cccc(-c7ccc8c(c7)c7ccccc7n8-c7ccccc7)c6)c5)ccc43)c2)cc1 ; c1ccc(-c2nc(-c3ccccc3)nc(-n3c4ccccc4c4cc(-c5cccc(-c6ccccc6-c6ccc7c(c6)c6ccccc6n7-c6ccccc6)c5)ccc43)n2)cc1]

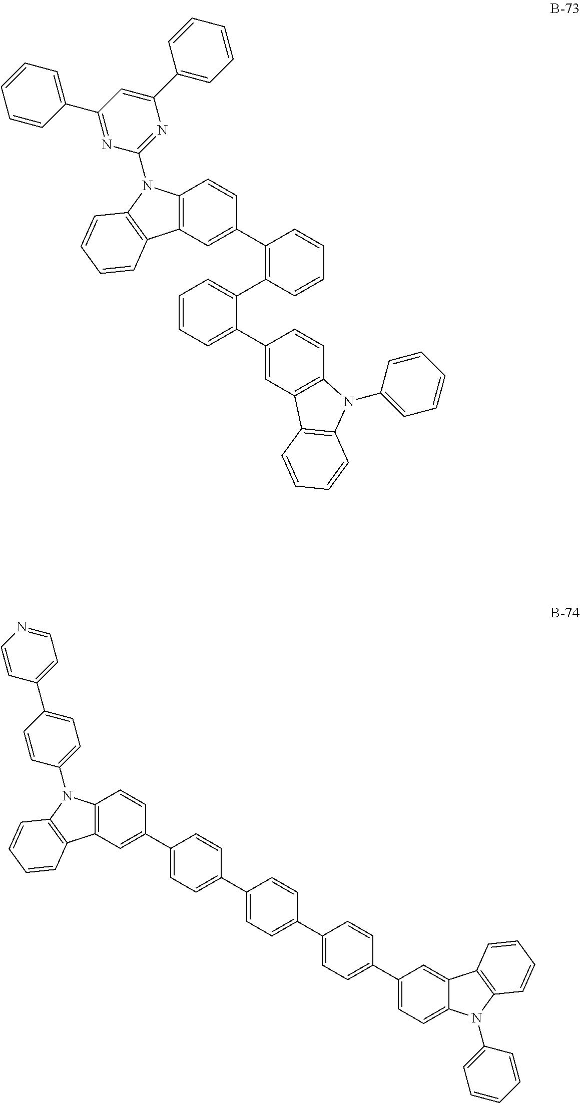 [SMILES: c1ccc(-c2cc(-c3ccccc3)nc(-n3c4ccccc4c4cc(-c5ccccc5-c5ccccc5-c5ccc6c(c5)c5ccccc5n6-c5ccccc5)ccc43)n2)cc1 ; c1ccc(-n2c3ccccc3c3cc(-c4ccc(-c5ccc(-c6ccc(-c7ccc8c(c7)c7ccccc7n8-c7ccc(-c8ccncc8)cc7)cc6)cc5)cc4)ccc32)cc1]

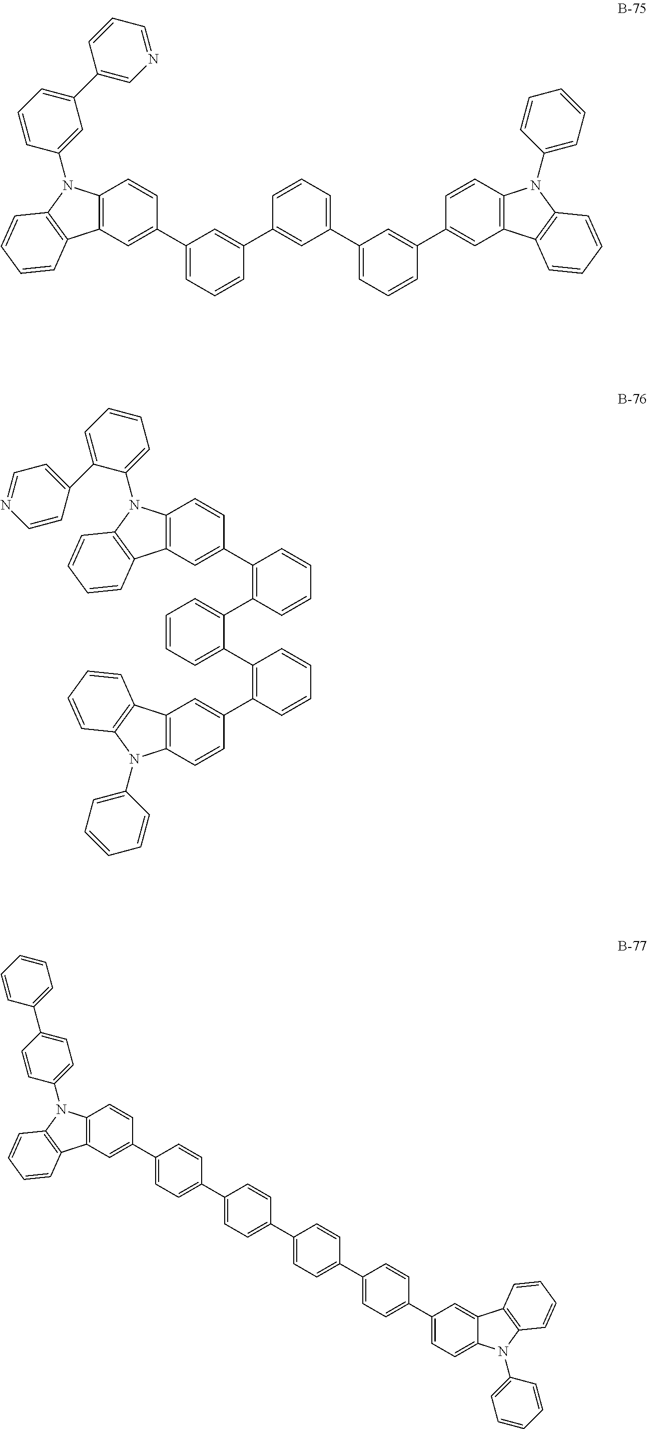 [SMILES: c1ccc(-n2c3ccccc3c3cc(-c4cccc(-c5cccc(-c6cccc(-c7ccc8c(c7)c7ccccc7n8-c7cccc(-c8cccnc8)c7)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4ccccc4-c4ccncc4)ccc32)cc1 ; c1ccc(-c2ccc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)cc2)cc1]

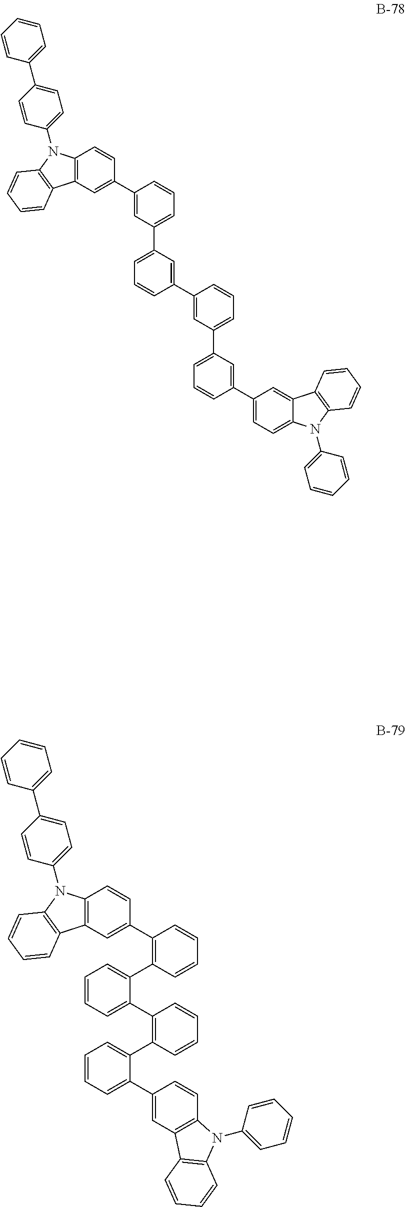 [SMILES: c1ccc(-c2ccc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)cc2)cc1 ; c1ccc(-c2ccc(-n3c4ccccc4c4cc(-c5ccccc5-c5ccccc5-c5ccccc5-c5ccccc5-c5ccc6c(c5)c5ccccc5n6-c5ccccc5)ccc43)cc2)cc1]

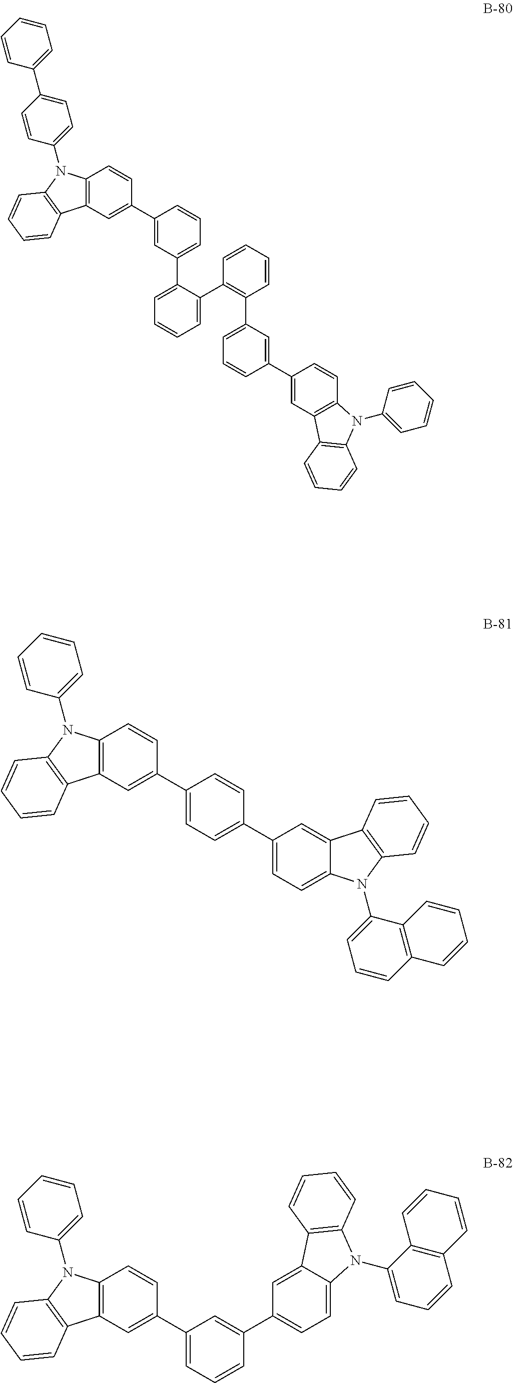 [SMILES: c1ccc(-c2ccc(-n3c4ccccc4c4cc(-c5cccc(-c6ccccc6-c6ccccc6-c6cccc(-c7ccc8c(c7)c7ccccc7n8-c7ccccc7)c6)c5)ccc43)cc2)cc1 ; c1ccc(-n2c3ccccc3c3cc(-c4ccc(-c5ccc6c(c5)c5ccccc5n6-c5cccc6ccccc56)cc4)ccc32)cc1 ; c1ccc(-n2c3ccccc3c3cc(-c4cccc(-c5ccc6c(c5)c5ccccc5n6-c5cccc6ccccc56)c4)ccc32)cc1]

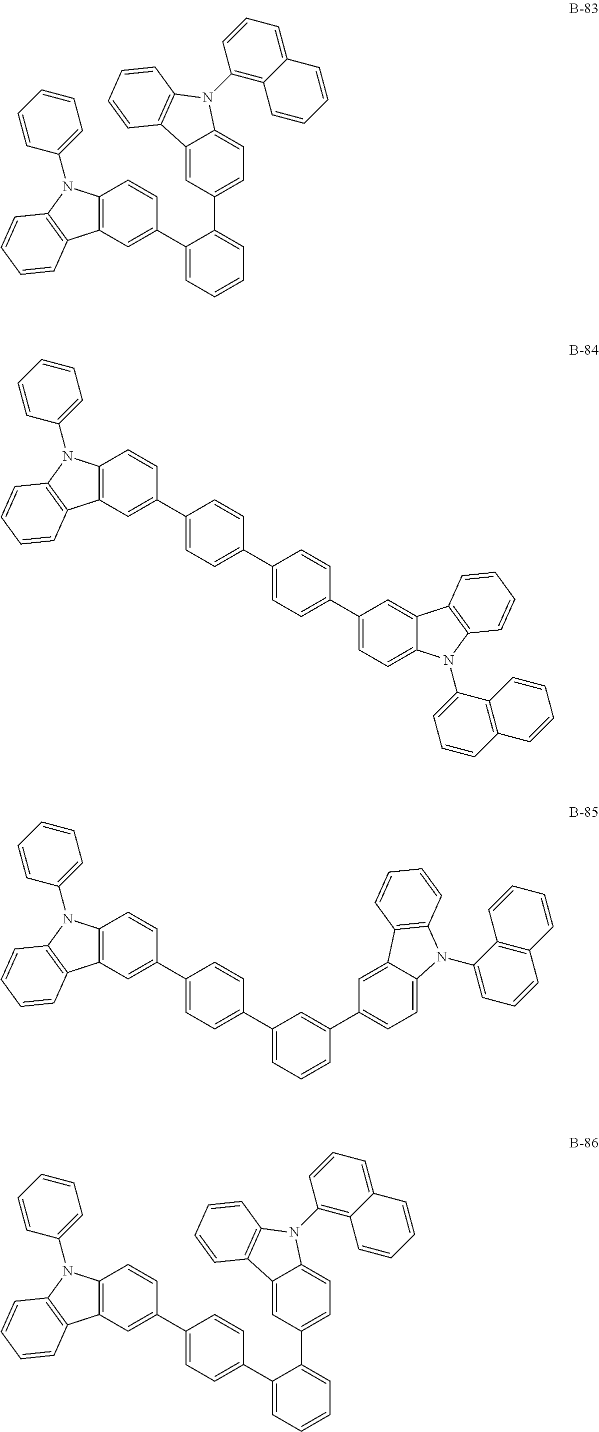 [SMILES: c1ccc(-n2c3ccccc3c3cc(-c4ccccc4-c4ccc5c(c4)c4ccccc4n5-c4cccc5ccccc45)ccc32)cc1 ; c1ccc(-n2c3ccccc3c3cc(-c4ccc(-c5ccc(-c6ccc7c(c6)c6ccccc6n7-c6cccc7ccccc67)cc5)cc4)ccc32)cc1 ; c1ccc(-n2c3ccccc3c3cc(-c4ccc(-c5cccc(-c6ccc7c(c6)c6ccccc6n7-c6cccc7ccccc67)c5)cc4)ccc32)cc1 ; c1ccc(-n2c3ccccc3c3cc(-c4ccc(-c5ccccc5-c5ccc6c(c5)c5ccccc5n6-c5cccc6ccccc56)cc4)ccc32)cc1]

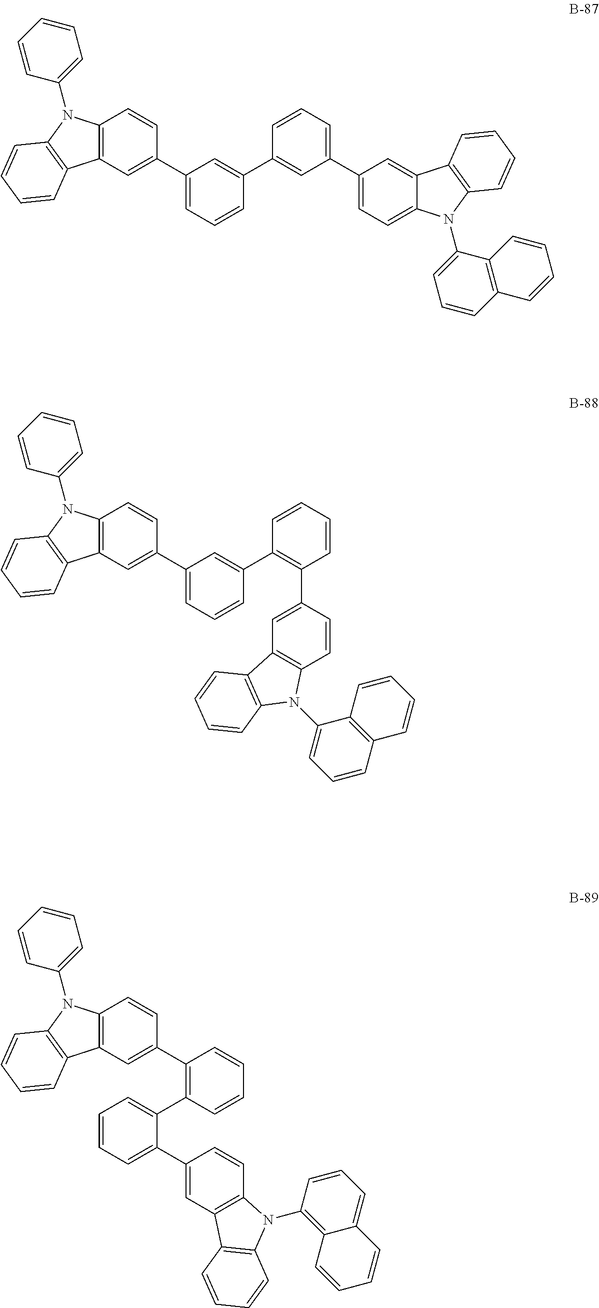 [SMILES: c1ccc(-n2c3ccccc3c3cc(-c4cccc(-c5cccc(-c6ccc7c(c6)c6ccccc6n7-c6cccc7ccccc67)c5)c4)ccc32)cc1 ; c1ccc(-n2c3ccccc3c3cc(-c4cccc(-c5ccccc5-c5ccc6c(c5)c5ccccc5n6-c5cccc6ccccc56)c4)ccc32)cc1 ; c1ccc(-n2c3ccccc3c3cc(-c4ccccc4-c4ccccc4-c4ccc5c(c4)c4ccccc4n5-c4cccc5ccccc45)ccc32)cc1]

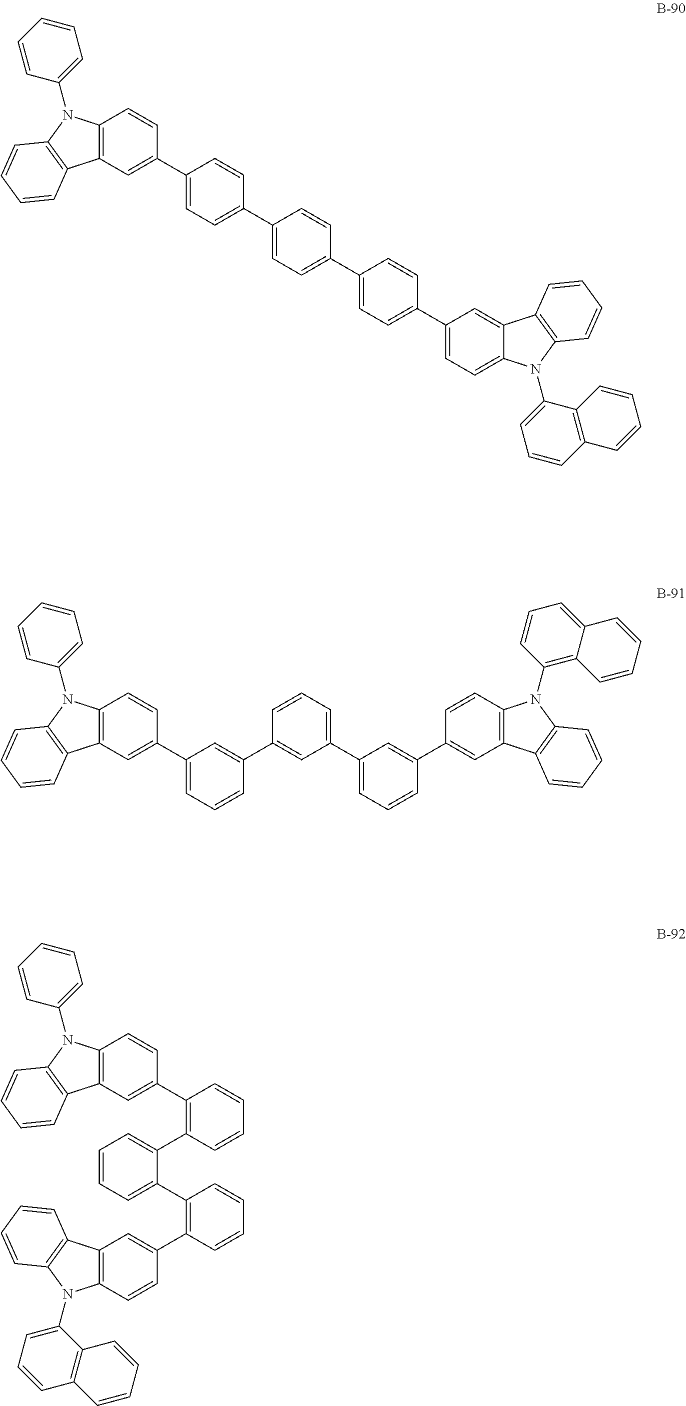 [SMILES: c1ccc(-n2c3ccccc3c3cc(-c4ccc(-c5ccc(-c6ccc(-c7ccc8c(c7)c7ccccc7n8-c7cccc8ccccc78)cc6)cc5)cc4)ccc32)cc1 ; c1ccc(-n2c3ccccc3c3cc(-c4cccc(-c5cccc(-c6cccc(-c7ccc8c(c7)c7ccccc7n8-c7cccc8ccccc78)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4cccc5ccccc45)ccc32)cc1]

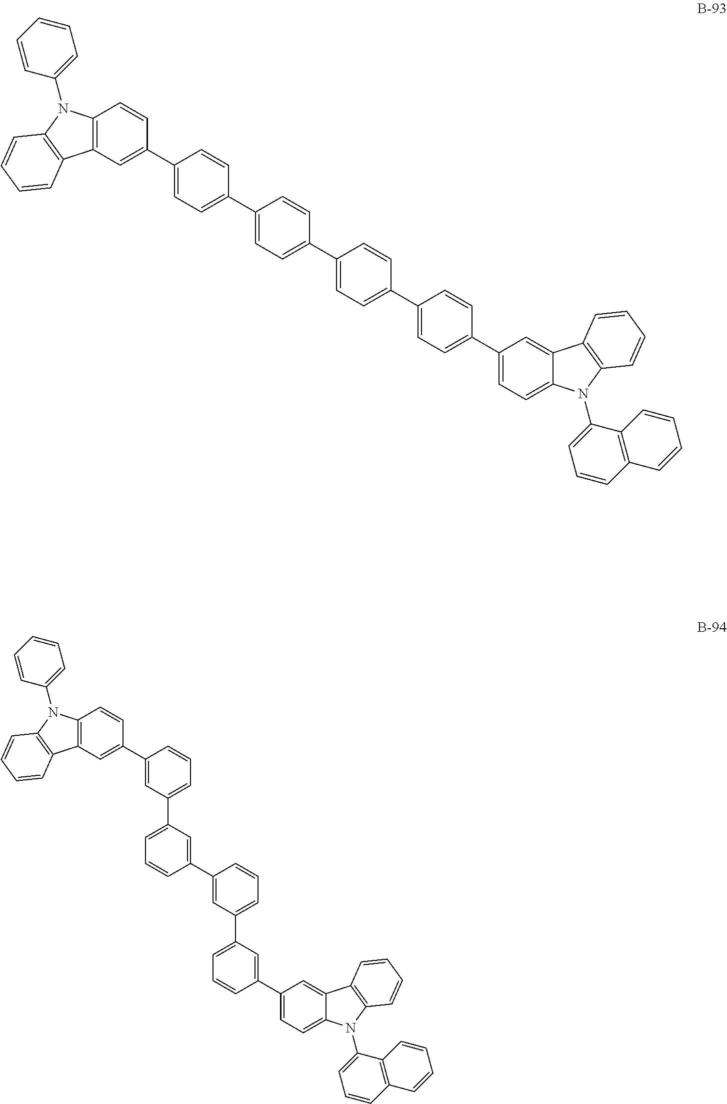 [SMILES: c1ccc(-n2c3ccccc3c3cc(-c4ccc(-c5ccc(-c6ccc(-c7ccc(-c8ccc9c(c8)c8ccccc8n9-c8cccc9ccccc89)cc7)cc6)cc5)cc4)ccc32)cc1 ; c1ccc(-n2c3ccccc3c3cc(-c4cccc(-c5cccc(-c6cccc(-c7cccc(-c8ccc9c(c8)c8ccccc8n9-c8cccc9ccccc89)c7)c6)c5)c4)ccc32)cc1]

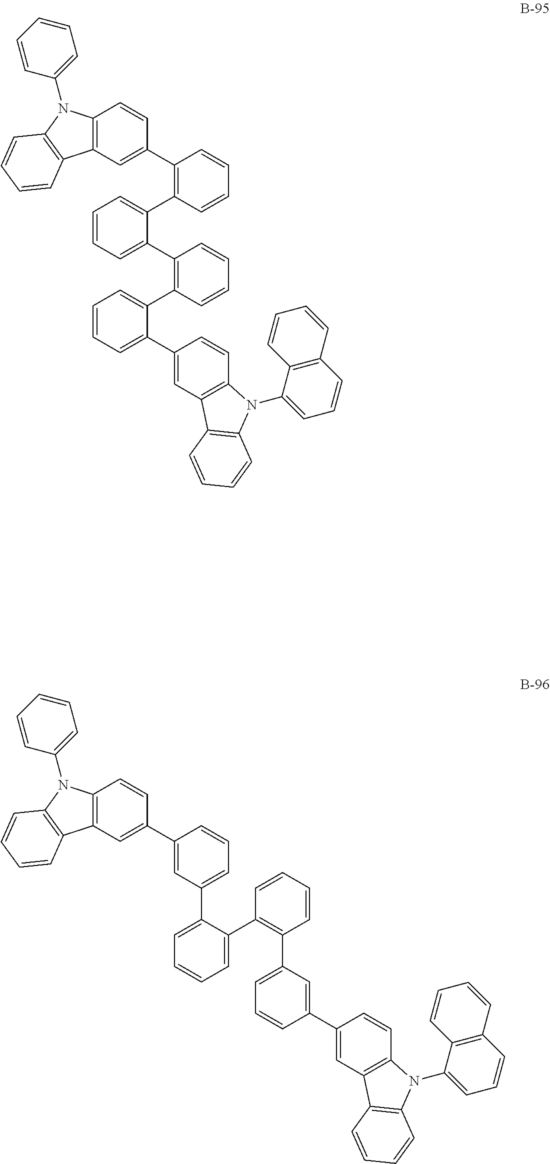 [SMILES: c1ccc(-n2c3ccccc3c3cc(-c4ccccc4-c4ccccc4-c4ccccc4-c4ccccc4-c4ccc5c(c4)c4ccccc4n5-c4cccc5ccccc45)ccc32)cc1 ; c1ccc(-n2c3ccccc3c3cc(-c4cccc(-c5ccccc5-c5ccccc5-c5cccc(-c6ccc7c(c6)c6ccccc6n7-c6cccc7ccccc67)c5)c4)ccc32)cc1]

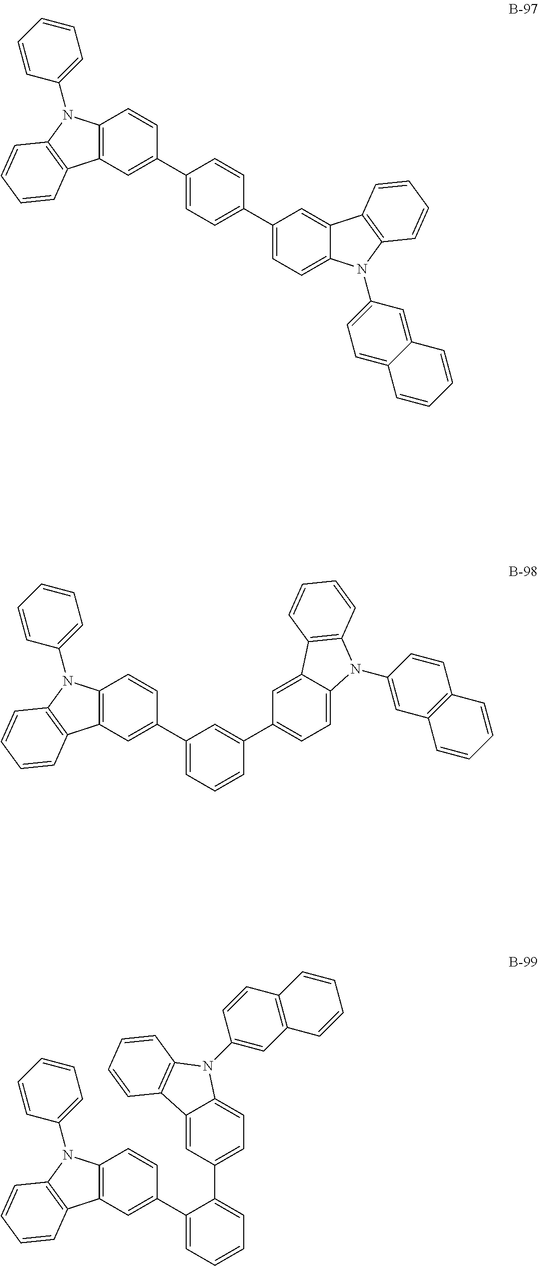 [SMILES: c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4cccc(-c5ccc6c(c5)c5ccccc5n6-c5ccc6ccccc6c5)c4)ccc32)cc1 ; c1ccc(-n2c3ccccc3c3cc(-c4ccccc4-c4ccc5c(c4)c4ccccc4n5-c4ccc5ccccc5c4)ccc32)cc1]

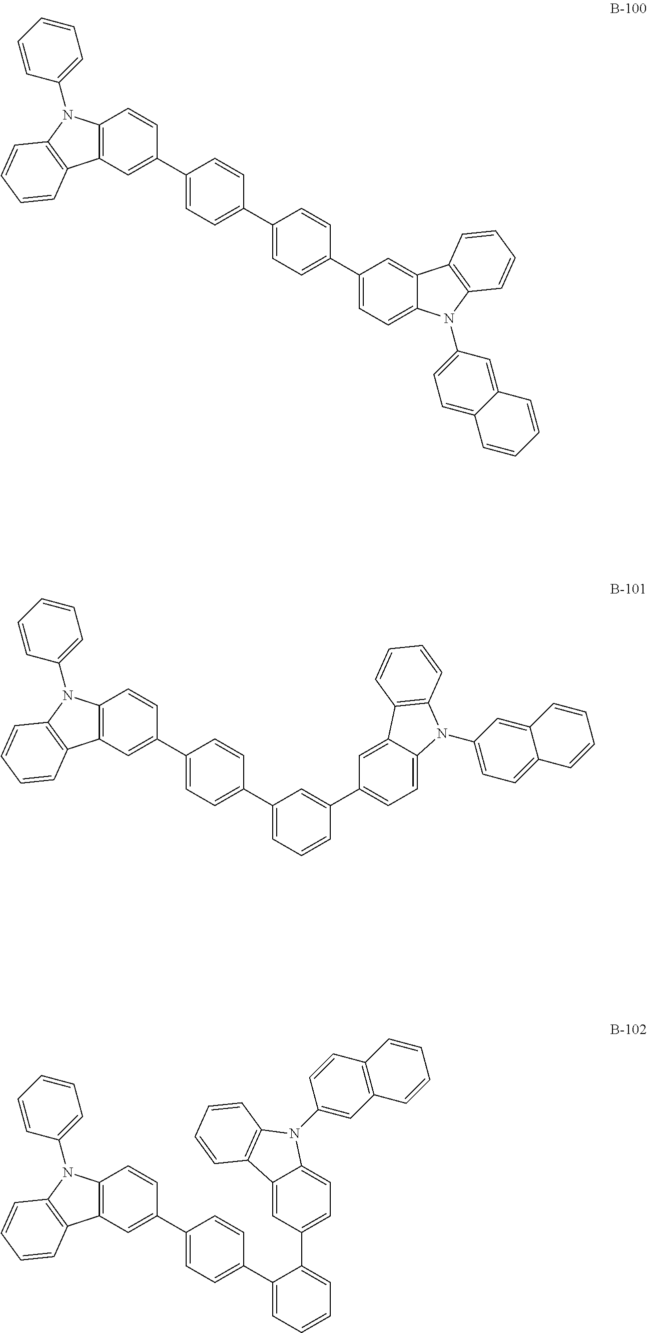 [SMILES: c1ccc(-n2c3ccccc3c3cc(-c4ccc(-c5ccc(-c6ccc7c(c6)c6ccccc6n7-c6ccc7ccccc7c6)cc5)cc4)ccc32)cc1 ; c1ccc(-n2c3ccccc3c3cc(-c4ccc(-c5cccc(-c6ccc7c(c6)c6ccccc6n7-c6ccc7ccccc7c6)c5)cc4)ccc32)cc1 ; c1ccc(-n2c3ccccc3c3cc(-c4ccc(-c5ccccc5-c5ccc6c(c5)c5ccccc5n6-c5ccc6ccccc6c5)cc4)ccc32)cc1]

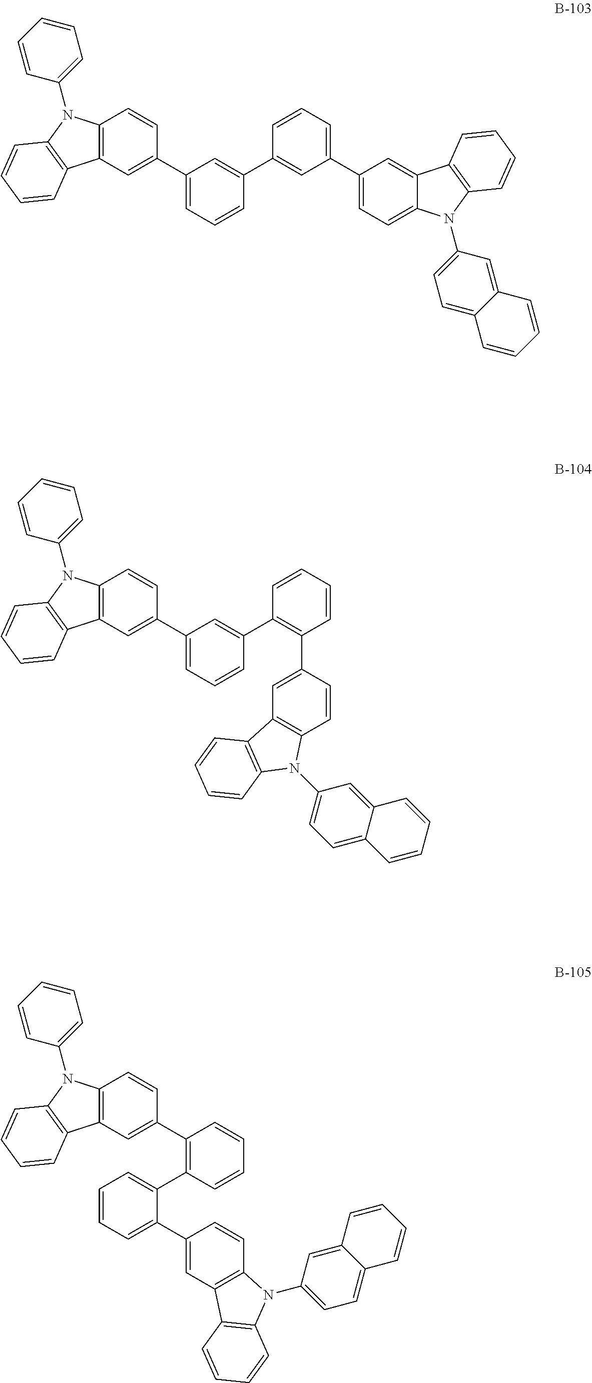 [SMILES: c1ccc(-n2c3ccccc3c3cc(-c4cccc(-c5cccc(-c6ccc7c(c6)c6ccccc6n7-c6ccc7ccccc7c6)c5)c4)ccc32)cc1 ; c1ccc(-n2c3ccccc3c3cc(-c4cccc(-c5ccccc5-c5ccc6c(c5)c5ccccc5n6-c5ccc6ccccc6c5)c4)ccc32)cc1 ; c1ccc(-n2c3ccccc3c3cc(-c4ccccc4-c4ccccc4-c4ccc5c(c4)c4ccccc4n5-c4ccc5ccccc5c4)ccc32)cc1]

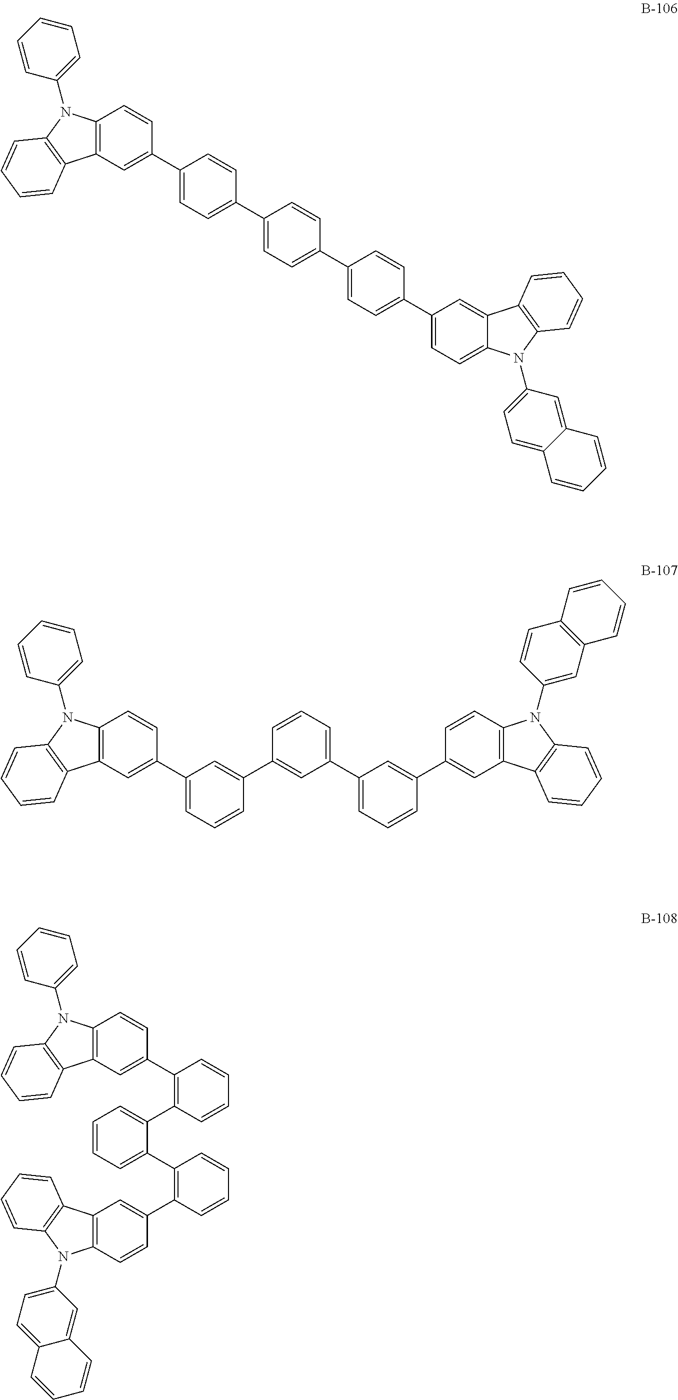 [SMILES: c1ccc(-n2c3ccccc3c3cc(-c4ccc(-c5ccc(-c6ccc(-c7ccc8c(c7)c7ccccc7n8-c7ccc8ccccc8c7)cc6)cc5)cc4)ccc32)cc1 ; c1ccc(-n2c3ccccc3c3cc(-c4cccc(-c5cccc(-c6cccc(-c7ccc8c(c7)c7ccccc7n8-c7ccc8ccccc8c7)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4ccc5ccccc5c4)ccc32)cc1]

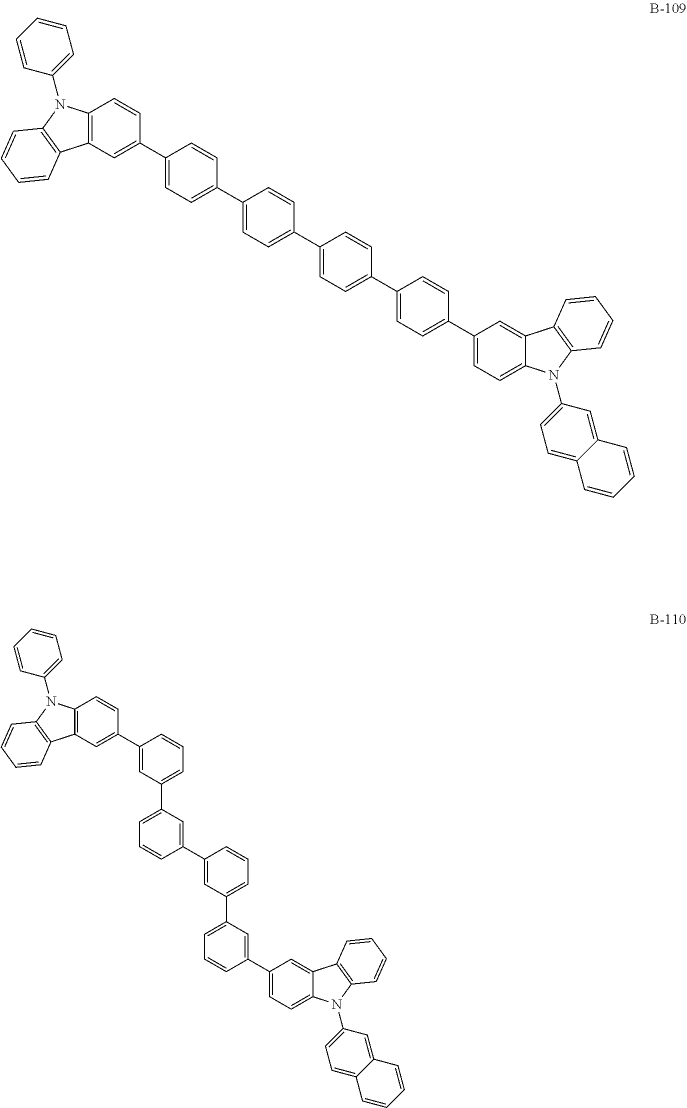 [SMILES: c1ccc(-n2c3ccccc3c3cc(-c4ccc(-c5ccc(-c6ccc(-c7ccc(-c8ccc9c(c8)c8ccccc8n9-c8ccc9ccccc9c8)cc7)cc6)cc5)cc4)ccc32)cc1 ; c1ccc(-n2c3ccccc3c3cc(-c4cccc(-c5cccc(-c6cccc(-c7cccc(-c8ccc9c(c8)c8ccccc8n9-c8ccc9ccccc9c8)c7)c6)c5)c4)ccc32)cc1]

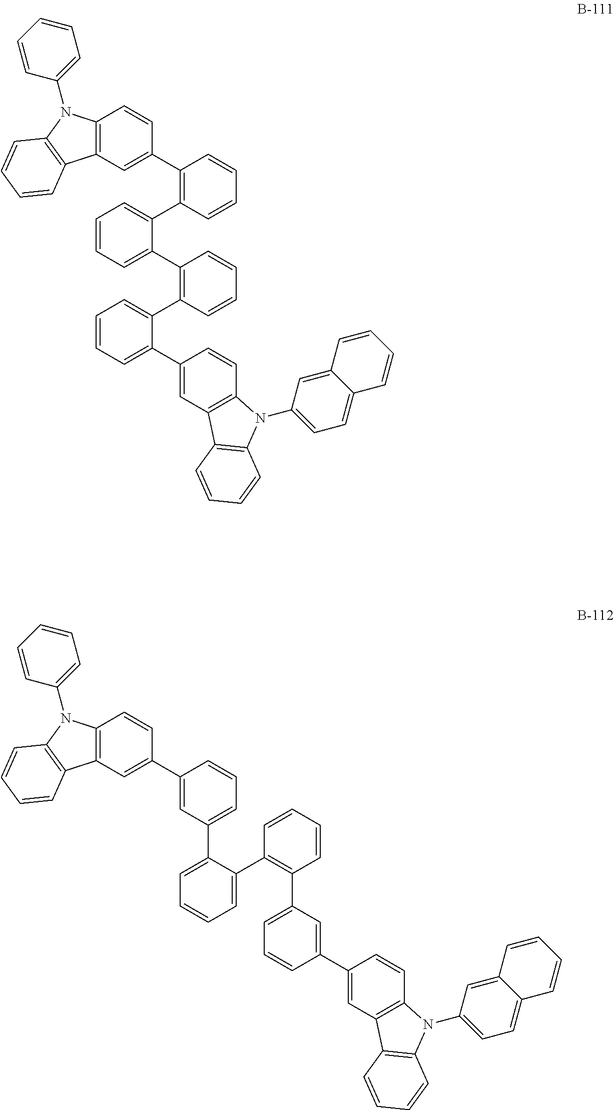 [SMILES: c1ccc(-n2c3ccccc3c3cc(-c4ccccc4-c4ccccc4-c4ccccc4-c4ccccc4-c4ccc5c(c4)c4ccccc4n5-c4ccc5ccccc5c4)ccc32)cc1 ; c1ccc(-n2c3ccccc3c3cc(-c4cccc(-c5ccccc5-c5ccccc5-c5cccc(-c6ccc7c(c6)c6ccccc6n7-c6ccc7ccccc7c6)c5)c4)ccc32)cc1]

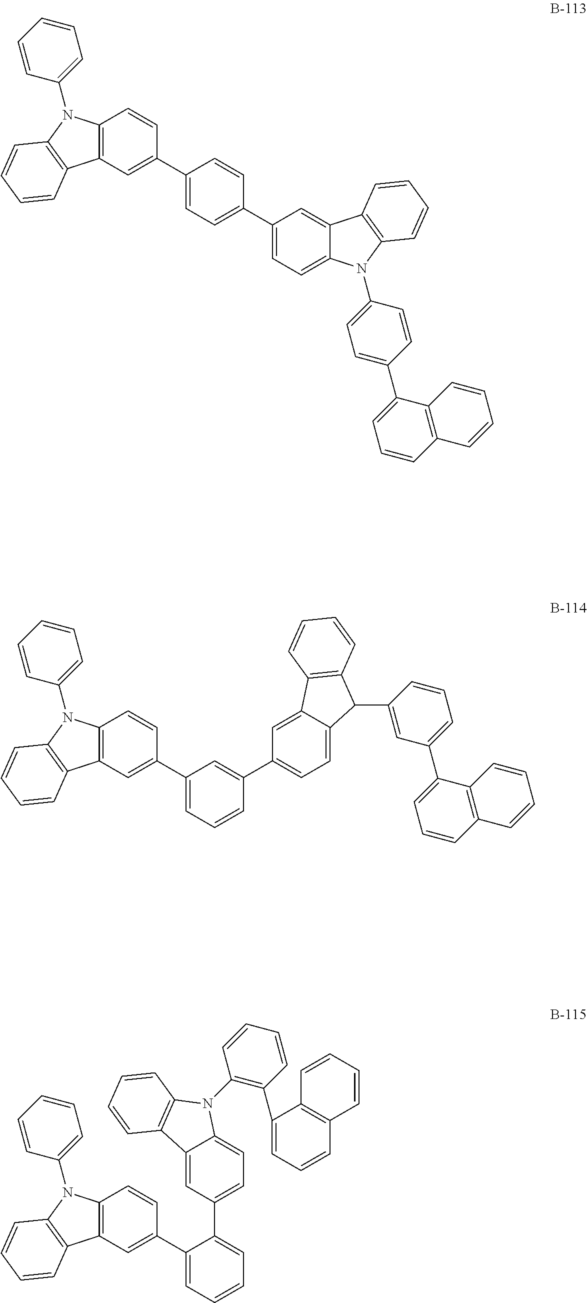 [SMILES: c1ccc(-n2c3ccccc3c3cc(-c4ccc(-c5ccc6c(c5)c5ccccc5n6-c5ccc(-c6cccc7ccccc67)cc5)cc4)ccc32)cc1 ; c1ccc(-n2c3ccccc3c3cc(-c4cccc(-c5ccc6c(c5)-c5ccccc5C6c5cccc(-c6cccc7ccccc67)c5)c4)ccc32)cc1 ; c1ccc(-n2c3ccccc3c3cc(-c4ccccc4-c4ccc5c(c4)c4ccccc4n5-c4ccccc4-c4cccc5ccccc45)ccc32)cc1]

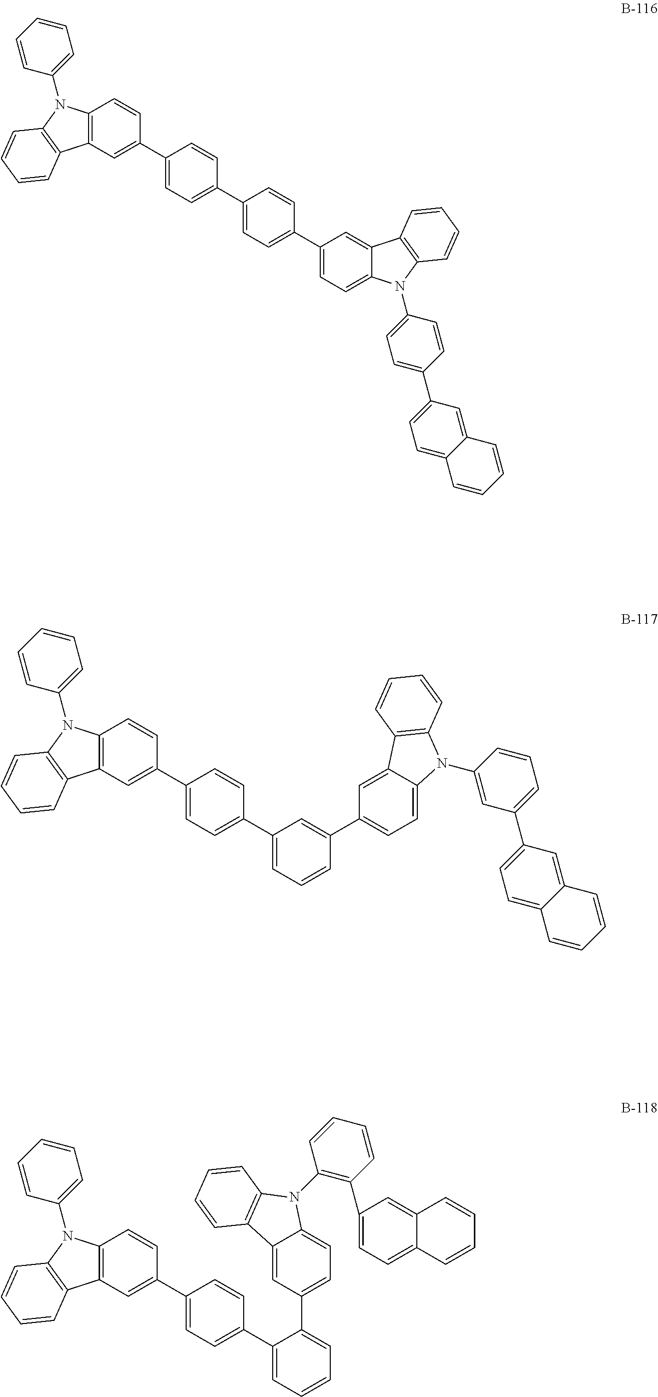 [SMILES: c1ccc(-n2c3ccccc3c3cc(-c4ccc(-c5ccc(-c6ccc7c(c6)c6ccccc6n7-c6ccc(-c7ccc8ccccc8c7)cc6)cc5)cc4)ccc32)cc1 ; c1ccc(-n2c3ccccc3c3cc(-c4ccc(-c5cccc(-c6ccc7c(c6)c6ccccc6n7-c6cccc(-c7ccc8ccccc8c7)c6)c5)cc4)ccc32)cc1 ; c1ccc(-n2c3ccccc3c3cc(-c4ccc(-c5ccccc5-c5ccc6c(c5)c5ccccc5n6-c5ccccc5-c5ccc6ccccc6c5)cc4)ccc32)cc1]

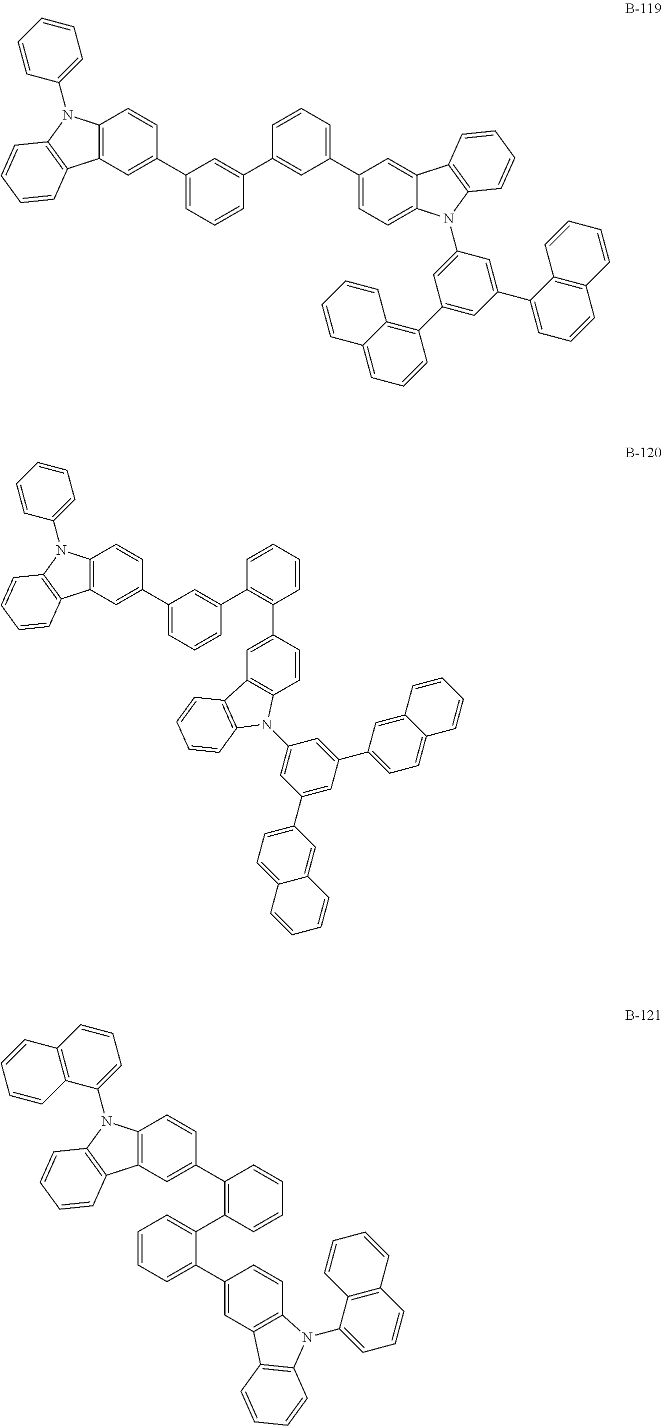 [SMILES: c1ccc(-n2c3ccccc3c3cc(-c4cccc(-c5cccc(-c6ccc7c(c6)c6ccccc6n7-c6cc(-c7cccc8ccccc78)cc(-c7cccc8ccccc78)c6)c5)c4)ccc32)cc1 ; c1ccc(-n2c3ccccc3c3cc(-c4cccc(-c5ccccc5-c5ccc6c(c5)c5ccccc5n6-c5cc(-c6ccc7ccccc7c6)cc(-c6ccc7ccccc7c6)c5)c4)ccc32)cc1 ; c1ccc(-c2ccccc2-c2ccc3c(c2)c2ccccc2n3-c2cccc3ccccc23)c(-c2ccc3c(c2)c2ccccc2n3-c2cccc3ccccc23)c1]

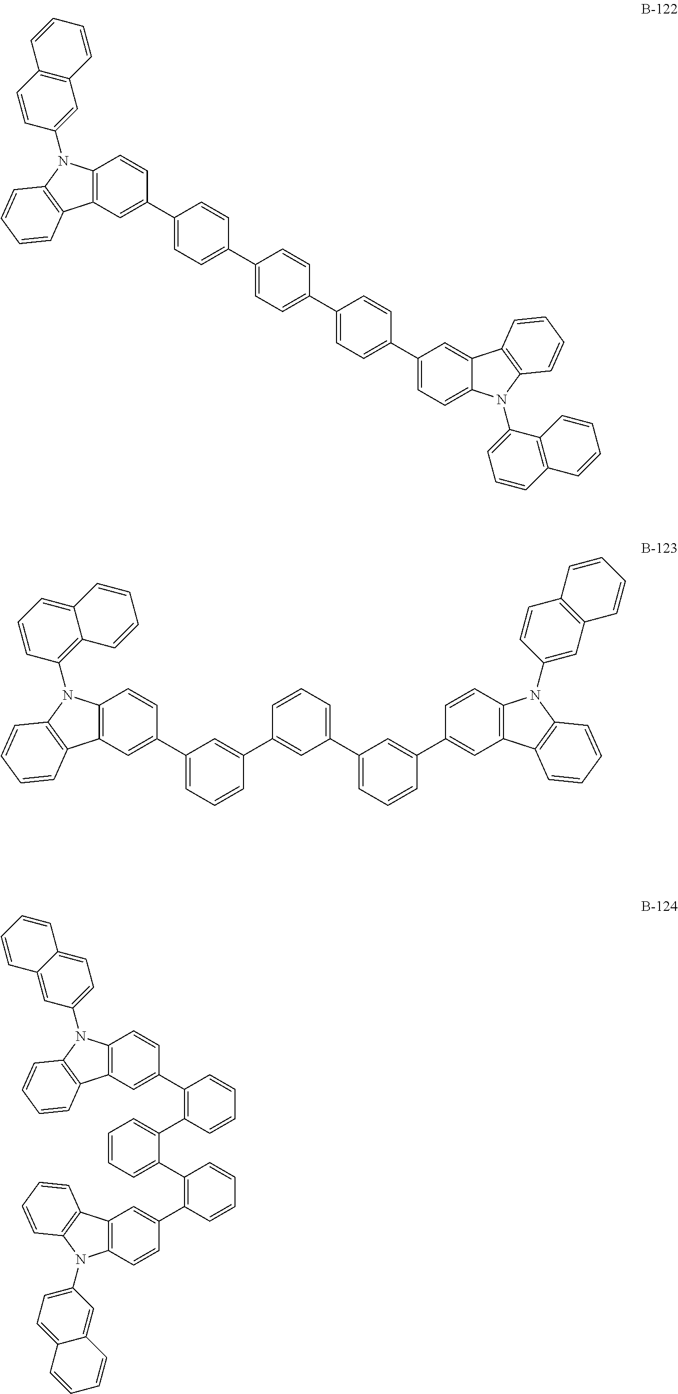 [SMILES: c1ccc2cc(-n3c4ccccc4c4cc(-c5ccc(-c6ccc(-c7ccc(-c8ccc9c(c8)c8ccccc8n9-c8cccc9ccccc89)cc7)cc6)cc5)ccc43)ccc2c1 ; c1cc(-c2cccc(-c3ccc4c(c3)c3ccccc3n4-c3ccc4ccccc4c3)c2)cc(-c2cccc(-c3ccc4c(c3)c3ccccc3n4-c3cccc4ccccc34)c2)c1 ; c1ccc(-c2ccccc2-c2ccccc2-c2ccc3c(c2)c2ccccc2n3-c2ccc3ccccc3c2)c(-c2ccc3c(c2)c2ccccc2n3-c2ccc3ccccc3c2)c1]

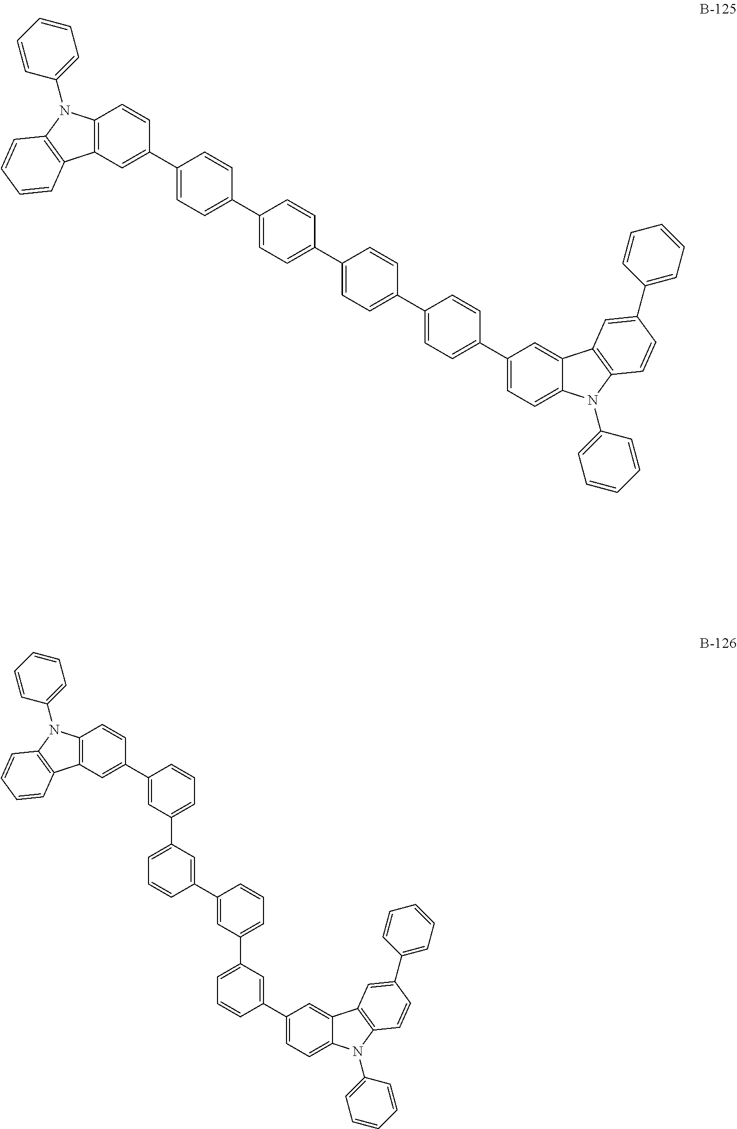 [SMILES: c1ccc(-c2ccc3c(c2)c2cc(-c4ccc(-c5ccc(-c6ccc(-c7ccc(-c8ccc9c(c8)c8ccccc8n9-c8ccccc8)cc7)cc6)cc5)cc4)ccc2n3-c2ccccc2)cc1 ; c1ccc(-c2ccc3c(c2)c2cc(-c4cccc(-c5cccc(-c6cccc(-c7cccc(-c8ccc9c(c8)c8ccccc8n9-c8ccccc8)c7)c6)c5)c4)ccc2n3-c2ccccc2)cc1]

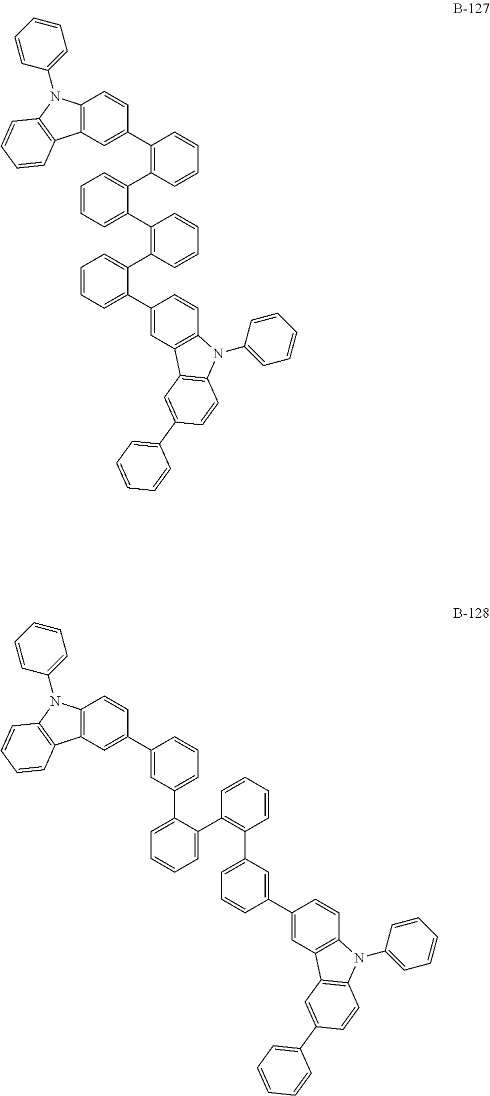 [SMILES: c1ccc(-c2ccc3c(c2)c2cc(-c4ccccc4-c4ccccc4-c4ccccc4-c4ccccc4-c4ccc5c(c4)c4ccccc4n5-c4ccccc4)ccc2n3-c2ccccc2)cc1 ; c1ccc(-c2ccc3c(c2)c2cc(-c4cccc(-c5ccccc5-c5ccccc5-c5cccc(-c6ccc7c(c6)c6ccccc6n7-c6ccccc6)c5)c4)ccc2n3-c2ccccc2)cc1]

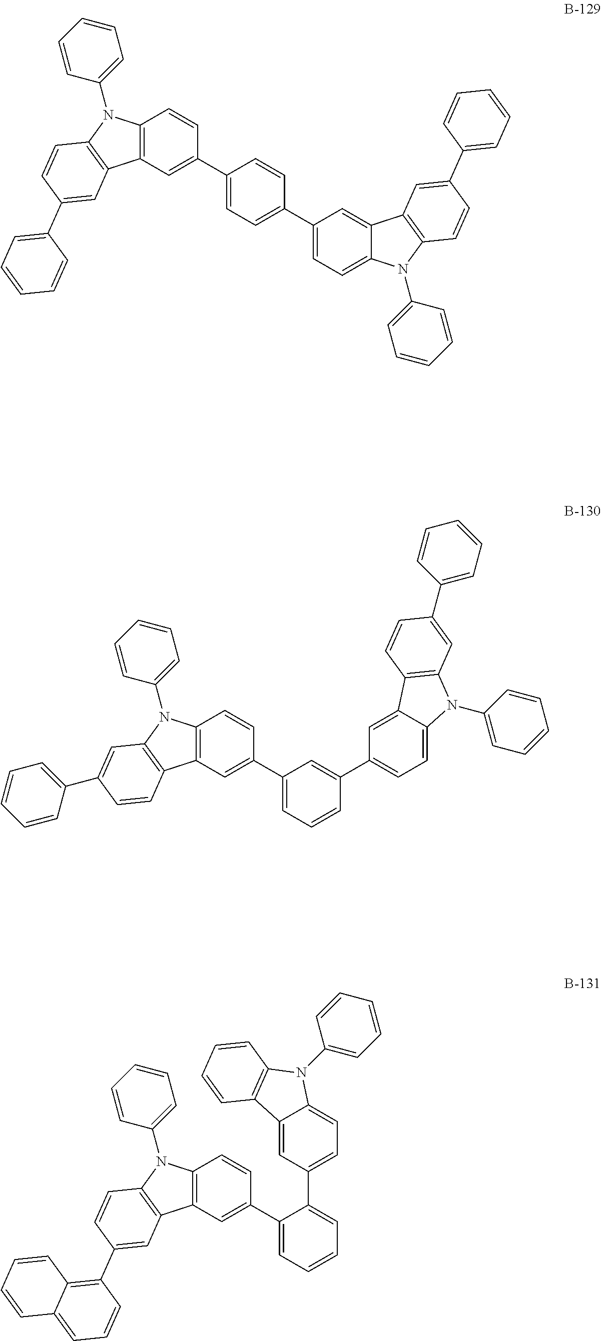 [SMILES: c1ccc(-c2ccc3c(c2)c2cc(-c4ccc(-c5ccc6c(c5)c5cc(-c7ccccc7)ccc5n6-c5ccccc5)cc4)ccc2n3-c2ccccc2)cc1 ; c1ccc(-c2ccc3c4cc(-c5cccc(-c6ccc7c(c6)c6ccc(-c8ccccc8)cc6n7-c6ccccc6)c5)ccc4n(-c4ccccc4)c3c2)cc1 ; c1ccc(-n2c3ccccc3c3cc(-c4ccccc4-c4ccc5c(c4)c4cc(-c6cccc7ccccc67)ccc4n5-c4ccccc4)ccc32)cc1]

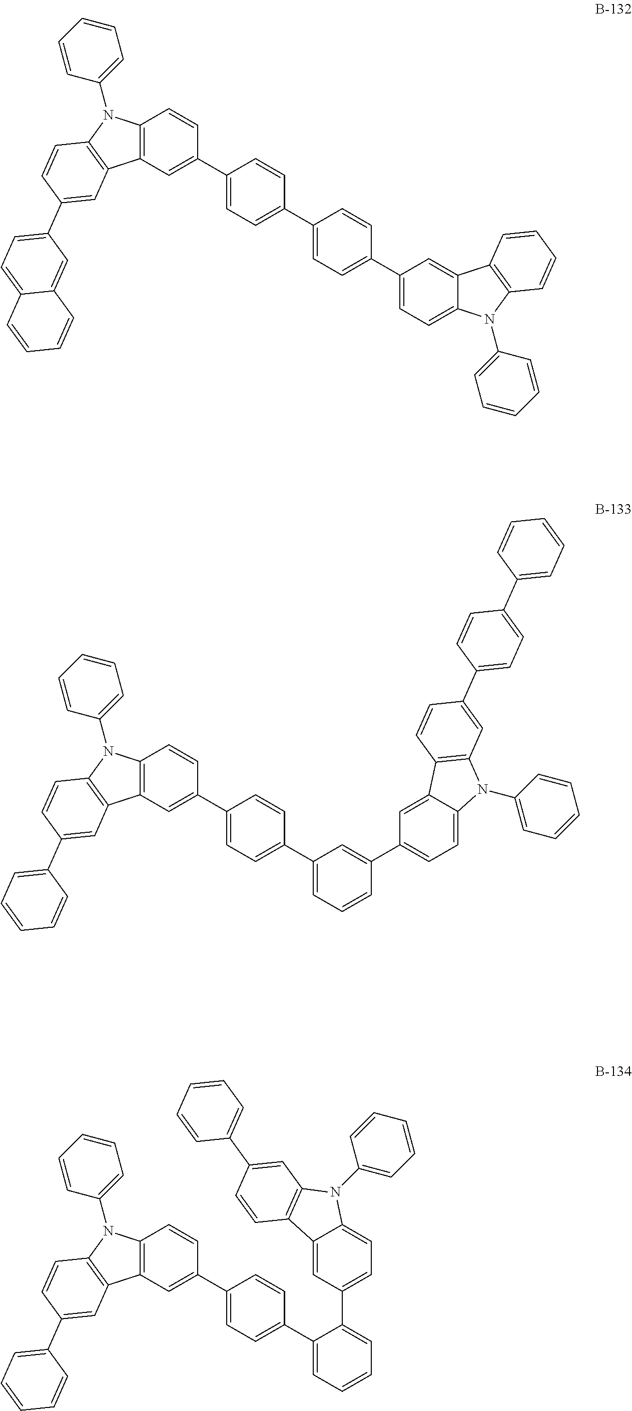 [SMILES: c1ccc(-n2c3ccccc3c3cc(-c4ccc(-c5ccc(-c6ccc7c(c6)c6cc(-c8ccc9ccccc9c8)ccc6n7-c6ccccc6)cc5)cc4)ccc32)cc1 ; c1ccc(-c2ccc(-c3ccc4c5cc(-c6cccc(-c7ccc(-c8ccc9c(c8)c8cc(-c%10ccccc%10)ccc8n9-c8ccccc8)cc7)c6)ccc5n(-c5ccccc5)c4c3)cc2)cc1 ; c1ccc(-c2ccc3c(c2)c2cc(-c4ccc(-c5ccccc5-c5ccc6c(c5)c5ccc(-c7ccccc7)cc5n6-c5ccccc5)cc4)ccc2n3-c2ccccc2)cc1]

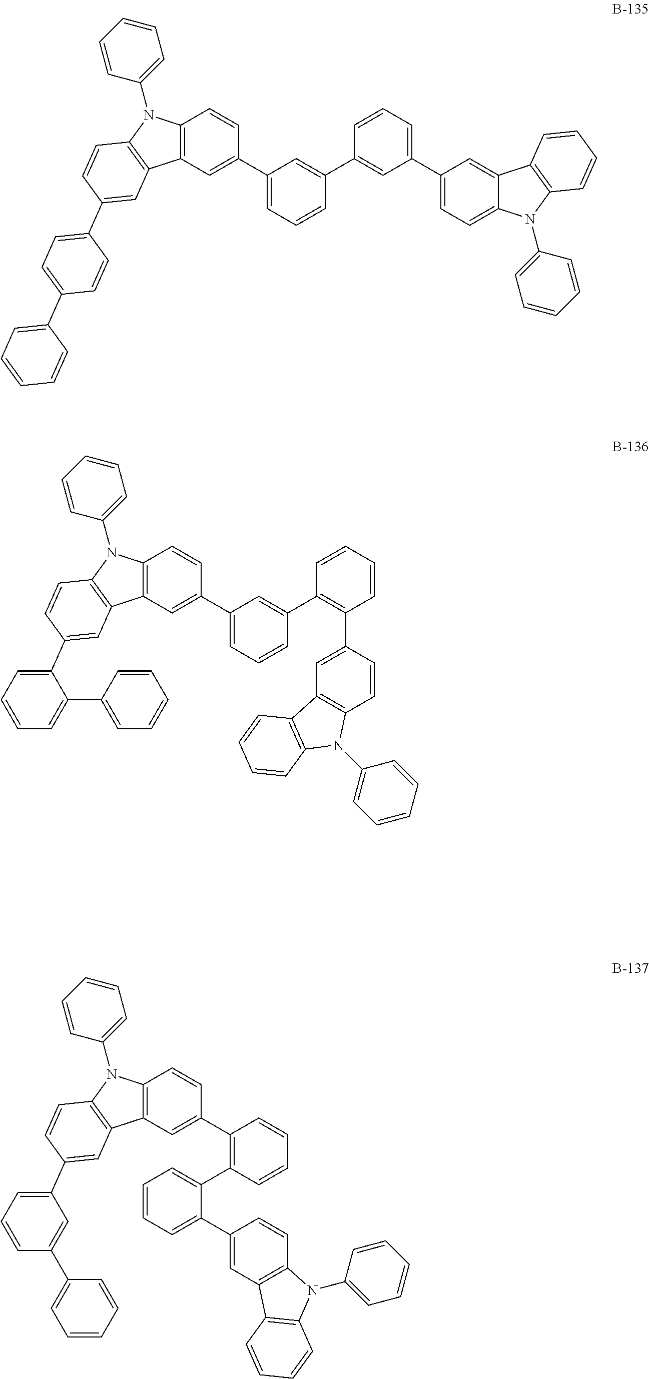 [SMILES: c1ccc(-c2ccc(-c3ccc4c(c3)c3cc(-c5cccc(-c6cccc(-c7ccc8c(c7)c7ccccc7n8-c7ccccc7)c6)c5)ccc3n4-c3ccccc3)cc2)cc1 ; c1ccc(-c2ccccc2-c2ccc3c(c2)c2cc(-c4cccc(-c5ccccc5-c5ccc6c(c5)c5ccccc5n6-c5ccccc5)c4)ccc2n3-c2ccccc2)cc1 ; c1ccc(-c2cccc(-c3ccc4c(c3)c3cc(-c5ccccc5-c5ccccc5-c5ccc6c(c5)c5ccccc5n6-c5ccccc5)ccc3n4-c3ccccc3)c2)cc1]

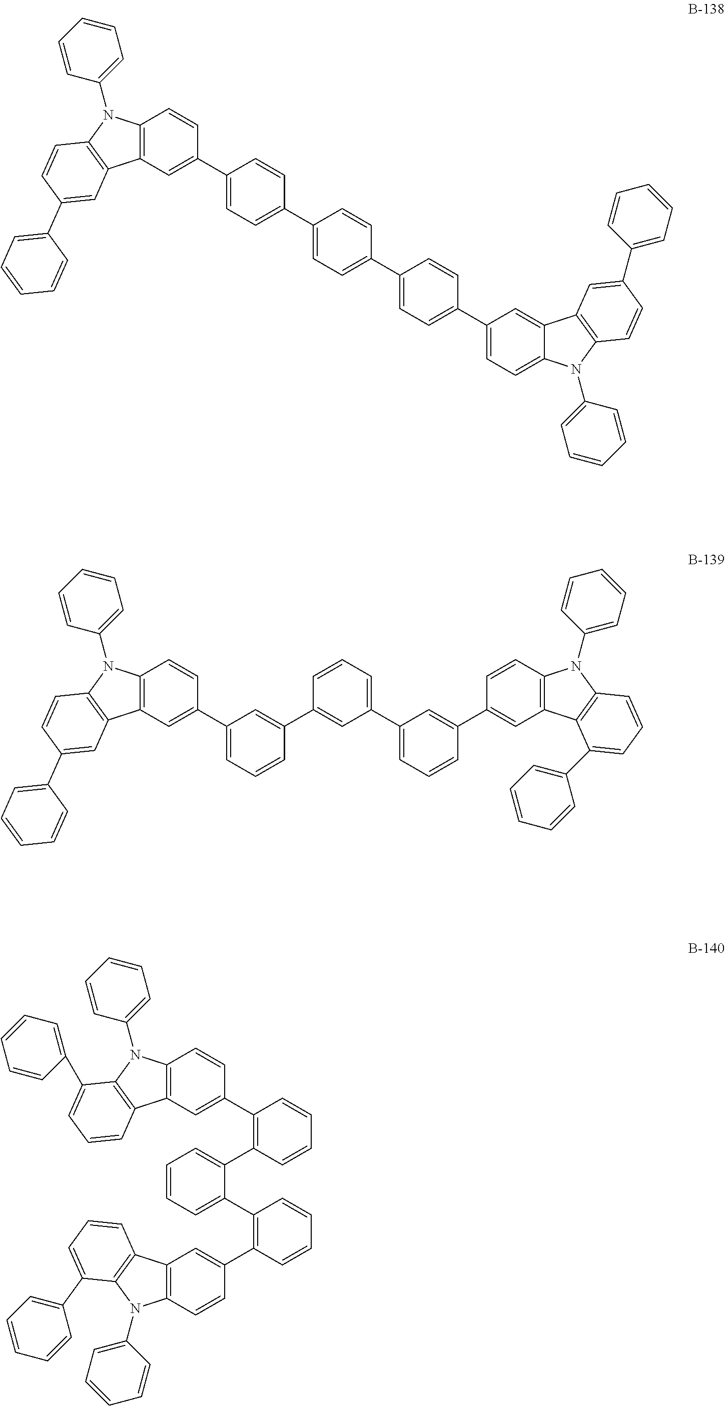 [SMILES: c1ccc(-c2ccc3c(c2)c2cc(-c4ccc(-c5ccc(-c6ccc(-c7ccc8c(c7)c7cc(-c9ccccc9)ccc7n8-c7ccccc7)cc6)cc5)cc4)ccc2n3-c2ccccc2)cc1 ; c1ccc(-c2ccc3c(c2)c2cc(-c4cccc(-c5cccc(-c6cccc(-c7ccc8c(c7)c7c(-c9ccccc9)cccc7n8-c7ccccc7)c6)c5)c4)ccc2n3-c2ccccc2)cc1 ; c1ccc(-c2cccc3c4cc(-c5ccccc5-c5ccccc5-c5ccccc5-c5ccc6c(c5)c5cccc(-c7ccccc7)c5n6-c5ccccc5)ccc4n(-c4ccccc4)c23)cc1]

The first host compound and the second host compound may variously be combined to provide various compositions.

The first host compound is a compound having a relatively strong electron characteristics and the second host compound is a compound having a relatively strong hole characteristics, and they simultaneously are desirable for a deposition process, and they are used together and thus improves luminous efficiency due to increased mobility of electrons and holes compared with the compounds alone.

When a material having biased electron or hole characteristics is used to form a light-emitting layer, excitons in a device including the light-emitting layer are relatively more generated due to recombination of carriers on the interface between a light-emitting layer and an electron transport layer (ETL) or a hole transport layer (HTL). As a result, the molecular excitons in the light-emitting layer interact with charges on the interface of the transport layers and thus, cause a roll-off of sharply deteriorating efficiency and also, sharply deteriorate light emitting life-span characteristics. In order to solve the problems, the first and second hosts are simultaneously included in the light-emitting layer to make a light emitting region not be biased to either of the electron transport layer or the hole transport layer and a device capable of adjusting carrier balance in the light-emitting layer may be provided and thereby roll-off may be improved and life-span characteristics may be remarkably improved.

The first host compound and the second host compound may be, for example included in a weight ratio of 1:10 to 10:1. Within the ranges, bipolar characteristics may be effectively realized to improve efficiency and life-span simultaneously.

The composition may further include at least one compound in addition to the first host compound and the second host compound.

The composition may further include a dopant. The dopant may be a red, green, or blue dopant, for example a phosphorescent dopant.

The dopant is mixed with the first host compound and the second host compound in a small amount to cause light emission, and may be generally a material such as a metal complex that emits light by multiple excitation into a triplet or more. The dopant may be, for example an inorganic, organic, or organic/inorganic compound, and one or more kinds thereof may be used.

The phosphorescent dopant may be an organometal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L₂MX [Chemical Formula Z]

In Chemical Formula Z, M is a metal, and L and X are the same or different, and are a ligand to form a complex compound with M.

The M may be, for example, Ir, Pt, Os, Ti, Zr, Hf, Eu, Tb, Tm, Fe, Co, Ni, Ru, Rh, Pd, or a combination thereof, and the L and X may be, for example a bidendate ligand.

The composition may be formed using a dry film formation method such as chemical vapor deposition (CVD) or a solution process.

Hereinafter, an organic optoelectric diode including the composition is described.

The organic optoelectric diode may be any device to convert electrical energy into photoenergy and vice versa without particular limitation, and may be, for example an organic photoelectric diode, an organic light emitting diode, an organic solar cell, and an organic photo conductor drum.

The organic optoelectric diode may include an anode and a cathode facing each other, at least one organic layer between the anode and the cathode, and the organic layer includes the composition.

Herein, an organic light emitting diode as one example of an organic optoelectric diode is described referring to drawings.

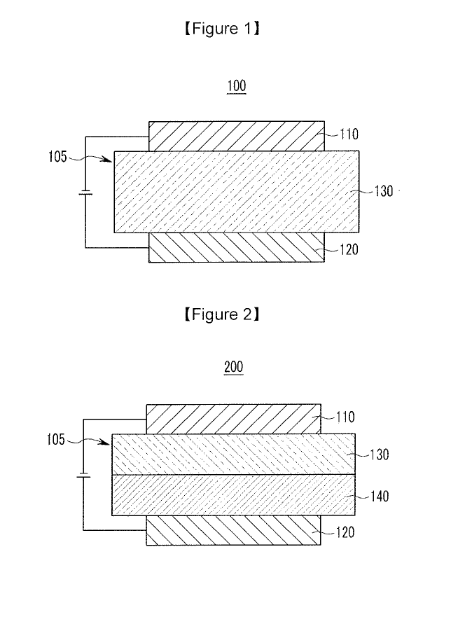

FIGS. 1 and 2 are cross-sectional views showing organic light emitting diodes according to an embodiment.

Referring to FIG. 1, an organic optoelectric diode 100 according to an embodiment includes an anode 120 and a cathode 110 and an organic layer 105 between the anode 120 and the cathode 110.

The anode 120 may be made of a conductor having a large work function to help hole injection, and may be for example metal, metal oxide and/or a conductive polymer. The anode 120 may be, for example a metal nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline, but is not limited thereto.

The cathode 110 may be made of a conductor having a small work function to help electron injection, and may be for example metal, metal oxide and/or a conductive polymer. The cathode 110 may be for example a metal or an alloy thereof such as magnesium, calcium, sodium, potassium, titanium, indium, yttrium, lithium, gadolinium, aluminum silver, tin, lead, cesium, barium, and the like; a multi-layer structure material such as LiF/Al, LiO₂/Al, LiF/Ca, LiF/Al and BaF₂/Ca, but is not limited thereto.

The organic layer 105 includes a light-emitting layer 130 including the composition.

The light-emitting layer 130 may include, for example the composition.

Referring to FIG. 2, an organic light emitting diode 200 further includes a hole auxiliary layer 140 in addition to the light-emitting layer 130. The hole auxiliary layer 140 may improve hole injection and/or hole mobility and block electrons between the anode 120 and the light-emitting layer 130. The hole auxiliary layer 140 may improve hole injection and/or hole mobility and block electrons between the anode 120 and the light-emitting layer 130.

In an embodiment of the present invention, in FIG. 1 or 2, the organic thin layer 105 of the light emitting diode may further include an electron transport layer (ETL), an electron injection layer (EIL), a hole injection layer.

The organic light emitting diodes 100 and 200 may be manufactured by forming an anode or a cathode on a substrate, forming an organic layer in accordance with a dry coating method such as evaporation, sputtering, plasma plating, and ion plating; and forming a cathode or an anode thereon.

The organic light emitting diode may be applied to an organic light emitting display device.

### MODE FOR INVENTION

Hereinafter, the embodiments are illustrated in more detail with reference to examples. These examples, however, are not in any sense to be interpreted as limiting the scope of the invention.

### Synthesis of First Host Compound

### Synthesis Example 1: Synthesis of Intermediate I-1

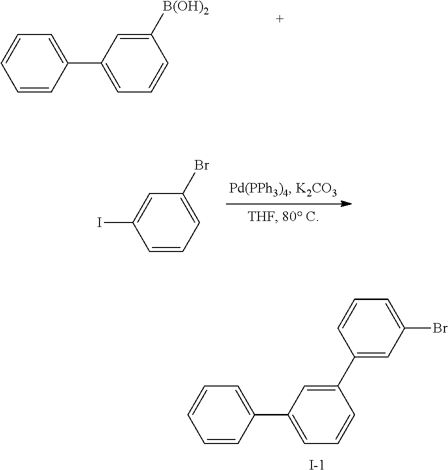 [SMILES: OB(O)c1cccc(-c2ccccc2)c1 ; Brc1cccc(I)c1 ; Brc1cccc(-c2cccc(-c3ccccc3)c2)c1 ; [I-]]

Biphenyl-3-ylboronic acid (100 g, 505 mmol) was dissolved in 1.4 L of tetrahydrofuran (THF) under a nitrogen environment, 1-bromo-3-iodobenzene (171 g, 606 mmol) and tetrakis(triphenylphosphine)palladium (5.83 g, 5.05 mmol) were added thereto, and the mixture was stirred. Potassium carbonate saturated in water (186 g, 1.26 mmol) was added thereto, and the obtained mixture was heated and refluxed at 80° C. for 6 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-1 (142 g, 91%).

HRMS (70 eV, EI+): m/z calcd for C18H13Br: 308.0201. found: 308.

Elemental Analysis: C, 70%; H, 4%.

### Synthesis Example 2: Synthesis of Intermediate I-2

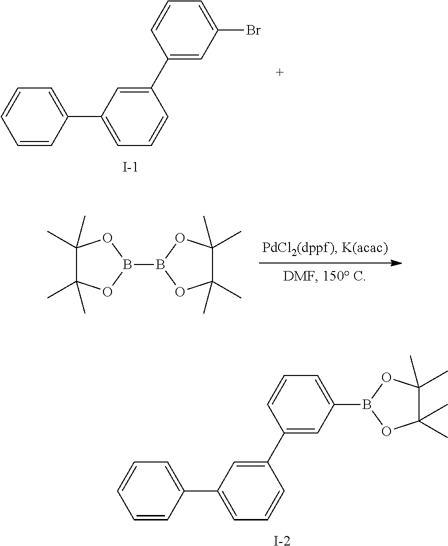

The Intermediate I-1 (140 g, 453 mmol) was dissolved in 3 L of dimethylformamide (DMF) under a nitrogen environment, bis(pinacolato)diboron (138 g, 543 mmol), (1,1′-bis(diphenylphosphine)ferrocene)dichloropalladium (II) (3.70 g, 4.53 mmol), and potassium acetate (133 g, 1,359 mmol) were added thereto, and the mixture was heated and refluxed at 150° C. for 4 hours. When the reaction was completed, water was added to the reaction solution, and a mixture was filtered and dried in a vacuum oven. This obtained residue was separated and purified through flash column chromatography to obtain Intermediate I-22 (145 g, 90%).

HRMS (70 eV, EI+): m/z calcd for C24H25BO2: 356.1948. found: 356.

Elemental Analysis: C, 81%; H, 7%.

### Synthesis Example 3: Synthesis of Intermediate I-3

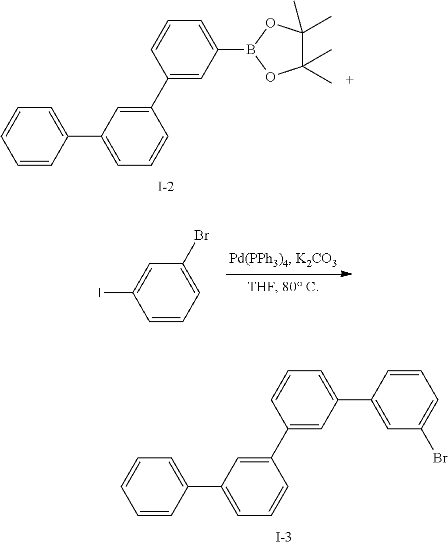

The Intermediate I-2 (100 g, 281 mmol) was dissolved in 1.0 L of tetrahydrofuran (THF) under a nitrogen environment, 1-bromo-3-iodobenzene (95.4 g, 337 mmol) and tetrakis(triphenylphosphine)palladium (3.25 g, 2.81 mmol) were added thereto, and the mixture was stirred. Potassium carbonate saturated in water (103 g, 703 mmol) was added thereto, and the obtained mixture was heated and refluxed at 80° C. for 8 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-3 (85.5 g, 79%).

HRMS (70 eV, EI+): m/z calcd for C24H17Br: 384.0514. found: 384.

Elemental Analysis: C, 75%; H, 4%.

### Synthesis Example 4: Synthesis of Intermediate I-4

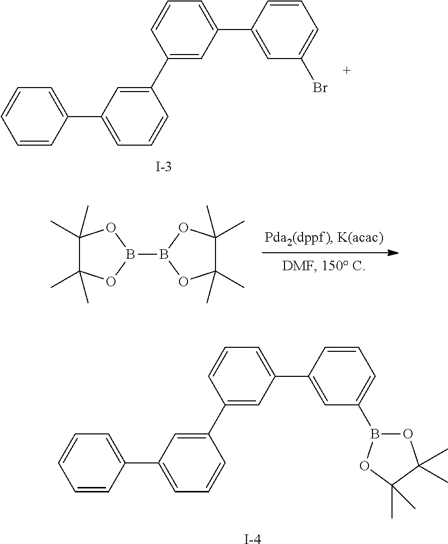

The Intermediate I-3 (80 g, 208 mmol) was dissolved in 0.7 L of dimethylformamide (DMF) under a nitrogen environment, bis(pinacolato)diboron (63.2 g, 249 mmol), (1,1′-bis(diphenylphosphine)ferrocene)dichloropalladium (II) (1.70 g, 2.08 mmol), and potassium acetate (61.2 g, 624 mmol) were added thereto, and the mixture was heated and refluxed at 150° C. for 12 hours. When the reaction was completed, water was added to the reaction solution, and a mixture was filtered and dried in a vacuum oven. This obtained residue was separated and purified through flash column chromatography to obtain Intermediate I-4 (67.4 g, 75%).

HRMS (70 eV, EI+): m/z calcd for C30H29BO2: 432.2261. found: 432.

Elemental Analysis: C, 83%; H, 7%.

### Synthesis Example 5: Synthesis of Intermediate I-5

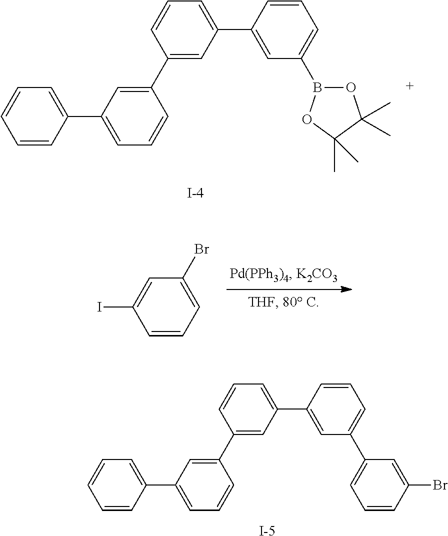

The Intermediate I-4 (65 g, 150 mmol) was dissolved in 0.6 L of tetrahydrofuran (THF) under a nitrogen environment, 1-bromo-3-iodobenzene (51.0 g, 180 mmol) and tetrakis(triphenylphosphine)palladium (1.73 g, 1.50 mmol) were added thereto, and the mixture was stirred. Potassium carbonate (55.2 g, 375 mmol) was added thereto, and the obtained mixture was heated and refluxed at 80° C. for 15 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-5 (49.1 g, 71%).

HRMS (70 eV, EI+): m/z calcd for C30H21Br: 460.0827. found: 460.

Elemental Analysis: C, 78%; H, 5%.

### Synthesis Example 6: Synthesis of Intermediate I-6

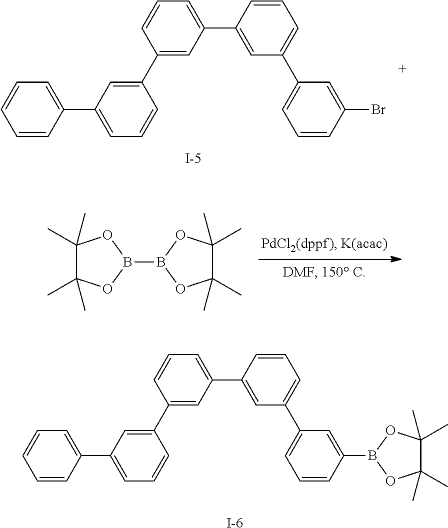

The Intermediate I-5 (45 g, 97.5 mmol) was dissolved in 0.7 L of dimethylformamide (DMF) under a nitrogen environment, bis(pinacolato)diboron (29.7 g, 117 mmol), (1,1′-bis(diphenylphosphine)ferrocene)dichloropalladium (II) (0.8 g, 0.98 mmol), and potassium acetate (28.7 g, 293 mmol) were added thereto, and the mixture was heated and refluxed at 150° C. for 8 hours. When the reaction was completed, water was added to the reaction solution, and a mixture was filtered and dried in a vacuum oven. This obtained residue was separated and purified through flash column chromatography to obtain Intermediate I-6 (34.7 g, 70%).

HRMS (70 eV, EI+): m/z calcd for C36H33BO2: 508.2574. found: 508.

Elemental Analysis: C, 85%; H, 7%.

### Synthesis Example 7: Synthesis of Intermediate I-7

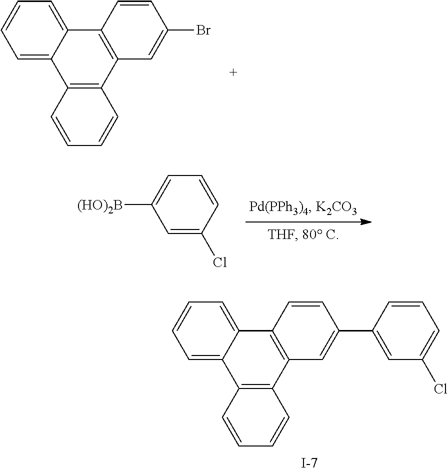

2-bromotriphenylene (32.7 g, 107 mmol) was dissolved in 0.3 L of tetrahydrofuran (THF) under a nitrogen environment, 3-chlorophenylboronic acid (20 g, 128 mmol) and tetrakis(triphenylphosphine)palladium (1.23 g, 1.07 mmol) were added thereto, and the mixture was stirred. Potassium carbonate saturated in water (36.8 g, 267 mmol) was added thereto, and the obtained mixture was heated and refluxed at 80° C. for 24 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-7 (22.6 g, 63%).

HRMS (70 eV, EI+): m/z calcd for C24H15Cl: 338.0862. found: 338.

Elemental Analysis: C, 85%; H, 5%.

### Synthesis Example 8: Synthesis of Intermediate I-8

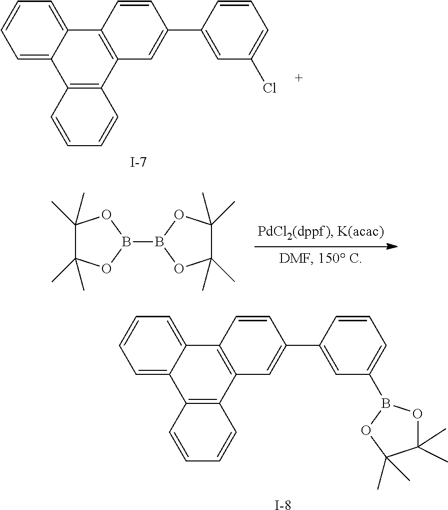

The Intermediate I-7 (22.6 g, 66.7 mmol) was dissolved in 0.3 L of dimethylformamide (DMF) under a nitrogen environment, bis(pinacolato)diboron (25.4 g, 100 mmol), (1,1′-bis(diphenylphosphine)ferrocene)dichloropalladium (II) (0.54 g, 0.67 mmol), and potassium acetate (16.4 g, 167 mmol) were added thereto, and the mixture was heated and refluxed at 150° C. for 48 hours. When the reaction was completed, water was added to the reaction solution, and a mixture was filtered and dried in a vacuum oven. This obtained residue was separated and purified through flash column chromatography to obtain Intermediate I-8 (18.6 g, 65%).

HRMS (70 eV, EI+): m/z calcd for C30H27BO2: 430.2104. found: 430.

Elemental Analysis: C, 84%; H, 6%.

### Synthesis Example 9: Synthesis of Intermediate I-9

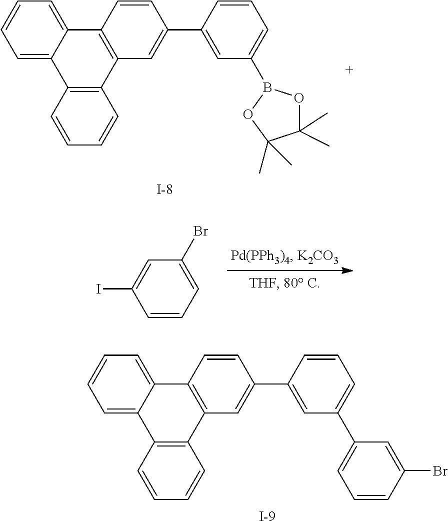

The Intermediate I-8 (50 g, 116 mmol) was dissolved in 0.5 L of tetrahydrofuran (THF) under a nitrogen environment, 1-bromo-3-iodobenzene (39.4 g, 139 mmol) and tetrakis(triphenylphosphine)palladium (1.34 g, 1.16 mmol) were added thereto, and the mixture was stirred. Potassium carbonate saturated in water (40.1 g, 290 mmol) was added thereto, and the obtained mixture was heated and refluxed at 80° C. for 12 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-9 (42.6 g, 80%).

HRMS (70 eV, EI+): m/z calcd for C30H19Br: 458.0670. found: 458.

Elemental Analysis: C, 78%; H, 4%.

### Synthesis Example 10: Synthesis of Intermediate I-10

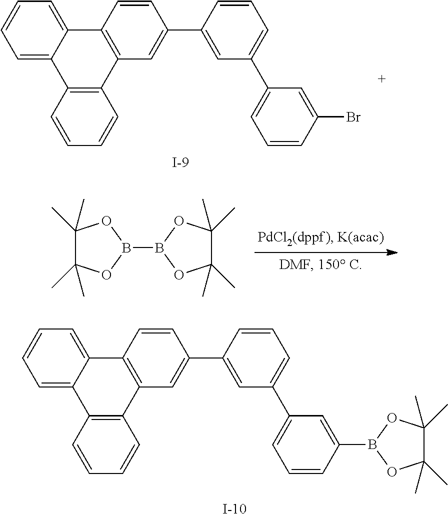

The Intermediate I-9 (40 g, 87.1 mmol) was dissolved in 0.3 L of dimethylformamide (DMF) under a nitrogen environment, bis(pinacolato)diboron (26.5 g, 104 mmol), (1,1′-bis(diphenylphosphine)ferrocene)dichloropalladium (II) (0.71 g, 0.87 mmol), and potassium acetate (21.4 g, 218 mmol) were added thereto, and the mixture was heated and refluxed at 150° C. for 26 hours. When the reaction was completed, water was added to the reaction solution, and a mixture was filtered and dried in a vacuum oven. This obtained residue was separated and purified through flash column chromatography to obtain Intermediate I-10 (34 g, 77%).

HRMS (70 eV, EI+): m/z calcd for C36H31BO2: 506.2417. found: 506.

Elemental Analysis: C, 85%; H, 6%.

### Synthesis Example 11: Synthesis of Intermediate I-11

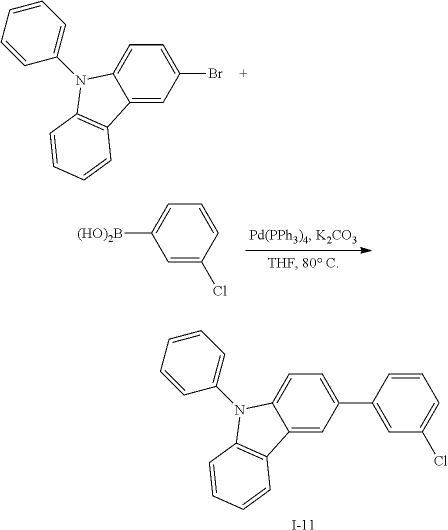

3-bromo-9-phenyl-9H-carbazole (100 g, 310 mmol) was dissolved in 0.8 L of tetrahydrofuran (THF) under a nitrogen environment, 3-chlorophenylboronic acid (53.4 g, 341 mmol) and tetrakis(triphenylphosphine)palladium (3.58 g, 3.10 mmol) were added thereto, and the mixture was stirred. Potassium carbonate saturated in water (114 g, 775 mmol) was added thereto, and the obtained mixture was heated and refluxed at 80° C. for 8 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-11 (104 g, 95%).

HRMS (70 eV, EI+): m/z calcd for C24H16ClN: 353.0971. found: 353.

Elemental Analysis: C, 81%; H, 5%.

### Synthesis Example 12: Synthesis of Intermediate I-12

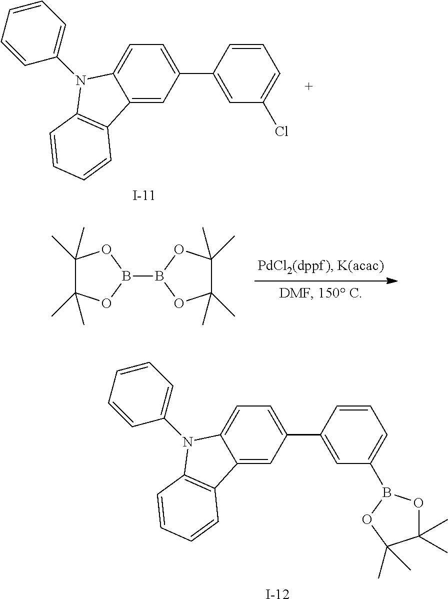

The Intermediate I-11 (100 g, 283 mmol) was dissolved in 0.9 L of dimethylformamide (DMF) under a nitrogen environment, bis(pinacolato)diboron (86.1 g, 339 mmol), (1,1′-bis(diphenylphosphine)ferrocene)dichloropalladium (II) (2.31 g, 2.83 mmol), and potassium acetate (83.3 g, 849 mmol) were added thereto, and the mixture was heated and refluxed at 150° C. for 48 hours. When the reaction was completed, water was added to the reaction solution, and a mixture was filtered and dried in a vacuum oven. This obtained residue was separated and purified through flash column chromatography to obtain Intermediate I-12 (83.2 g, 66%).

HRMS (70 eV, EI+): m/z calcd for C30H28BNO2: 445.2213. found: 445.

Elemental Analysis: C, 81%; H, 6%.

### Synthesis Example 13: Synthesis of Intermediate I-13

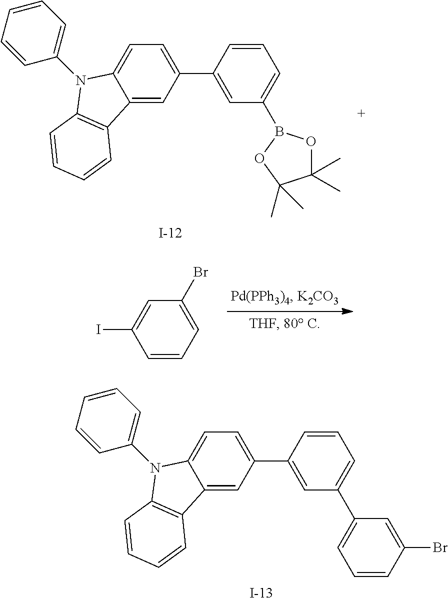

The Intermediate I-12 (80 g, 180 mmol) was dissolved in 0.7 L of tetrahydrofuran (THF) under a nitrogen environment, 1-bromo-3-iodobenzene (61.0 g, 216 mmol) and tetrakis(triphenylphosphine)palladium (2.08 g, 1.80 mmol) were added thereto, and the mixture was stirred. Potassium carbonate saturated in water (66.3 g, 450 mmol) was added thereto, and the obtained mixture was heated and refluxed at 80° C. for 15 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-13 (70.9 g, 83%).

HRMS (70 eV, EI+): m/z calcd for C30H20BrN: 473.0779. found: 473.

Elemental Analysis: C, 76%; H, 4%.

### Synthesis Example 14: Synthesis of Intermediate I-14

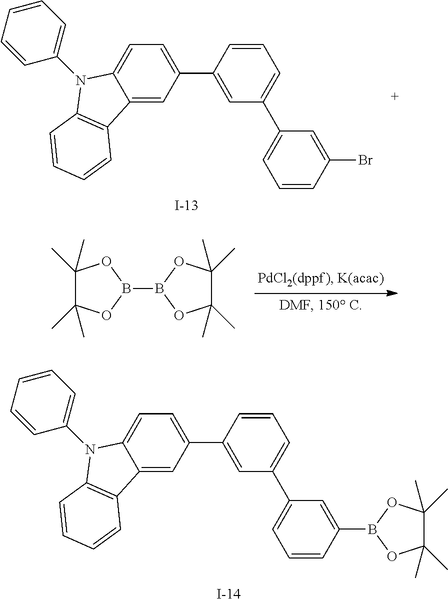

The Intermediate I-13 (65 g, 137 mmol) was dissolved in 0.5 L of dimethylformamide (DMF) under a nitrogen environment, bis(pinacolato)diboron (41.8 g, 164 mmol), (1,1′-bis(diphenylphosphine)ferrocene)dichloropalladium (II) (1.12 g, 1.37 mmol), and potassium acetate (40.3 g, 411 mmol) were added thereto, and the mixture was heated and refluxed at 150° C. for 12 hours. When the reaction was completed, water was added to the reaction solution, and a mixture was filtered and dried in a vacuum oven. This obtained residue was separated and purified through flash column chromatography to obtain Intermediate I-14 (50.0 g, 70%).

HRMS (70 eV, EI+): m/z calcd for C36H32BNO2: 521.2526. found: 521.

Elemental Analysis: C, 90%; H, 6%.

### Synthesis Example 15: Synthesis of Intermediate I-15

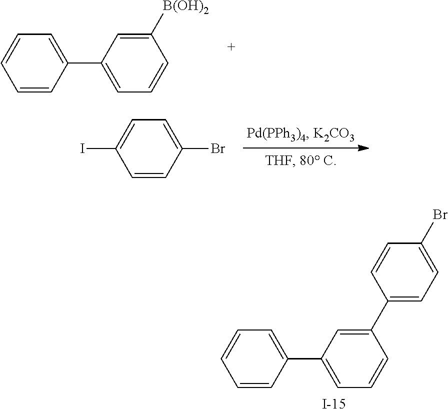

Biphenyl-3-ylboronic acid (100 g, 505 mmol) was dissolved in 1.4 L of tetrahydrofuran (THF) under a nitrogen environment, 1-bromo-4-iodobenzene (171 g, 606 mmol) and tetrakis(triphenylphosphine)palladium (5.83 g, 5.05 mmol) were added thereto, and the mixture was stirred. Potassium carbonate saturated in water (186 g, 1.26 mmol) was added thereto, and the obtained mixture was heated and refluxed at 80° C. for 8 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-15 (148 g, 95%).

HRMS (70 eV, EI+): m/z calcd for C18H13Br: 308.0201. found: 308.

Elemental Analysis: C, 70%; H, 4%.

### Synthesis Example 16: Synthesis of Intermediate I-16

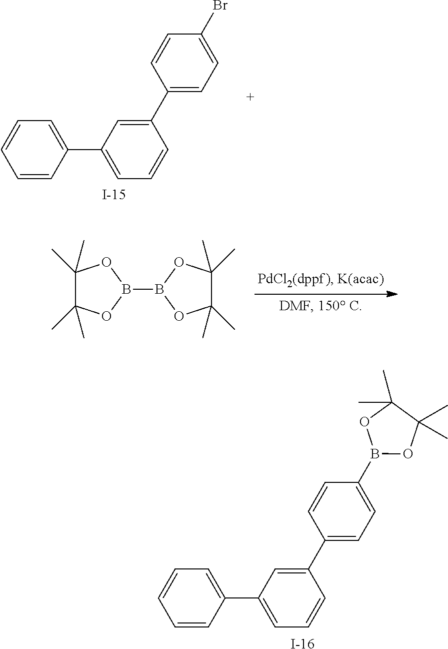

The Intermediate I-15 (140 g, 453 mmol) was dissolved in 1.4 L of dimethylformamide (DMF) under a nitrogen environment, bis(pinacolato)diboron (138 g, 543 mmol), (1,1′-bis(diphenylphosphine)ferrocene)dichloropalladium (II) (3.70 g, 4.53 mmol), and potassium acetate (133 g, 1,359 mmol) were added thereto, and the mixture was heated and refluxed at 150° C. for 8 hours. When the reaction was completed, water was added to the reaction solution, and a mixture was filtered and dried in a vacuum oven. This obtained residue was separated and purified through flash column chromatography to obtain Intermediate I-16 (127 g, 79%).

HRMS (70 eV, EI+): m/z calcd for C24H25BO2: 356.1948. found: 356.

Elemental Analysis: C, 81%; H, 7%.

### Synthesis Example 17: Synthesis of Intermediate I-17

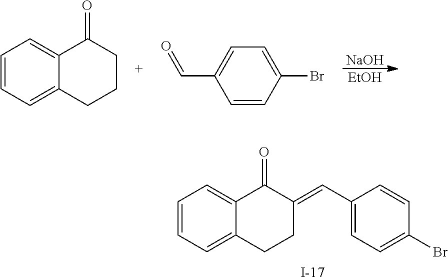

α-tetralone (100 g, 684 mmol) was dissolved in 1 L of ethanol under a nitrogen environment, 4-bromobenzaldehyde (127 g, 684 mmol) and sodium hydroxide (41.0 g, 1026 mmol) were added thereto, and the mixture was stirred at room temperature for 2 hours. When the reaction was complete, the reaction solution was filtered and then, washed with a small amount of ethanol. In this way, Intermediate I-17 (179 g, 83%) was obtained.

HRMS (70 eV, EI+): m/z calcd for C17H13BrO: 312.0150. found: 312.

Elemental Analysis: C, 65%; H, 4%.

### Synthesis Example 18: Synthesis of Intermediate I-18

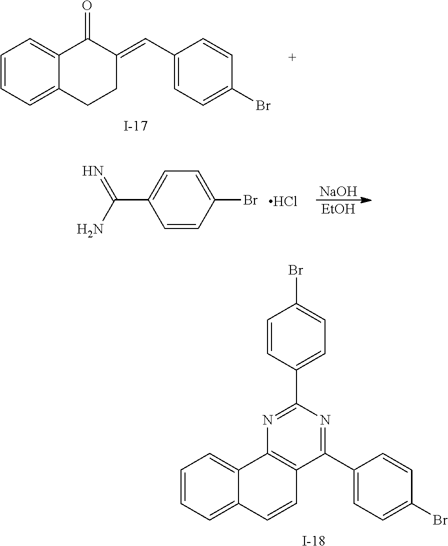

The Intermediate I-17 (170 g, 543 mmol) was dissolved in 1.5 L of ethanol under a nitrogen environment, 4-bromobenzimidamide hydrochloride (128 g, 543 mmol) and sodium hydroxide (65.2 g, 1,629 mmol) were added thereto, and the mixture was stirred at room temperature for 17 hours. When the reaction was complete, the reaction solution was filtered and then, washed with a small amount of ethanol. In this way, Intermediate I-18 (120 g, 45%) was obtained.

HRMS (70 eV. EI+): m/z calcd for C24H16Br2N2: 489.9680. found: 490.

Elemental Analysis: C, 59%; H, 3%.

### Synthesis Example 19: Synthesis of Intermediate I-19

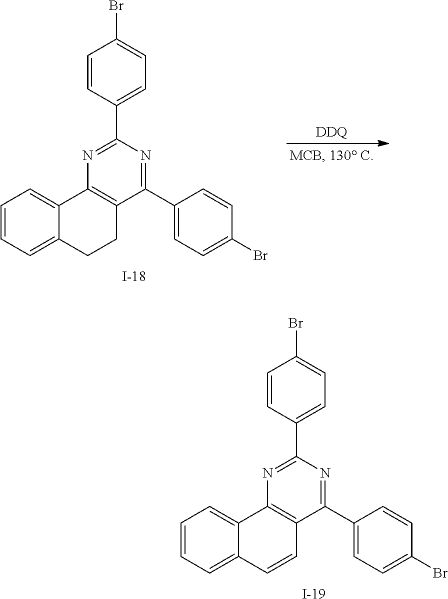

The Intermediate I-18 (110 g, 223 mmol) was dissolved in 1 L of monochlorobenzene (MCB) under a nitrogen environment, 2,3-dichloro-5,6-dicyano-1,4-benzoquinone (DDQ, 101 g, 446 mmol) was added thereto, and the mixture was heated and refluxed at 130° C. for 15 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-9 (76.5 g, 70%).

HRMS (70 eV, EI+): m/z calcd for C24H14Br2N2: 487.9524. found: 488.

Elemental Analysis: C, 59%; H, 3%.

### Synthesis Example 20: Synthesis of Compound 6

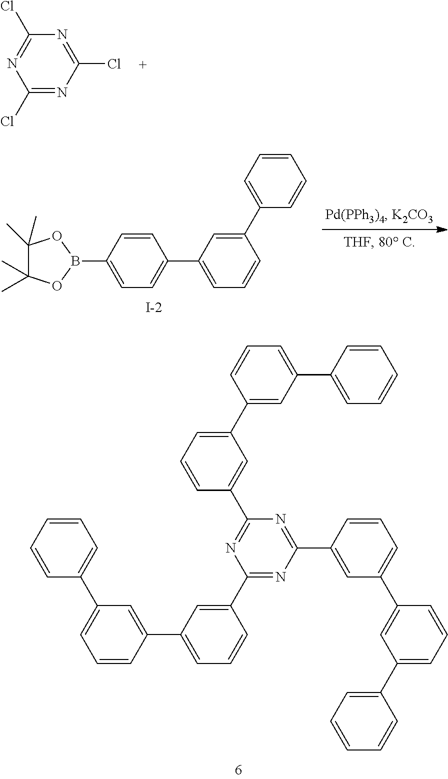

2,4,6-trichloro-1,3,5-triazine (20 g, 108 mmol) was dissolved in 0.8 L of tetrahydrofuran (THF) under a nitrogen environment, the intermediate I-2 (135 g, 380 mmol) and tetrakis(triphenylphosphine)palladium (3.74 g, 3.24 mmol) were added thereto, and the mixture was stirred. Potassium carbonate saturated in water (95.4 g, 648 mmol) was added thereto, and the mixture was heated and refluxed at 80° C. for 24 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound 6 (60.4 g, 73%).

HRMS (70 eV, EI+): m/z calcd for C57H39N3: 765.3144. found: 765.

Elemental Analysis: C, 89%; H, 5%.

### Synthesis Example 21: Synthesis of Compound 7

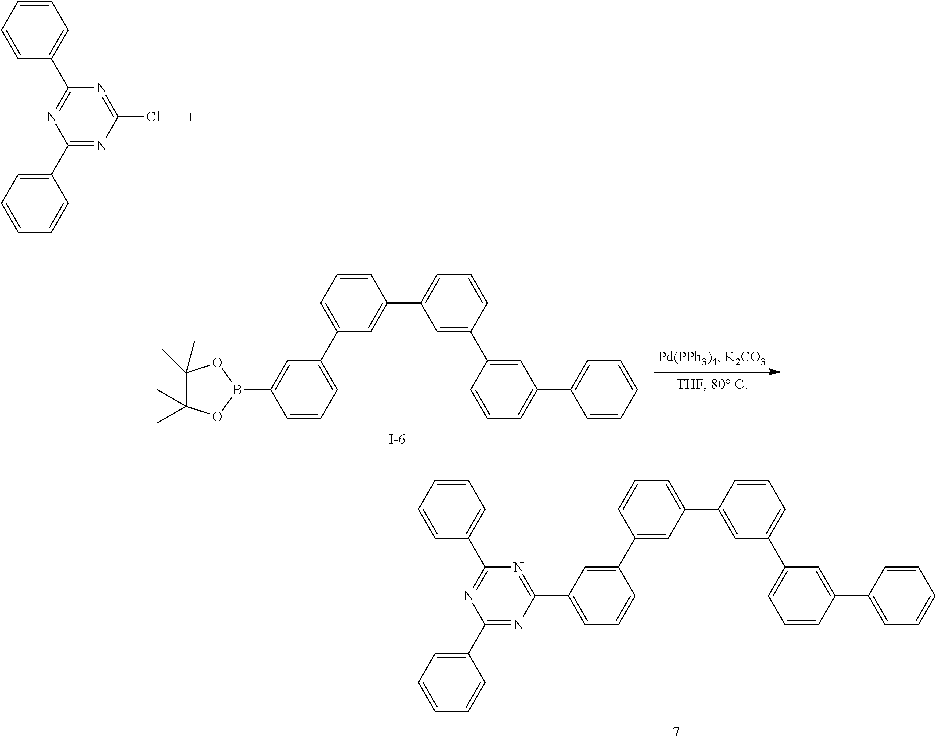

2-chloro-4,6-diphenyl-1,3,5-triazine (20 g, 74.7 mmol) made by Shenzhen gre-syn Chemical Technology (http://www.gre-syn.com/) was dissolved in 0.8 L of tetrahydrofuran (THF) under a nitrogen environment, the Intermediate I-6 (38.0 g, 74.7 mmol) and tetrakis(triphenylphosphine)palladium (0.87 g, 0.75 mmol) were added thereto, and the mixture was stirred. Potassium carbonate saturated in water (27.5 g, 187 mmol) was added thereto, and the obtained mixture was heated and refluxed at 80° C. for 14 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound 7 (40.3 g, 88%).

HRMS (70 eV, EI+): m/z calcd for C45H31N3: 613.2518. found: 613.

Elemental Analysis: C, 88%; H, 5%.

### Synthesis Example 22: Synthesis of Compound 13

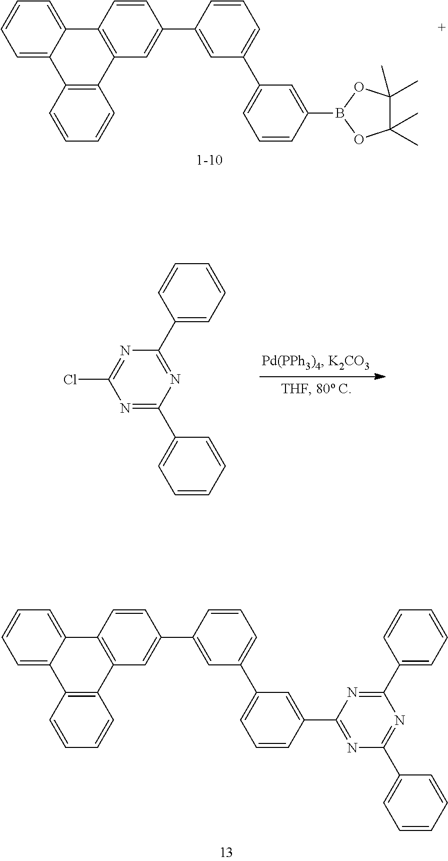 [SMILES: CC1(C)OB(c2cccc(-c3cccc(-c4ccc5c6ccccc6c6ccccc6c5c4)c3)c2)OC1(C)C ; Clc1nc(-c2ccccc2)nc(-c2ccccc2)n1 ; c1ccc(-c2nc(-c3ccccc3)nc(-c3cccc(-c4cccc(-c5ccc6c7ccccc7c7ccccc7c6c5)c4)c3)n2)cc1]

The Intermediate I-10 (20 g, 39.5 mmol) was dissolved in 0.2 L of tetrahydrofuran (THF) under a nitrogen environment, 2-chloro-4,6-diphenyl-1,3,5-triazine made by Shenzhen gre-syn Chemical Technology (http://www.gre-syn.com/) (10.6 g, 39.5 mmol) and tetrakis(triphenylphosphine)palladium (0.46 g, 0.4 mmol) were added thereto, and the mixture was stirred. Potassium carbonate saturated in water (13.6 g, 98.8 mmol) was added thereto, and the obtained mixture was heated and refluxed at 80° C. for 23 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound 13 (17.9 g, 74%).

HRMS (70 eV. EI+): m/z calcd for C45H29N3: 611.2361. found: 611.

Elemental Analysis: C, 88%; H, 5%.

### Synthesis Example 23: Synthesis of Compound 14

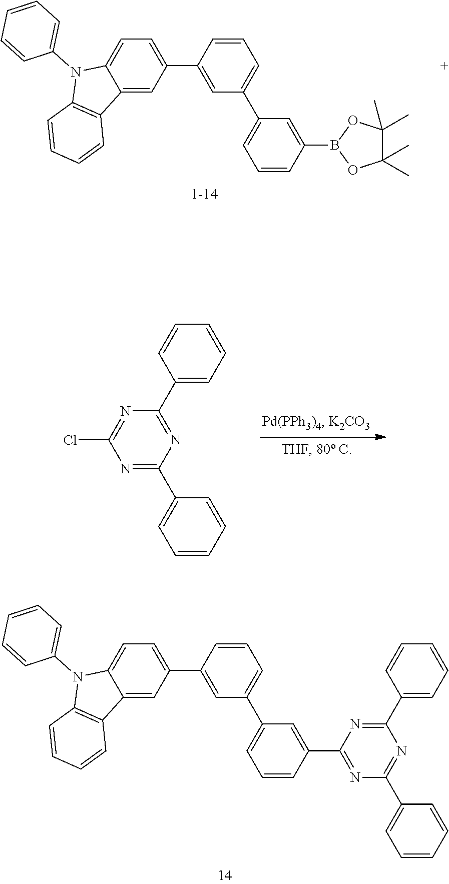 [SMILES: CC1(C)OB(c2cccc(-c3cccc(-c4ccc5c(c4)c4ccccc4n5-c4ccccc4)c3)c2)OC1(C)C ; Clc1nc(-c2ccccc2)nc(-c2ccccc2)n1 ; c1ccc(-c2nc(-c3ccccc3)nc(-c3cccc(-c4cccc(-c5ccc6c(c5)c5ccccc5n6-c5ccccc5)c4)c3)n2)cc1]

The Intermediate I-14 (20 g, 38.4 mmol) was dissolved in 0.2 L of tetrahydrofuran (THF) under a nitrogen environment, 2-chloro-4,6-diphenyl-1,3,5-triazine made by Shenzhen gre-syn Chemical Technology (http://www.gre-syn.com/) (10.3 g, 38.4 mmol) and tetrakis(triphenylphosphine)palladium (0.44 g, 0.38 mmol) were added thereto, and the mixture was stirred. Potassium carbonate saturated in water (14.1 g, 96.0 mmol) was added thereto, and the obtained mixture was heated and refluxed at 80° C. for 18 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound 14 (19.5 g, 81%).

HRMS (70 eV, EI+): m/z calcd for C45H30N4: 626.2470. found: 626.

Elemental Analysis: C, 86%; H, 5%.

### Synthesis Example 24: Synthesis of Compound 21

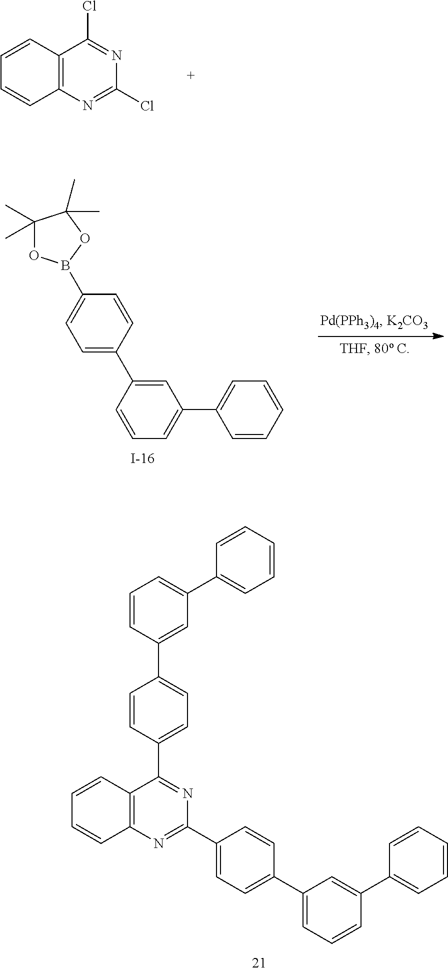

2,4-dichloroquinazoline made by Shenzhen gre-syn Chemical Technology (http://www.gre-syn.com/) (20 g, 100 mmol) was dissolved in 0.8 L of tetrahydrofuran (THF) under a nitrogen environment, the Intermediate I-16 (78.4 g, 220 mmol) and tetrakis(triphenylphosphine)palladium (3.47 g, 3.0 mmol) were added thereto, and the mixture was stirred. Potassium carbonate saturated in water (73.6 g, 500 mmol) was added thereto, and the obtained mixture was heated and refluxed at 80° C. for 15 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound 21 (46.9 g, 80%).

HRMS (70 eV, EI+): m/z calcd for C44H30N2: 586.2409 found: 586.

Elemental Analysis: C, 90%; H, 5%.

### Synthesis Example 25: Synthesis of Compound 22

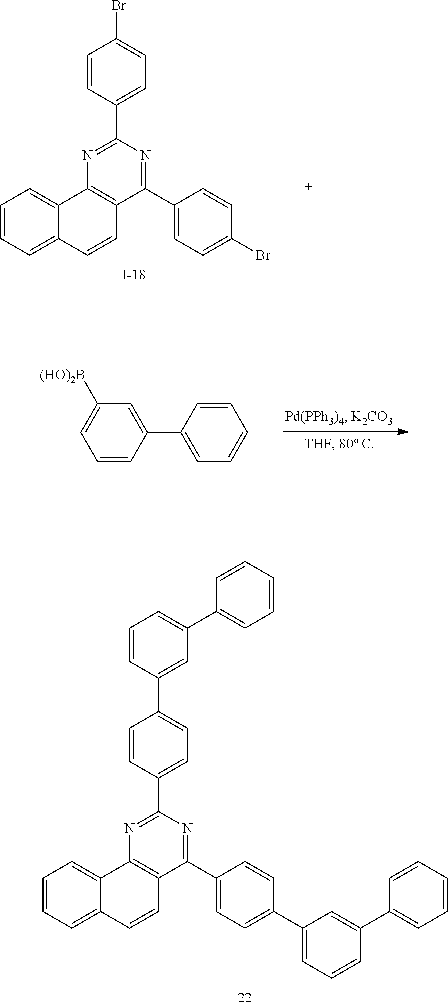

The Intermediate I-18 (20 g, 40.8 mmol) was dissolved in 0.2 L of tetrahydrofuran (THF) under a nitrogen environment, biphenyl-3-ylboronic acid (16.2 g, 81.6 mmol) and tetrakis(triphenylphosphine)palladium (0.94 g, 0.82 mmol) were added thereto, and the mixture was stirred. Potassium carbonate saturated in water (28.2 g, 204 mmol) was added thereto, and the obtained mixture was heated and refluxed at 80° C. for 12 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound 22 (24.9 g, 96%).

HRMS (70 eV, EI+): m/z calcd for C48H32N2: 636.2565. found: 636.

Elemental Analysis: C, 91%; H, 5%.

### Synthesis of Second Host Compound

### Synthesis Example 26: Synthesis of Intermediate I-20

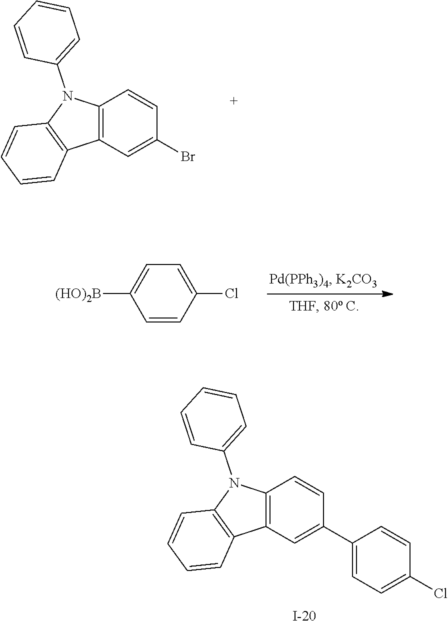

3-bromo-9-phenyl-9H-carbazole (100 g, 310 mmol) was dissolved in 0.8 L of tetrahydrofuran (THF) under a nitrogen environment, 4-chlorophenylboronic acid (53.4 g, 341 mmol) and tetrakis(triphenylphosphine)palladium (3.58 g, 3.10 mmol) were added thereto, and the mixture was stirred. Potassium carbonate saturated in water (114 g, 775 mmol) was added thereto, and the obtained mixture was heated and refluxed at 80° C. for 18 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-20 (97.6 g, 89%).

HRMS (70 eV, EI+): m/z calcd for C24H16ClN: 353.0971. found: 353.

Elemental Analysis: C, 81%; H, 5%.

### Synthesis Example 27: Synthesis of Intermediate I-21

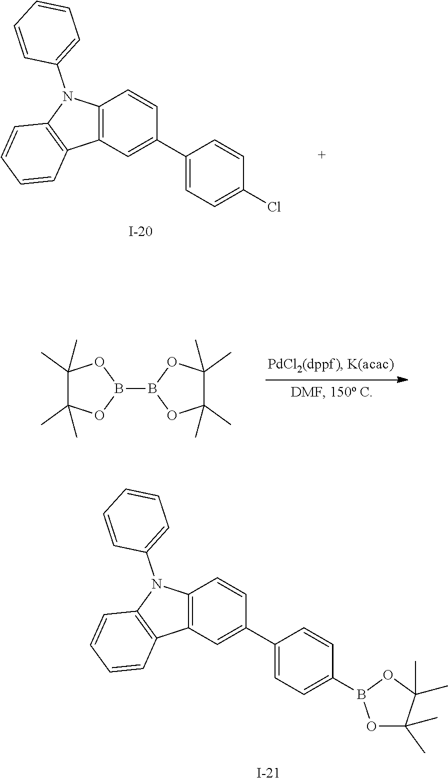

The Intermediate I-20 (90 g, 254 mmol) was dissolved in 0.8 L of dimethylformamide (DMF) under a nitrogen environment, bis(pinacolato)diboron (77.5 g, 305 mmol), (1,1′-bis(diphenylphosphine)ferrocene)dichloropalladium (II) (2.70 g, 2.54 mmol), and potassium acetate (74.8 g, 762 mmol) were added thereto, and the mixture was heated and refluxed at 150° C. for 20 hours. When the reaction was completed, water was added to the reaction solution, and a mixture was filtered and dried in a vacuum oven. This obtained residue was separated and purified through flash column chromatography to obtain Intermediate I-21 (75.8 g, 67%).

HRMS (70 eV, EI+): m/z calcd for C30H28BNO2: 445.2213. found: 445.

Elemental Analysis: C, 81%; H, 6%.

### Synthesis Example 28: Synthesis of Intermediate I-22

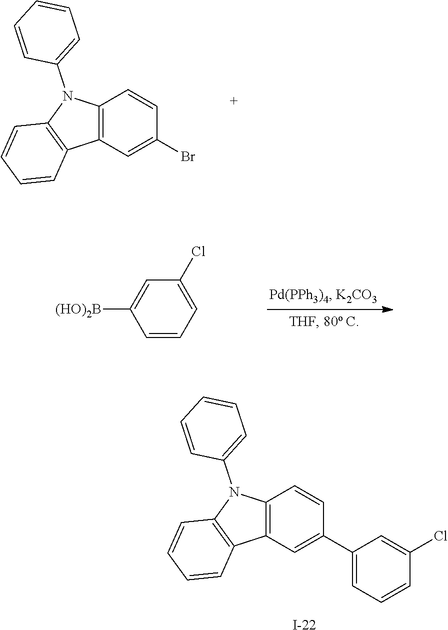

3-bromo-9-phenyl-9H-carbazole (100 g, 310 mmol) was dissolved in 0.8 L of tetrahydrofuran (THF) under a nitrogen environment, 3-chlorophenylboronic acid (53.4 g, 341 mmol) and tetrakis(triphenylphosphine)palladium (3.58 g, 3.10 mmol) were added thereto, and the mixture was stirred. Potassium carbonate saturated in water (114 g, 775 mmol) was added thereto, and the obtained mixture was heated and refluxed at 80° C. for 16 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-22 (91.0 g, 83%).

HRMS (70 eV, EI+): m/z calcd for C24H16ClN: 353.0971. found: 353.

Elemental Analysis: C, 81%; H, 5%.

### Synthesis Example 29: Synthesis of Intermediate I-23

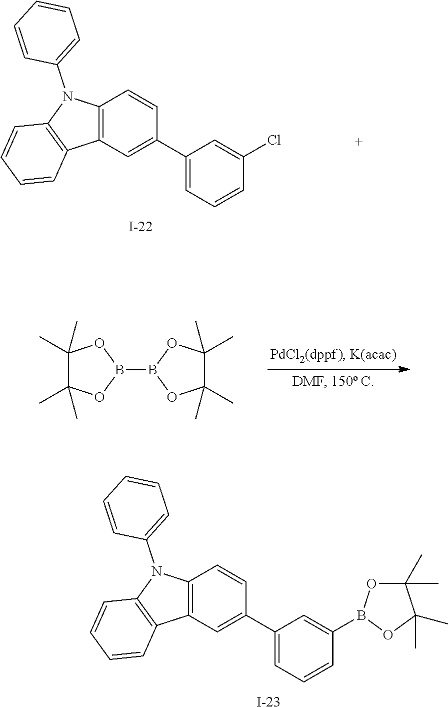

The Intermediate I-22 (90 g, 254 mmol) was dissolved in 0.8 L of dimethylformamide (DMF) under a nitrogen environment, bis(pinacolato)diboron (77.5 g, 305 mmol), (1,1′-bis(diphenylphosphine)ferrocene)dichloropalladium (II) (2.70 g, 2.54 mmol), and potassium acetate (74.8 g, 762 mmol) were added thereto, and the mixture was heated and refluxed at 150° C. for 25 hours. When the reaction was completed, water was added to the reaction solution, and a mixture was filtered and dried in a vacuum oven. This obtained residue was separated and purified through flash column chromatography to obtain Intermediate I-23 (67.9 g, 60%).

HRMS (70 eV, EI+): m/z calcd for C30H28BNO2: 445.2213. found: 445.

Elemental Analysis: C, 81%; H, 6%.

### Synthesis Example 30: Synthesis of Intermediate I-24

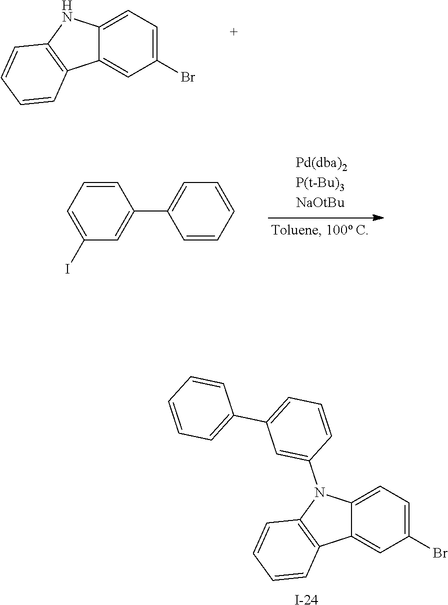

3-bromo-9H-carbazole (100 g, 406 mmol) was dissolved in 1.2 L of toluene under a nitrogen environment, 3-iodobiphenyl (137 g, 488 mmol), bis(dibenzylideneacetone)palladium (0) (2.33 g, 4.06 mmol), tris-tert butylphosphine (4.11 g, 20.3 mmol), and sodium tert-butoxide (46.8 g, 487 mmol) were sequentially added thereto, and the mixture was heated and refluxed at 100° C. for 10 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Intermediate I-24 (82.5 g, 51%).

HRMS (70 eV, EI+): m/z calcd for C24H16BrN: 397.0466. found: 397.

Elemental Analysis: C, 72%; H, 4%.

### Synthesis Example 31: Synthesis of Compound B-1

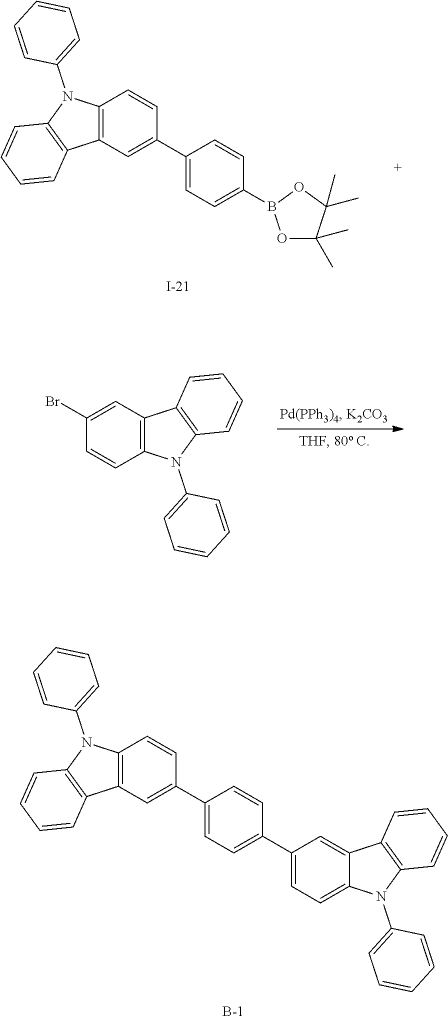

The Intermediate I-21 (20 g, 44.9 mmol) was dissolved in 0.2 L of tetrahydrofuran (THF) under a nitrogen environment, 3-bromo-9-phenyl-9H-carbazole (14.5 g, 44.9 mmol) and tetrakis(triphenylphosphine)palladium (0.52 g, 0.45 mmol) were added thereto, and the mixture was stirred. Potassium carbonate saturated in water (16.5 g, 112 mmol) was added thereto, and the obtained mixture was heated and refluxed at 80° C. for 15 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound B-1 (22.7 g, 90%).

HRMS (70 eV, EI+): m/z calcd for C42H28N2: 560.2252. found: 560.

Elemental Analysis: C, 90%; H, 5%.

### Synthesis Example 32: Synthesis of Compound B-2

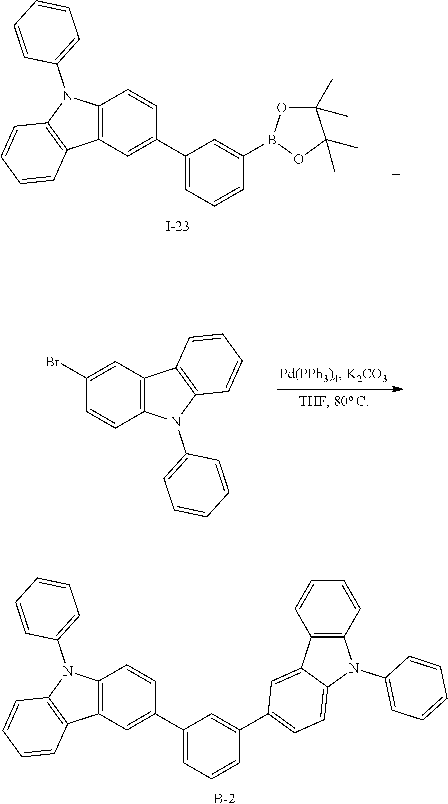

The Intermediate I-23 (20 g, 44.9 mmol) was dissolved in 0.2 L of tetrahydrofuran (THF) under a nitrogen environment, 3-bromo-9-phenyl-9H-carbazole (14.5 g, 44.9 mmol) and tetrakis(triphenylphosphine)palladium (0.52 g, 0.45 mmol) were added thereto, and the mixture was stirred. Potassium carbonate saturated in water (16.5 g, 112 mmol) was added thereto, and the obtained mixture was heated and refluxed at 80° C. for 17 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound B-2 (21.4 g, 85%).

HRMS (70 eV, EI+): m/z calcd for C42H28N2: 560.2252. found: 560.

Elemental Analysis: C, 90%; H, 5%.

### Synthesis Example 33: Synthesis of Compound B-33

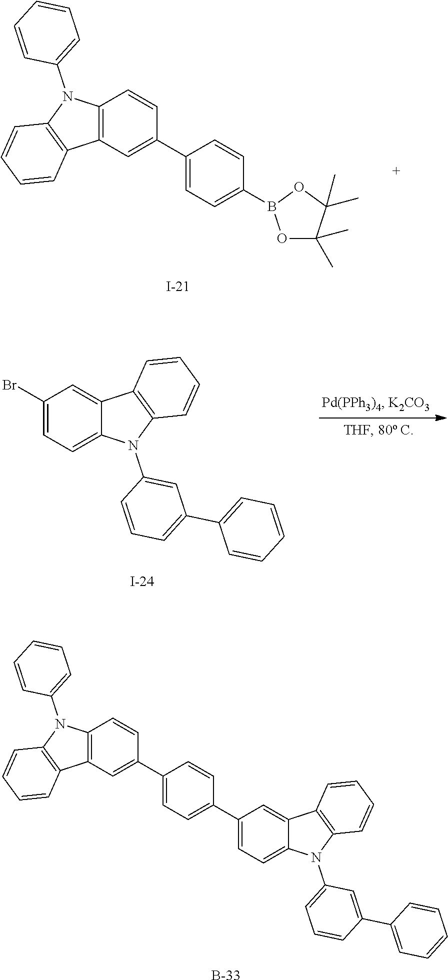

The Intermediate I-21 (20 g, 44.9 mmol) was dissolved in 0.2 L of tetrahydrofuran (THF) under a nitrogen environment, the intermediate I-24 (17.9 g, 44.9 mmol) and tetrakis(triphenylphosphine)palladium (0.52 g, 0.45 mmol) were added thereto, and the mixture was stirred. Potassium carbonate saturated in water (16.5 g, 112 mmol) was added thereto, and the obtained mixture was heated and refluxed at 80° C. for 18 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound B-33 (24.6 g, 86%).

HRMS (70 eV, EI+): m/z calcd for C48H32N2: 636.2565. found: 636.

Elemental Analysis: C, 91%; H, 5%.

### Synthesis Example 34: Synthesis of Compound B-34

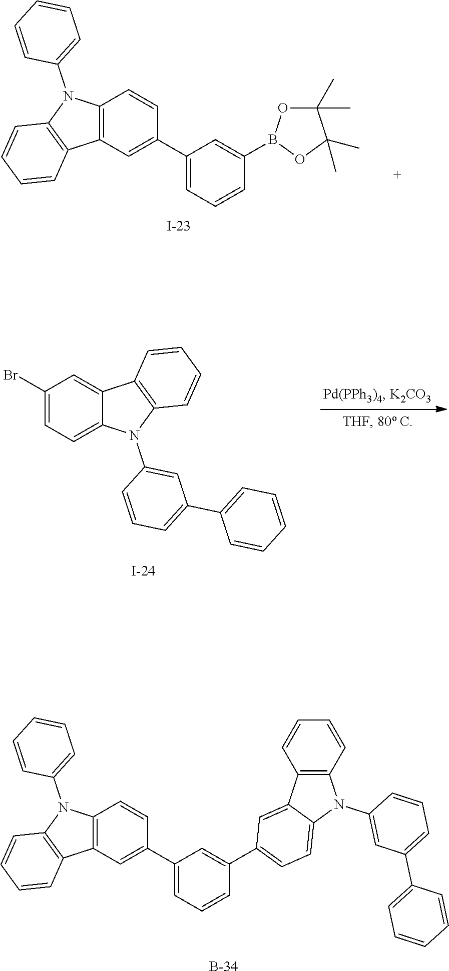

The Intermediate I-23 (20 g, 44.9 mmol) was dissolved in 0.2 L of tetrahydrofuran (THF) under a nitrogen environment, the Intermediate I-24 (17.9 g, 44.9 mmol) and tetrakis(triphenylphosphine)palladium (0.52 g, 0.45 mmol) were added thereto, and the mixture was stirred. Potassium carbonate saturated in water (16.5 g, 112 mmol) was added thereto, and the obtained mixture was heated and refluxed at 80° C. for 18 hours. When the reaction was completed, water was added to the reaction solution, dichloromethane (DCM) was used for an extraction and an extract therefrom was filtered after removing moisture with anhydrous MgSO₄ and then, concentrated under a reduced pressure. This obtained residue was separated and purified through flash column chromatography to obtain Compound B-34 (25.7 g, 90%).

HRMS (70 eV, EI+): m/z calcd for C42H32N2: 636.2565. found: 636.

Elemental Analysis: C, 91%; H, 5%.

### Manufacture of Organic Light Emitting Diode (Green)

### Example 1

A glass substrate coated with ITO (indium tin oxide) to be 1500 Å thick was ultrasonic wave-washed with a distilled water. Subsequently, the glass substrate was ultrasonic wave-washed with a solvent such as isopropyl alcohol, acetone, methanol, and the like, moved to a plasma cleaner, cleaned by using oxygen plasma for 10 minutes, and then, moved to a vacuum depositor. This ITO transparent electrode was used as an anode, a 700 Å-thick hole injection layer was formed thereon by vacuum-depositing Compound A, a hole transport layer was formed on the hole injection layer by depositing Compound B to be 50 Å thick and then Compound C to be 1020 Å thick. On the hole transport layer, a 400 Å-thick light-emitting layer was formed by vacuum-depositing Compound 6 of Synthesis Example 20 and Compound B-1 of Synthesis Example 31 as a second host and 10 wt % of tris(2-phenylpyridinato)iridium (III) [Ir(ppy)3] as a dopant. Herein, Compound 6 and Compound B-1 were used in a ratio of 1:1. Then, on the light-emitting layer, a 300 Å-thick electron transport layer was formed by simultaneously vacuum-depositing Compound D and Liq in a 1:1 ratio, and a cathode was formed by sequentially vacuum-depositing Liq to be 15 Å thick and Al to be 1200 Å thick on the electron transport layer to manufacture an organic light emitting diode.

The organic light emitting diode had a five-layered organic thin film structure and specifically,

a structure of ITO/Compound A (700 Å)/Compound B (50 Å)/Compound C (1020 Å)/EML [Compound 6:Compound B-1:Ir(ppy)3=X:X:10%] 400 Å/Compound D:Liq 300 Å/Liq 15 Å/Al (1200 Å). (X=weight ratio)
Compound A: N4,N4′-diphenyl-N4,N4′-bis(9-phenyl-9H-carbazol-3-yl)biphenyl-4,4′-diamine
Compound B: 1,4,5,8,9,11-hexaazatriphenylene-hexacarbonitrile (HAT-CN),
Compound C: N-(biphenyl-4-yl)-9,9-dimethyl-N-(4-(9-phenyl-9H-carbazol-3-yl)phenyl)-9H-fluoren-2-amine
Compound D: 8-(4-(4,6-di(naphthalen-2-yl)-1,3,5-triazin-2-yl)phenyl)quinolone

### Example 2

An organic light emitting diode was manufactured according to the same method as Example 1 except for using Compound 7 instead of Compound 6.

### Example 3

An organic light emitting diode was manufactured according to the same method as Example 1 except for using Compound 13 instead of Compound 6.

### Example 4

An organic light emitting diode was manufactured according to the same method as Example 1 except for using Compound 14 instead of Compound 6.

### Example 5

An organic light emitting diode was manufactured according to the same method as Example 2 except for using Compound B-2 instead of Compound B-1.

### Example 6

An organic light emitting diode was manufactured according to the same method as Example 2 except for using Compound B-33 instead of Compound B-1.

### Example 7

An organic light emitting diode was manufactured according to the same method as Example 2 except for using Compound B-34 instead of Compound B-1.

### Comparative Example 1

An organic light emitting diode was manufactured according to the same method as Example 1 except for using 4,4′-di(9H-carbazol-9-yl)biphenyl (CBP) as a single host instead of two hosts of Compound 6 and Compound B-1.

### Comparative Example 2

An organic light emitting diode was manufactured according to the same method as Example 1 except for using Compound 6 as a single host instead of two hosts of Compound 6 and Compound B-1.

### Comparative Example 3

An organic light emitting diode was manufactured according to the same method as Example 1 except for using Compound 7 as a single host instead of two hosts of Compound 6 and Compound B-1.

### Comparative Example 4

An organic light emitting diode was manufactured according to the same method as Example 1 except for using Compound 13 as a single host instead of two hosts of Compound 6 and Compound B-1.

### Comparative Example 5

An organic light emitting diode was manufactured according to the same method as Example 1 except for using Compound 14 as a single host instead of two hosts of Compound 6 and Compound B-1.

### Comparative Example 6

An organic light emitting diode was manufactured according to the same method as Example 1 except for using Compound B-1 as a single host instead of two hosts of Compound 6 and Compound B-1.

### Comparative Example 7

An organic light emitting diode was manufactured according to the same method as Example 1 except for using Compound B-2 as a single host instead of two hosts of Compound 6 and Compound B-1.

### Comparative Example 8

An organic light emitting diode was manufactured according to the same method as Example 1 except for using Compound B-33 as a single host instead of two hosts of Compound 6 and Compound B-1.

### Comparative Example 9

An organic light emitting diode was manufactured according to the same method as Example 1 except for using Compound B-34 as a single host instead of two hosts of Compound 6 and Compound B-1.

### Evaluation

Luminous efficiency and life-span of each organic light emitting diode according to Examples 1 to 7 and Comparative Examples 1 to 9 were measured.

Specific measurement methods were as follows, and the results were provided in Table 1.

(1) Measurement of Current Density Change Depending on Voltage Change

Current values flowing in the unit devices of the obtained organic light emitting diodes were measured while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm²) were calculated by using the luminance, current density, and voltages from the items (1) and (2).

(4) Measurement of Life-Span

Life-span was obtained by emitting organic light emitting diodes at initial luminance of 6000 cd/m², measuring luminance decrease as time goes, and measuring a time taken until the luminance decreased by 97% relative to the initial luminance.

TABLE 1

| | | | | Luminous | Life-span |
|---|---|---|---|---|---|
| | | Second | | efficiency | T97% |
| | First host | host | First host:Second host | (cd/A) | (h) |
| Example 1 | Compound 6 | B-1 | 1:1 | 59.1 | 680 |
| Example 2 | Compound 7 | B-1 | 1:1 | 55.2 | 720 |
| Example 3 | Compound 13 | B-1 | 1:1 | 57.6 | 700 |
| Example 4 | Compound 14 | B-1 | 1:1 | 56.1 | 750 |
| Example 5 | Compound 7 | B-2 | 1:1 | 58.8 | 710 |
| Example 6 | Compound 7 | B-33 | 1:1 | 53.1 | 760 |
| Example 7 | Compound 7 | B-34 | 1:1 | 54.3 | 750 |

| | | | | |
|---|---|---|---|---|
| Comparative | CBP | — | 19.3 | 0.5 |
| Example 1 | | | | |
| Comparative | Compound 6 | — | 28.7 | 480 |
| Example 2 | | | | |
| Comparative | Compound 7 | — | 33.5 | 550 |
| Example 3 | | | | |
| Comparative | Compound 13 | — | 30.1 | 500 |
| Example 4 | | | | |
| Comparative | Compound 14 | — | 35.7 | 400 |
| Example 5 | | | | |
| Comparative | B-1 | — | 7.8 | 10 |
| Example 6 | | | | |
| Comparative | B-2 | — | 12.8 | 10 |
| Example 7 | | | | |
| Comparative | B-33 | — | 8.9 | 30 |
| Example 8 | | | | |
| Comparative | B-34 | — | 11.7 | 30 |
| Example 9 | | | | |

Referring to Table 1, the organic light emitting diodes according to Examples 1 to 7 exhibited remarkably improved luminous efficiency and life-span characteristics compared with the organic light emitting diodes according to Comparative Examples 1 to 9. When the organic light emitting diodes having satisfactory life-span characteristics and luminous efficiency according to Comparative Examples 2 to 5 were mixed with the organic light emitting diodes having excellent hole characteristics according to Comparative Examples 6 to 9, luminous efficiency and life-span characteristics may be remarkably improved due to a synergy effect of each luminous efficiency and life-span characteristics.

The present invention is not limited to the example embodiments and may be implemented in various embodiments, and one having an ordinary skill in this art of the present invention may understand that the present invention may be embodied in other specific embodiments within the spirit and scope of the appended claims. Therefore, the aforementioned embodiments should be understood to be exemplary but not limiting the present invention in any way.

### Manufacture of Organic Light Emitting Diode (Red)

### Example 8

An organic light emitting diode was manufactured by using Compound 21 of Synthesis Example 24 as a host and acetylacetonato bis(2-phenylquinolinato)iridium (Ir(pq)₂acac) as a dopant.

As for an anode, 1500 Å-thick ITO was used, and as for a cathode, 1000 Å-thick aluminum (Al) was used. Specifically, illustrating a method of manufacturing the organic light emitting diode, the anode is manufactured by cutting an ITO glass substrate having 15 Ω/cm² of a sheet resistance into a size of 50 mm×50 mm×0.7 mm, ultrasonic wave-washing them in acetone, isopropylalcohol, and pure water for 15 minutes respectively, and UV ozone cleaning them for 30 minutes.

On the substrate, a 600 Å-thick hole transport layer (HTL) was formed by vacuum-depositing 4,4′-bis[N-[4-{N,N-bis(3-methylphenyl)amino}-phenyl]-N-phenylamino]biphenyl [DNTPD] under a vacuum degree of 650×10⁻⁷ Pa at a deposition rate of 0.1 to 0.3 nm/s. Subsequently, a 300 Å-thick hole transport layer was formed by vacuum-depositing HT-1 under the same vacuum deposition condition. Then, a 300 Å-thick light-emitting layer was formed by using Compound 21 of Synthesis Example 24 and Compound B-1 of Synthesis Example 31 as a second host under the same vacuum deposition condition, and Compound 21 and Compound B-1 were used in a 1:1 ratio. When the hosts were deposited, a phosphorescent dopant, acetylacetonatobis(2-phenylquinolinato)iridium (Ir(pq)₂acac) was simultaneously deposited. Herein, the phosphorescent dopant was deposited to be 7 wt % based on 100 wt % of a total weight of the light-emitting layer by adjusting a deposition rate of the phosphorescent dopant.

On the light-emitting layer, a 50 Å-thick hole blocking layer was formed by depositing bis(2-methyl-8-quinolinolate)-4-(phenylphenolato)aluminum (BAlq) under the same vacuum deposition condition. Subsequently, a 250 Å-thick electron transport layer was formed by depositing tris(8-hydroxyquinolinato)aluminum (Alq3) under the same vacuum deposition condition. On the electron transport layer, a cathode is formed by sequentially depositing LiF and Al to manufacture an organic light emitting diode.

A structure of the organic light emitting diode was ITO/DNTPD (60 nm)/HT-1 (30 nm)/EML (Compound 24:B-1=1:1 of a weight ratio) (93 wt %)+Ir(pq)₂acac (7 wt %), 30 nm)/Balq (5 nm)/Alq3 (25 nm)/LiF (1 nm)/Al (100 nm).

### Example 9

An organic light emitting diode was manufactured according to the same method as Example 8 except for using Compound 22 instead of Compound 21.

### Example 10

An organic light emitting diode was manufactured according to the same method as Example 9 except for using Compound B-2 instead of Compound B-1.

### Example 11

An organic light emitting diode was manufactured according to the same method as Example 9 except for using Compound B-33 instead of Compound B-1.

### Example 12

An organic light emitting diode was manufactured according to the same method as Example 9 except for using Compound B-34 instead of Compound B-1.

### Comparative Example 10

An organic light emitting diode was manufactured according to the same method as Example 8 except for using CBP as a single host instead of two hosts of Compound 21 and Compound B-1.

### Comparative Example 11

An organic light emitting diode was manufactured according to the same method as Example 8 except for using Compound 21 as a single host instead of two hosts of Compound 21 and Compound B-1.

### Comparative Example 12

An organic light emitting diode was manufactured according to the same method as Example 8 except for using Compound 22 as a single host instead of two hosts of Compound 21 and Compound B-1.

### Comparative Example 13

An organic light emitting diode was manufactured according to the same method as Example 8 except for using Compound B-1 as a single host instead of two hosts of Compound 21 and Compound B-1.

### Comparative Example 14

An organic light emitting diode was manufactured according to the same method as Example 8 except for using Compound B-2 as a single host instead of two hosts of Compound 21 and Compound B-1.

### Comparative Example 15

An organic light emitting diode was manufactured according to the same method as Example 8 except for using Compound B-33 as a single host instead of two hosts of Compound 21 and Compound B-1.

### Comparative Example 16

An organic light emitting diode was manufactured according to the same method as Example 8 except for using Compound B-34 as a single host instead of two hosts of Compound 21 and Compound B-1.

DNTPD, BAlq, HT-1, CBP, and Ir(pq)₂acac used to manufacture the organic light emitting diode have the following structures.

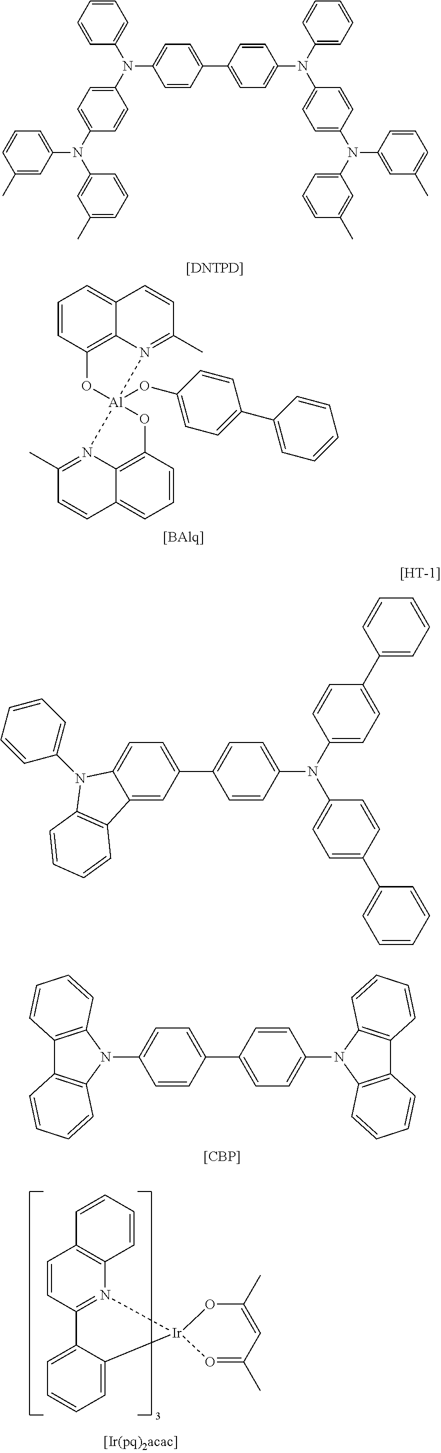

Evaluation

Luminous efficiency and life-span of each organic light emitting diode according to Examples 8 to 12 and Comparative Examples 10 to 16 were measured.

Specific measurement methods were as follows, and the results were provided in Table 2.

(1) Measurement of Current Density Change Depending on Voltage Change

The obtained organic light emitting diodes were measured for current value flowing in the unit device while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm²) were calculated by using the luminance, current density, and voltages from the items (1) and (2).

(4) Measurement of Life-Span

Life-span was obtained by emitting organic light emitting diodes at initial luminance of 3000 cd/m², measuring luminance decrease as time goes, and measuring a time taken until the luminance decreased by 50% relative to the initial luminance.

TABLE 2

| | | | | | 50% life-span |
|---|---|---|---|---|---|
| | | Second | First host:Second | Efficiency | (h) |
| | First host | host | host | (cd/A) | at 3000 cd/m² |
| Example 8 | Compound 21 | B-1 | 1:1 | 51.3 | 490 |
| Example 9 | Compound 22 | B-1 | 1:1 | 50.0 | 510 |
| Example 10 | Compound 22 | B-2 | 1:1 | 49.5 | 500 |
| Example 11 | Compound 22 | B-33 | 1:1 | 48.2 | 550 |
| Example 12 | Compound 22 | B-34 | 1:1 | 48.5 | 540 |

| | | | | |
|---|---|---|---|---|
| Comparative | CBP | — | 37.2 | 220 |
| Example 10 | | | | |
| Comparative | Compound 21 | — | 41.2 | 150 |
| Example 11 | | | | |
| Comparative | Compound 22 | — | 40.4 | 250 |
| Example 12 | | | | |
| Comparative | B-1 | — | 10 | 0 |
| Example 13 | | | | |
| Comparative | B-2 | — | 10 | 0 |
| Example 14 | | | | |
| Comparative | B-33 | — | 20 | 0 |
| Example 15 | | | | |
| Comparative | B-34 | — | 10 | 0 |
| Example 16 | | | | |

Referring to Table 2, the organic light emitting diodes according to Examples 8 to 12 exhibited remarkably improved luminous efficiency and life-span characteristics compared with the organic light emitting diodes according to Comparative Examples 10 to 16. When the organic light emitting diodes having satisfactory life-span characteristics and luminous efficiency according to Comparative Examples 11 and 12 were appropriately mixed with the organic light emitting diodes having excellent hole characteristics according to Comparative Examples 13 to 16, luminous efficiency and life-span characteristics may be remarkably improved due to a synergy effect of each luminous efficiency and life-span characteristics.

### (Energy Level Using Gaussian Tool)

An energy level of each material was measured in a B3LYP/6-31G method by using a program Gaussian 09 with a super computer, GAIA (IBM power 6), and the results are shown in Table 3.

TABLE 3

| Material | HOMO (eV) | LUMO (eV) |
|---|---|---|
| Compound 6 | −5.99 | −1.87 |
| Compound 7 | −5.92 | −1.81 |
| Compound 13 | −5.76 | −1.82 |
| Compound 14 | −5.28 | −1.82 |
| Compound 21 | −5.65 | −1.88 |
| Compound 22 | −5.65 | −1.87 |
| B-1 | −5.04 | −0.77 |
| B-2 | −5.17 | −0.73 |
| B-33 | −5.04 | −0.98 |
| B-34 | −5.17 | −0.97 |

According to the results, Compounds 6, 7, 13, 14, 21, and 22 had a lower LUMO energy level than Compounds B-1, B-2, B-33, and B-34. Thereby, electron injection is more easily in Compounds 6, 7, 13, 14, 21, and 22 than Compounds B-1, B-2, B-33, and B-34.

In addition, Compounds B-1, B-2, B-33, and B-34 had a higher HOMO energy level than Compounds 6, 7, 13, 14, 21, and 22. Thereby, hole injection is more easily carried out in Compounds B-1, B-2, B-33, and B-34 than Compounds 6, 7, 13, 14, 21, and 22. When these materials facilitating hole/electron flows were used together as shown in Tables 1 and 2, a synergy effect may be generated and thus provide a device having high efficiency/long life-span.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

1. A composition for an organic optoelectric diode, comprising
a first host compound represented by Chemical Formula I, and
a second host compound represented by Chemical Formula II: 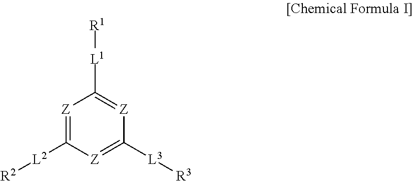
wherein, in Chemical Formula I,
Z's are independently N or CR^{a},
at least two of three Z's are N,
R¹ to R³ and R^{a} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C2 to C30 alkoxycarbonyl group, a substituted or unsubstituted C2 to C30 alkoxycarbonylamino group, a substituted or unsubstituted C7 to C30 aryloxycarbonylamino group, a substituted or unsubstituted C1 to C30 sulfamoylamino group, a substituted or unsubstituted C2 to C30 alkenyl group, a substituted or unsubstituted C2 to C30 alkynyl group, a substituted or unsubstituted C3 to C40 silyl group, a substituted or unsubstituted C3 to C40 silyloxy group, a substituted or unsubstituted C1 to C30 acyl group, a substituted or unsubstituted C1 to C20 acyloxy group, a substituted or unsubstituted C1 to C20 acylamino group, a substituted or unsubstituted C1 to C30 sulfonyl group, a substituted or unsubstituted C to C30 alkylthiol group, a substituted or unsubstituted C6 to C30 arylthiol group, a substituted or unsubstituted C1 to C30 ureide group, a halogen, a halogen-containing group, a cyano group, a hydroxyl group, an amino group, a nitro group, a carboxyl group, a ferrocenyl group, or a combination thereof,
adjacent two selected from R¹ to R³ and R^{a} are linked to each other to provide a ring,
L¹ to L³ are independently a single bond, a substituted or unsubstituted C1 to C30 alkylene group, a substituted or unsubstituted C3 to C30 cycloalkylene group, a substituted or unsubstituted C6 to C30 arylene group, a substituted or unsubstituted C2 to C30 heteroarylene group, a substituted or unsubstituted C6 to C30 aryleneamine group, a substituted or unsubstituted C1 to C30 alkoxylene group, a substituted or unsubstituted C1 to C30 aryloxylene group, a substituted or unsubstituted C2 to C30 alkenylene group, a substituted or unsubstituted C2 to C30 alkynylene group, or a combination thereof, and
when the L¹ to L³ are all single bonds, all the R¹ to R³ are not hydrogen, 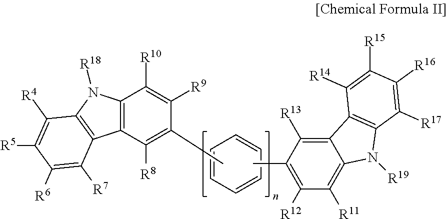
wherein, in Chemical Formula II,
R⁴ to R¹⁷ are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,
adjacent two of R⁴ to R¹⁰ and R¹¹ to R¹⁷ are linked to each other to provide a ring,
R¹⁸ and R¹⁹ are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heteroaryl group, a substituted or unsubstituted C6 to C30 arylamine group, a substituted or unsubstituted C1 to C30 alkoxy group, a substituted or unsubstituted C2 to C30 alkoxycarbonyl group, a substituted or unsubstituted C2 to C30 alkoxycarbonylamino group, a substituted or unsubstituted C7 to C30 aryloxycarbonylamino group, a substituted or unsubstituted C1 to C30 sulfamoylamino group, a substituted or unsubstituted C2 to C30 alkenyl group, a substituted or unsubstituted C2 to C30 alkynyl group, a substituted or unsubstituted C3 to C40 silyl group, a substituted or unsubstituted C3 to C40 silyloxy group, a substituted or unsubstituted C1 to C30 acyl group, a substituted or unsubstituted C1 to C20 acyloxy group, a substituted or unsubstituted C1 to C20 acylamino group, a substituted or unsubstituted C1 to C30 sulfonyl group, a substituted or unsubstituted C to C30 alkylthiol group, a substituted or unsubstituted C6 to C30 arylthiol group, a substituted or unsubstituted C1 to C30 ureide group, a halogen, a halogen-containing group, a cyano group, a hydroxyl group, an amino group, a nitro group, a carboxyl group, a ferrocenyl group, or a combination thereof,
n is an integer ranging from 1 to 4.

2. The composition for an organic optoelectric diode of claim 1, wherein
the first host compound is represented by one of Chemical Formulae I-1 to I-5: 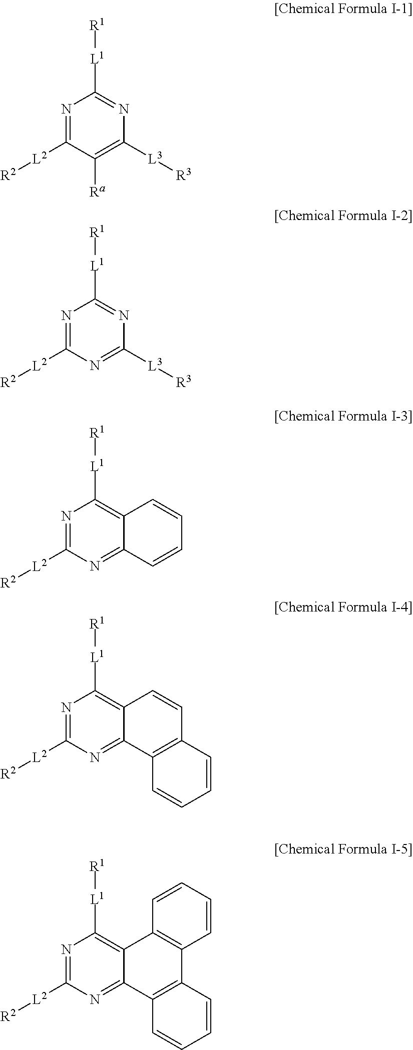
wherein, in Chemical Formulae I-1 to I-5,
R¹ to R³ and R^{a} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C3 to C40 silyl group, a substituted or unsubstituted C1 to C30 alkylthiol group, a substituted or unsubstituted C6 to C30 arylthiol group, a substituted or unsubstituted C1 to C30 ureide group, a halogen, a cyano group, a hydroxyl group, an amino group, a nitro group, a carboxyl group, a ferrocenyl group, or a combination thereof,
L¹ to L³ are independently a single bond, a substituted or unsubstituted C1 to C30 alkylene group, a substituted or unsubstituted C3 to C30 cycloalkylene group, a substituted or unsubstituted C6 to C30 arylene group, a substituted or unsubstituted C2 to C30 heteroarylene group, or a combination thereof, and
when the L¹ to L³ are all single bonds, all the R¹ to R³ are not hydrogen.

3. The composition for an organic optoelectric diode of claim 1, wherein L¹ to L³ of Chemical Formula I are independently a single bond or selected from substituted or unsubstituted groups of Group I: 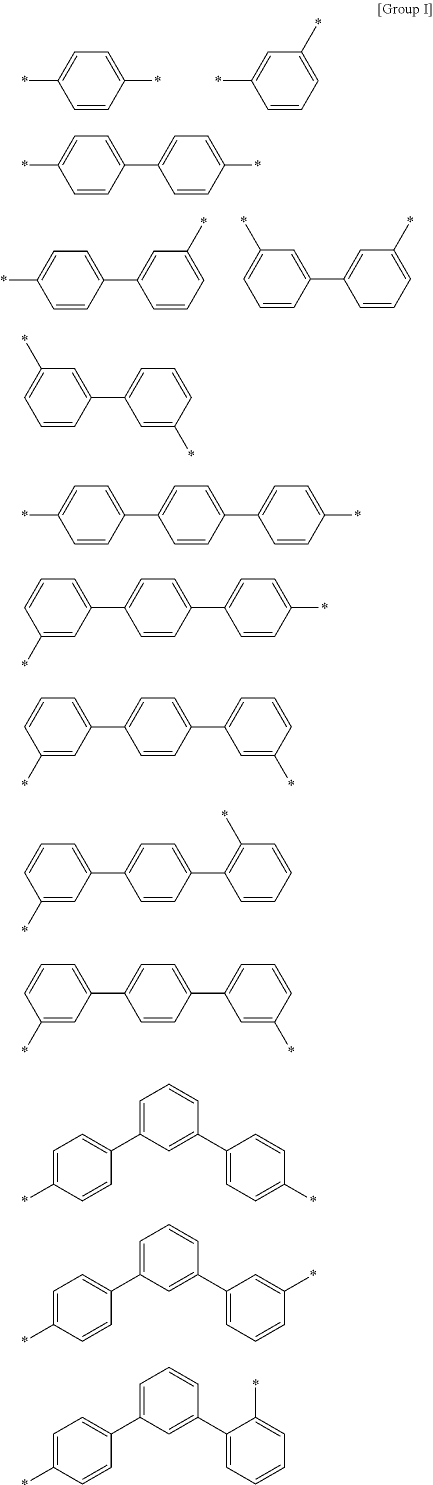
wherein, in Group I,
* is a linking point.

4. The composition for an organic optoelectric diode of claim 1, wherein the R¹ to R³ and R^{a} are independently hydrogen, a substituted or unsubstituted C6 to C30 aryl group, a substituted or unsubstituted C2 to C30 heterocyclic group, or a combination thereof.

5. The composition for an organic optoelectric diode of claim 4, wherein the substituted or unsubstituted C6 to C30 aryl group is a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted 1H-phenalenyl group, a substituted or unsubstituted pyrenyl group, a substituted or unsubstituted fluorenyl group, a substituted or unsubstituted triphenylene group, or a combination thereof, and
the substituted or unsubstituted C2 to C30 heterocyclic group is a substituted or unsubstituted carbazolyl group, a substituted or unsubstituted benzofuranyl group, a substituted or unsubstituted benzothiophenyl group, a substituted or unsubstituted dibenzofuranyl group, a substituted or unsubstituted dibenzothiophenyl group, or a combination thereof.

6. The composition for an organic optoelectric diode of claim 4, wherein the substituted or unsubstituted C6 to C30 aryl group and the substituted or unsubstituted C2 to C30 heterocyclic group are selected from substituted or unsubstituted groups of Group II: 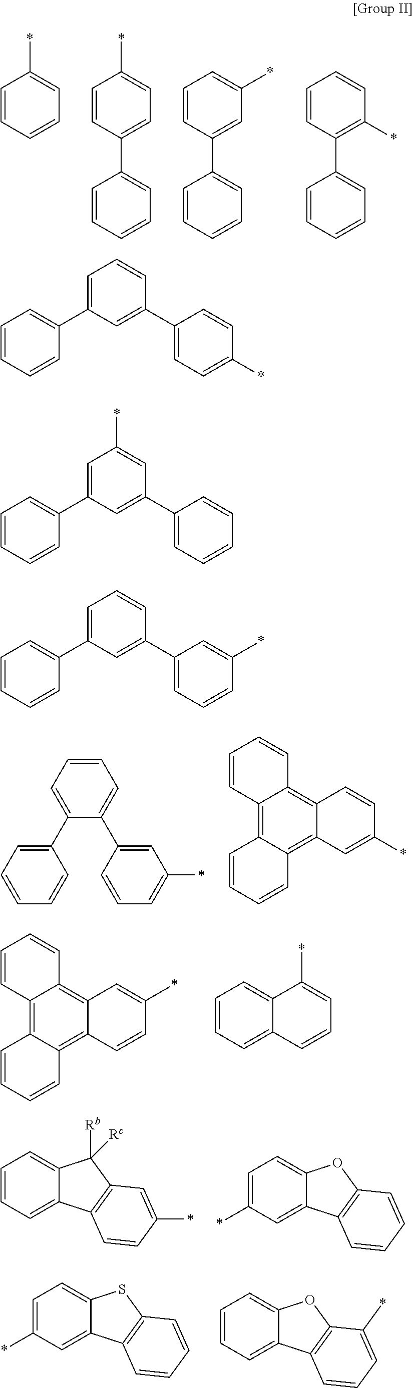 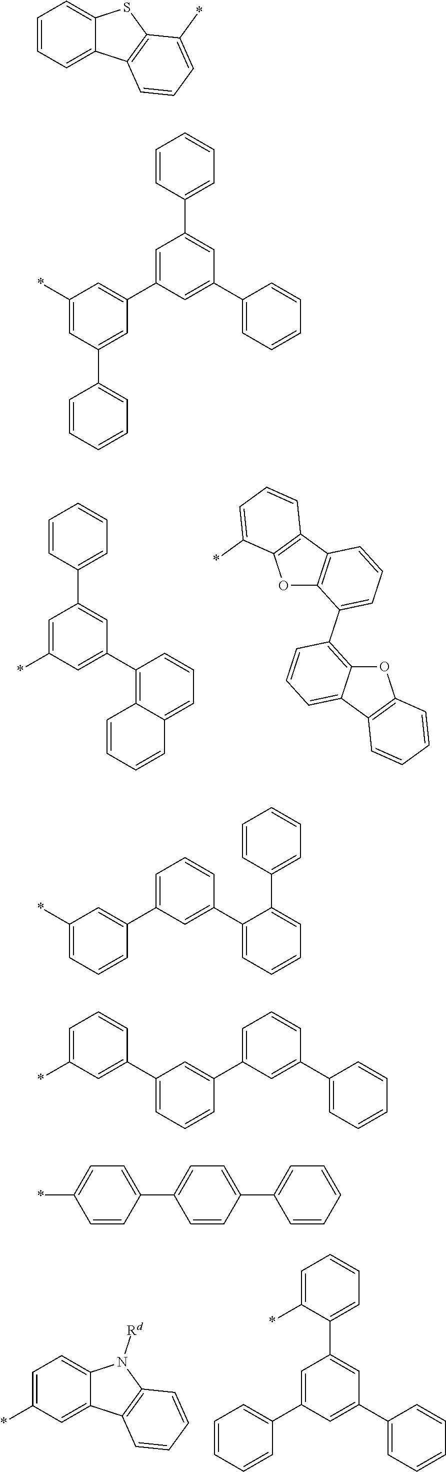 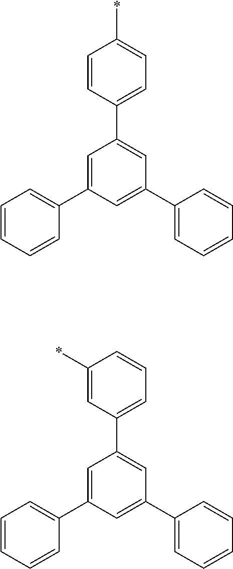
wherein, in Group II,
R^{b} to R^{d} are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof, and
* is a linking point.

7. The composition for an organic optoelectric diode of claim 1, wherein the second host compound is represented by one of Chemical Formulae II-1 to II-16: 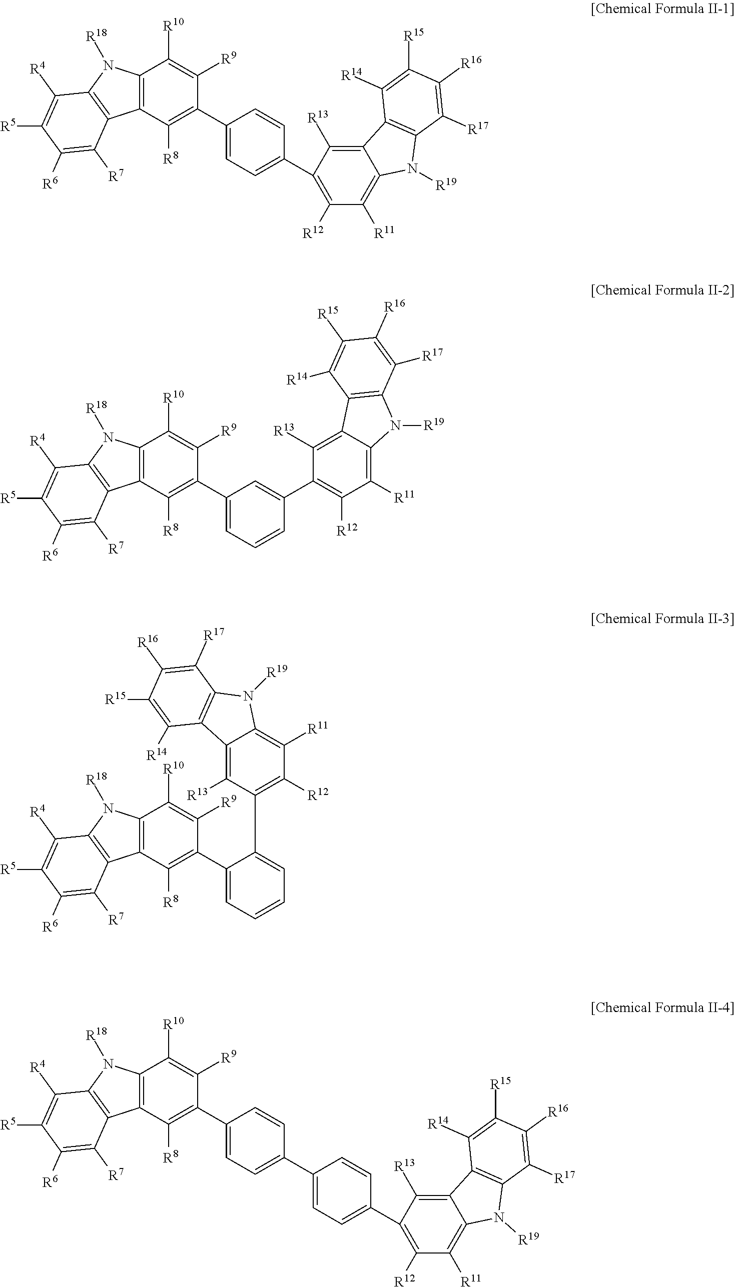 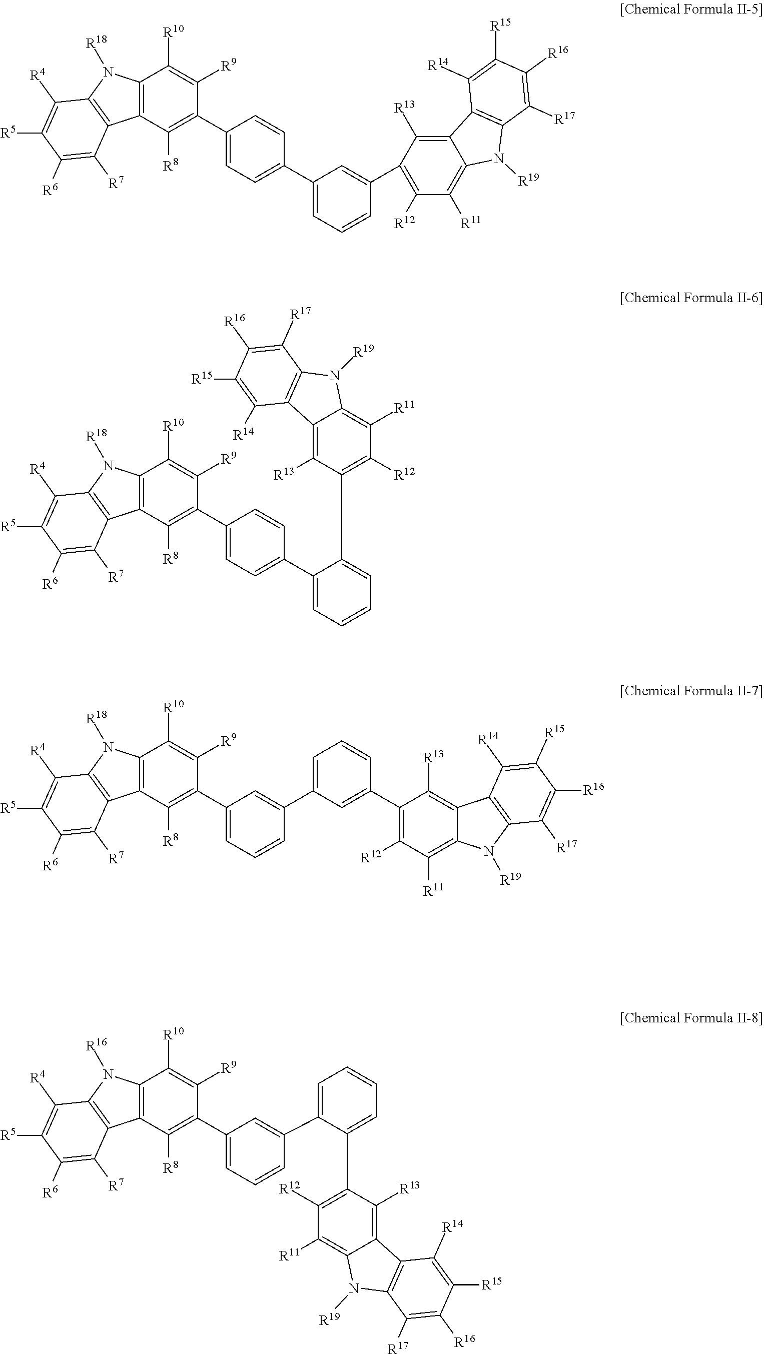 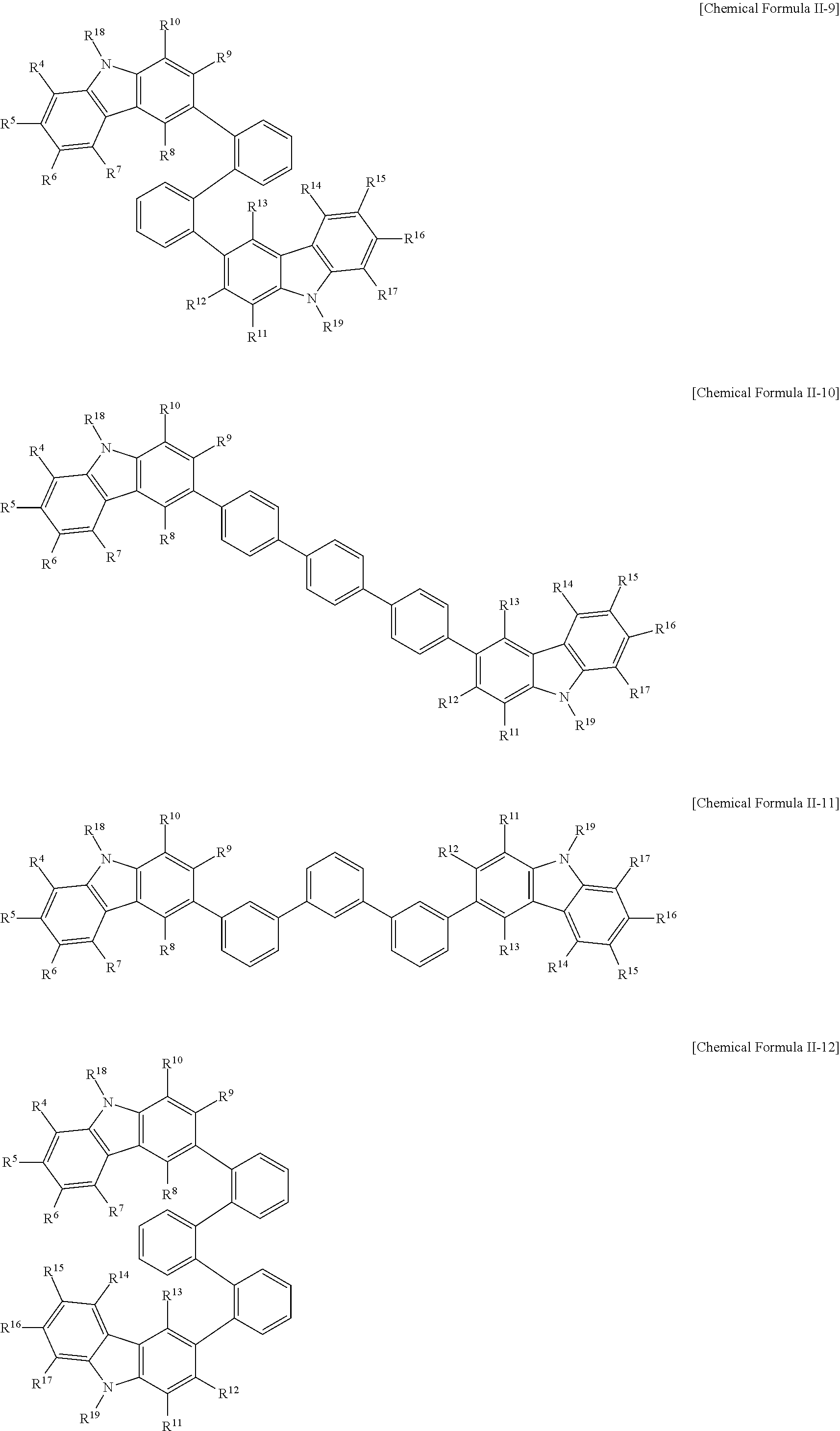 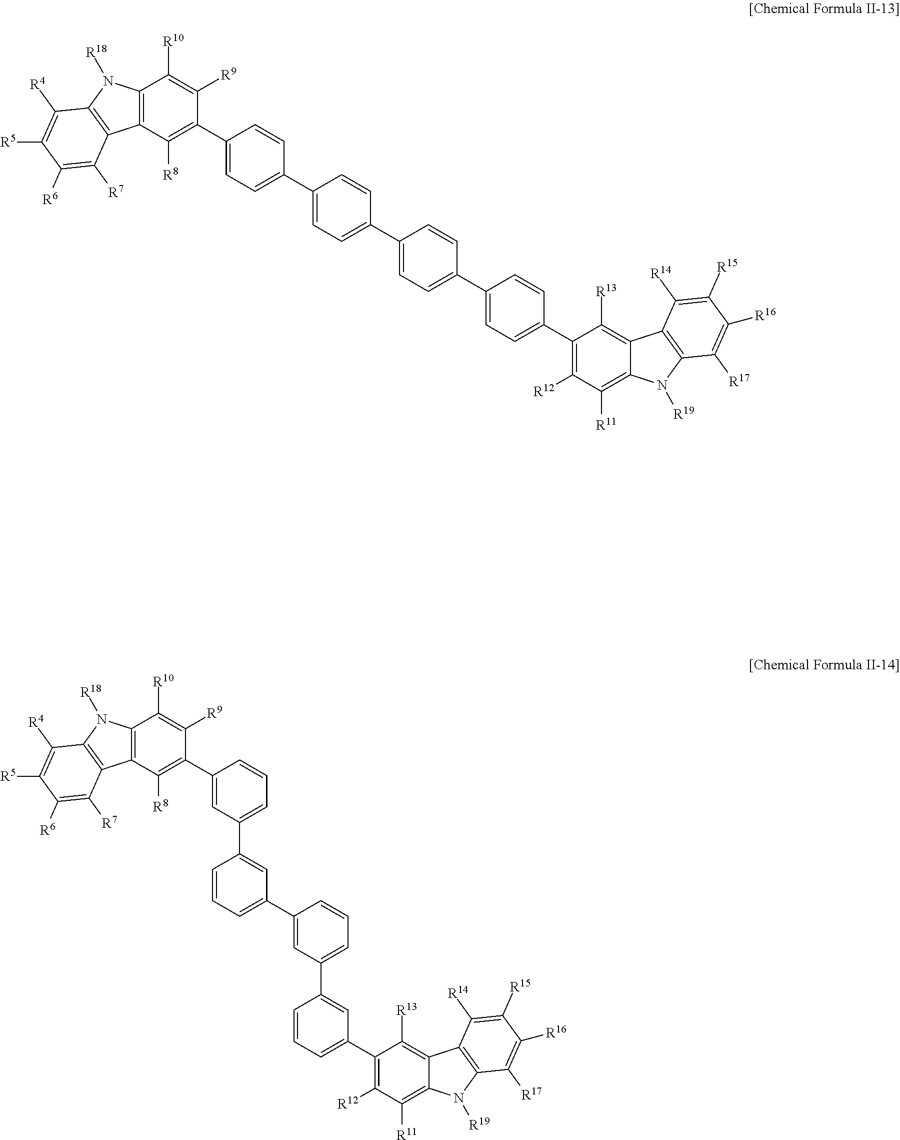 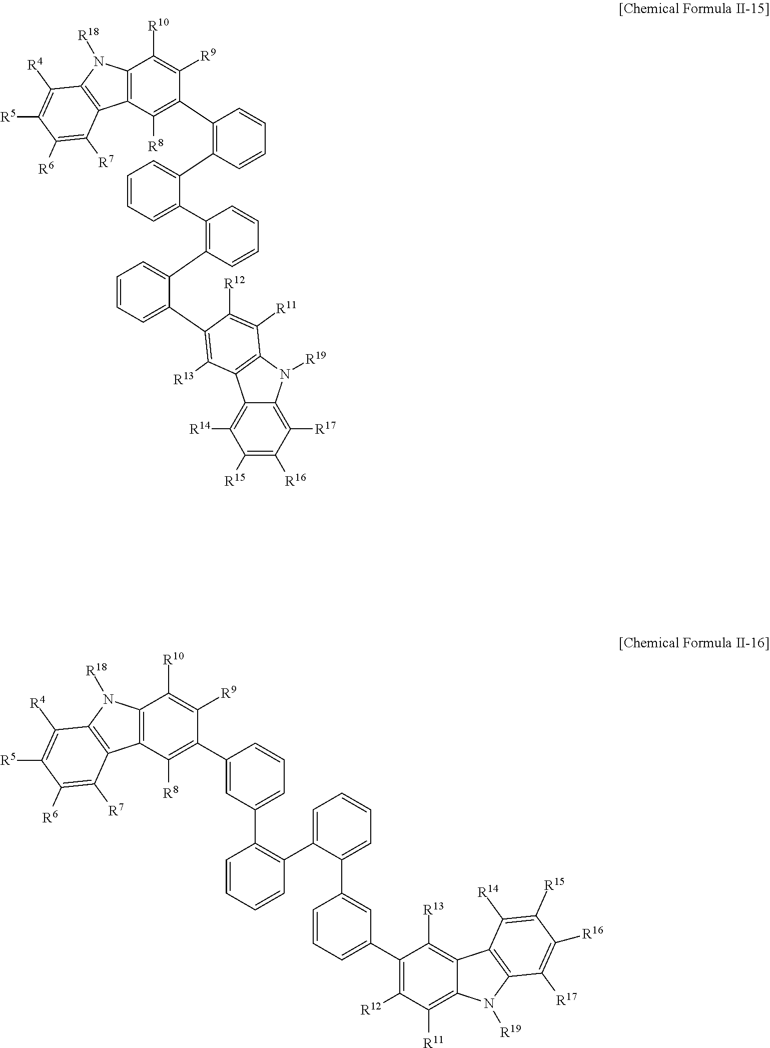
wherein, in Chemical Formula Chemical Formulae II-1 to II-16,
R⁴ to R¹⁷ are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,
adjacent two of R⁴ to R¹⁰ and R¹¹ to R¹⁷ are linked to each other to provide a ring, and
R¹⁸ and R¹⁹ are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heteroaryl group, a substituted or unsubstituted C6 to C30 arylamine group, a substituted or unsubstituted C1 to C30 alkylthiol group, a substituted or unsubstituted C6 to C30 arylthiol group, or a combination thereof.

8. The composition for an organic photoelectric device of claim 1, wherein R¹⁸ and R¹⁹ are independently hydrogen, deuterium, a substituted or unsubstituted C6 to C30 aryl group, or a substituted or unsubstituted C2 to C30 heteroaryl group.

9. The composition for an organic photoelectric device of claim 8, wherein the substituted or unsubstituted C6 to C30 aryl group is a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted 1H-phenalenyl group, a substituted or unsubstituted pyrenyl group, a substituted or unsubstituted fluorenyl group, a substituted or unsubstituted triphenylene group, or a combination thereof,
wherein the substituted or unsubstituted C2 to C30 heteroaryl group is a substituted or unsubstituted pyridyl group, a substituted or unsubstituted pyrimidinyl group, a substituted or unsubstituted triazinyl group, or a combination thereof.

10. The composition for an organic photoelectric device of claim 1, wherein the second host compound is represented be one of Chemical Formulae II-17 to II-39: 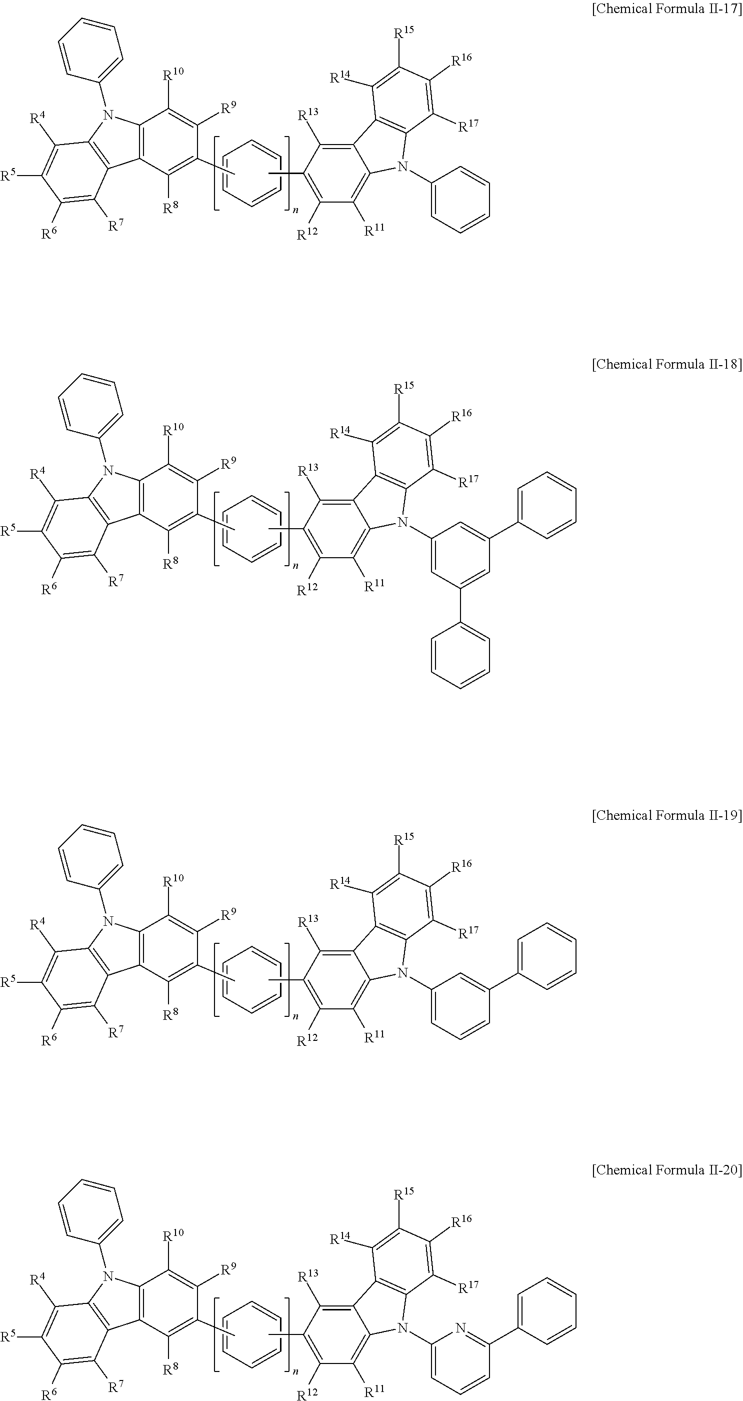 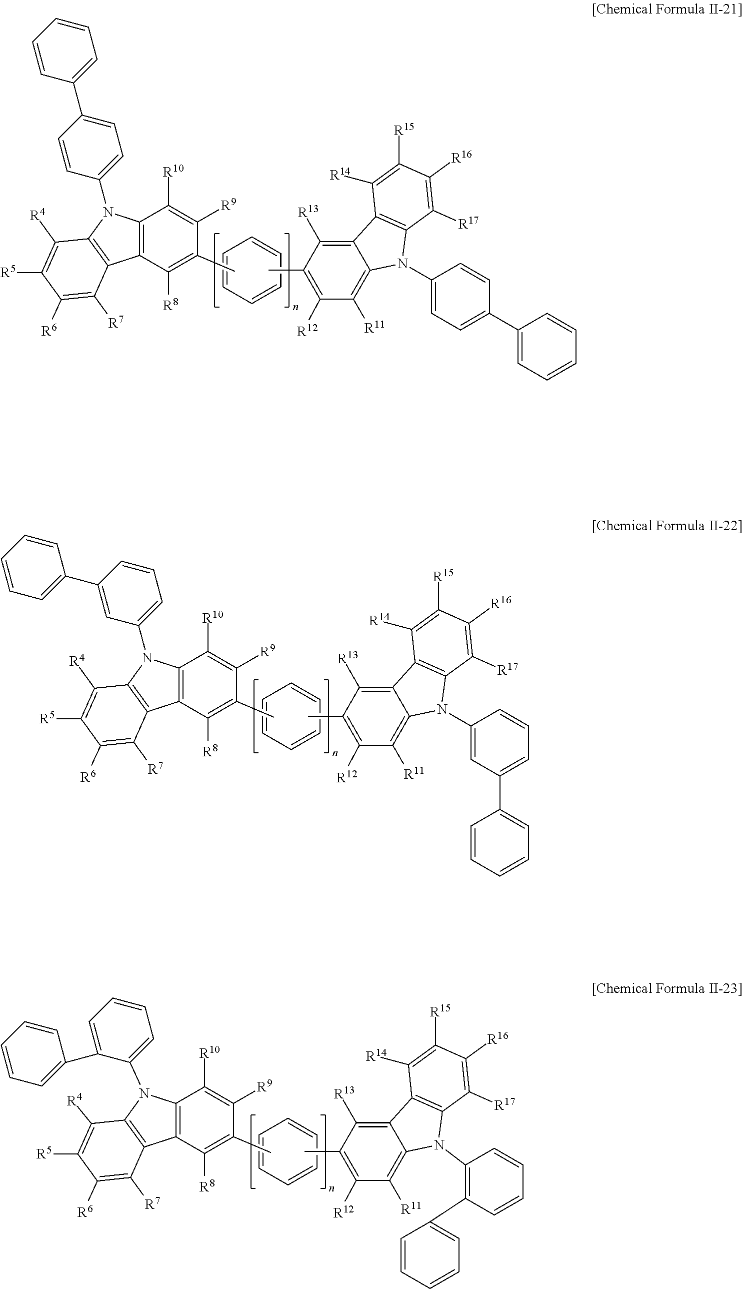 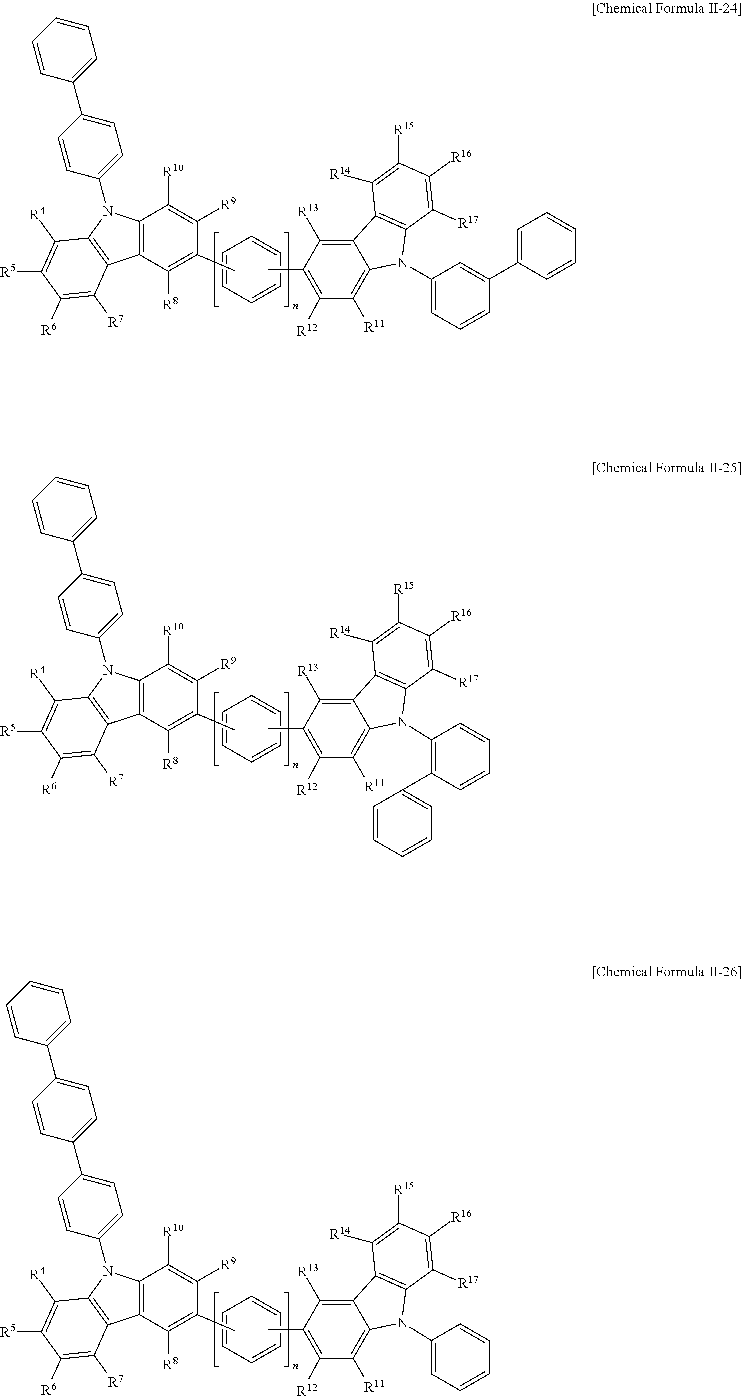 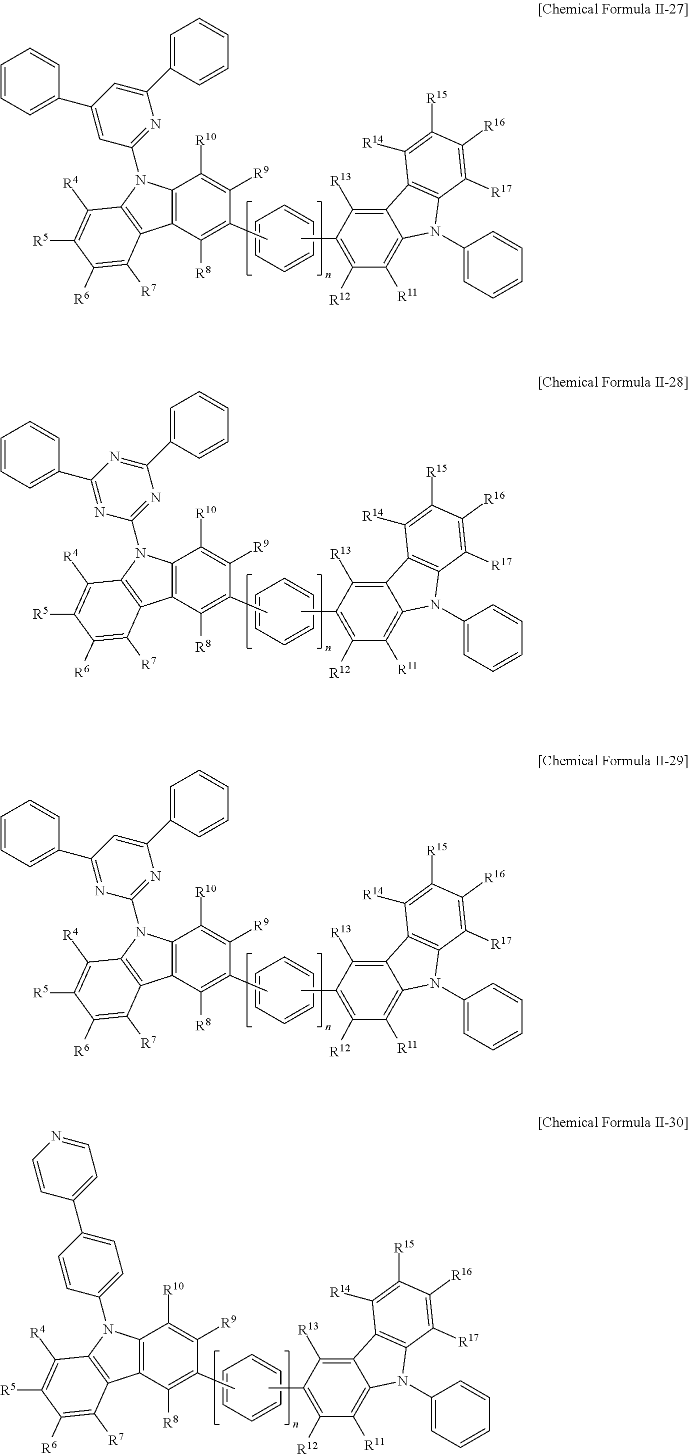 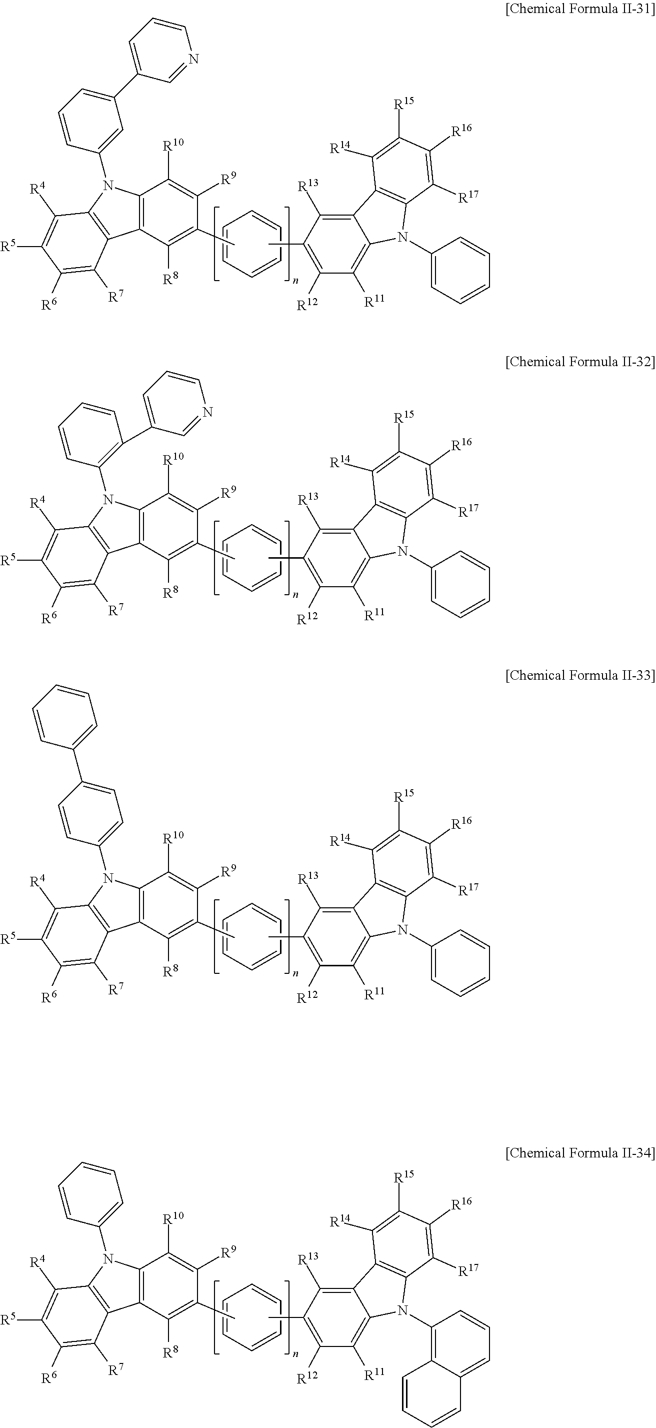 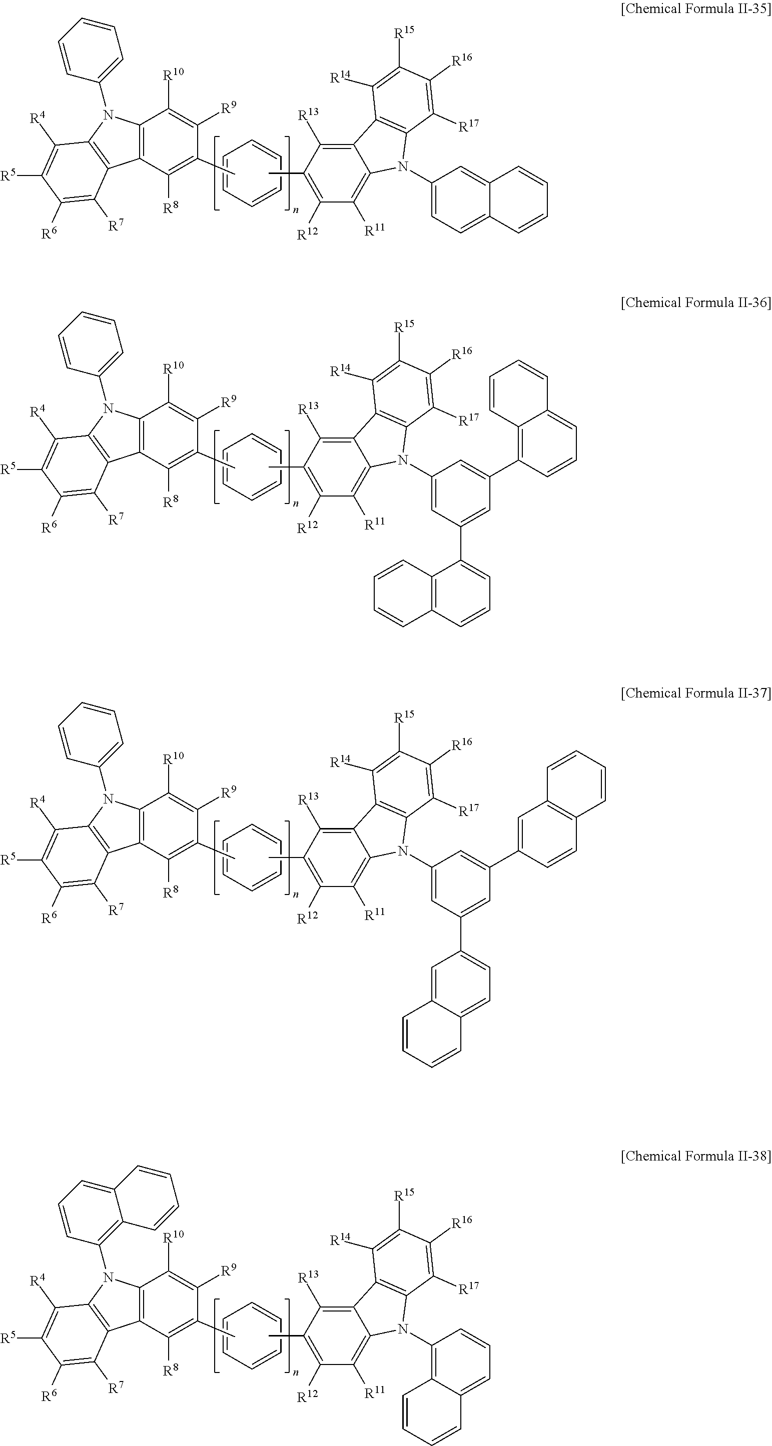 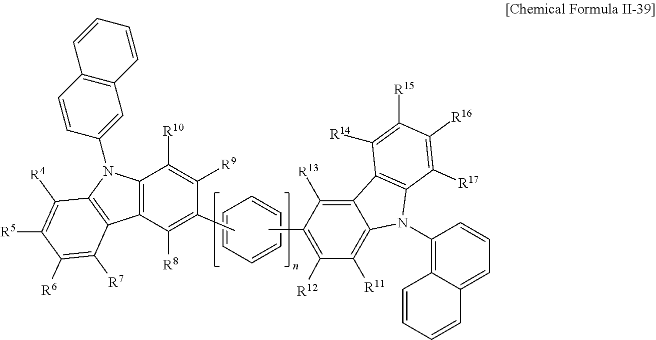
wherein in Chemical Formulae II-17 to II-39,
R⁴ to R¹⁷ are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,
adjacent two of R⁴ to R¹⁰ and R¹¹ to R¹⁷ are linked to each other to provide a ring, and
n is an integer ranging from 1 to 4.

11. The composition for an organic photoelectric device of claim 1, wherein R⁴ to R¹⁷ of Chemical Formula II are independently hydrogen, deuterium, or a substituted or unsubstituted C6 to C30 aryl group.

12. The composition for an organic photoelectric device of claim 1, wherein the first host compound is selected from compounds of Group III:

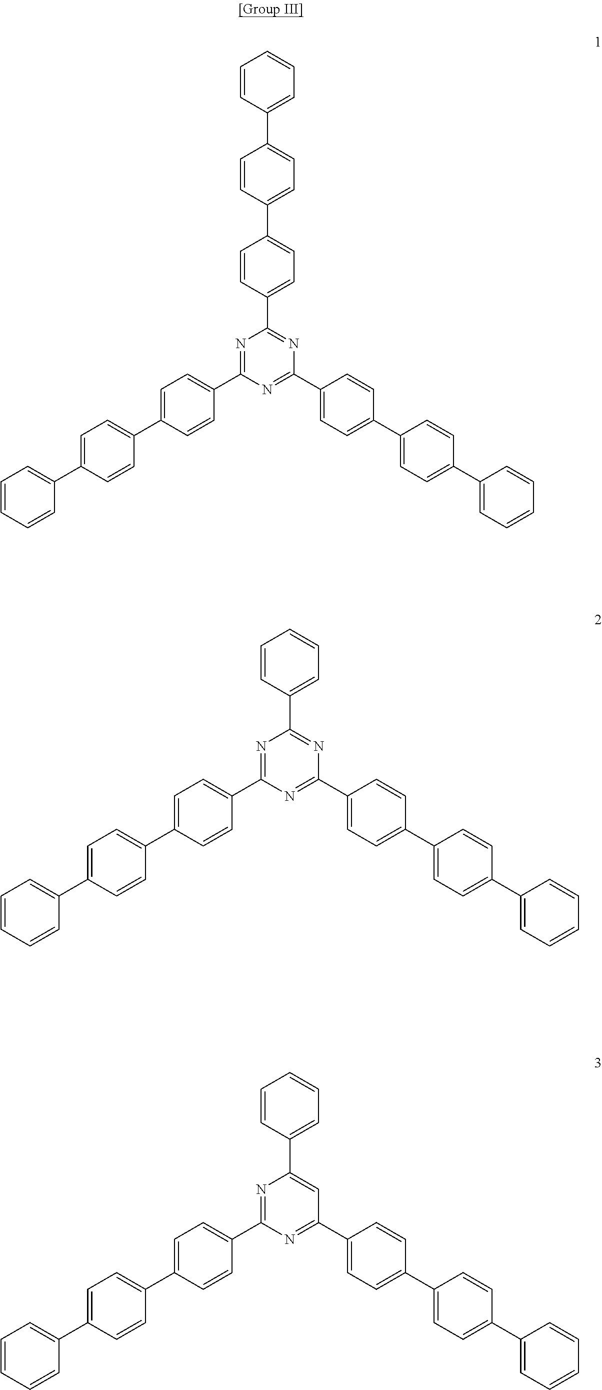 [SMILES: c1ccc(-c2ccc(-c3ccc(-c4nc(-c5ccc(-c6ccc(-c7ccccc7)cc6)cc5)nc(-c5ccc(-c6ccc(-c7ccccc7)cc6)cc5)n4)cc3)cc2)cc1 ; c1ccc(-c2ccc(-c3ccc(-c4nc(-c5ccccc5)nc(-c5ccc(-c6ccc(-c7ccccc7)cc6)cc5)n4)cc3)cc2)cc1 ; c1ccc(-c2ccc(-c3ccc(-c4cc(-c5ccccc5)nc(-c5ccc(-c6ccc(-c7ccccc7)cc6)cc5)n4)cc3)cc2)cc1]

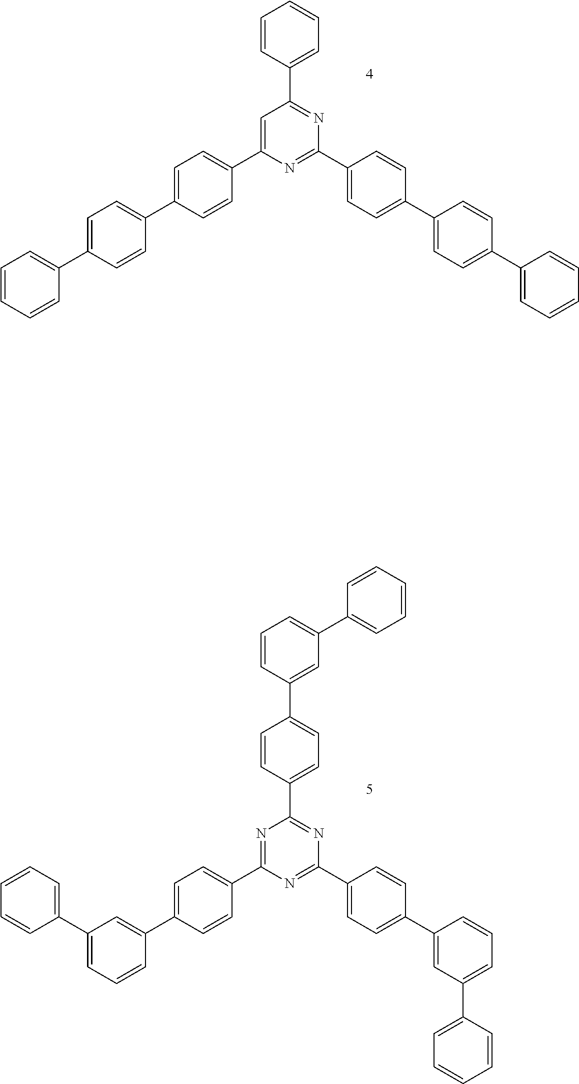 [SMILES: c1ccc(-c2ccc(-c3ccc(-c4cc(-c5ccccc5)nc(-c5ccc(-c6ccc(-c7ccccc7)cc6)cc5)n4)cc3)cc2)cc1 ; c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1]

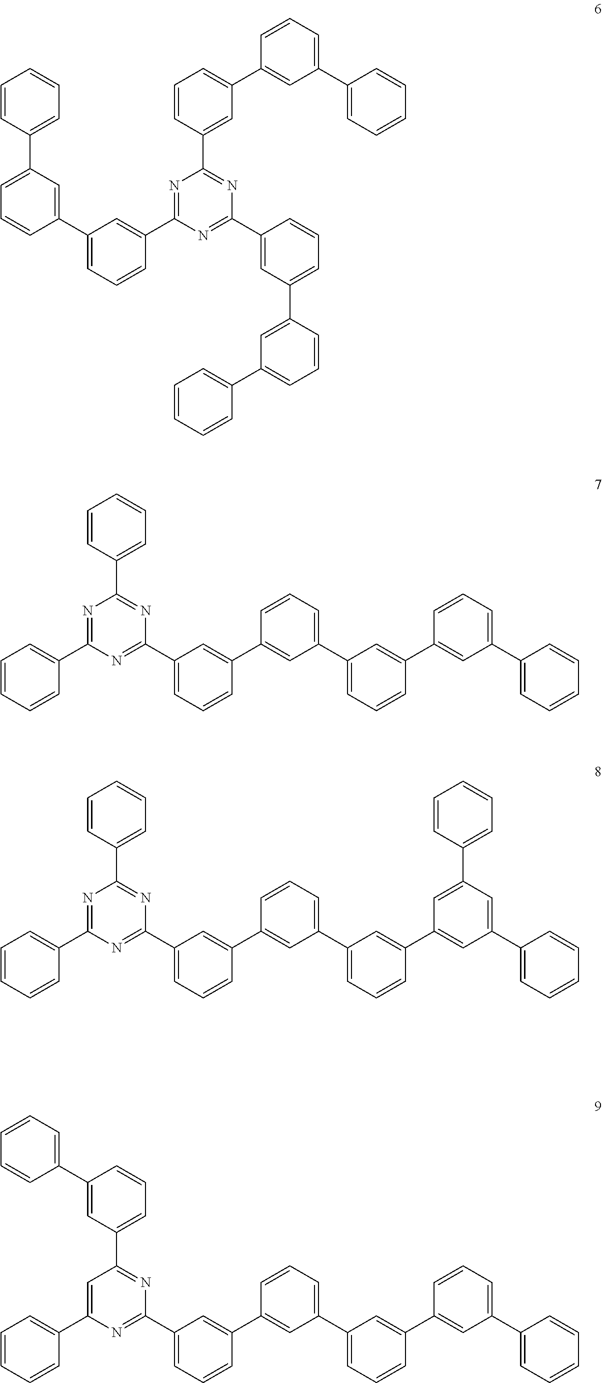 [SMILES: c1ccc(-c2cccc(-c3cccc(-c4nc(-c5cccc(-c6cccc(-c7ccccc7)c6)c5)nc(-c5cccc(-c6cccc(-c7ccccc7)c6)c5)n4)c3)c2)cc1 ; c1ccc(-c2cccc(-c3cccc(-c4cccc(-c5cccc(-c6nc(-c7ccccc7)nc(-c7ccccc7)n6)c5)c4)c3)c2)cc1 ; c1ccc(-c2cc(-c3ccccc3)cc(-c3cccc(-c4cccc(-c5cccc(-c6nc(-c7ccccc7)nc(-c7ccccc7)n6)c5)c4)c3)c2)cc1 ; c1ccc(-c2cccc(-c3cccc(-c4cccc(-c5cccc(-c6nc(-c7ccccc7)cc(-c7cccc(-c8ccccc8)c7)n6)c5)c4)c3)c2)cc1]

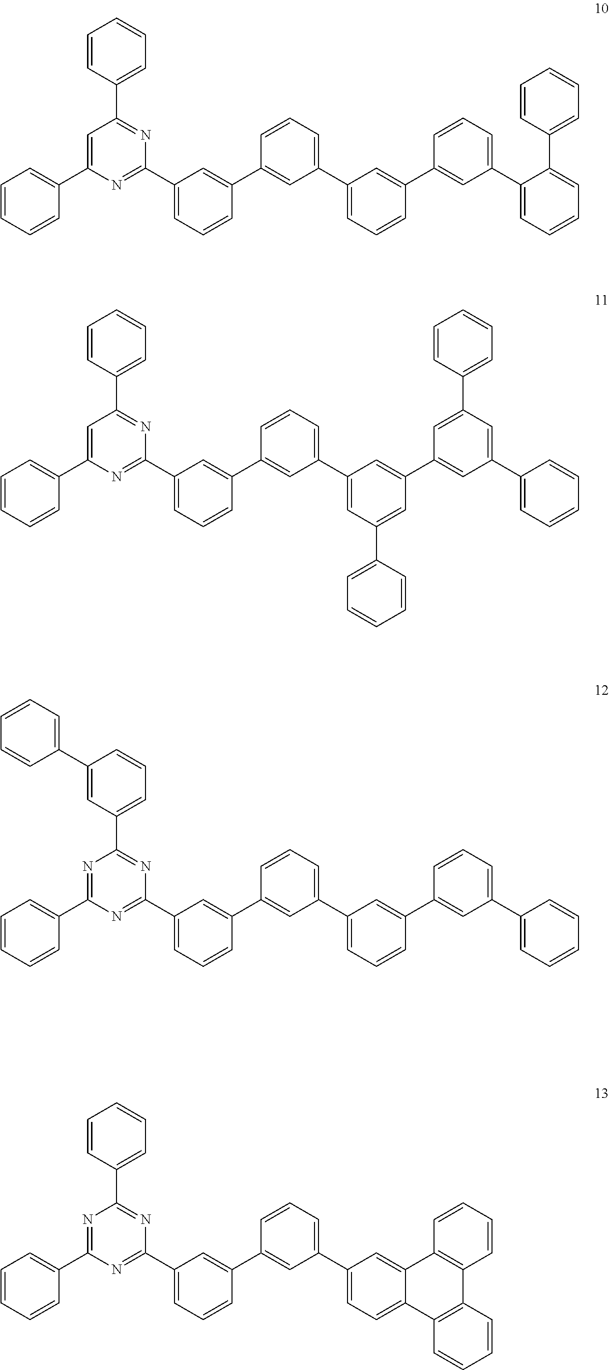 [SMILES: c1ccc(-c2cc(-c3ccccc3)nc(-c3cccc(-c4cccc(-c5cccc(-c6cccc(-c7ccccc7-c7ccccc7)c6)c5)c4)c3)n2)cc1 ; c1ccc(-c2cc(-c3ccccc3)cc(-c3cc(-c4ccccc4)cc(-c4cccc(-c5cccc(-c6nc(-c7ccccc7)cc(-c7ccccc7)n6)c5)c4)c3)c2)cc1 ; c1ccc(-c2cccc(-c3cccc(-c4cccc(-c5cccc(-c6nc(-c7ccccc7)nc(-c7cccc(-c8ccccc8)c7)n6)c5)c4)c3)c2)cc1 ; c1ccc(-c2nc(-c3ccccc3)nc(-c3cccc(-c4cccc(-c5ccc6c7ccccc7c7ccccc7c6c5)c4)c3)n2)cc1]

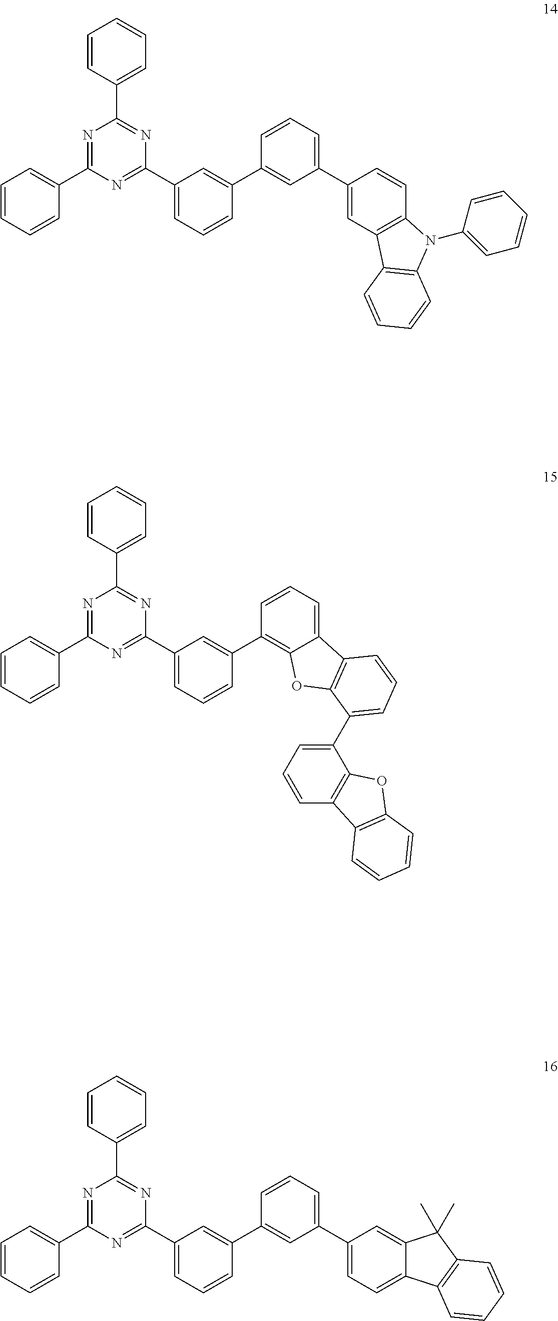 [SMILES: c1ccc(-c2nc(-c3ccccc3)nc(-c3cccc(-c4cccc(-c5ccc6c(c5)c5ccccc5n6-c5ccccc5)c4)c3)n2)cc1 ; c1ccc(-c2nc(-c3ccccc3)nc(-c3cccc(-c4cccc5c4oc4c(-c6cccc7c6oc6ccccc67)cccc45)c3)n2)cc1 ; CC1(C)c2ccccc2-c2ccc(-c3cccc(-c4cccc(-c5nc(-c6ccccc6)nc(-c6ccccc6)n5)c4)c3)cc21]

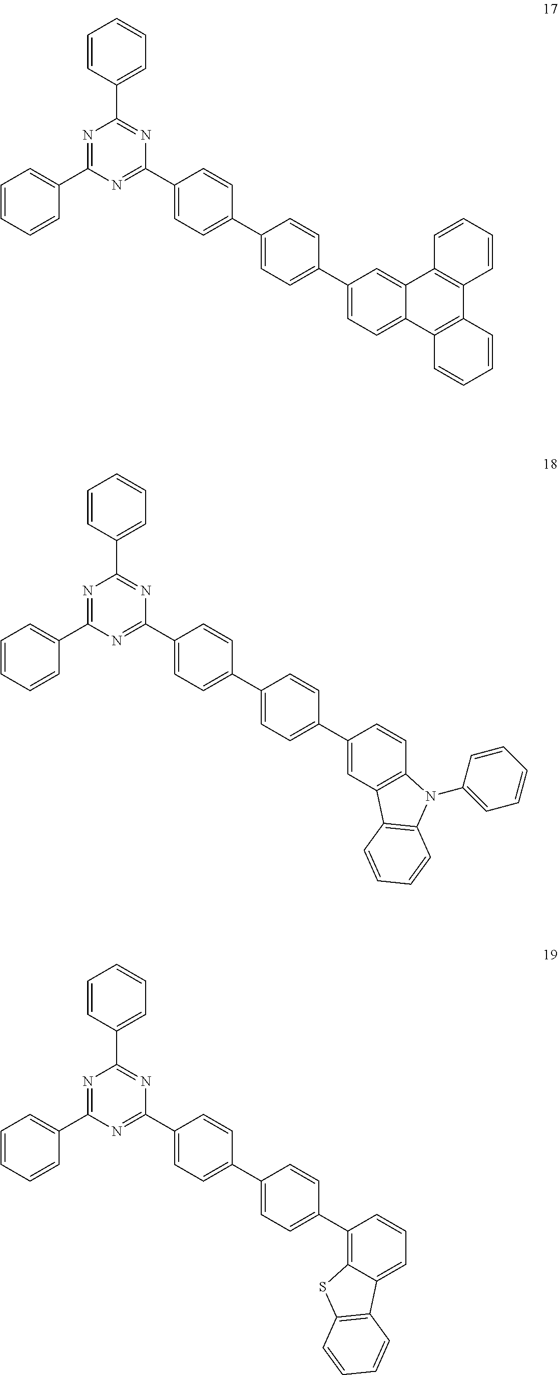 [SMILES: c1ccc(-c2nc(-c3ccccc3)nc(-c3ccc(-c4ccc(-c5ccc6c7ccccc7c7ccccc7c6c5)cc4)cc3)n2)cc1 ; c1ccc(-c2nc(-c3ccccc3)nc(-c3ccc(-c4ccc(-c5ccc6c(c5)c5ccccc5n6-c5ccccc5)cc4)cc3)n2)cc1 ; c1ccc(-c2nc(-c3ccccc3)nc(-c3ccc(-c4ccc(-c5cccc6c5sc5ccccc56)cc4)cc3)n2)cc1]

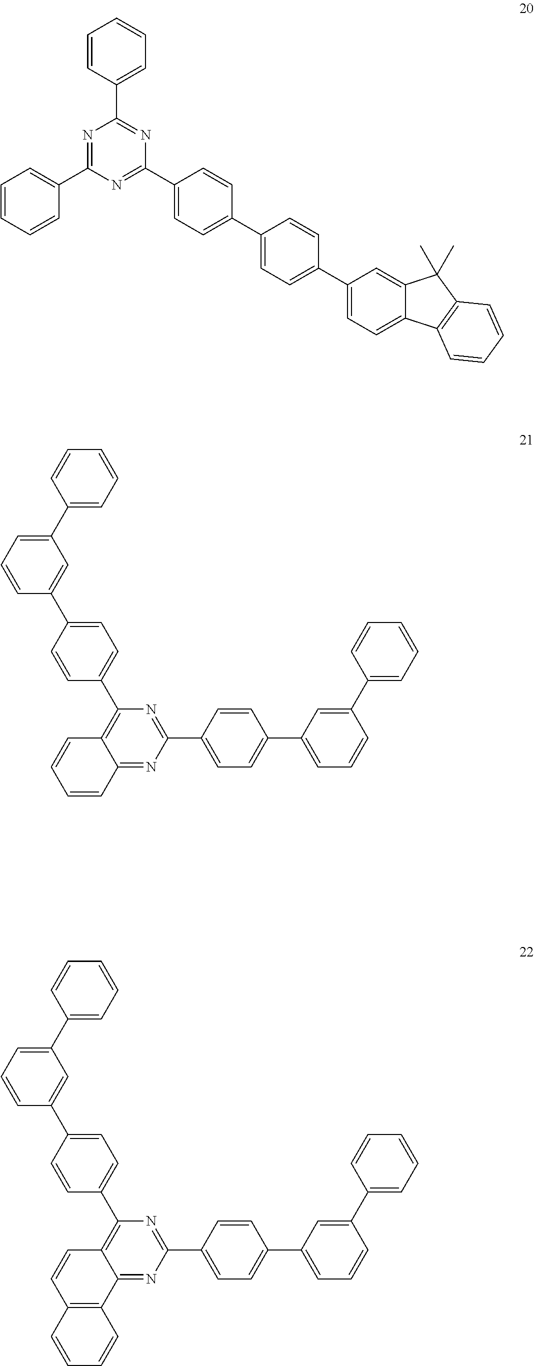 [SMILES: CC1(C)c2ccccc2-c2ccc(-c3ccc(-c4ccc(-c5nc(-c6ccccc6)nc(-c6ccccc6)n5)cc4)cc3)cc21 ; c1ccc(-c2cccc(-c3ccc(-c4nc(-c5ccc(-c6cccc(-c7ccccc7)c6)cc5)c5ccccc5n4)cc3)c2)cc1 ; c1ccc(-c2cccc(-c3ccc(-c4nc(-c5ccc(-c6cccc(-c7ccccc7)c6)cc5)c5ccc6ccccc6c5n4)cc3)c2)cc1]

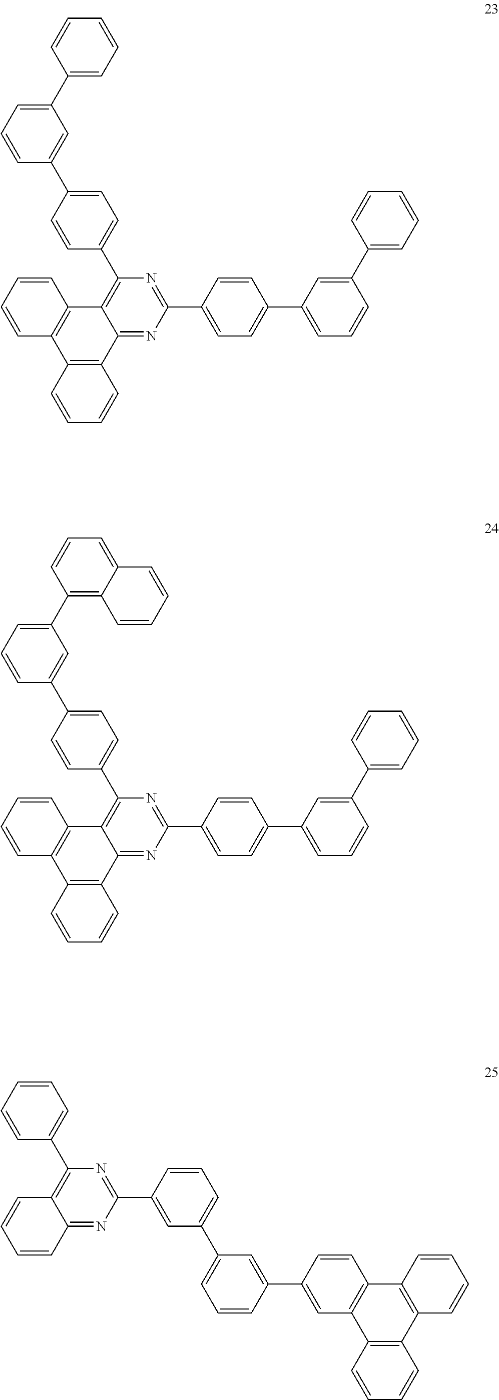 [SMILES: c1ccc(-c2cccc(-c3ccc(-c4nc(-c5ccc(-c6cccc(-c7ccccc7)c6)cc5)c5c6ccccc6c6ccccc6c5n4)cc3)c2)cc1 ; c1ccc(-c2cccc(-c3ccc(-c4nc(-c5ccc(-c6cccc(-c7cccc8ccccc78)c6)cc5)c5c6ccccc6c6ccccc6c5n4)cc3)c2)cc1 ; c1ccc(-c2nc(-c3cccc(-c4cccc(-c5ccc6c7ccccc7c7ccccc7c6c5)c4)c3)nc3ccccc23)cc1]

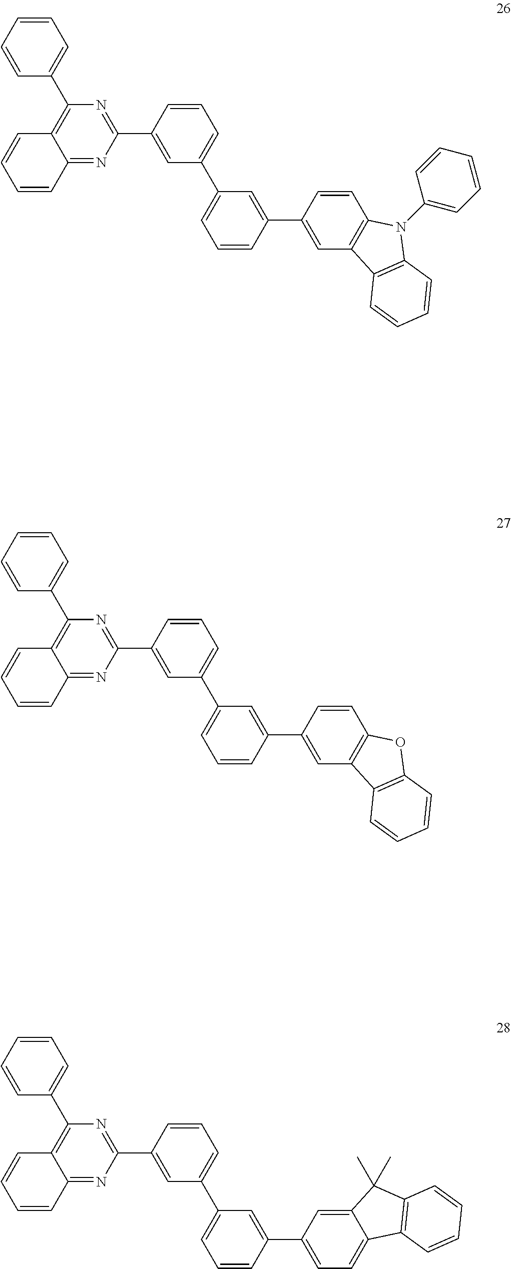 [SMILES: c1ccc(-c2nc(-c3cccc(-c4cccc(-c5ccc6c(c5)c5ccccc5n6-c5ccccc5)c4)c3)nc3ccccc23)cc1 ; c1ccc(-c2nc(-c3cccc(-c4cccc(-c5ccc6oc7ccccc7c6c5)c4)c3)nc3ccccc23)cc1 ; CC1(C)c2ccccc2-c2ccc(-c3cccc(-c4cccc(-c5nc(-c6ccccc6)c6ccccc6n5)c4)c3)cc21]

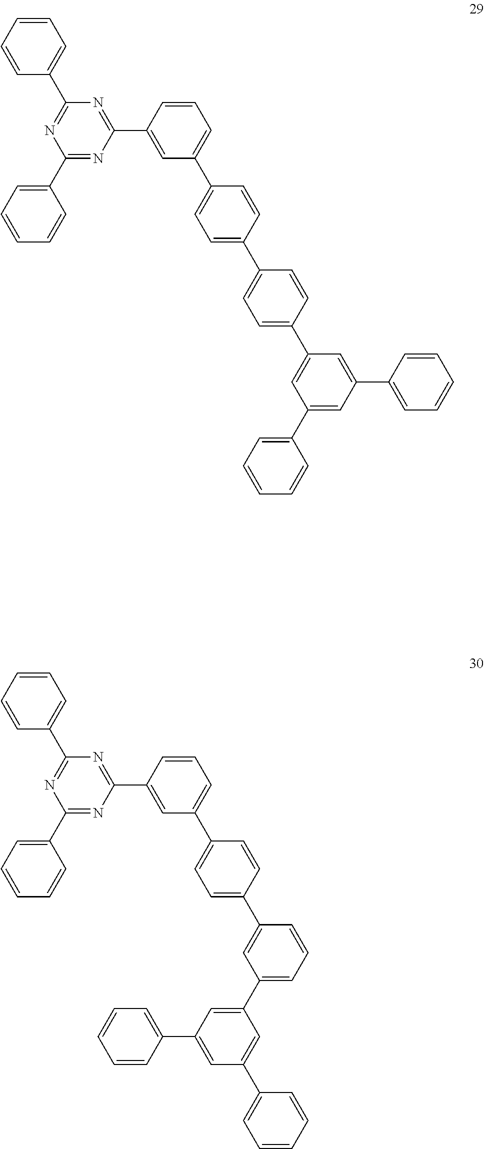 [SMILES: c1ccc(-c2cc(-c3ccccc3)cc(-c3ccc(-c4ccc(-c5cccc(-c6nc(-c7ccccc7)nc(-c7ccccc7)n6)c5)cc4)cc3)c2)cc1 ; c1ccc(-c2cc(-c3ccccc3)cc(-c3cccc(-c4ccc(-c5cccc(-c6nc(-c7ccccc7)nc(-c7ccccc7)n6)c5)cc4)c3)c2)cc1]

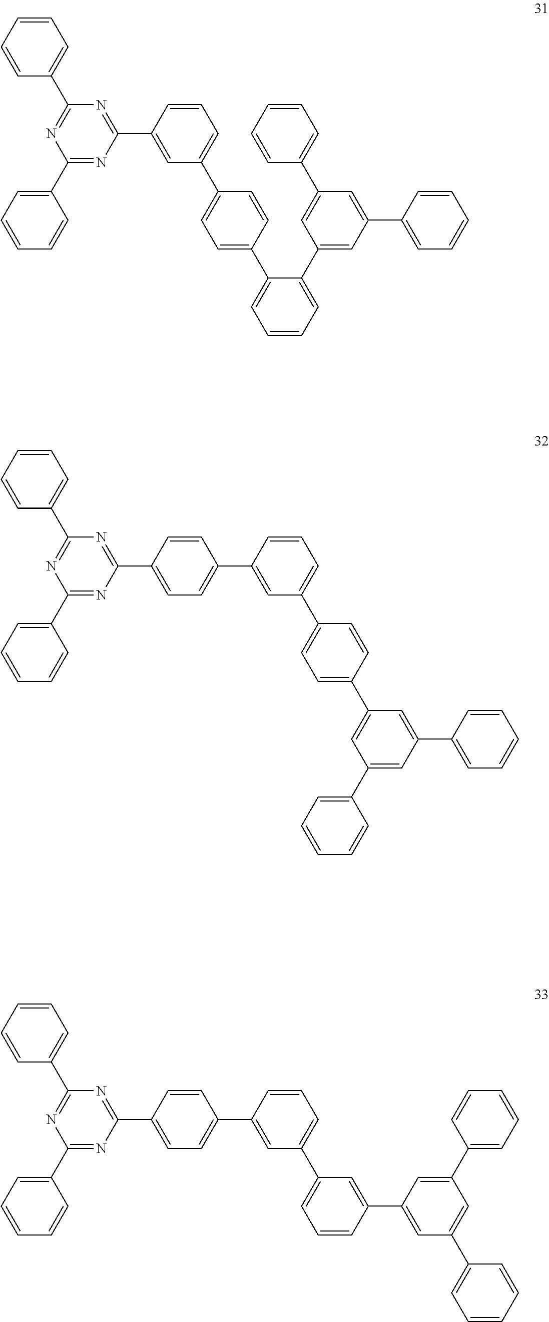 [SMILES: c1ccc(-c2cc(-c3ccccc3)cc(-c3ccccc3-c3ccc(-c4cccc(-c5nc(-c6ccccc6)nc(-c6ccccc6)n5)c4)cc3)c2)cc1 ; c1ccc(-c2cc(-c3ccccc3)cc(-c3ccc(-c4cccc(-c5ccc(-c6nc(-c7ccccc7)nc(-c7ccccc7)n6)cc5)c4)cc3)c2)cc1 ; c1ccc(-c2cc(-c3ccccc3)cc(-c3cccc(-c4cccc(-c5ccc(-c6nc(-c7ccccc7)nc(-c7ccccc7)n6)cc5)c4)c3)c2)cc1]

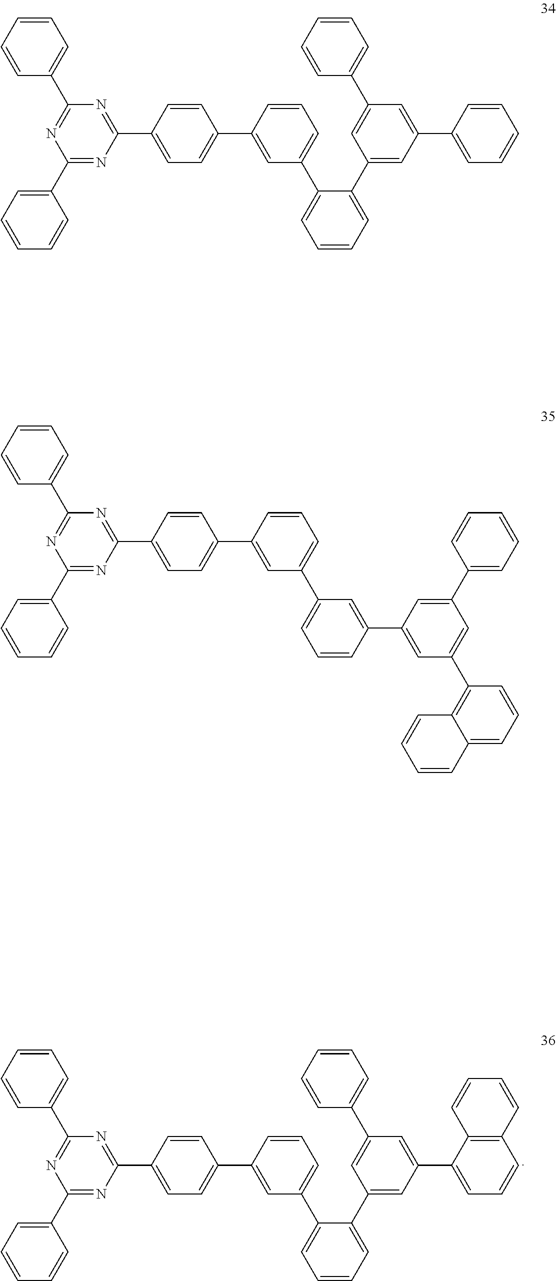 [SMILES: c1ccc(-c2cc(-c3ccccc3)cc(-c3ccccc3-c3cccc(-c4ccc(-c5nc(-c6ccccc6)nc(-c6ccccc6)n5)cc4)c3)c2)cc1 ; c1ccc(-c2cc(-c3cccc(-c4cccc(-c5ccc(-c6nc(-c7ccccc7)nc(-c7ccccc7)n6)cc5)c4)c3)cc(-c3cccc4ccccc34)c2)cc1 ; c1ccc(-c2cc(-c3ccccc3-c3cccc(-c4ccc(-c5nc(-c6ccccc6)nc(-c6ccccc6)n5)cc4)c3)cc(-c3cccc4ccccc34)c2)cc1]

13. The composition for an organic optoelectric diode of claim 1, wherein the second host compound is selected from the compounds shown in Group IV:

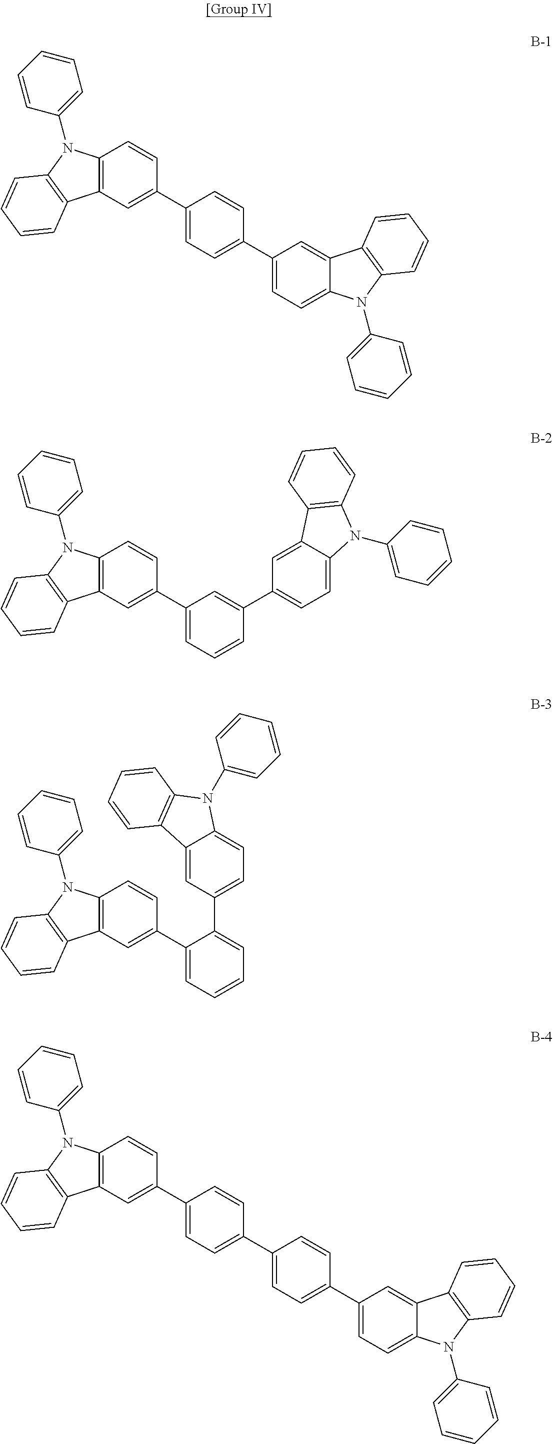 [SMILES: c1ccc(-n2c3ccccc3c3cc(-c4ccc(-c5ccc6c(c5)c5ccccc5n6-c5ccccc5)cc4)ccc32)cc1 ; c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccccc4-c4ccc5c(c4)c4ccccc4n5-c4ccccc4)ccc32)cc1 ; c1ccc(-n2c3ccccc3c3cc(-c4ccc(-c5ccc(-c6ccc7c(c6)c6ccccc6n7-c6ccccc6)cc5)cc4)ccc32)cc1]

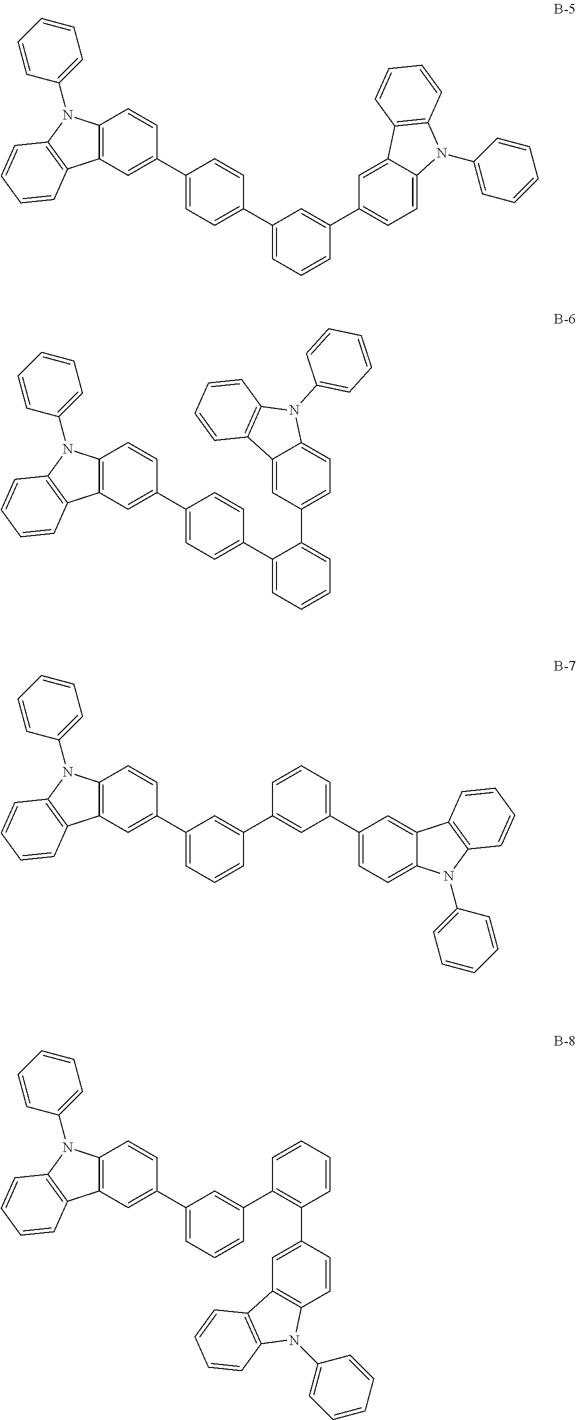 [SMILES: c1ccc(-n2c3ccccc3c3cc(-c4ccc(-c5cccc(-c6ccc7c(c6)c6ccccc6n7-c6ccccc6)c5)cc4)ccc32)cc1 ; c1ccc(-n2c3ccccc3c3cc(-c4ccc(-c5ccccc5-c5ccc6c(c5)c5ccccc5n6-c5ccccc5)cc4)ccc32)cc1 ; c1ccc(-n2c3ccccc3c3cc(-c4cccc(-c5cccc(-c6ccc7c(c6)c6ccccc6n7-c6ccccc6)c5)c4)ccc32)cc1 ; c1ccc(-n2c3ccccc3c3cc(-c4cccc(-c5ccccc5-c5ccc6c(c5)c5ccccc5n6-c5ccccc5)c4)ccc32)cc1]

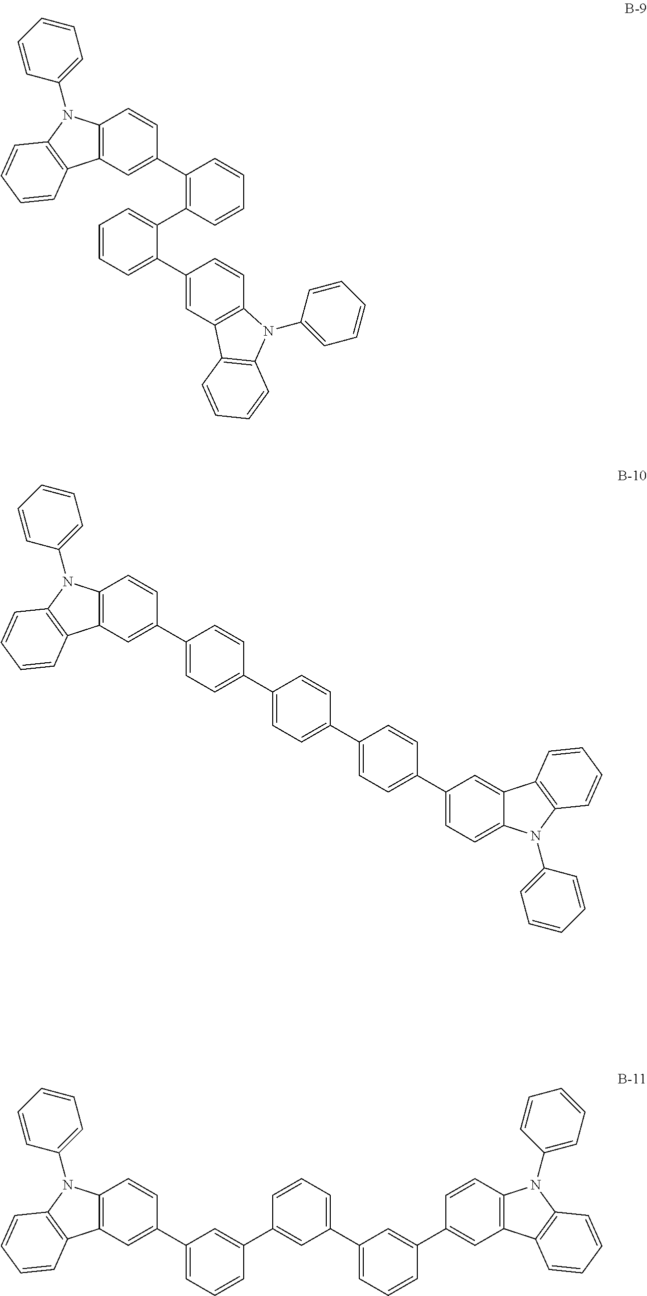 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-n2c3ccccc3c3cc(-c4ccc(-c5ccc(-c6ccc(-c7ccc8c(c7)c7ccccc7n8-c7ccccc7)cc6)cc5)cc4)ccc32)cc1 ; c1ccc(-n2c3ccccc3c3cc(-c4cccc(-c5cccc(-c6cccc(-c7ccc8c(c7)c7ccccc7n8-c7ccccc7)c6)c5)c4)ccc32)cc1]

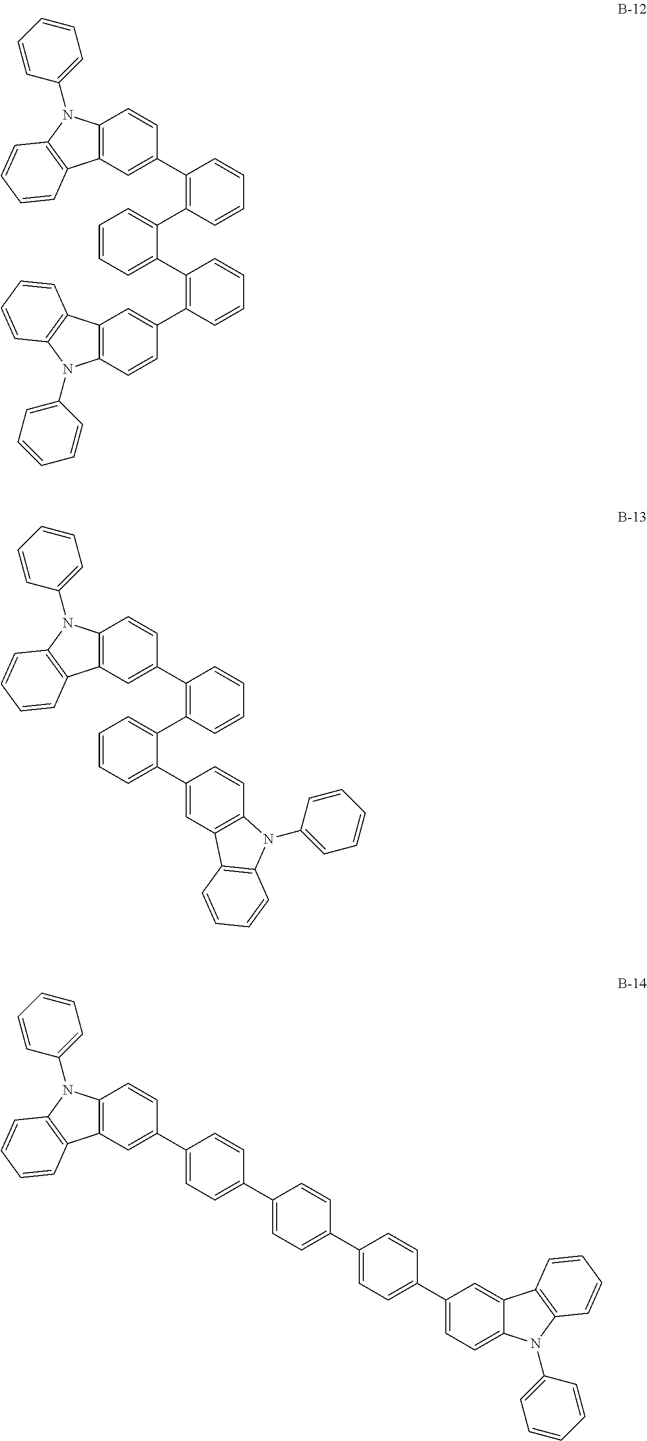 [SMILES: c1ccc(-n2c3ccccc3c3cc(-c4ccccc4-c4ccccc4-c4ccccc4-c4ccc5c(c4)c4ccccc4n5-c4ccccc4)ccc32)cc1 ; c1ccc(-n2c3ccccc3c3cc(-c4ccccc4-c4ccccc4-c4ccc5c(c4)c4ccccc4n5-c4ccccc4)ccc32)cc1 ; c1ccc(-n2c3ccccc3c3cc(-c4ccc(-c5ccc(-c6ccc(-c7ccc8c(c7)c7ccccc7n8-c7ccccc7)cc6)cc5)cc4)ccc32)cc1]

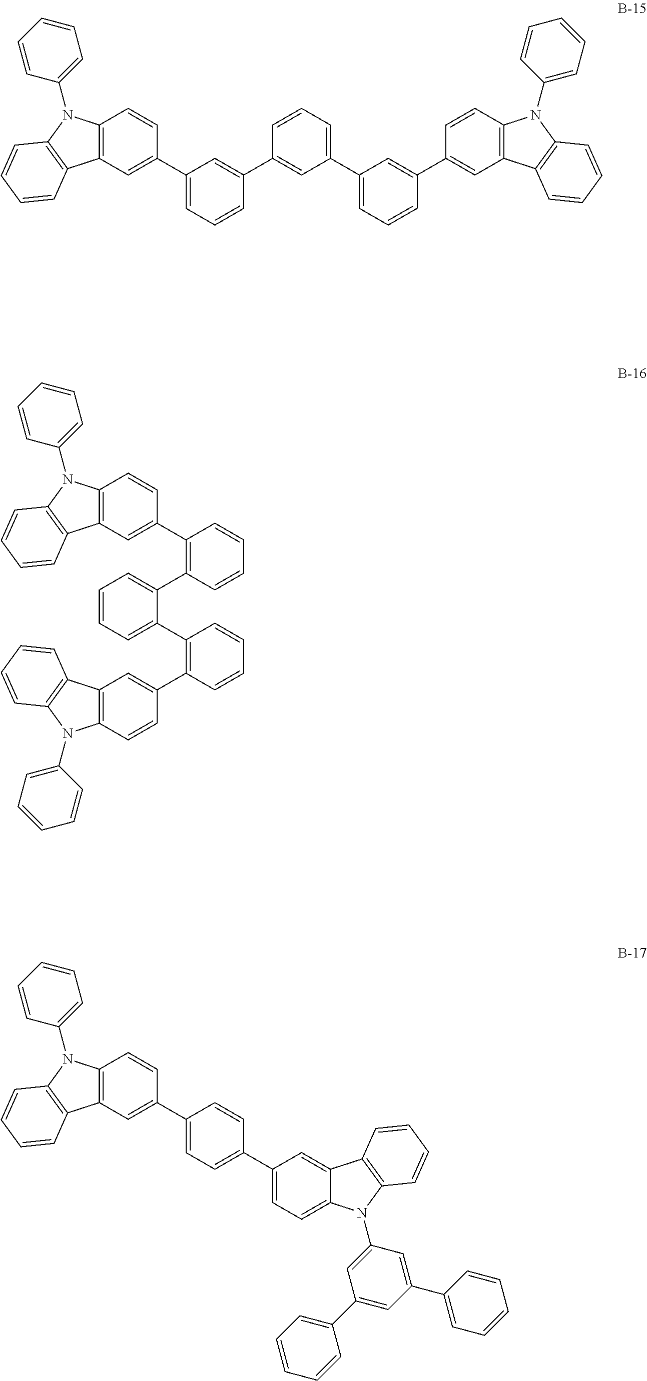 [SMILES: c1ccc(-n2c3ccccc3c3cc(-c4cccc(-c5cccc(-c6cccc(-c7ccc8c(c7)c7ccccc7n8-c7ccccc7)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4ccccc4)ccc32)cc1 ; c1ccc(-c2cc(-c3ccccc3)cc(-n3c4ccccc4c4cc(-c5ccc(-c6ccc7c(c6)c6ccccc6n7-c6ccccc6)cc5)ccc43)c2)cc1]

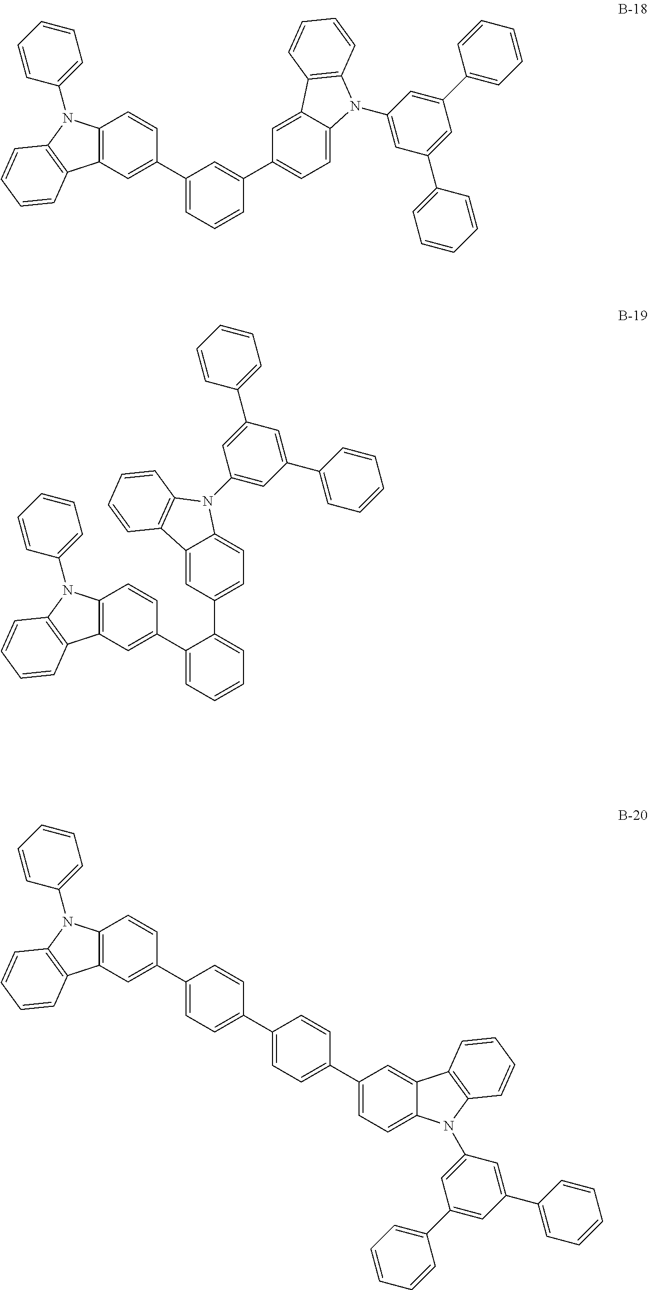 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2cc(-c3ccccc3)cc(-n3c4ccccc4c4cc(-c5ccccc5-c5ccc6c(c5)c5ccccc5n6-c5ccccc5)ccc43)c2)cc1 ; c1ccc(-c2cc(-c3ccccc3)cc(-n3c4ccccc4c4cc(-c5ccc(-c6ccc(-c7ccc8c(c7)c7ccccc7n8-c7ccccc7)cc6)cc5)ccc43)c2)cc1]

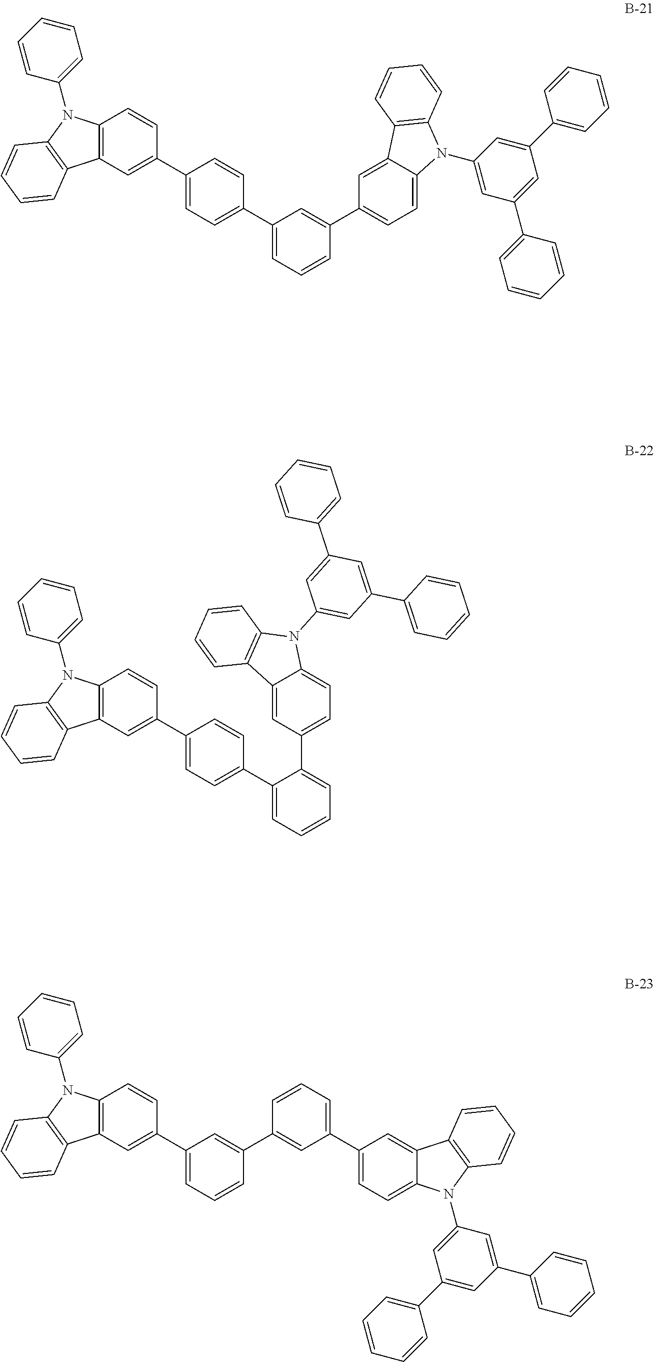 [SMILES: c1ccc(-c2cc(-c3ccccc3)cc(-n3c4ccccc4c4cc(-c5cccc(-c6ccc(-c7ccc8c(c7)c7ccccc7n8-c7ccccc7)cc6)c5)ccc43)c2)cc1 ; c1ccc(-c2cc(-c3ccccc3)cc(-n3c4ccccc4c4cc(-c5ccccc5-c5ccc(-c6ccc7c(c6)c6ccccc6n7-c6ccccc6)cc5)ccc43)c2)cc1 ; c1ccc(-c2cc(-c3ccccc3)cc(-n3c4ccccc4c4cc(-c5cccc(-c6cccc(-c7ccc8c(c7)c7ccccc7n8-c7ccccc7)c6)c5)ccc43)c2)cc1]

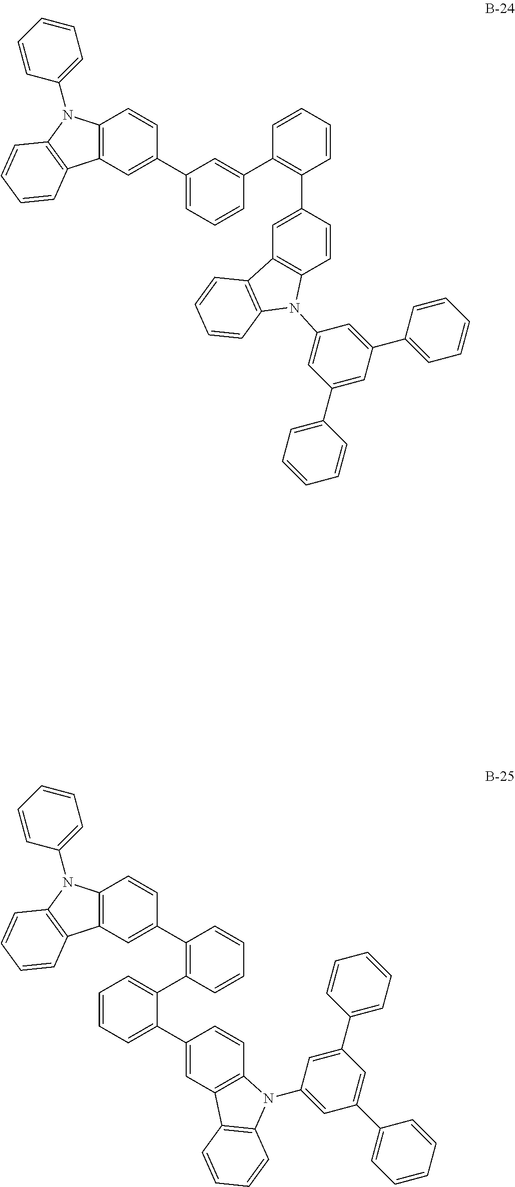 [SMILES: c1ccc(-c2cc(-c3ccccc3)cc(-n3c4ccccc4c4cc(-c5ccccc5-c5cccc(-c6ccc7c(c6)c6ccccc6n7-c6ccccc6)c5)ccc43)c2)cc1 ; c1ccc(-c2cc(-c3ccccc3)cc(-n3c4ccccc4c4cc(-c5ccccc5-c5ccccc5-c5ccc6c(c5)c5ccccc5n6-c5ccccc5)ccc43)c2)cc1]

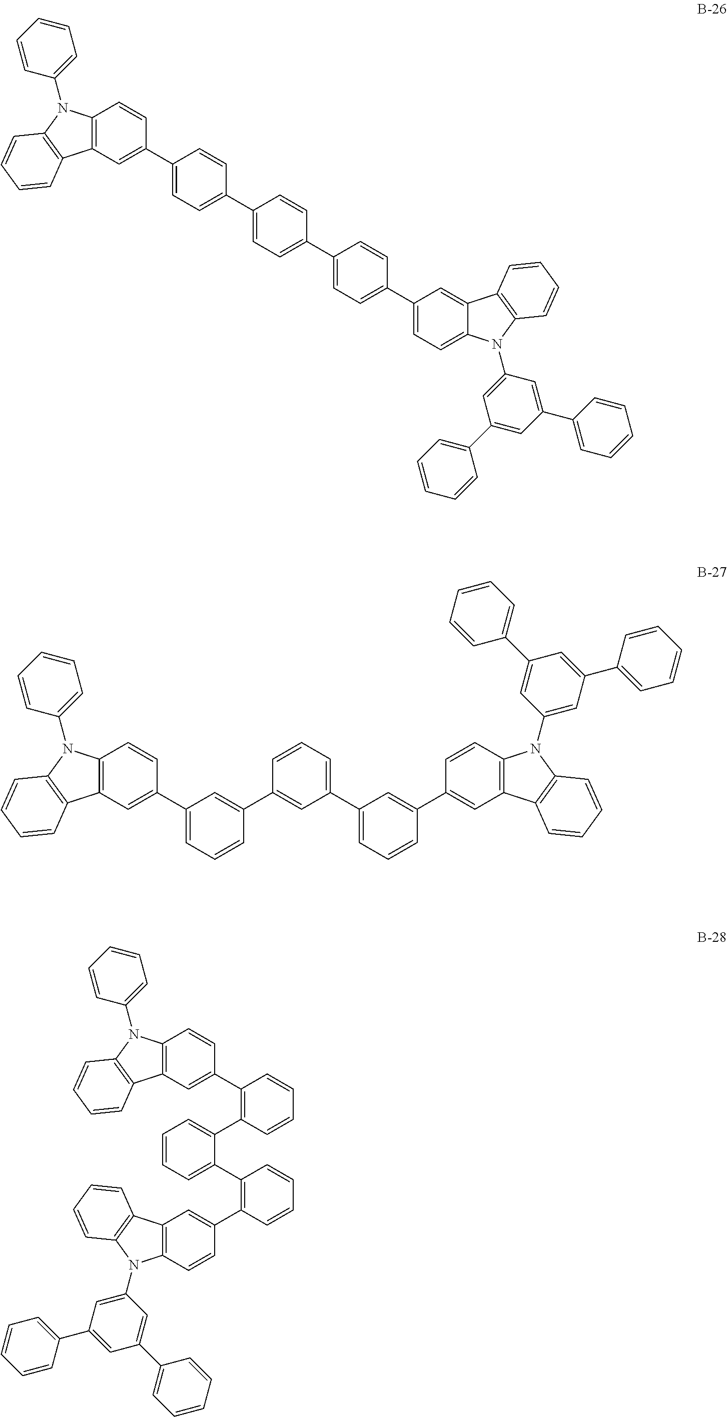 [SMILES: c1ccc(-c2cc(-c3ccccc3)cc(-n3c4ccccc4c4cc(-c5ccc(-c6ccc(-c7ccc(-c8ccc9c(c8)c8ccccc8n9-c8ccccc8)cc7)cc6)cc5)ccc43)c2)cc1 ; c1ccc(-c2cc(-c3ccccc3)cc(-n3c4ccccc4c4cc(-c5cccc(-c6cccc(-c7cccc(-c8ccc9c(c8)c8ccccc8n9-c8ccccc8)c7)c6)c5)ccc43)c2)cc1 ; c1ccc(-c2cc(-c3ccccc3)cc(-n3c4ccccc4c4cc(-c5ccccc5-c5ccccc5-c5ccccc5-c5ccc6c(c5)c5ccccc5n6-c5ccccc5)ccc43)c2)cc1]

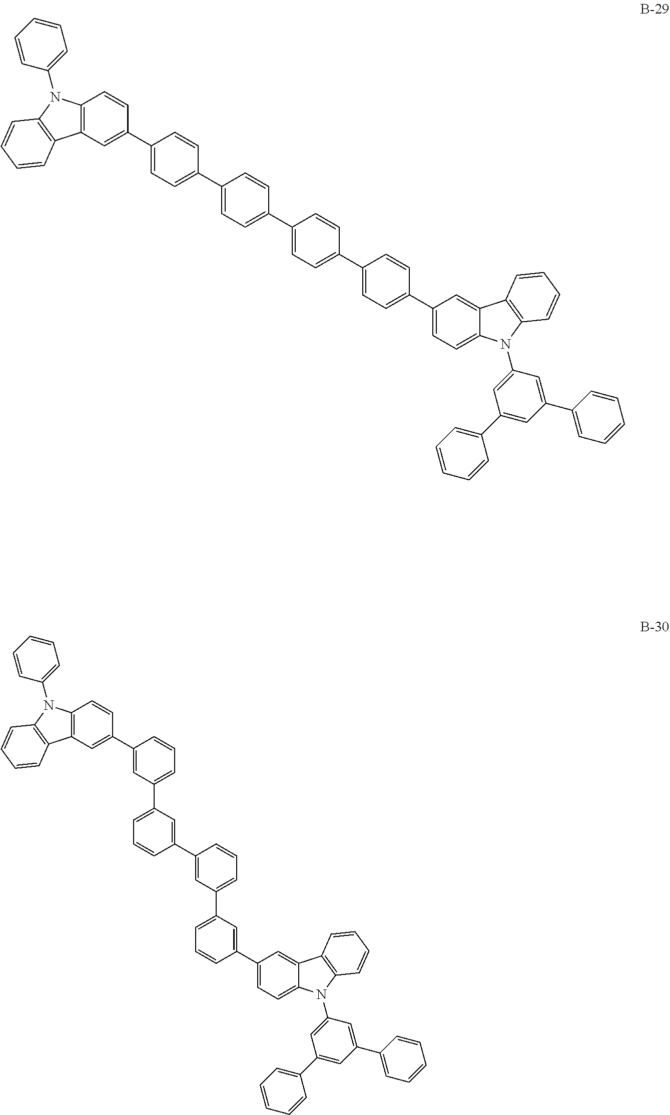 [SMILES: c1ccc(-c2cc(-c3ccccc3)cc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)c2)cc1 ; c1ccc(-c2cc(-c3ccccc3)cc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)c2)cc1]

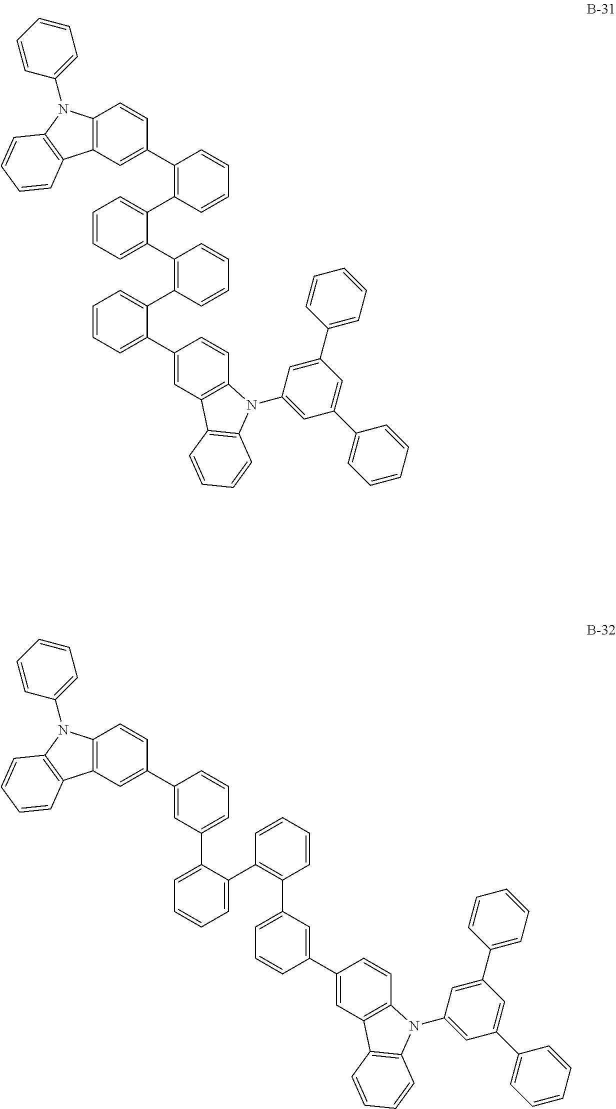 [SMILES: c1ccc(-c2cc(-c3ccccc3)cc(-n3c4ccccc4c4cc(-c5ccccc5-c5ccccc5-c5ccccc5-c5ccccc5-c5ccc6c(c5)c5ccccc5n6-c5ccccc5)ccc43)c2)cc1 ; c1ccc(-c2cc(-c3ccccc3)cc(-n3c4ccccc4c4cc(-c5cccc(-c6ccccc6-c6ccccc6-c6cccc(-c7ccc8c(c7)c7ccccc7n8-c7ccccc7)c6)c5)ccc43)c2)cc1]

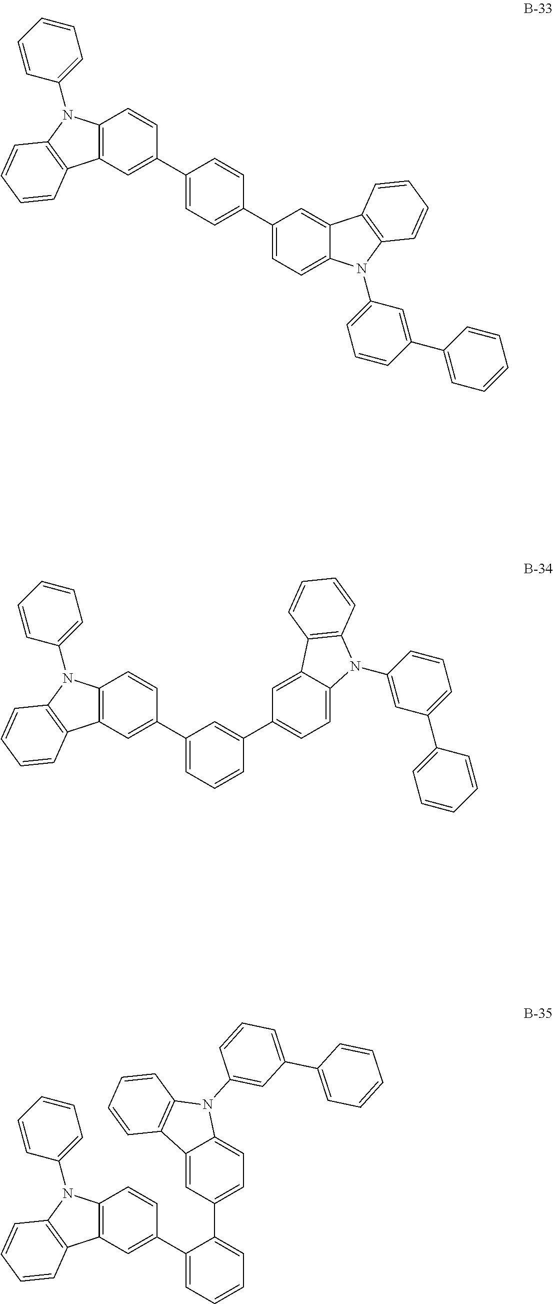 [SMILES: c1ccc(-c2cccc(-n3c4ccccc4c4cc(-c5ccc(-c6ccc7c(c6)c6ccccc6n7-c6ccccc6)cc5)ccc43)c2)cc1 ; c1ccc(-c2cccc(-n3c4ccccc4c4cc(-c5cccc(-c6ccc7c(c6)c6ccccc6n7-c6ccccc6)c5)ccc43)c2)cc1 ; c1ccc(-c2cccc(-n3c4ccccc4c4cc(-c5ccccc5-c5ccc6c(c5)c5ccccc5n6-c5ccccc5)ccc43)c2)cc1]

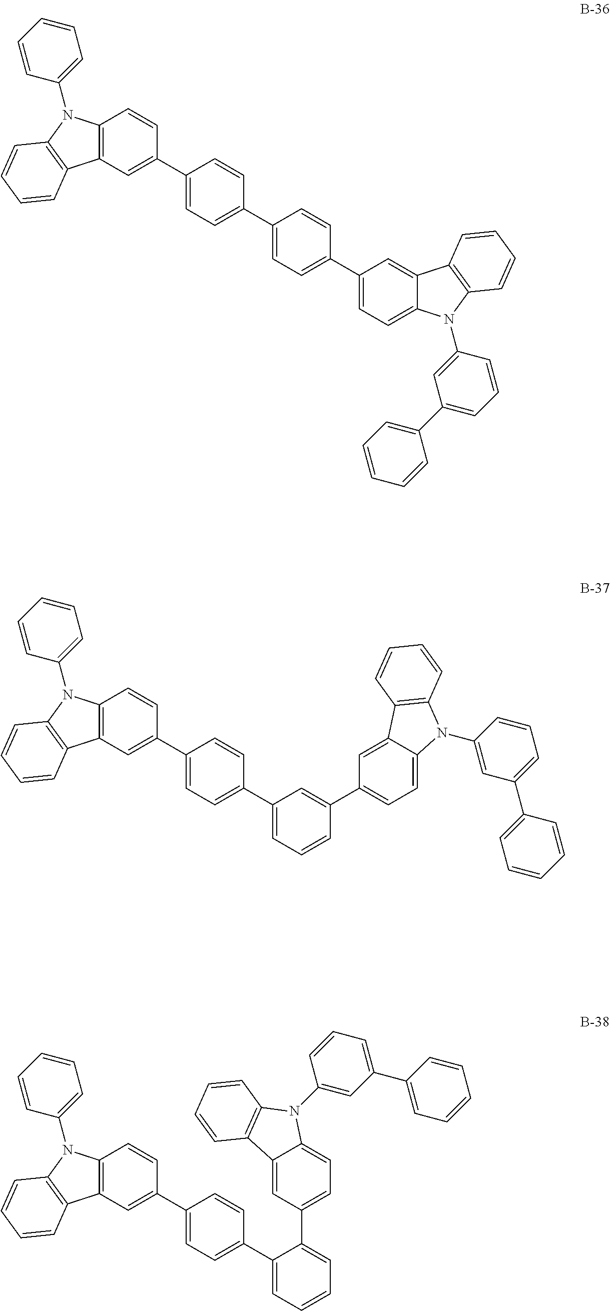 [SMILES: c1ccc(-c2cccc(-n3c4ccccc4c4cc(-c5ccc(-c6ccc(-c7ccc8c(c7)c7ccccc7n8-c7ccccc7)cc6)cc5)ccc43)c2)cc1 ; c1ccc(-c2cccc(-n3c4ccccc4c4cc(-c5cccc(-c6ccc(-c7ccc8c(c7)c7ccccc7n8-c7ccccc7)cc6)c5)ccc43)c2)cc1 ; c1ccc(-c2cccc(-n3c4ccccc4c4cc(-c5ccccc5-c5ccc(-c6ccc7c(c6)c6ccccc6n7-c6ccccc6)cc5)ccc43)c2)cc1]

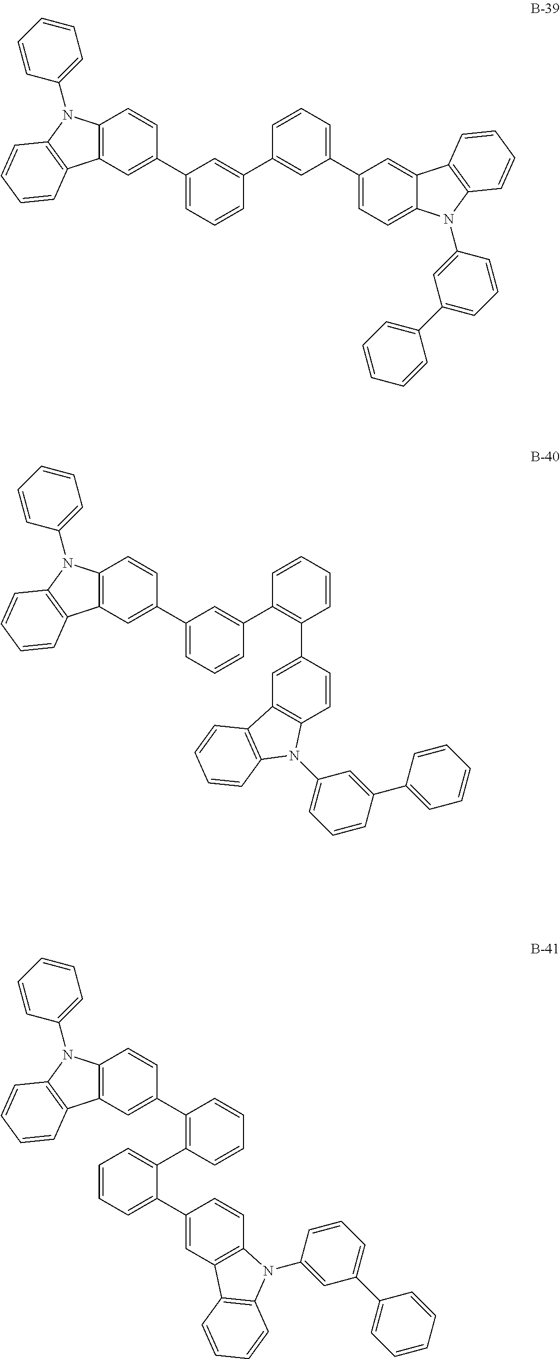 [SMILES: c1ccc(-c2cccc(-n3c4ccccc4c4cc(-c5cccc(-c6cccc(-c7ccc8c(c7)c7ccccc7n8-c7ccccc7)c6)c5)ccc43)c2)cc1 ; c1ccc(-c2cccc(-n3c4ccccc4c4cc(-c5ccccc5-c5cccc(-c6ccc7c(c6)c6ccccc6n7-c6ccccc6)c5)ccc43)c2)cc1 ; c1ccc(-c2cccc(-n3c4ccccc4c4cc(-c5ccccc5-c5ccccc5-c5ccc6c(c5)c5ccccc5n6-c5ccccc5)ccc43)c2)cc1]

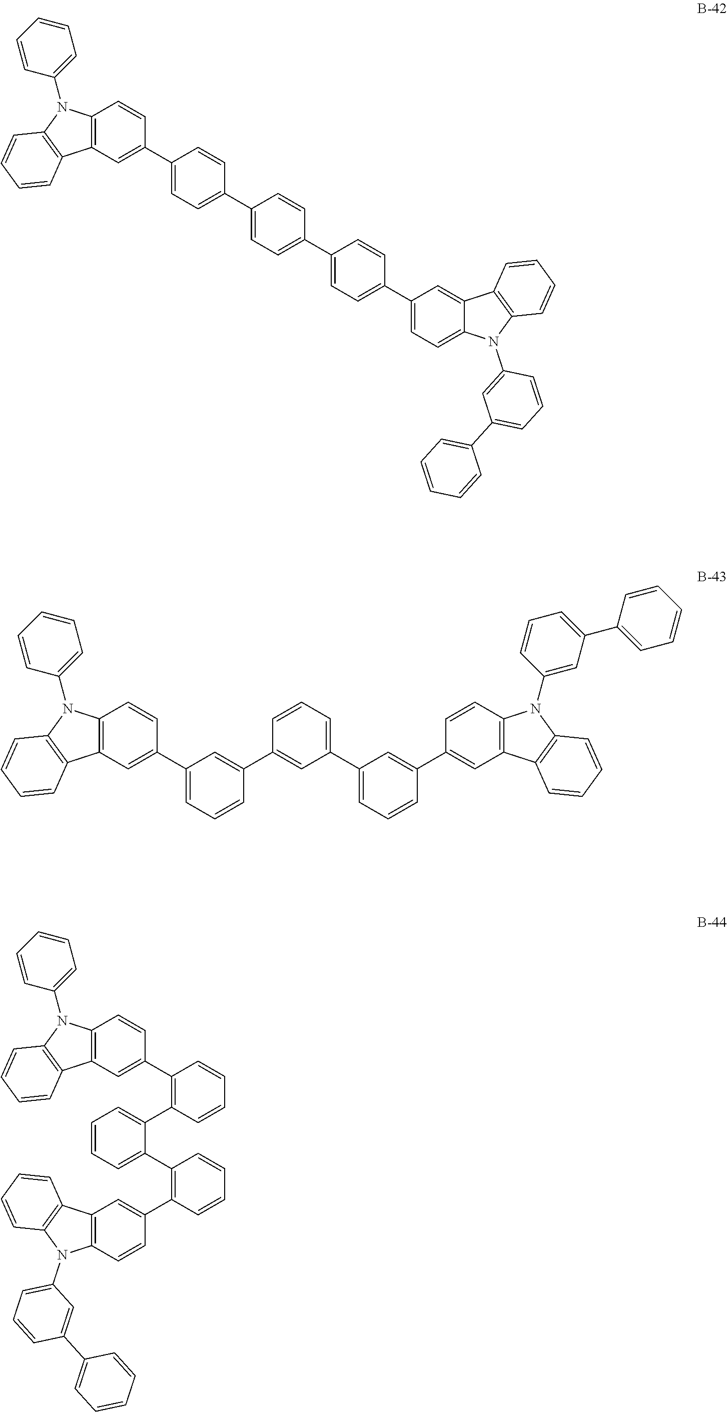 [SMILES: c1ccc(-c2cccc(-n3c4ccccc4c4cc(-c5ccc(-c6ccc(-c7ccc(-c8ccc9c(c8)c8ccccc8n9-c8ccccc8)cc7)cc6)cc5)ccc43)c2)cc1 ; c1ccc(-c2cccc(-n3c4ccccc4c4cc(-c5cccc(-c6cccc(-c7cccc(-c8ccc9c(c8)c8ccccc8n9-c8ccccc8)c7)c6)c5)ccc43)c2)cc1 ; c1ccc(-c2cccc(-n3c4ccccc4c4cc(-c5ccccc5-c5ccccc5-c5ccccc5-c5ccc6c(c5)c5ccccc5n6-c5ccccc5)ccc43)c2)cc1]

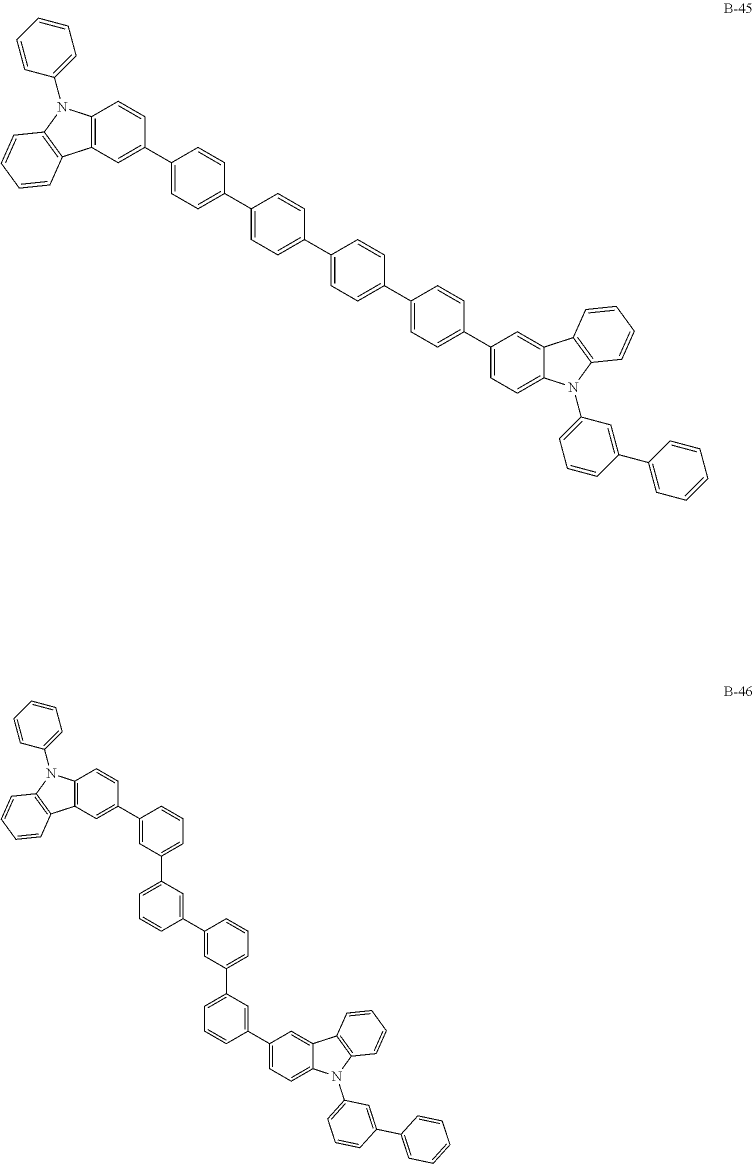 [SMILES: c1ccc(-c2cccc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)c2)cc1 ; c1ccc(-c2cccc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)c2)cc1]

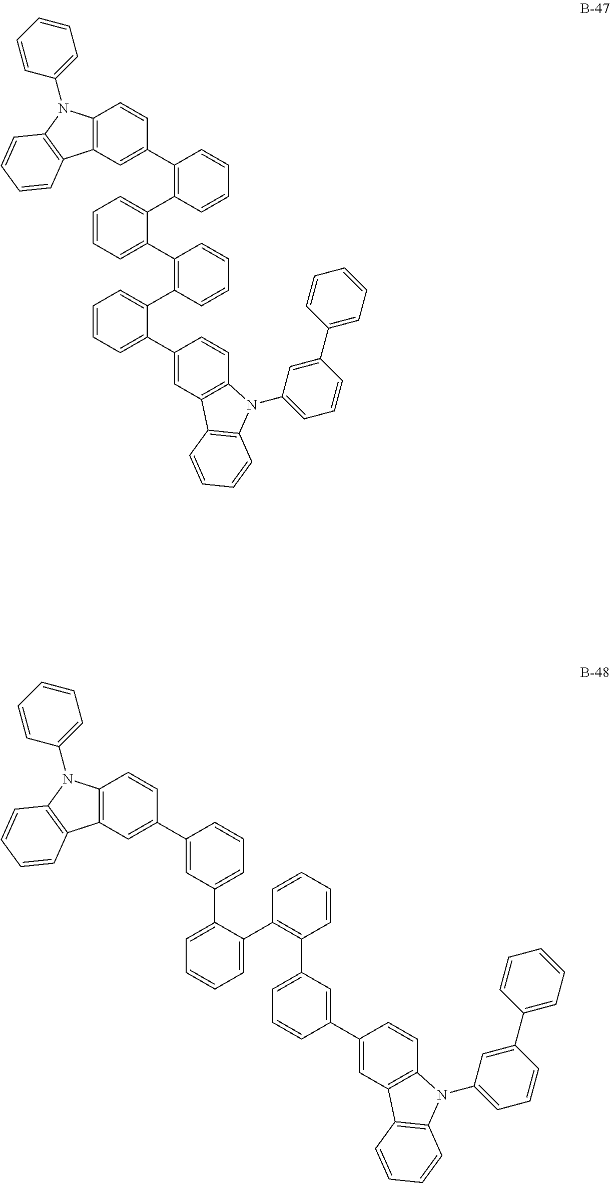 [SMILES: c1ccc(-c2cccc(-n3c4ccccc4c4cc(-c5ccccc5-c5ccccc5-c5ccccc5-c5ccccc5-c5ccc6c(c5)c5ccccc5n6-c5ccccc5)ccc43)c2)cc1 ; c1ccc(-c2cccc(-n3c4ccccc4c4cc(-c5cccc(-c6ccccc6-c6ccccc6-c6cccc(-c7ccc8c(c7)c7ccccc7n8-c7ccccc7)c6)c5)ccc43)c2)cc1]

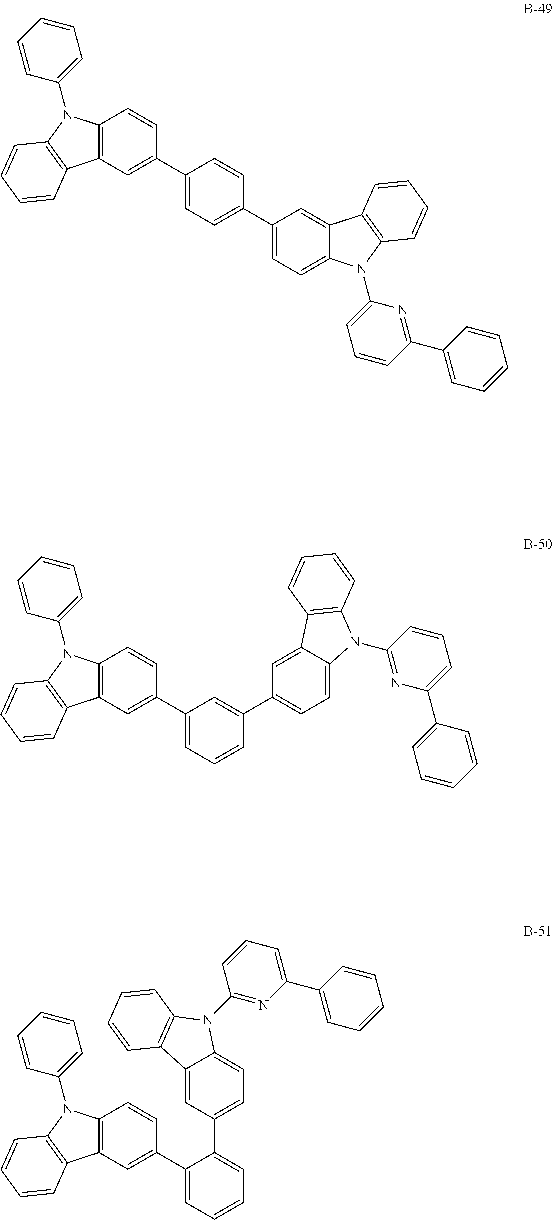 [SMILES: c1ccc(-c2cccc(-n3c4ccccc4c4cc(-c5ccc(-c6ccc7c(c6)c6ccccc6n7-c6ccccc6)cc5)ccc43)n2)cc1 ; c1ccc(-c2cccc(-n3c4ccccc4c4cc(-c5cccc(-c6ccc7c(c6)c6ccccc6n7-c6ccccc6)c5)ccc43)n2)cc1 ; c1ccc(-c2cccc(-n3c4ccccc4c4cc(-c5ccccc5-c5ccc6c(c5)c5ccccc5n6-c5ccccc5)ccc43)n2)cc1]

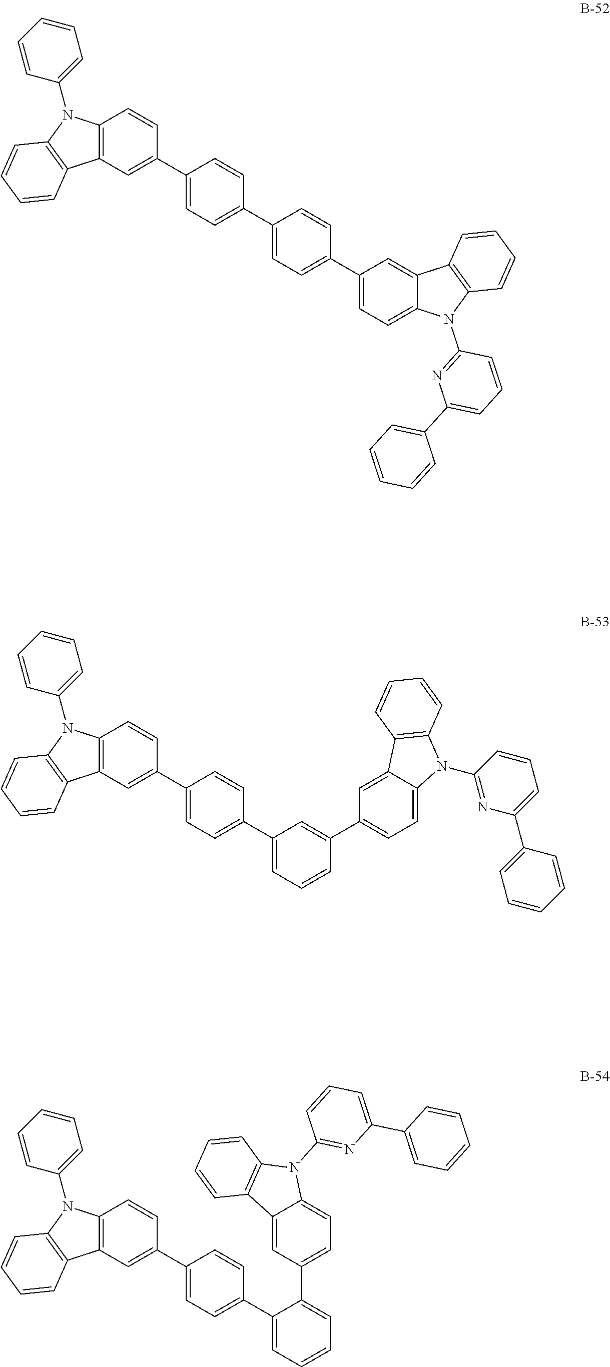 [SMILES: c1ccc(-c2cccc(-n3c4ccccc4c4cc(-c5ccc(-c6ccc(-c7ccc8c(c7)c7ccccc7n8-c7ccccc7)cc6)cc5)ccc43)n2)cc1 ; c1ccc(-c2cccc(-n3c4ccccc4c4cc(-c5cccc(-c6ccc(-c7ccc8c(c7)c7ccccc7n8-c7ccccc7)cc6)c5)ccc43)n2)cc1 ; c1ccc(-c2cccc(-n3c4ccccc4c4cc(-c5ccccc5-c5ccc(-c6ccc7c(c6)c6ccccc6n7-c6ccccc6)cc5)ccc43)n2)cc1]

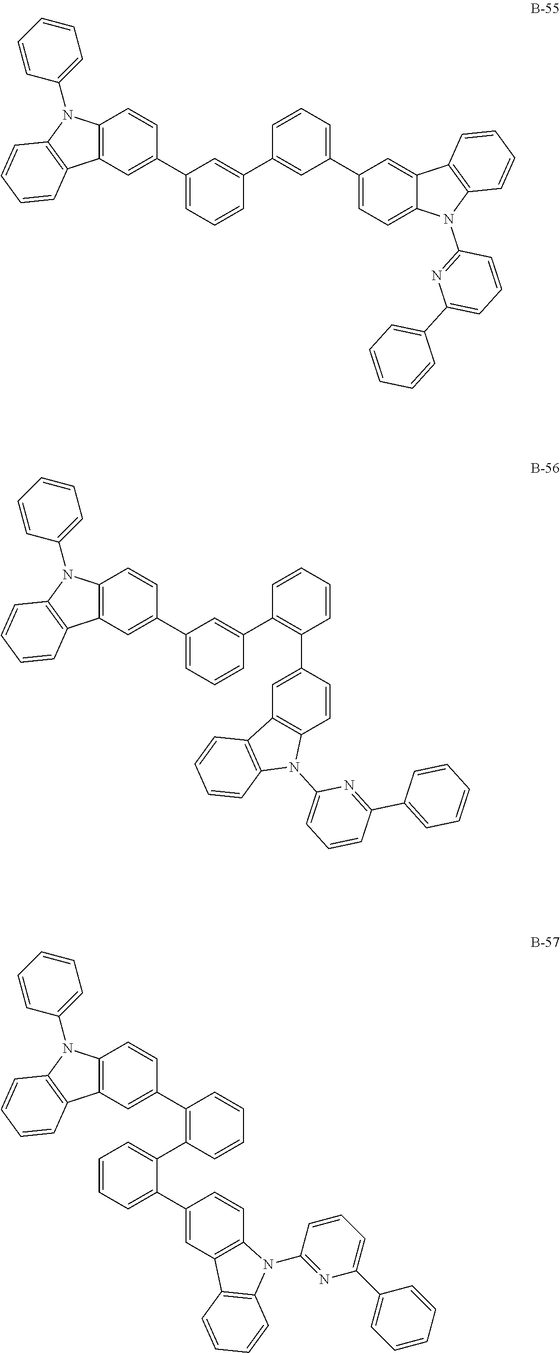 [SMILES: c1ccc(-c2cccc(-n3c4ccccc4c4cc(-c5cccc(-c6cccc(-c7ccc8c(c7)c7ccccc7n8-c7ccccc7)c6)c5)ccc43)n2)cc1 ; c1ccc(-c2cccc(-n3c4ccccc4c4cc(-c5ccccc5-c5cccc(-c6ccc7c(c6)c6ccccc6n7-c6ccccc6)c5)ccc43)n2)cc1 ; c1ccc(-c2cccc(-n3c4ccccc4c4cc(-c5ccccc5-c5ccccc5-c5ccc6c(c5)c5ccccc5n6-c5ccccc5)ccc43)n2)cc1]

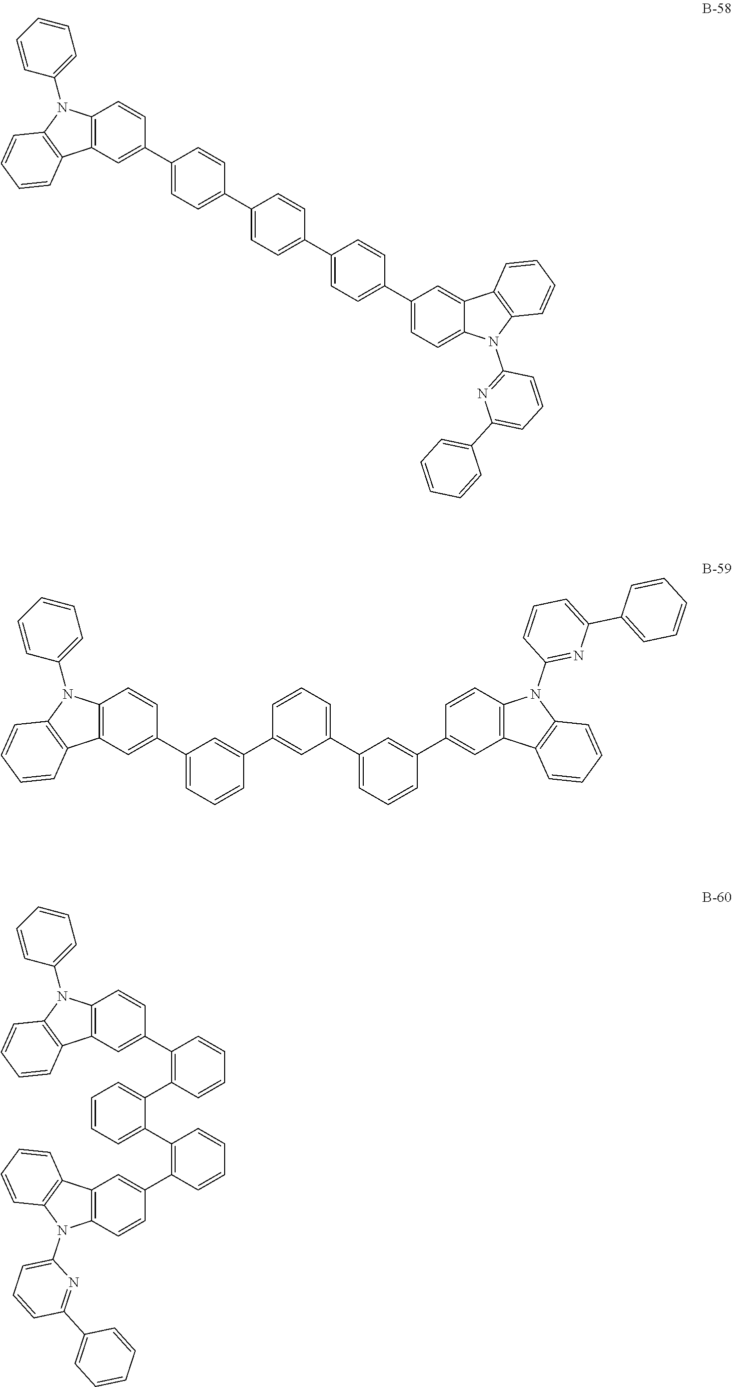 [SMILES: c1ccc(-c2cccc(-n3c4ccccc4c4cc(-c5ccc(-c6ccc(-c7ccc(-c8ccc9c(c8)c8ccccc8n9-c8ccccc8)cc7)cc6)cc5)ccc43)n2)cc1 ; c1ccc(-c2cccc(-n3c4ccccc4c4cc(-c5cccc(-c6cccc(-c7cccc(-c8ccc9c(c8)c8ccccc8n9-c8ccccc8)c7)c6)c5)ccc43)n2)cc1 ; c1ccc(-c2cccc(-n3c4ccccc4c4cc(-c5ccccc5-c5ccccc5-c5ccccc5-c5ccc6c(c5)c5ccccc5n6-c5ccccc5)ccc43)n2)cc1]

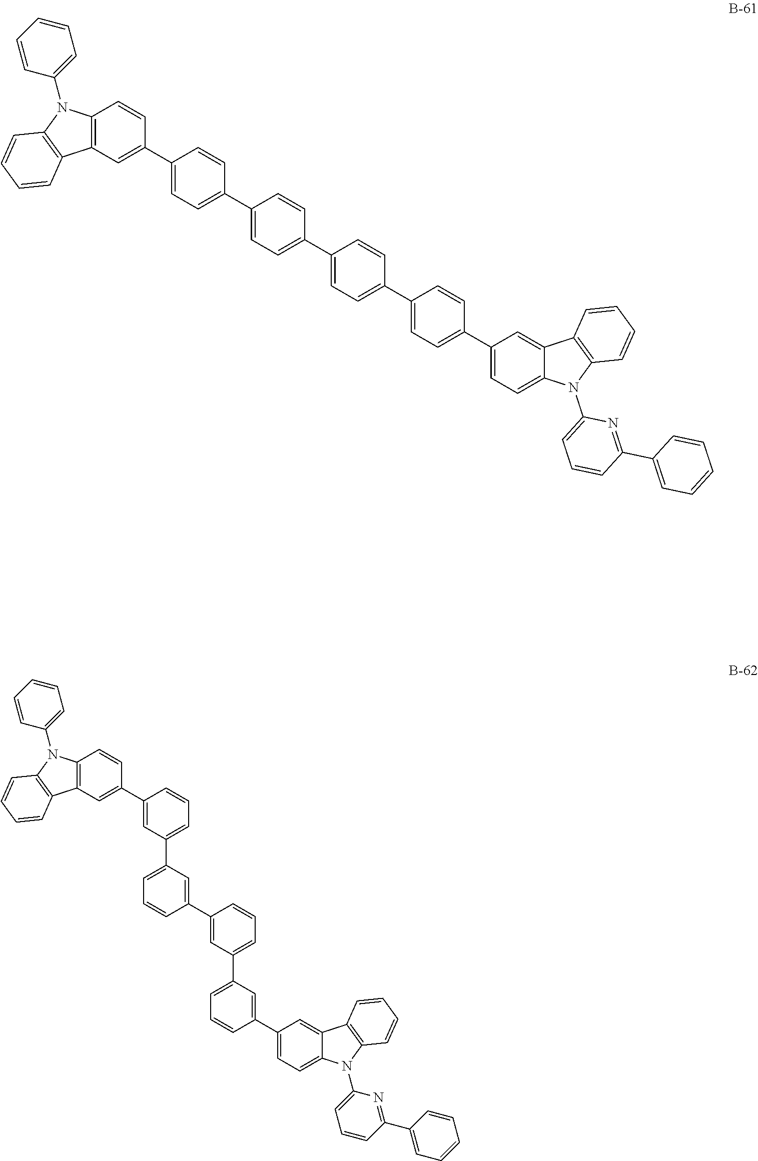 [SMILES: c1ccc(-c2cccc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)n2)cc1 ; c1ccc(-c2cccc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)n2)cc1]

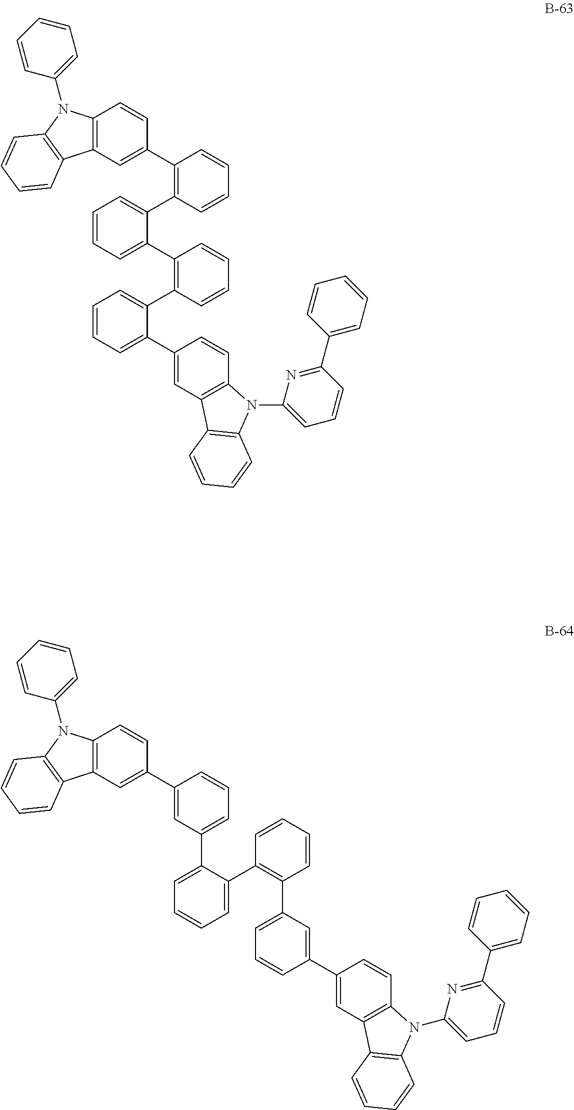 [SMILES: c1ccc(-c2cccc(-n3c4ccccc4c4cc(-c5ccccc5-c5ccccc5-c5ccccc5-c5ccccc5-c5ccc6c(c5)c5ccccc5n6-c5ccccc5)ccc43)n2)cc1 ; c1ccc(-c2cccc(-n3c4ccccc4c4cc(-c5cccc(-c6ccccc6-c6ccccc6-c6cccc(-c7ccc8c(c7)c7ccccc7n8-c7ccccc7)c6)c5)ccc43)n2)cc1]

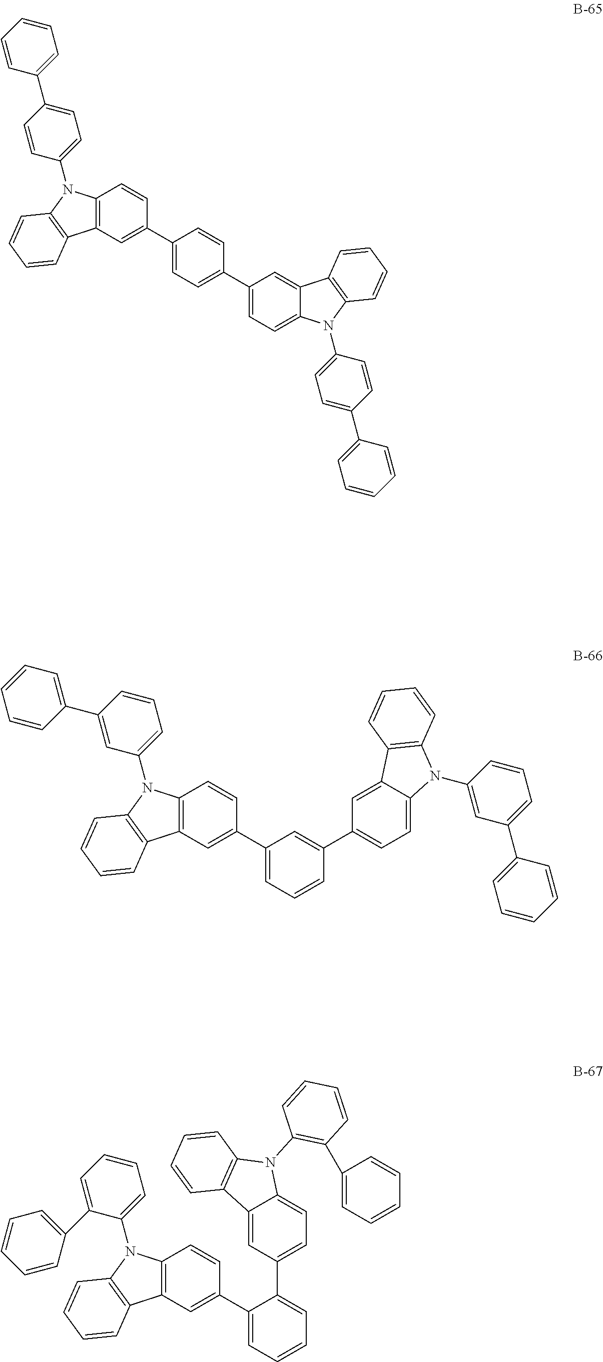 [SMILES: c1ccc(-c2ccc(-n3c4ccccc4c4cc(-c5ccc(-c6ccc7c(c6)c6ccccc6n7-c6ccc(-c7ccccc7)cc6)cc5)ccc43)cc2)cc1 ; c1ccc(-c2cccc(-n3c4ccccc4c4cc(-c5cccc(-c6ccc7c(c6)c6ccccc6n7-c6cccc(-c7ccccc7)c6)c5)ccc43)c2)cc1 ; c1ccc(-c2ccccc2-n2c3ccccc3c3cc(-c4ccccc4-c4ccc5c(c4)c4ccccc4n5-c4ccccc4-c4ccccc4)ccc32)cc1]

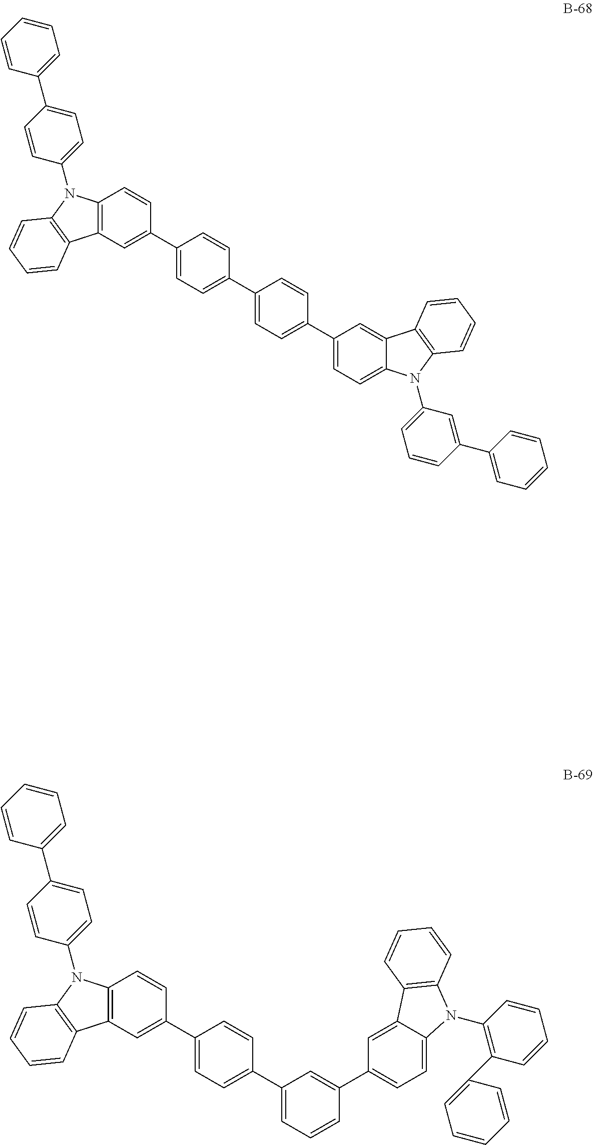 [SMILES: c1ccc(-c2ccc(-n3c4ccccc4c4cc(-c5ccc(-c6ccc(-c7ccc8c(c7)c7ccccc7n8-c7cccc(-c8ccccc8)c7)cc6)cc5)ccc43)cc2)cc1 ; c1ccc(-c2ccc(-n3c4ccccc4c4cc(-c5ccc(-c6cccc(-c7ccc8c(c7)c7ccccc7n8-c7ccccc7-c7ccccc7)c6)cc5)ccc43)cc2)cc1]

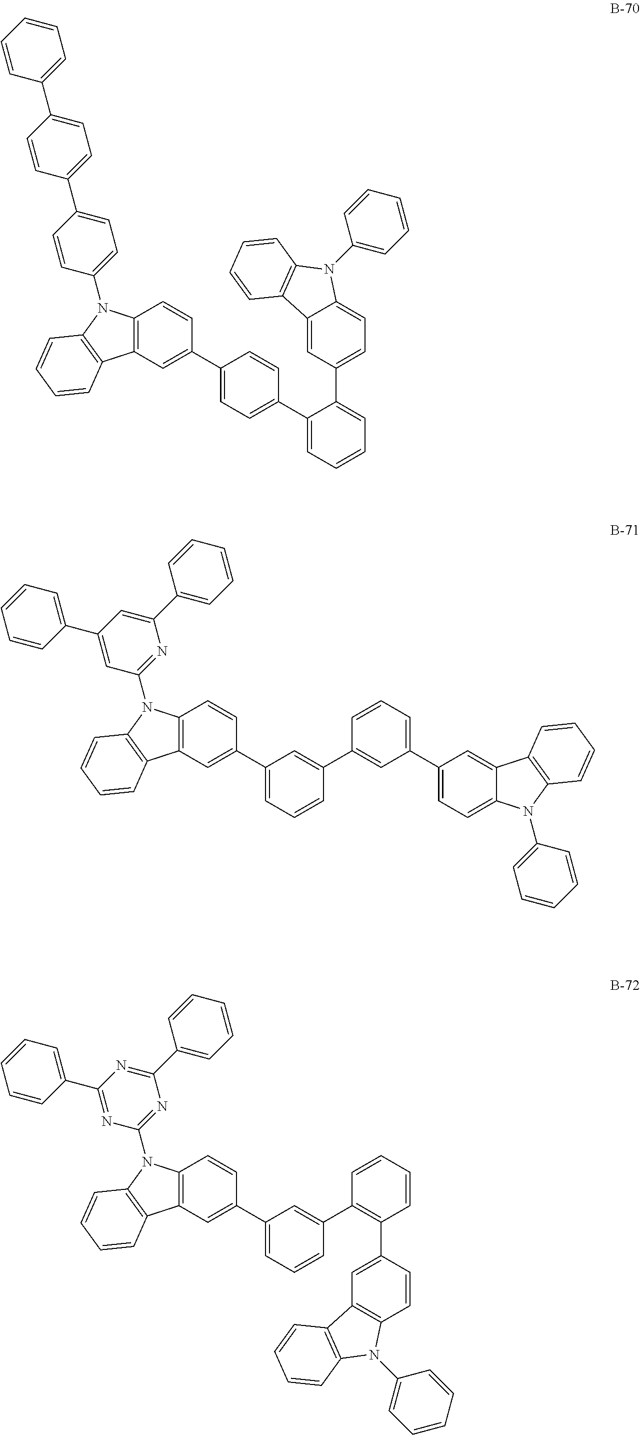 [SMILES: c1ccc(-c2ccc(-c3ccc(-n4c5ccccc5c5cc(-c6ccc(-c7ccccc7-c7ccc8c(c7)c7ccccc7n8-c7ccccc7)cc6)ccc54)cc3)cc2)cc1 ; c1ccc(-c2cc(-c3ccccc3)nc(-n3c4ccccc4c4cc(-c5cccc(-c6cccc(-c7ccc8c(c7)c7ccccc7n8-c7ccccc7)c6)c5)ccc43)c2)cc1 ; c1ccc(-c2nc(-c3ccccc3)nc(-n3c4ccccc4c4cc(-c5cccc(-c6ccccc6-c6ccc7c(c6)c6ccccc6n7-c6ccccc6)c5)ccc43)n2)cc1]

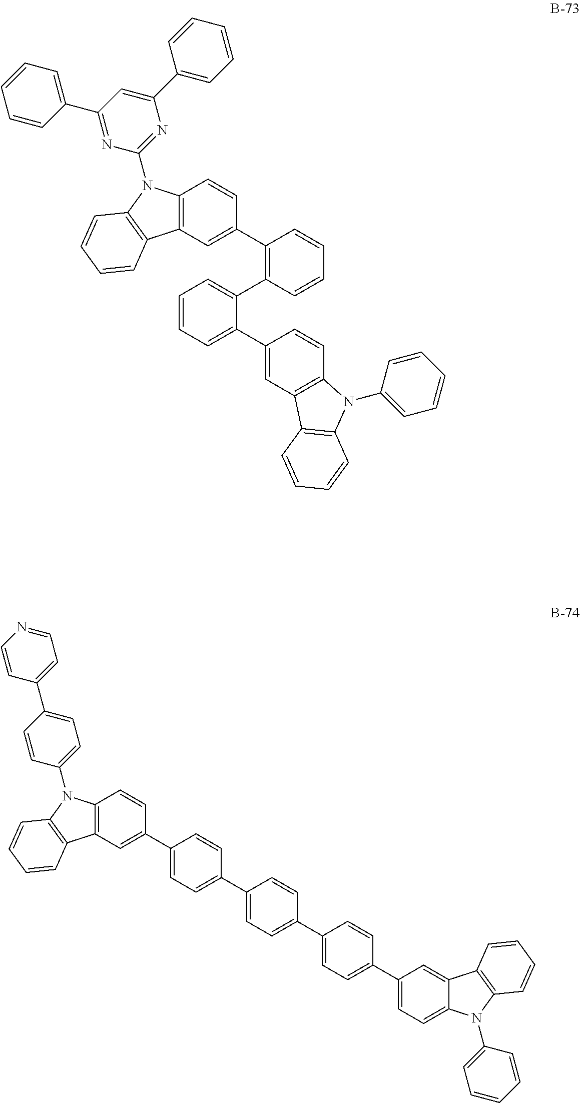 [SMILES: c1ccc(-c2cc(-c3ccccc3)nc(-n3c4ccccc4c4cc(-c5ccccc5-c5ccccc5-c5ccc6c(c5)c5ccccc5n6-c5ccccc5)ccc43)n2)cc1 ; c1ccc(-n2c3ccccc3c3cc(-c4ccc(-c5ccc(-c6ccc(-c7ccc8c(c7)c7ccccc7n8-c7ccc(-c8ccncc8)cc7)cc6)cc5)cc4)ccc32)cc1]

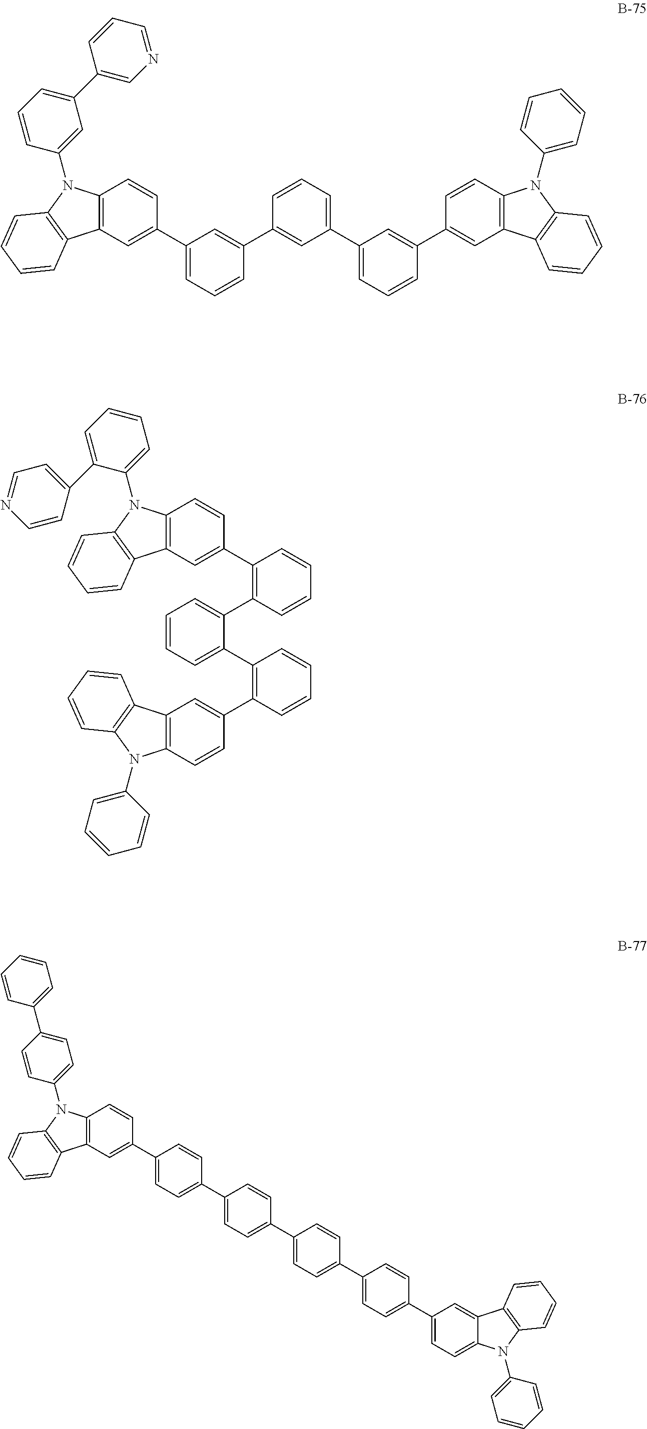 [SMILES: c1ccc(-n2c3ccccc3c3cc(-c4cccc(-c5cccc(-c6cccc(-c7ccc8c(c7)c7ccccc7n8-c7cccc(-c8cccnc8)c7)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4ccccc4-c4ccncc4)ccc32)cc1 ; c1ccc(-c2ccc(-n3c4ccccc4c4cc(-c5ccc(-c6ccc(-c7ccc(-c8ccc(-c9ccc%10c(c9)c9ccccc9n%10-c9ccccc9)cc8)cc7)cc6)cc5)ccc43)cc2)cc1]

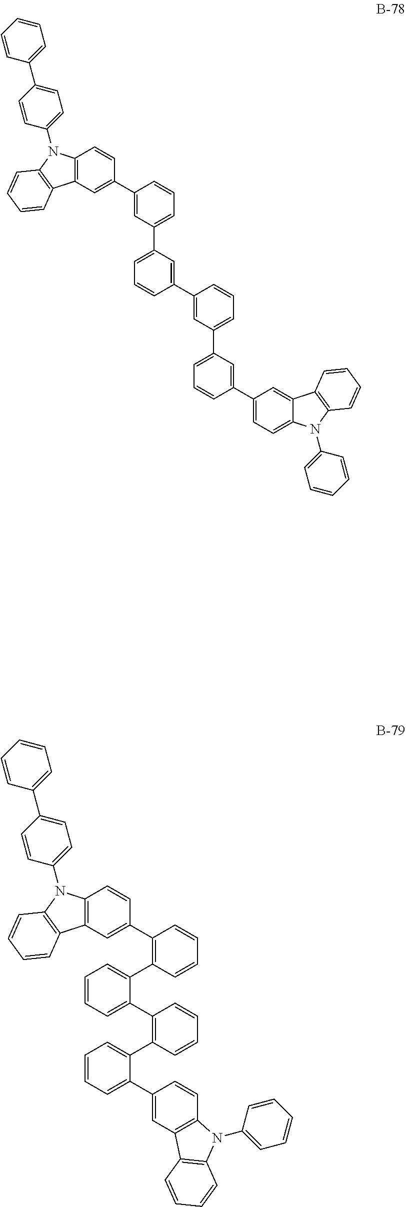 [SMILES: c1ccc(-c2ccc(-n3c4ccccc4c4cc(-c5cccc(-c6cccc(-c7cccc(-c8cccc(-c9ccc%10c(c9)c9ccccc9n%10-c9ccccc9)c8)c7)c6)c5)ccc43)cc2)cc1 ; c1ccc(-c2ccc(-n3c4ccccc4c4cc(-c5ccccc5-c5ccccc5-c5ccccc5-c5ccccc5-c5ccc6c(c5)c5ccccc5n6-c5ccccc5)ccc43)cc2)cc1]

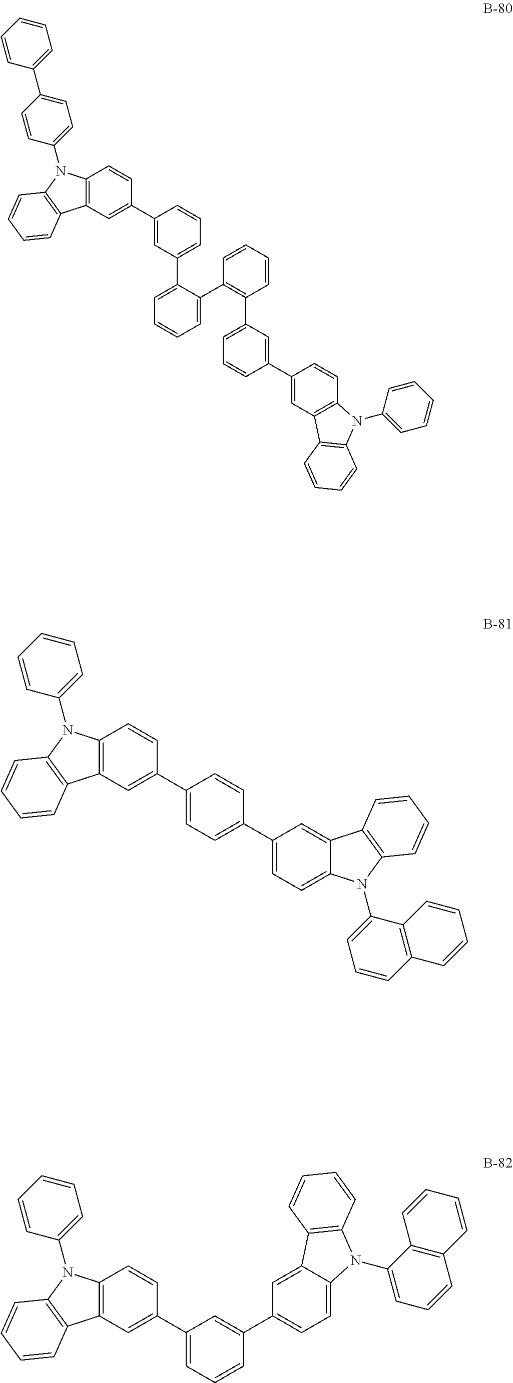 [SMILES: c1ccc(-c2ccc(-n3c4ccccc4c4cc(-c5cccc(-c6ccccc6-c6ccccc6-c6cccc(-c7ccc8c(c7)c7ccccc7n8-c7ccccc7)c6)c5)ccc43)cc2)cc1 ; c1ccc(-n2c3ccccc3c3cc(-c4ccc(-c5ccc6c(c5)c5ccccc5n6-c5cccc6ccccc56)cc4)ccc32)cc1 ; c1ccc(-n2c3ccccc3c3cc(-c4cccc(-c5ccc6c(c5)c5ccccc5n6-c5cccc6ccccc56)c4)ccc32)cc1]

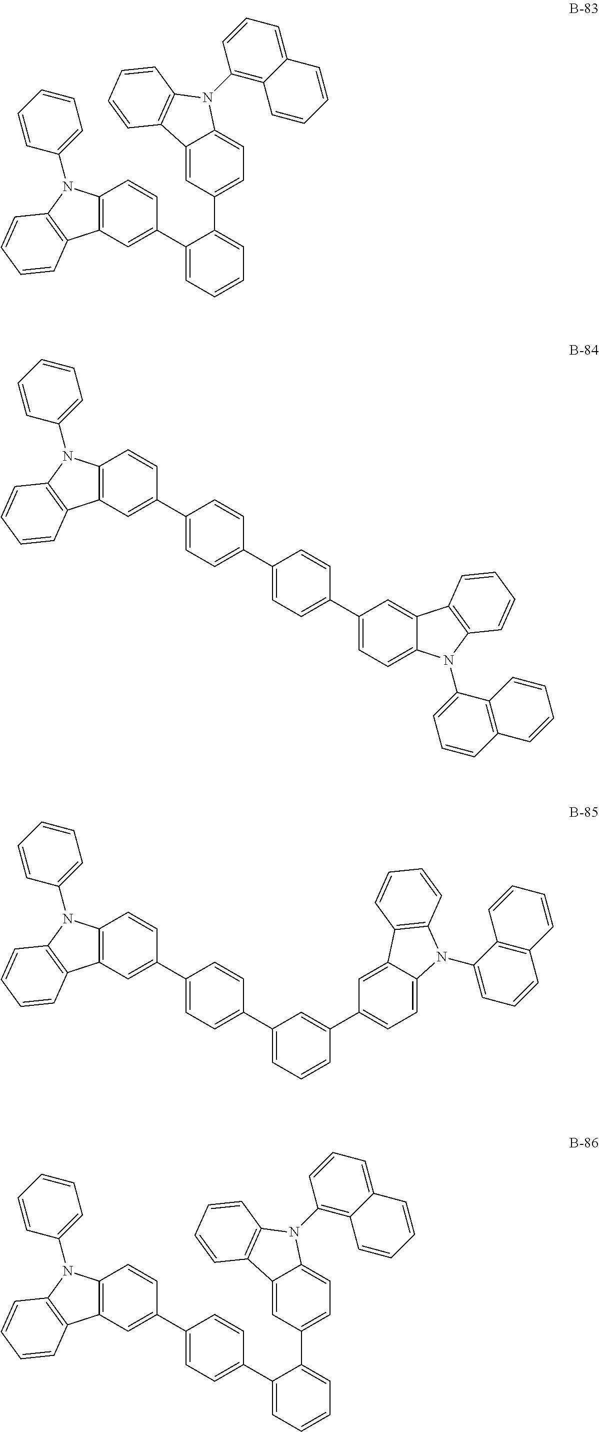 [SMILES: c1ccc(-n2c3ccccc3c3cc(-c4ccccc4-c4ccc5c(c4)c4ccccc4n5-c4cccc5ccccc45)ccc32)cc1 ; c1ccc(-n2c3ccccc3c3cc(-c4ccc(-c5ccc(-c6ccc7c(c6)c6ccccc6n7-c6cccc7ccccc67)cc5)cc4)ccc32)cc1 ; c1ccc(-n2c3ccccc3c3cc(-c4ccc(-c5cccc(-c6ccc7c(c6)c6ccccc6n7-c6cccc7ccccc67)c5)cc4)ccc32)cc1 ; c1ccc(-n2c3ccccc3c3cc(-c4ccc(-c5ccccc5-c5ccc6c(c5)c5ccccc5n6-c5cccc6ccccc56)cc4)ccc32)cc1]

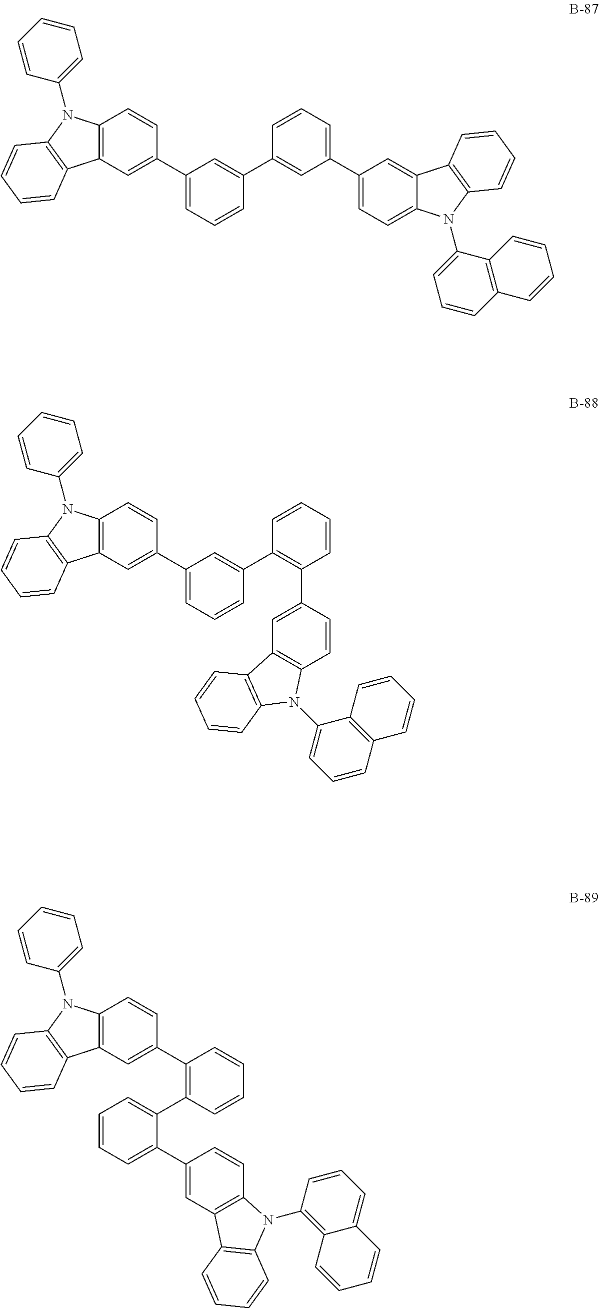 [SMILES: c1ccc(-n2c3ccccc3c3cc(-c4cccc(-c5cccc(-c6ccc7c(c6)c6ccccc6n7-c6cccc7ccccc67)c5)c4)ccc32)cc1 ; c1ccc(-n2c3ccccc3c3cc(-c4cccc(-c5ccccc5-c5ccc6c(c5)c5ccccc5n6-c5cccc6ccccc56)c4)ccc32)cc1 ; c1ccc(-n2c3ccccc3c3cc(-c4ccccc4-c4ccccc4-c4ccc5c(c4)c4ccccc4n5-c4cccc5ccccc45)ccc32)cc1]

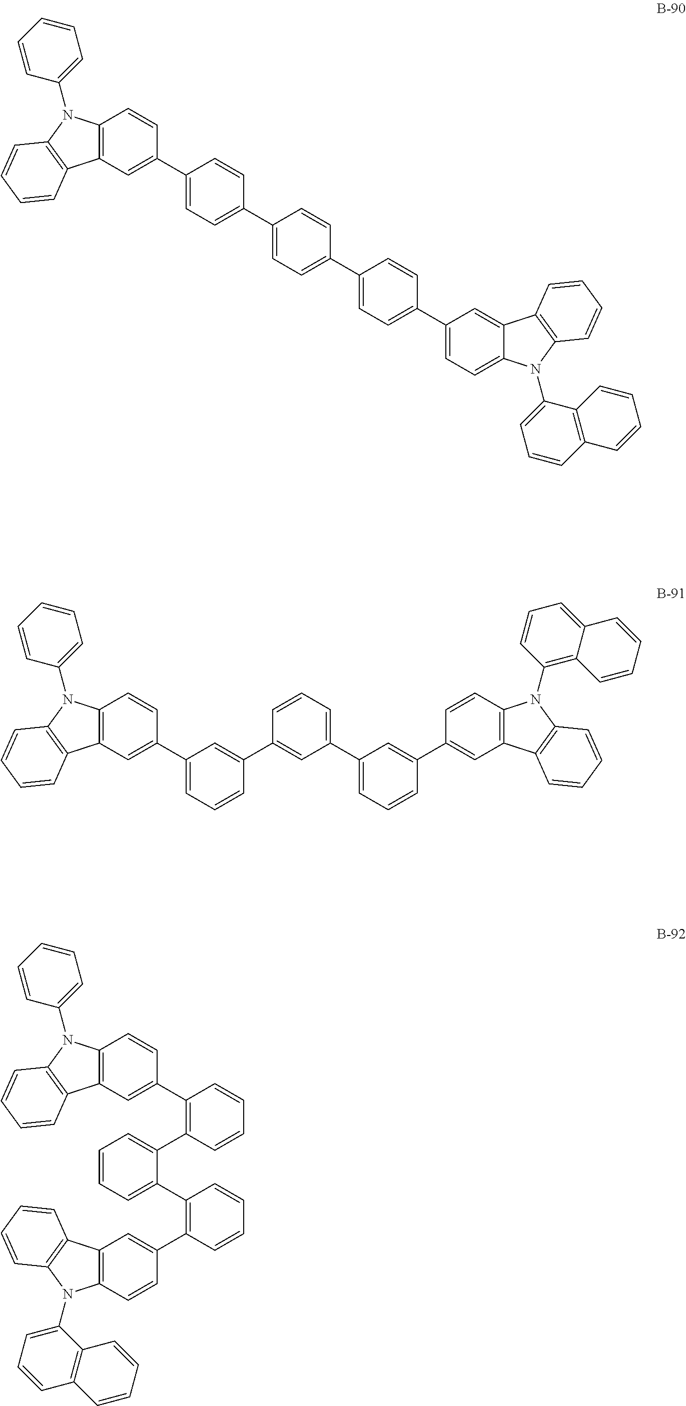 [SMILES: c1ccc(-n2c3ccccc3c3cc(-c4ccc(-c5ccc(-c6ccc(-c7ccc8c(c7)c7ccccc7n8-c7cccc8ccccc78)cc6)cc5)cc4)ccc32)cc1 ; c1ccc(-n2c3ccccc3c3cc(-c4cccc(-c5cccc(-c6cccc(-c7ccc8c(c7)c7ccccc7n8-c7cccc8ccccc78)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4cccc5ccccc45)ccc32)cc1]

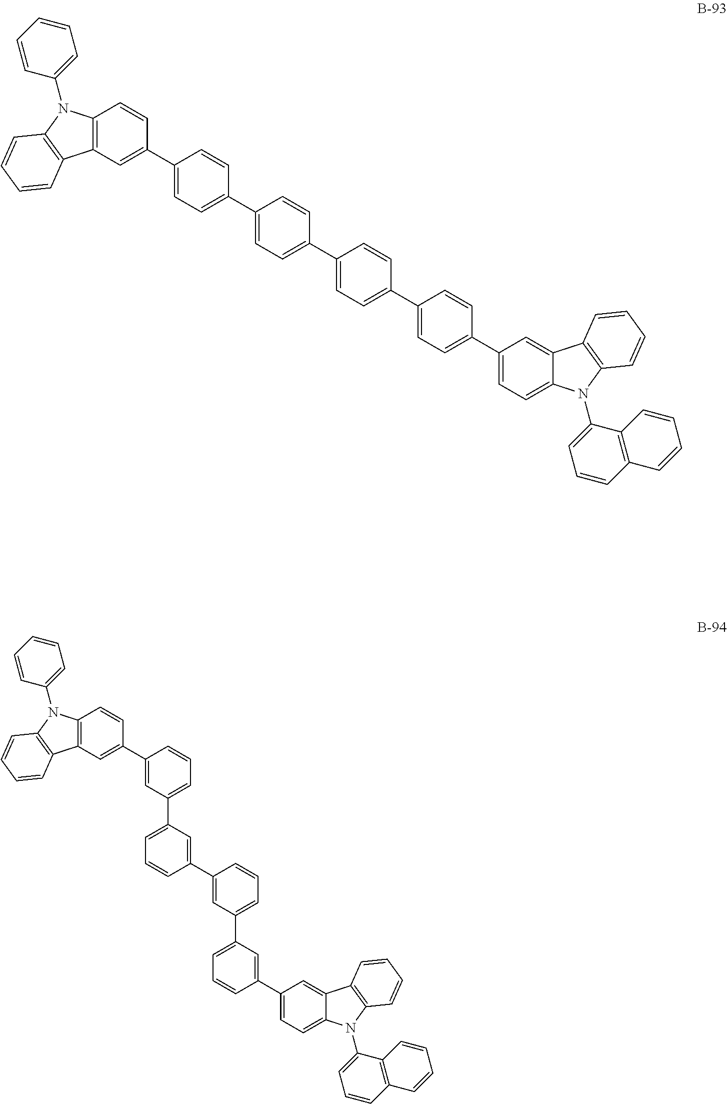 [SMILES: c1ccc(-n2c3ccccc3c3cc(-c4ccc(-c5ccc(-c6ccc(-c7ccc(-c8ccc9c(c8)c8ccccc8n9-c8cccc9ccccc89)cc7)cc6)cc5)cc4)ccc32)cc1 ; c1ccc(-n2c3ccccc3c3cc(-c4cccc(-c5cccc(-c6cccc(-c7cccc(-c8ccc9c(c8)c8ccccc8n9-c8cccc9ccccc89)c7)c6)c5)c4)ccc32)cc1]

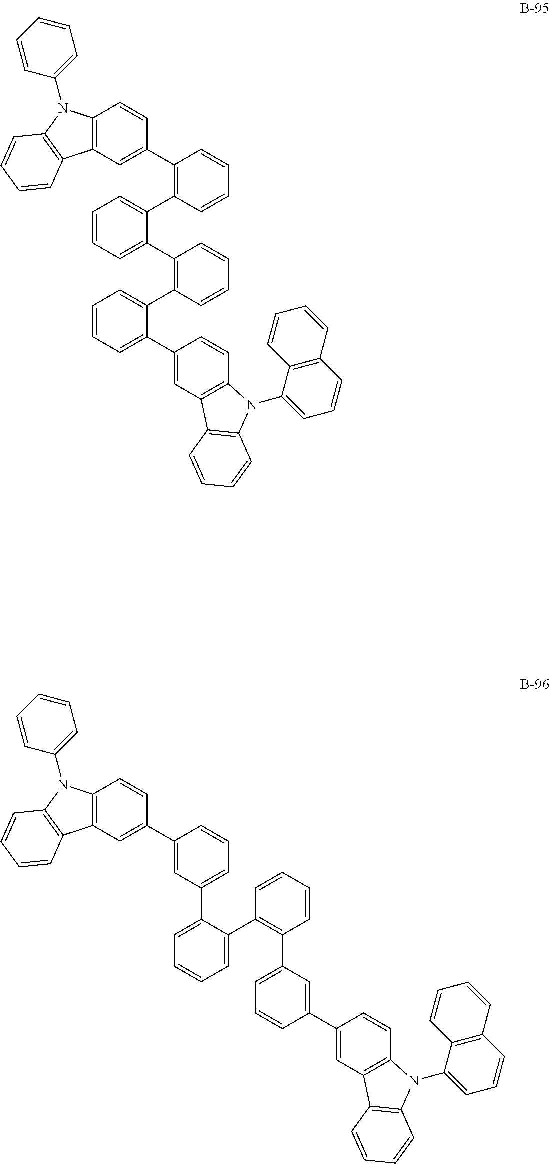 [SMILES: c1ccc(-n2c3ccccc3c3cc(-c4ccccc4-c4ccccc4-c4ccccc4-c4ccccc4-c4ccc5c(c4)c4ccccc4n5-c4cccc5ccccc45)ccc32)cc1 ; c1ccc(-n2c3ccccc3c3cc(-c4cccc(-c5ccccc5-c5ccccc5-c5cccc(-c6ccc7c(c6)c6ccccc6n7-c6cccc7ccccc67)c5)c4)ccc32)cc1]

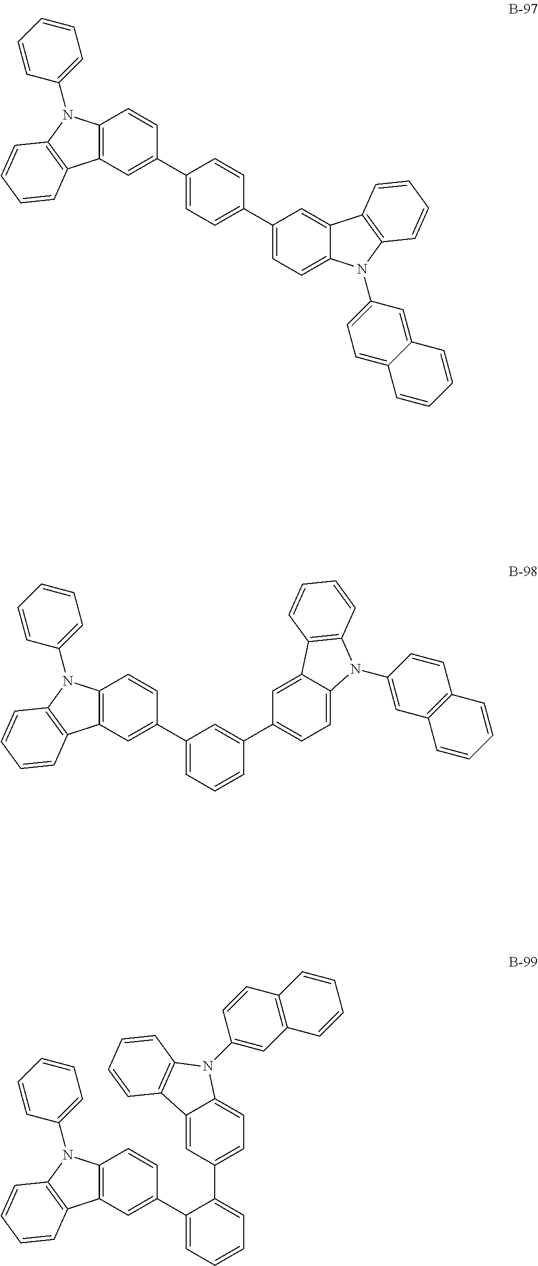 [SMILES: c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4cccc(-c5ccc6c(c5)c5ccccc5n6-c5ccc6ccccc6c5)c4)ccc32)cc1 ; c1ccc(-n2c3ccccc3c3cc(-c4ccccc4-c4ccc5c(c4)c4ccccc4n5-c4ccc5ccccc5c4)ccc32)cc1]

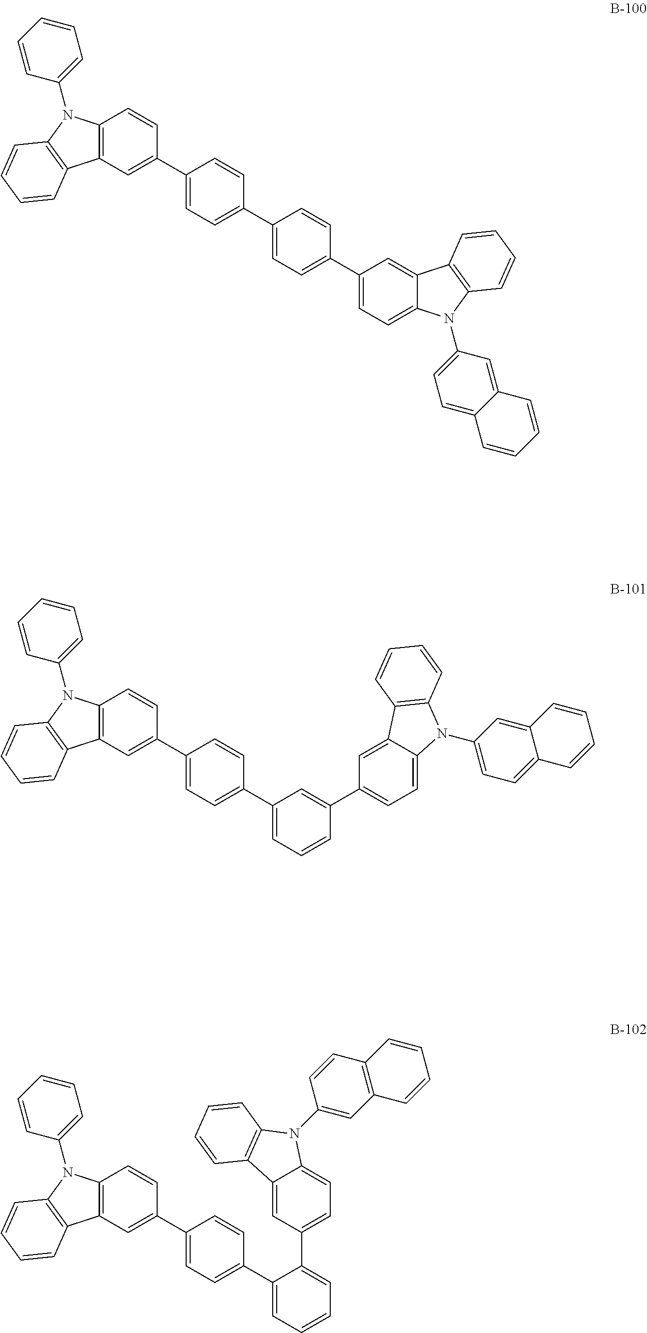 [SMILES: c1ccc(-n2c3ccccc3c3cc(-c4ccc(-c5ccc(-c6ccc7c(c6)c6ccccc6n7-c6ccc7ccccc7c6)cc5)cc4)ccc32)cc1 ; c1ccc(-n2c3ccccc3c3cc(-c4ccc(-c5cccc(-c6ccc7c(c6)c6ccccc6n7-c6ccc7ccccc7c6)c5)cc4)ccc32)cc1 ; c1ccc(-n2c3ccccc3c3cc(-c4ccc(-c5ccccc5-c5ccc6c(c5)c5ccccc5n6-c5ccc6ccccc6c5)cc4)ccc32)cc1]

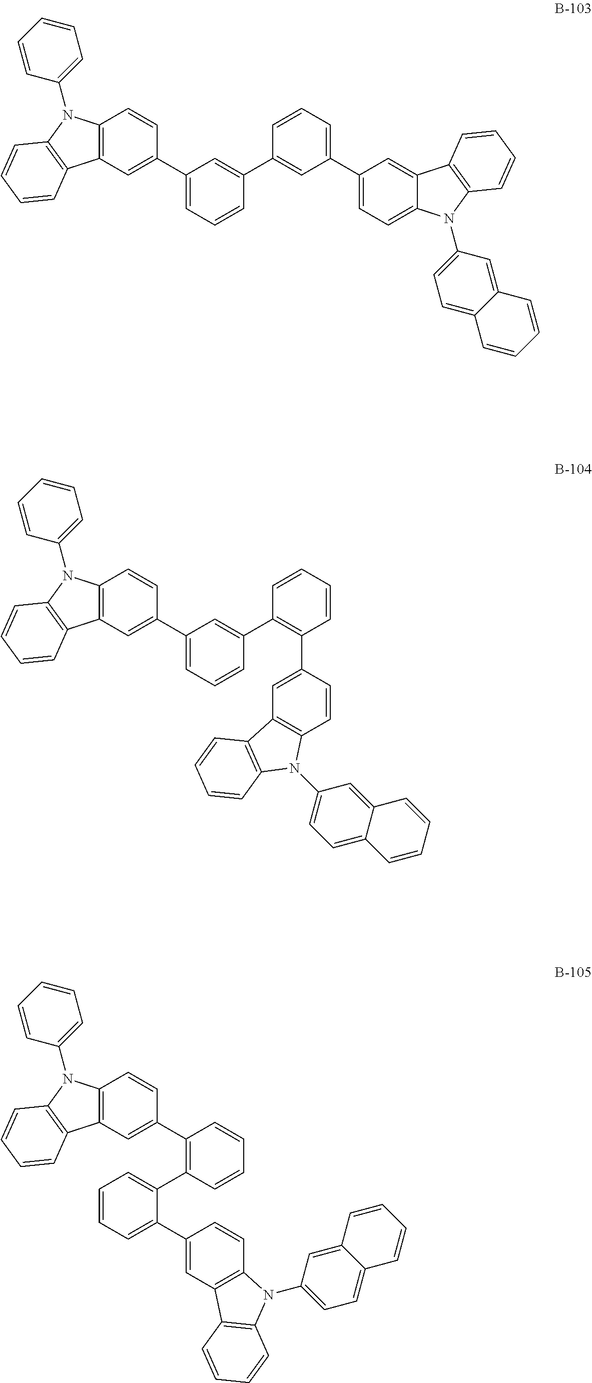 [SMILES: c1ccc(-n2c3ccccc3c3cc(-c4cccc(-c5cccc(-c6ccc7c(c6)c6ccccc6n7-c6ccc7ccccc7c6)c5)c4)ccc32)cc1 ; c1ccc(-n2c3ccccc3c3cc(-c4cccc(-c5ccccc5-c5ccc6c(c5)c5ccccc5n6-c5ccc6ccccc6c5)c4)ccc32)cc1 ; c1ccc(-n2c3ccccc3c3cc(-c4ccccc4-c4ccccc4-c4ccc5c(c4)c4ccccc4n5-c4ccc5ccccc5c4)ccc32)cc1]

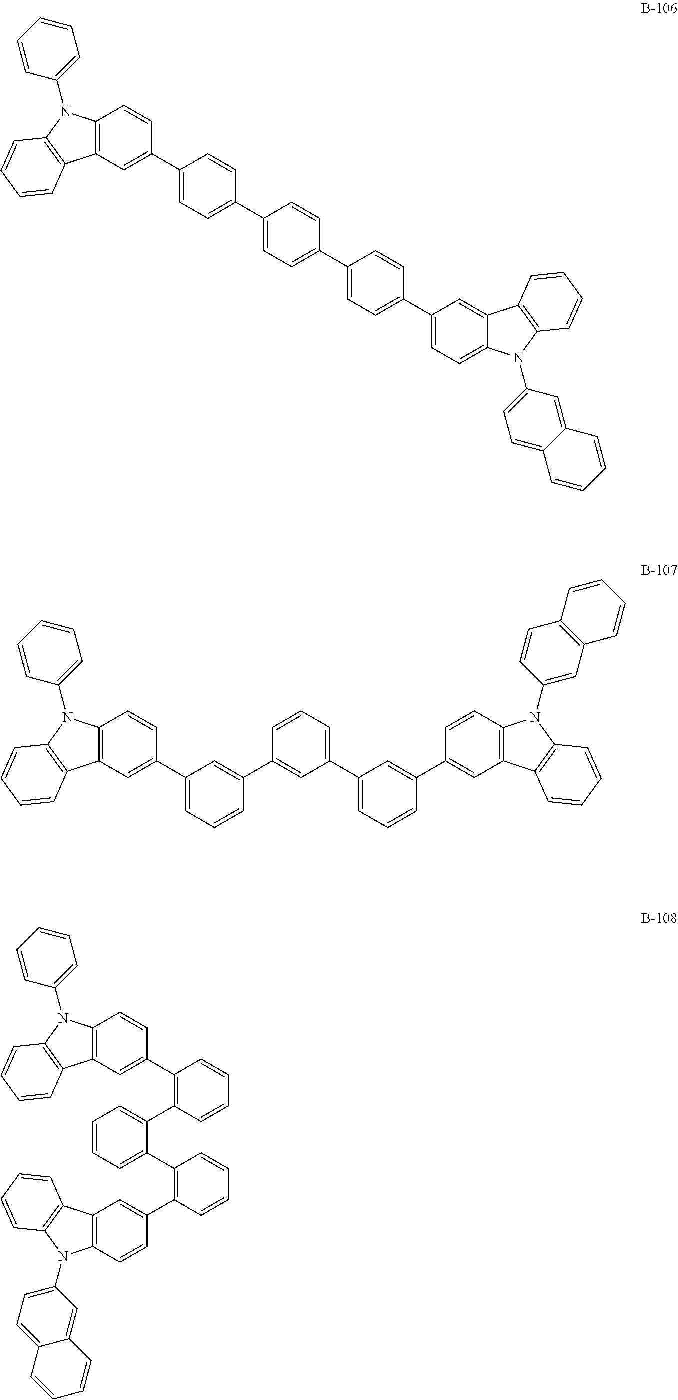 [SMILES: c1ccc(-n2c3ccccc3c3cc(-c4ccc(-c5ccc(-c6ccc(-c7ccc8c(c7)c7ccccc7n8-c7ccc8ccccc8c7)cc6)cc5)cc4)ccc32)cc1 ; c1ccc(-n2c3ccccc3c3cc(-c4cccc(-c5cccc(-c6cccc(-c7ccc8c(c7)c7ccccc7n8-c7ccc8ccccc8c7)c6)c5)c4)ccc32)cc1 ; c1ccc(-n2c3ccccc3c3cc(-c4ccccc4-c4ccccc4-c4ccccc4-c4ccc5c(c4)c4ccccc4n5-c4ccc5ccccc5c4)ccc32)cc1]

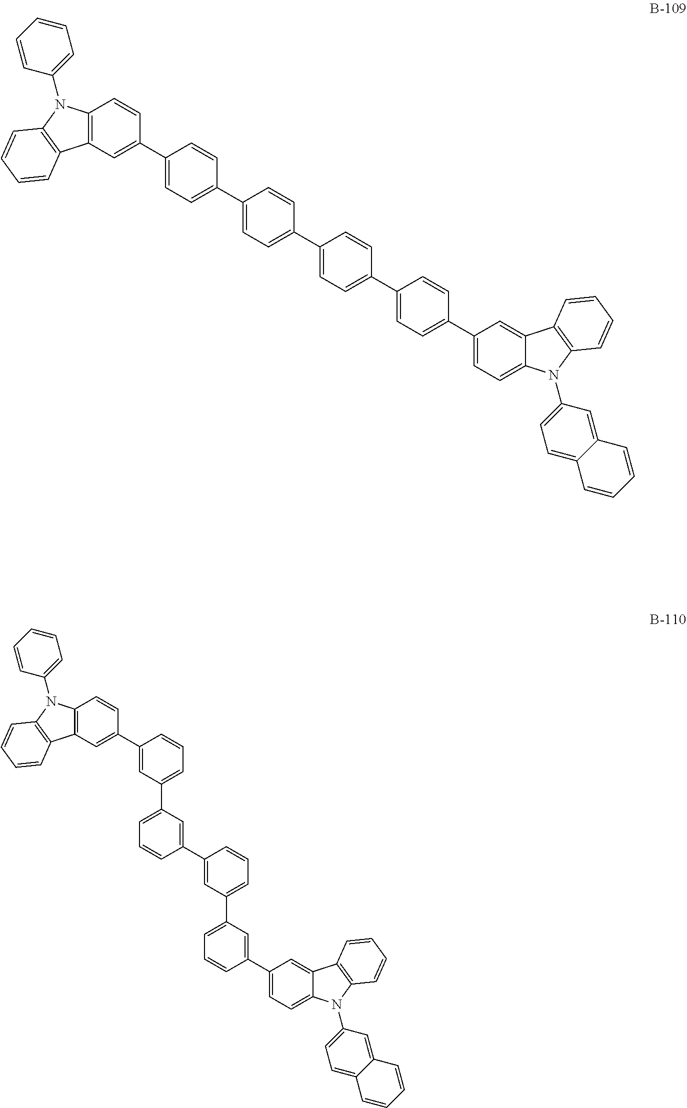 [SMILES: c1ccc(-n2c3ccccc3c3cc(-c4ccc(-c5ccc(-c6ccc(-c7ccc(-c8ccc9c(c8)c8ccccc8n9-c8ccc9ccccc9c8)cc7)cc6)cc5)cc4)ccc32)cc1 ; c1ccc(-n2c3ccccc3c3cc(-c4cccc(-c5cccc(-c6cccc(-c7cccc(-c8ccc9c(c8)c8ccccc8n9-c8ccc9ccccc9c8)c7)c6)c5)c4)ccc32)cc1]

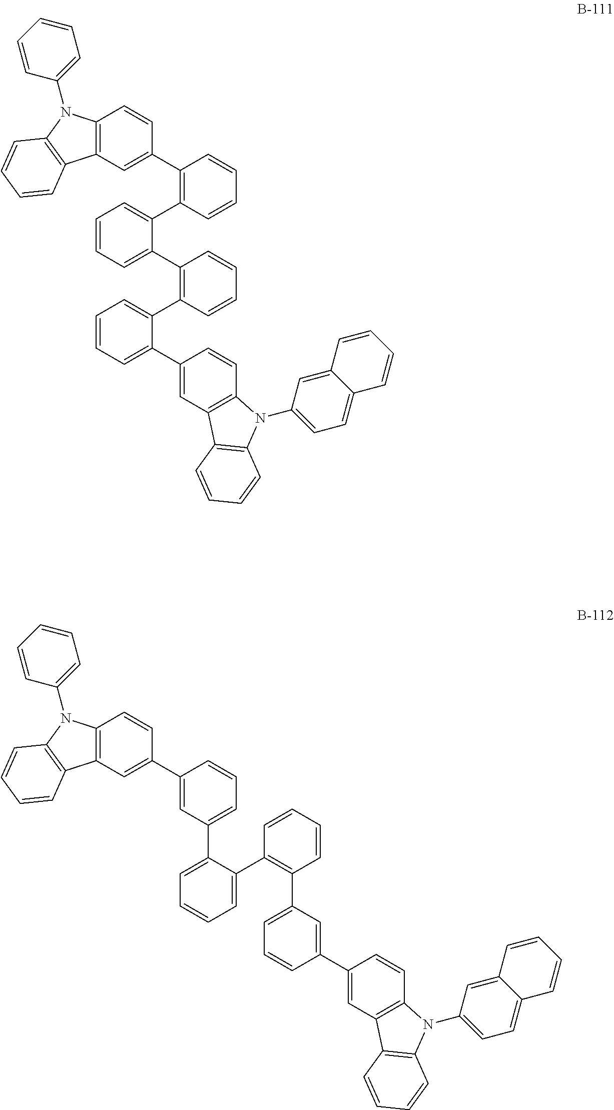 [SMILES: c1ccc(-n2c3ccccc3c3cc(-c4ccccc4-c4ccccc4-c4ccccc4-c4ccccc4-c4ccc5c(c4)c4ccccc4n5-c4ccc5ccccc5c4)ccc32)cc1 ; c1ccc(-n2c3ccccc3c3cc(-c4cccc(-c5ccccc5-c5ccccc5-c5cccc(-c6ccc7c(c6)c6ccccc6n7-c6ccc7ccccc7c6)c5)c4)ccc32)cc1]

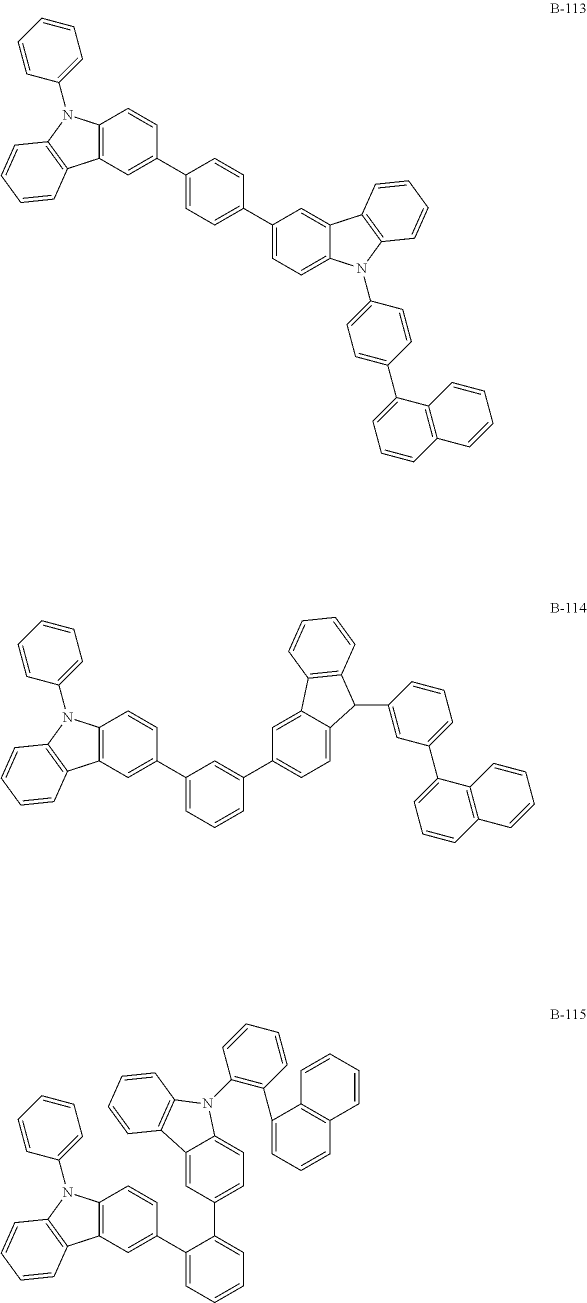 [SMILES: c1ccc(-n2c3ccccc3c3cc(-c4ccc(-c5ccc6c(c5)c5ccccc5n6-c5ccc(-c6cccc7ccccc67)cc5)cc4)ccc32)cc1 ; c1ccc(-n2c3ccccc3c3cc(-c4cccc(-c5ccc6c(c5)-c5ccccc5C6c5cccc(-c6cccc7ccccc67)c5)c4)ccc32)cc1 ; c1ccc(-n2c3ccccc3c3cc(-c4ccccc4-c4ccc5c(c4)c4ccccc4n5-c4ccccc4-c4cccc5ccccc45)ccc32)cc1]

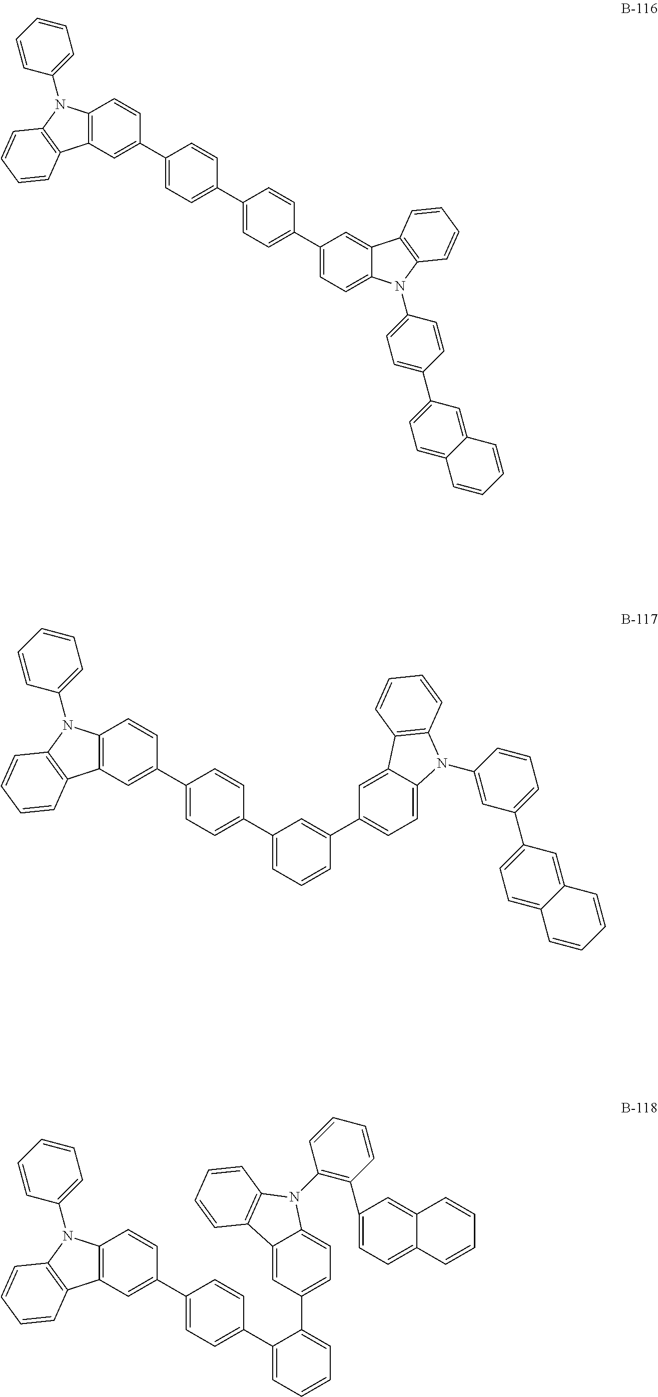 [SMILES: c1ccc(-n2c3ccccc3c3cc(-c4ccc(-c5ccc(-c6ccc7c(c6)c6ccccc6n7-c6ccc(-c7ccc8ccccc8c7)cc6)cc5)cc4)ccc32)cc1 ; c1ccc(-n2c3ccccc3c3cc(-c4ccc(-c5cccc(-c6ccc7c(c6)c6ccccc6n7-c6cccc(-c7ccc8ccccc8c7)c6)c5)cc4)ccc32)cc1 ; c1ccc(-n2c3ccccc3c3cc(-c4ccc(-c5ccccc5-c5ccc6c(c5)c5ccccc5n6-c5ccccc5-c5ccc6ccccc6c5)cc4)ccc32)cc1]

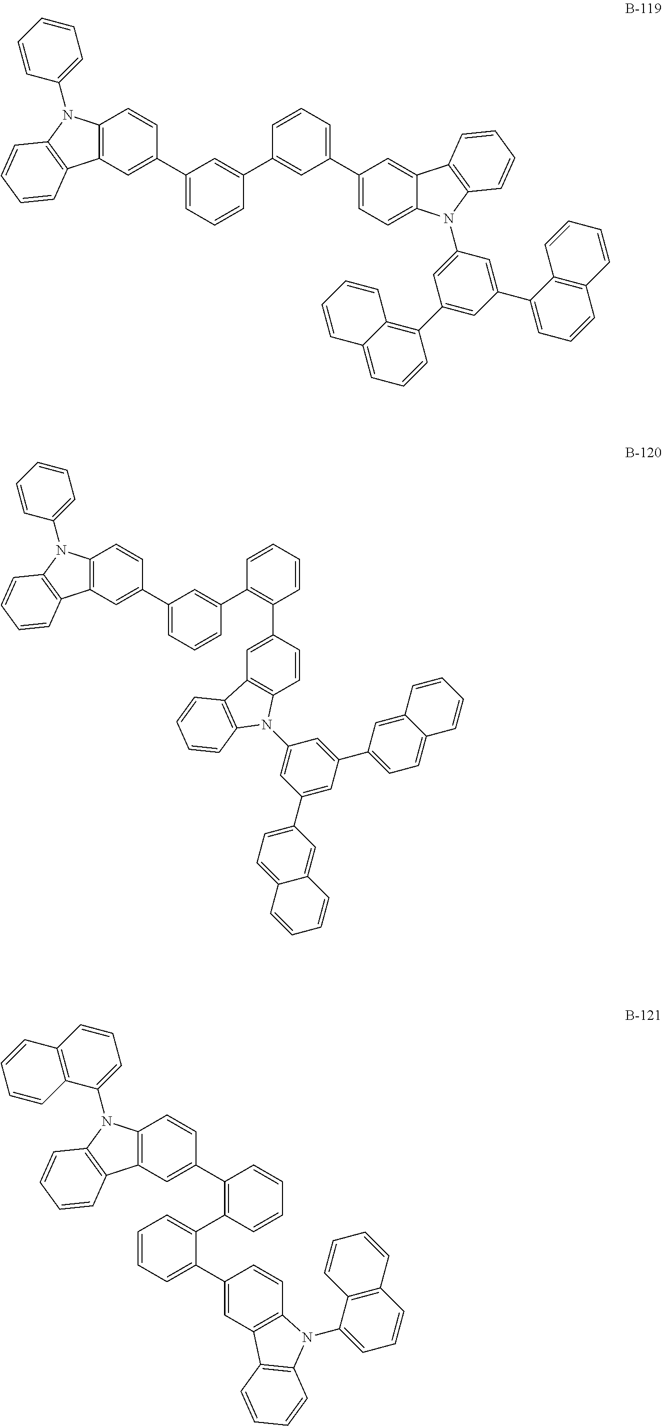 [SMILES: c1ccc(-n2c3ccccc3c3cc(-c4cccc(-c5cccc(-c6ccc7c(c6)c6ccccc6n7-c6cc(-c7cccc8ccccc78)cc(-c7cccc8ccccc78)c6)c5)c4)ccc32)cc1 ; c1ccc(-n2c3ccccc3c3cc(-c4cccc(-c5ccccc5-c5ccc6c(c5)c5ccccc5n6-c5cc(-c6ccc7ccccc7c6)cc(-c6ccc7ccccc7c6)c5)c4)ccc32)cc1 ; c1ccc(-c2ccccc2-c2ccc3c(c2)c2ccccc2n3-c2cccc3ccccc23)c(-c2ccc3c(c2)c2ccccc2n3-c2cccc3ccccc23)c1]

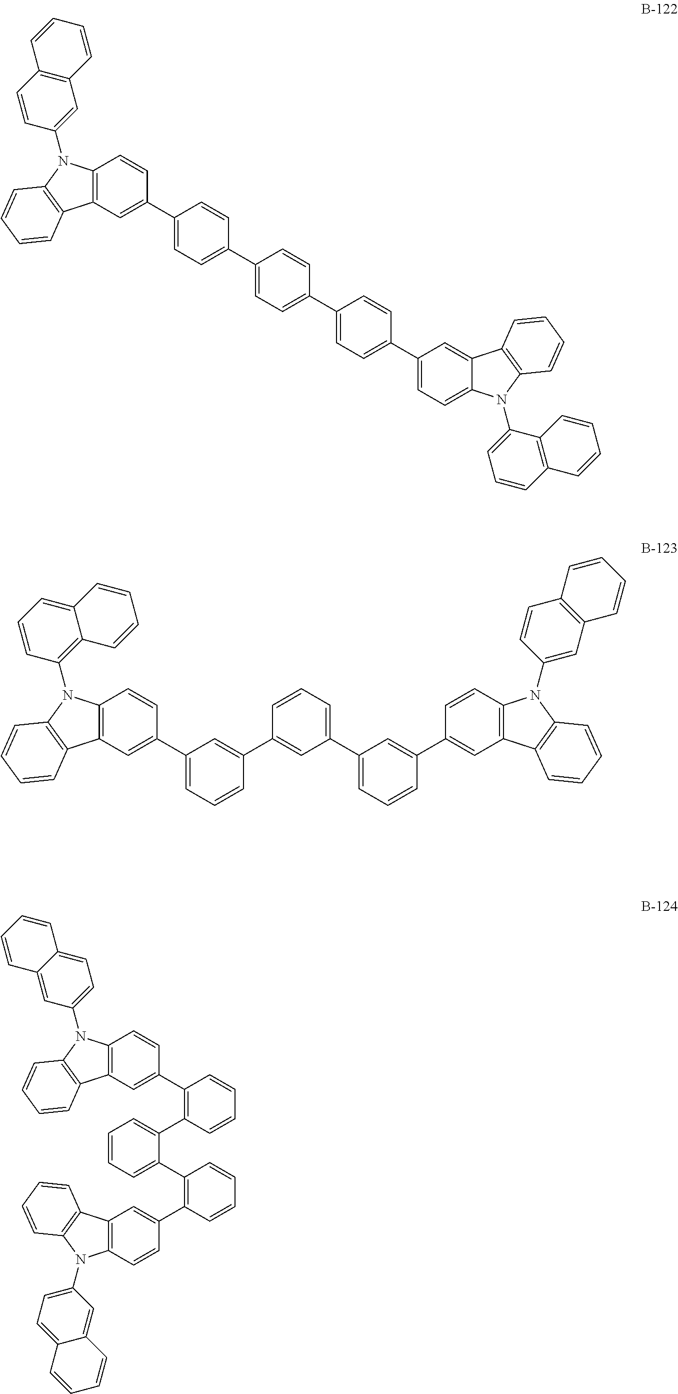 [SMILES: c1ccc2cc(-n3c4ccccc4c4cc(-c5ccc(-c6ccc(-c7ccc(-c8ccc9c(c8)c8ccccc8n9-c8cccc9ccccc89)cc7)cc6)cc5)ccc43)ccc2c1 ; c1cc(-c2cccc(-c3ccc4c(c3)c3ccccc3n4-c3ccc4ccccc4c3)c2)cc(-c2cccc(-c3ccc4c(c3)c3ccccc3n4-c3cccc4ccccc34)c2)c1 ; c1ccc(-c2ccccc2-c2ccccc2-c2ccc3c(c2)c2ccccc2n3-c2ccc3ccccc3c2)c(-c2ccc3c(c2)c2ccccc2n3-c2ccc3ccccc3c2)c1]

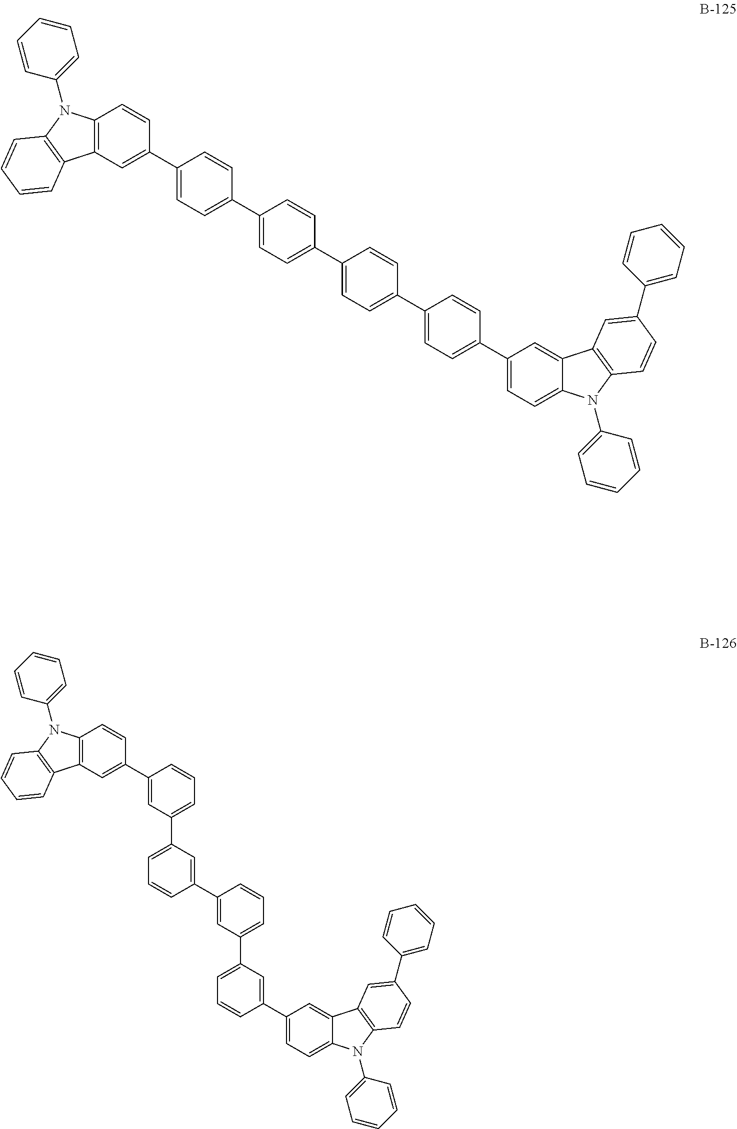 [SMILES: c1ccc(-c2ccc3c(c2)c2cc(-c4ccc(-c5ccc(-c6ccc(-c7ccc(-c8ccc9c(c8)c8ccccc8n9-c8ccccc8)cc7)cc6)cc5)cc4)ccc2n3-c2ccccc2)cc1 ; c1ccc(-c2ccc3c(c2)c2cc(-c4cccc(-c5cccc(-c6cccc(-c7cccc(-c8ccc9c(c8)c8ccccc8n9-c8ccccc8)c7)c6)c5)c4)ccc2n3-c2ccccc2)cc1]

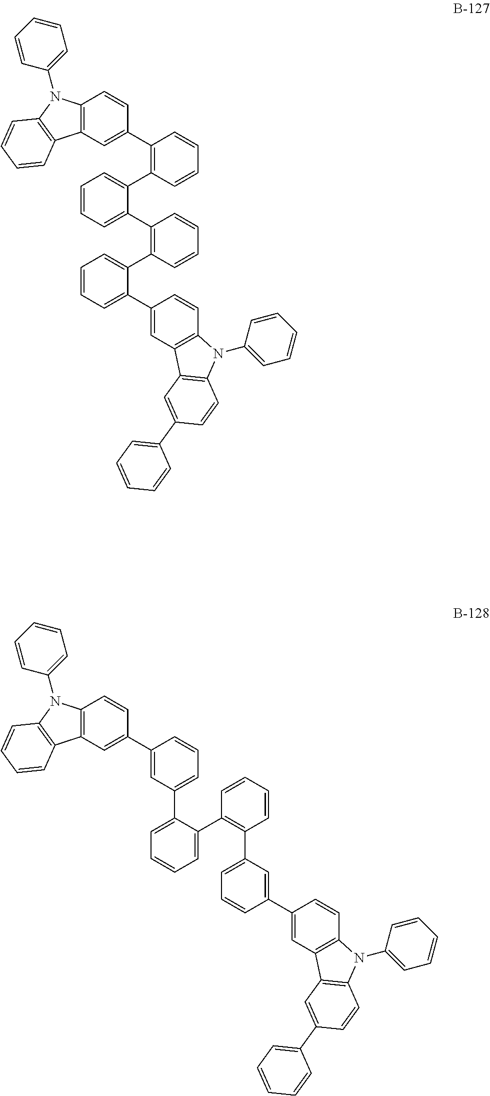 [SMILES: c1ccc(-c2ccc3c(c2)c2cc(-c4ccccc4-c4ccccc4-c4ccccc4-c4ccccc4-c4ccc5c(c4)c4ccccc4n5-c4ccccc4)ccc2n3-c2ccccc2)cc1 ; c1ccc(-c2ccc3c(c2)c2cc(-c4cccc(-c5ccccc5-c5ccccc5-c5cccc(-c6ccc7c(c6)c6ccccc6n7-c6ccccc6)c5)c4)ccc2n3-c2ccccc2)cc1]

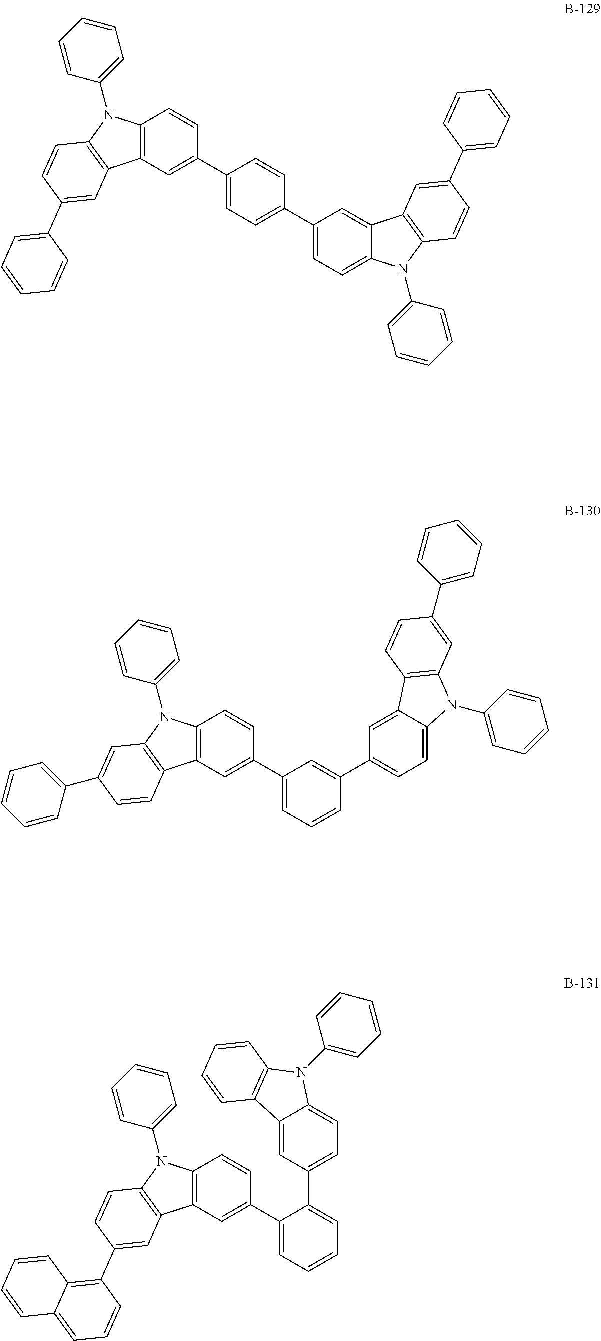 [SMILES: c1ccc(-c2ccc3c(c2)c2cc(-c4ccc(-c5ccc6c(c5)c5cc(-c7ccccc7)ccc5n6-c5ccccc5)cc4)ccc2n3-c2ccccc2)cc1 ; c1ccc(-c2ccc3c4cc(-c5cccc(-c6ccc7c(c6)c6ccc(-c8ccccc8)cc6n7-c6ccccc6)c5)ccc4n(-c4ccccc4)c3c2)cc1 ; c1ccc(-n2c3ccccc3c3cc(-c4ccccc4-c4ccc5c(c4)c4cc(-c6cccc7ccccc67)ccc4n5-c4ccccc4)ccc32)cc1]

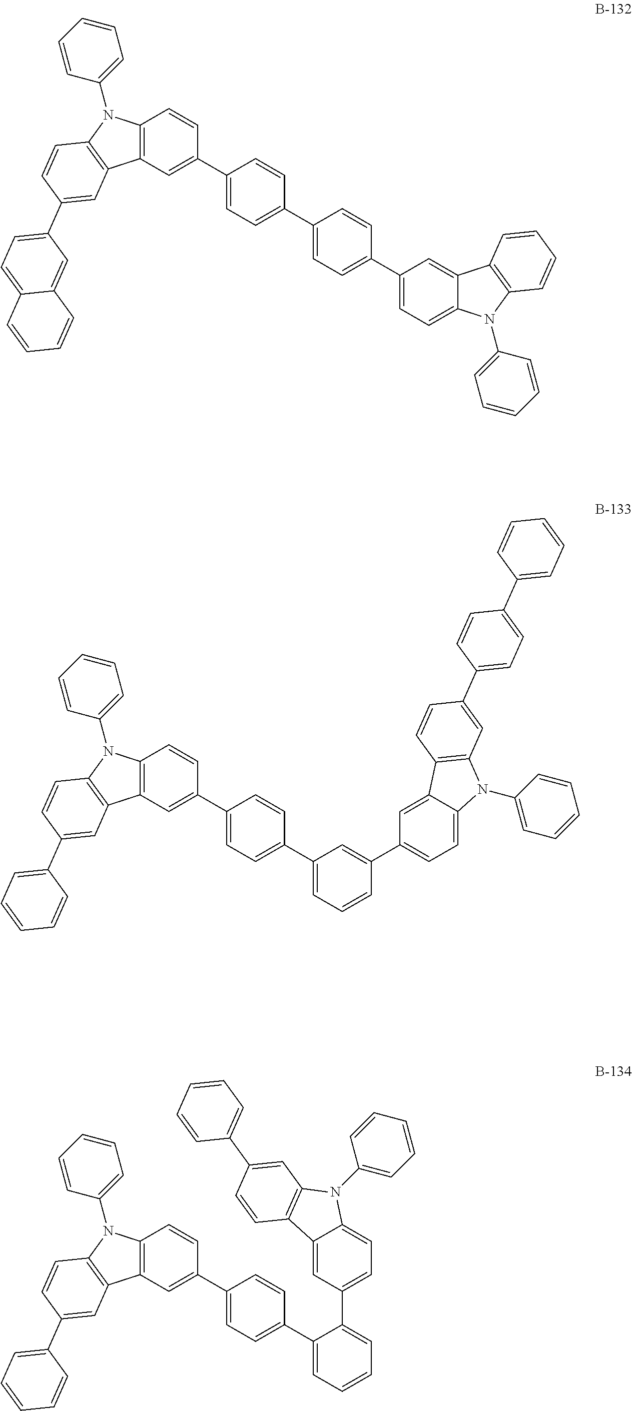 [SMILES: c1ccc(-n2c3ccccc3c3cc(-c4ccc(-c5ccc(-c6ccc7c(c6)c6cc(-c8ccc9ccccc9c8)ccc6n7-c6ccccc6)cc5)cc4)ccc32)cc1 ; c1ccc(-c2ccc(-c3ccc4c5cc(-c6cccc(-c7ccc(-c8ccc9c(c8)c8cc(-c%10ccccc%10)ccc8n9-c8ccccc8)cc7)c6)ccc5n(-c5ccccc5)c4c3)cc2)cc1 ; c1ccc(-c2ccc3c(c2)c2cc(-c4ccc(-c5ccccc5-c5ccc6c(c5)c5ccc(-c7ccccc7)cc5n6-c5ccccc5)cc4)ccc2n3-c2ccccc2)cc1]

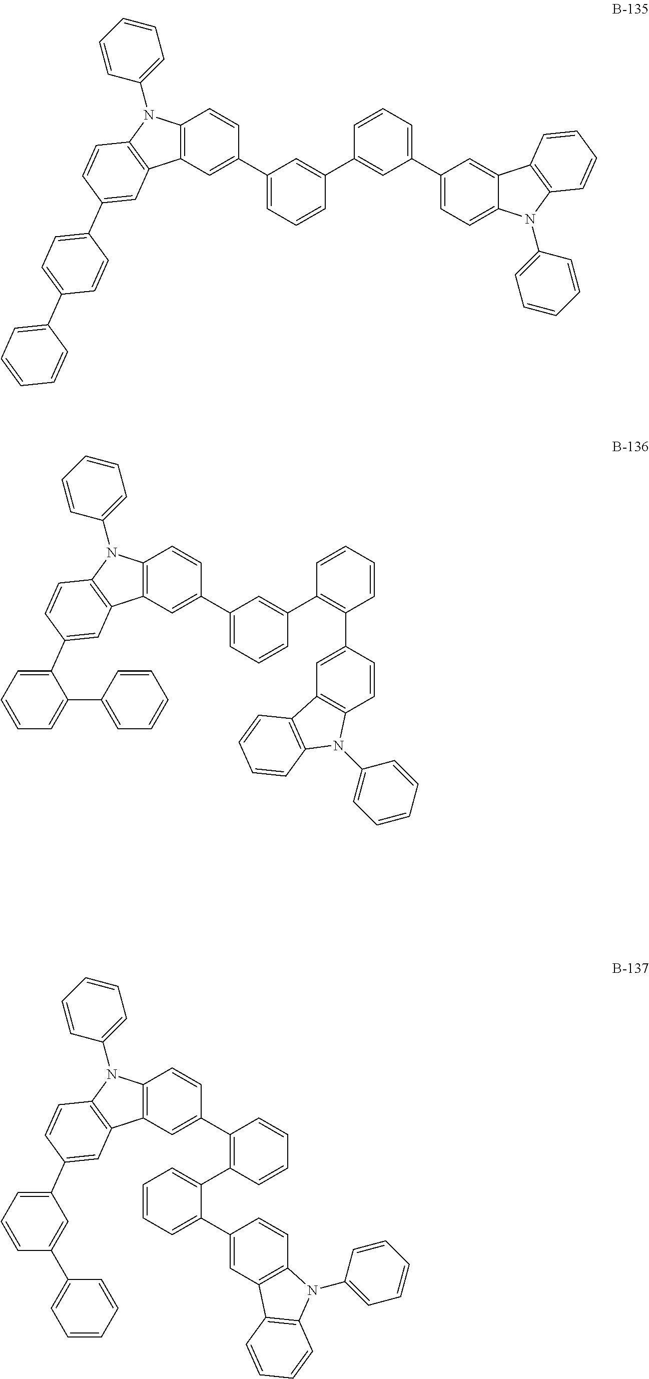 [SMILES: c1ccc(-c2ccc(-c3ccc4c(c3)c3cc(-c5cccc(-c6cccc(-c7ccc8c(c7)c7ccccc7n8-c7ccccc7)c6)c5)ccc3n4-c3ccccc3)cc2)cc1 ; c1ccc(-c2ccccc2-c2ccc3c(c2)c2cc(-c4cccc(-c5ccccc5-c5ccc6c(c5)c5ccccc5n6-c5ccccc5)c4)ccc2n3-c2ccccc2)cc1 ; c1ccc(-c2cccc(-c3ccc4c(c3)c3cc(-c5ccccc5-c5ccccc5-c5ccc6c(c5)c5ccccc5n6-c5ccccc5)ccc3n4-c3ccccc3)c2)cc1]

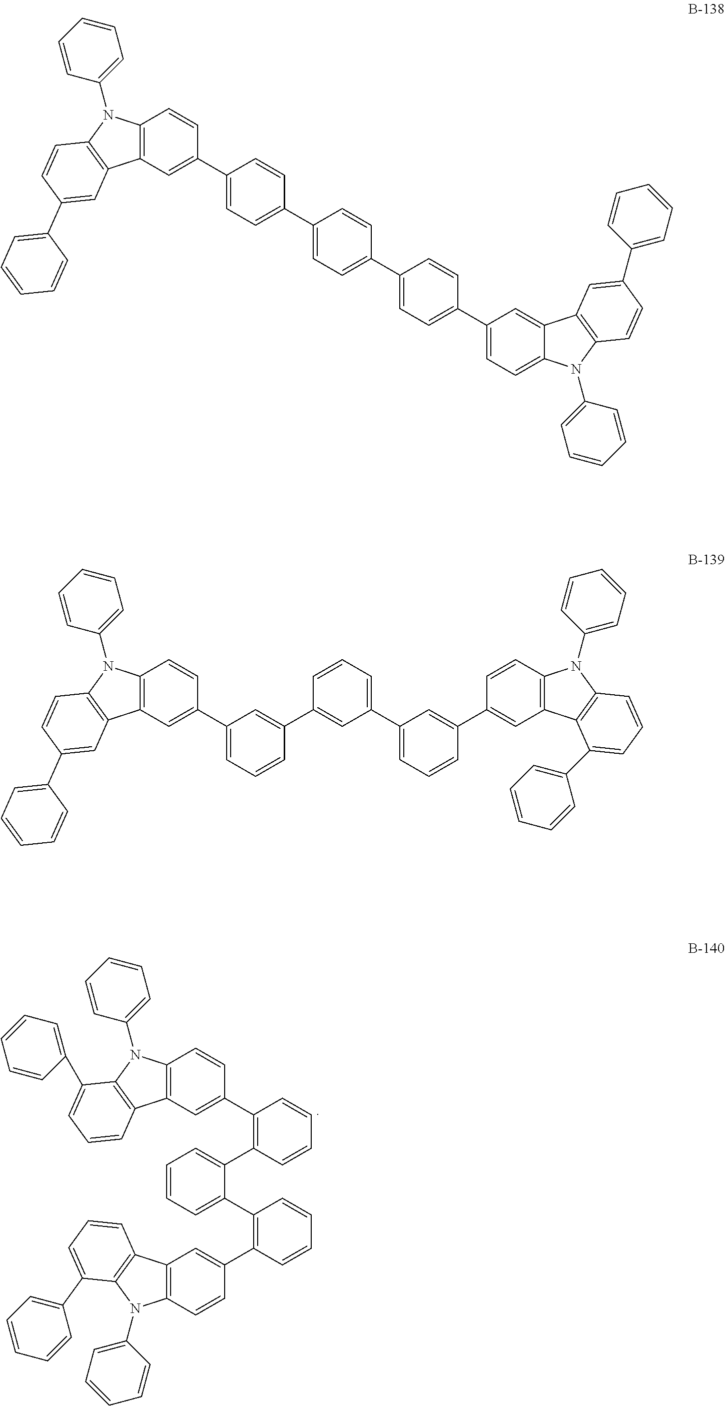 [SMILES: c1ccc(-c2ccc3c(c2)c2cc(-c4ccc(-c5ccc(-c6ccc(-c7ccc8c(c7)c7cc(-c9ccccc9)ccc7n8-c7ccccc7)cc6)cc5)cc4)ccc2n3-c2ccccc2)cc1 ; c1ccc(-c2ccc3c(c2)c2cc(-c4cccc(-c5cccc(-c6cccc(-c7ccc8c(c7)c7c(-c9ccccc9)cccc7n8-c7ccccc7)c6)c5)c4)ccc2n3-c2ccccc2)cc1 ; c1ccc(-c2cccc3c4cc(-c5ccccc5-c5ccccc5-c5ccccc5-c5ccc6c(c5)c5cccc(-c7ccccc7)c5n6-c5ccccc5)ccc4n(-c4ccccc4)c23)cc1]

14. The composition for an organic optoelectric diode of claim 1, wherein a LUMO energy level of the first host compound is −1.5 eV to −3.0 eV.

15. The composition for an organic optoelectric diode of claim 1, wherein a HOMO energy level of the first host compound is less than or equal to −5.8 eV.

16. The composition for an organic optoelectric diode of claim 1, wherein the first host compound and the second host compound are included in a weight ratio of 1:10 to 10:1.

17. The composition for an organic optoelectric diode of claim 1, wherein the composition further includes a phosphorescent dopant.

18. An organic optoelectric diode, comprising
an anode and a cathode facing each other, and
and at least one organic layer between the anode and the cathode
wherein the organic layer includes the composition of claim 1.

19. The organic optoelectric diode of claim 18, wherein the organic layer includes a light-emitting layer, and
the light-emitting layer includes the composition.

20. A display device comprising the organic optoelectric diode of claim 18.
